(19) United States
(12) Patent Application Publication
(10) Pub. No.: US 2025/0339529 A1
(43) Pub. Date: Nov. 6, 2025

(54) B-CELL MATURATION ANTIGEN (BCMA) CHIMERIC ANTIGEN RECEPTOR INVARIANT NATURAL KILLER T CELLS AND USES THEREOF

(71) Applicant: MINK THERAPEUTICS, INC., New York, NY (US)

(72) Inventors: Eleni CHANTZOURA, Cambridge (GB); Efrat ALTMAN-SHARONI, Cambridge (GB); Olivier LE TONQUEZE, New York, NY (US); Marc VAN DIJK, Cambridge (GB); Martyna POPIS, Cambridge (GB); Paul IBBETT, Cambridge (GB)

(73) Assignee: MINK THERAPEUTICS, INC., New York, NY (US)

(21) Appl. No.: 19/219,165

(22) Filed: May 27, 2025

Related U.S. Application Data
(63) Continuation of application No. 19/126,988, filed on Jan. 1, 0001.
(60) Provisional application No. 63/422,916, filed on Nov. 4, 2022.

(51) Int. Cl. A61K 40/15; A61K 40/31; A61K 40/35; A61K 40/42; A61P 35/00; C07K 14/725; C07K 16/28; C12N 5/0783; C12N 15/86

(52) U.S. Cl. CPC A61K 40/15; A61K 40/31; A61K 40/35; A61K 40/42; A61P 35/00; C07K 14/7051; C07K 16/2878; C12N 5/0646; C12N 15/86; C07K 2317/24; C12N 2510/00; C12N 2740/15043

(57) ABSTRACT

The present disclosure, at least in part, is based on the discovery of novel BCMA antibodies or antigen binding fragments thereof, and genetically modified cells (e.g., iNKT cells, CAR T cells, etc.) expressing chimeric antigen receptors comprising the anti-BCMA antibody or antigen binding fragment thereof demonstrate improved properties, including increased binding to BCMA, killing of BCMA-expressing cancer cells in vitro and in vivo; and enhanced persistent in a subject receiving the therapy.

### RELATED APPLICATIONS

This application is a continuation of U.S. patent application Ser. No. 19/126,988, filed May 2, 2025, which is a U.S. National Phase Application under 35 U.S.C. § 371 of International Patent App. No. PCT/US2023/078730, filed Nov. 3, 2023, which claims the benefit under 35 U.S.C. 119 (e) of the filing date of U.S. Provisional Patent App. No. 63/422,916, filed Nov. 4, 2022, the entire contents of which are incorporated by reference herein.

### REFERENCE TO AN ELECTRONIC SEQUENCE LISTING

The contents of the electronic sequence listing (A132770003WO00-SEQ-LJG.xml; Size: 202,613 bytes; and Date of Creation: Nov. 3, 2023) is herein incorporated by reference in its entirety.

### BACKGROUND

B-cell maturation antigen (BCMA) is a tumor necrosis factor receptor (TNFR) member expressed on the surface of cells of the B-cell lineage. The expression of BCMA has been linked to a number of cancers, autoimmune disorders, and infectious diseases. Cancers with increased expression of BCMA include some hematological cancers, such as multiple myeloma (MM), Hodgkin's and non-Hodgkin's lymphoma, various leukemias, and glioblastoma. Chimeric antigen receptors (CAR) proteins and CAR cells (e.g., CAR T cells or CAR iNKT cells) directed against BCMA have previously been described.

### SUMMARY

The present disclosure, at least in part, is based on novel BCMA antibodies or antigen binding fragments thereof, and genetically modified cells (e.g., iNKT cells, CAR T cells, etc.) expressing chimeric antigen receptors comprising the anti-BCMA antibody or antigen binding fragment thereof. In some embodiments, compositions described herein demonstrate improved properties, including increased binding to BCMA, killing of BCMA-expressing cancer cells in vitro and in vivo; and enhanced persistence in subjects receiving the compositions, relative to previously described anti-BCMA binding molecules. Aspects of the disclosure relate to methods of treating certain cancers (e.g., BCMA-expressing cancers) by administering compositions described herein to a subject in need thereof.

In some aspects, the present disclosure provides an invariant natural killer T (iNKT) cell engineered to express a chimeric antigen receptor comprising a B Cell Maturation Antigen (BCMA) binding moiety and IL-15.

In some embodiments, the IL-15 is a soluble IL-15. In some embodiments, the IL-15 is a human IL-15.

In some embodiments, the iNKT cell kills BCMA-expressing cells. In some embodiments, the iNKT cell kills BCMA expressing cells directly. In some embodiments, the iNKT cell kills BCMA expressing cells indirectly.

In some embodiments, the iNKT cell retains a response to CD1d and/or NK receptor ligand.

In some embodiments, the iNKT cell comprises a CAR comprising any one of the anti-BCMA antibodies set forth in Table 3. In some embodiments, the iNKT cell comprises a CAR comprising any one of the CARs set forth in Table 4.

In some aspects, the present disclosure provides a method for killing a cell, the method comprising contacting the cell with the iNKT cell engineered to express a chimeric antigen receptor comprising a B Cell Maturation Antigen (BCMA) binding moiety and IL-15.

In some aspects, the present disclosure provides a method for treating a tumor in a subject, the method comprising administering to a subject having or suspected of having cancer, the iNKT cell engineered to express a chimeric antigen receptor comprising a B Cell Maturation Antigen (BCMA) binding moiety and IL-15.

In some aspects, the present disclosure provides a method for reducing tumor growth, the method comprising contacting the tumor in a subject with the iNKT cell engineered to express a chimeric antigen receptor comprising a B Cell Maturation Antigen (BCMA) binding moiety and IL-15.

In some embodiments, the tumor cell express BCMA. In some embodiments, the administration of the iNKT cells leads to reduced tumor burden relative to tumor burden in the subject prior to the administration. In some embodiments, the administration of the iNKT cells leads to resistance to T cell exhaustion, enhancement of tissue homing of anti-BCMA iNKT cells, selective cytotoxicity towards M2 macrophages, and/or stimulation of dendritic cell maturation.

In some embodiments, the subject does not undergo lymphodepletion prior to administration of the iNKT cell engineered to express a chimeric antigen receptor comprising a B Cell Maturation Antigen (BCMA) binding moiety and IL-15.

In some aspects, the present disclosure provides an antibody or antigen binding fragment that specifically binds to an amino acid sequence having at least 85% identity to SEQ ID NOs: 38-48.

In some embodiments, the antibody specifically binds an amino acid sequence set forth as: SEQ ID NOs: 38-48.

In some aspects, the present disclosure provides an antibody or antigen binding fragment that comprises a heavy chain variable region having the sequence set forth as in any one of SEQ ID NOs: 7, 12, 19 or 21.

In some embodiments, the antibody comprises a heavy chain variable region having the sequence set forth as: SEQ ID NOs: 7, 12, 19 or 21 and a light chain variable region having a sequence set forth as: SEQ ID NO: 8, 10, 13, 15, or 22.

In some embodiments, the antibody comprises: (i) heavy chain variable region having the sequence set forth in SEQ ID NO: 7 and the light chain variable region having a sequence set forth in SEQ ID NO: 8; (ii) heavy chain variable region having the sequence set forth in SEQ ID NO: 7 and the light chain variable region having a sequence set forth in SEQ ID NO: 10; (iii) heavy chain variable region having the sequence set forth in SEQ ID NO: 12 and a light chain variable region having the sequence set forth in SEQ ID NO: 13; (iv) heavy chain variable region having the sequence set forth in SEQ ID NO: 12 and a light chain variable region having the sequence set forth in SEQ ID NO: 15; (v) heavy chain variable region having the sequence set forth in SEQ ID NO: 7 and a light chain variable region having the sequence set forth in SEQ ID NO: 13; (vi) heavy chain variable region having the sequence set forth in SEQ ID NO: 7 and a light chain variable region having the sequence set forth in SEQ ID NO: 15; (vii) heavy chain variable region having the sequence set forth in SEQ ID NO: 19 and a light chain variable region having the sequence set forth in SEQ ID NO: 10; or (viii) heavy chain variable region having the sequence set forth in SEQ ID NO: 21 and the light chain variable region having a sequence set forth in SEQ ID NO: 22.

In some aspects, the present disclosure provides an antibody or antigen binding fragment that comprises a variable heavy chain region comprising a complementarity determining region 3 (CDRH3) having the sequence set forth in SEQ ID NOs: 3 or 33.

In some embodiments, the antibody or antigen binding fragment further comprising a variable light chain region comprising a complementarity determining region 3 (CDRL3) having the sequence set forth in any one of SEQ ID NOs: 6, 29, 30, or 36.

In some aspects, the present disclosure provides an antibody or antigen binding fragment that comprises six complementarity determining regions (CDRs): CDRH1, CDRH2, CDRH3, CDRL1, CDRL2, and CDRL3, wherein (i) CDRH1 comprises the sequence as set forth in SEQ ID NO: 1, CDRH2 comprises the sequence as set forth in SEQ ID NO: 2, CDRH3 comprises the sequence as set forth in SEQ ID NO: 3, CDRL1 comprises the sequence as set forth in SEQ ID NO: 4, CDRL2 comprises the sequence as set forth in SEQ ID NO: 5, and CDRL3 comprises the sequence as set forth in SEQ ID NO: 6; (ii) CDRH1 comprises the sequence as set forth in SEQ ID NO: 1, CDRH2 comprises the sequence as set forth in SEQ ID NO: 2, CDRH3 comprises the sequence as set forth in SEQ ID NO: 3, CDRL1 comprises the sequence as set forth in SEQ ID NO: 4, CDRL2 comprises the sequence as set forth in SEQ ID NO: 5, and CDRL3 comprises the sequence as set forth in SEQ ID NO: 6; (iii) CDRH1 comprises the sequence as set forth in SEQ ID NO: 25, CDRH2 comprises the sequence as set forth in SEQ ID NO: 27, CDRH3 comprises the sequence as set forth in SEQ ID NO: 3, CDRL1 comprises the sequence as set forth in SEQ ID NO: 4, CDRL2 comprises the sequence as set forth in SEQ ID NO: 24, and CDRL3 comprises the sequence as set forth in SEQ ID NO: 30; (iv) CDRH1 comprises the sequence as set forth in SEQ ID NO: 25, CDRH2 comprises the sequence as set forth in SEQ ID NO: 27, CDRH3 comprises the sequence as set forth in SEQ ID NO: 3, CDRL1 comprises the sequence as set forth in SEQ ID NO: 4, CDRL2 comprises the sequence as set forth in SEQ ID NO: 24, and CDRL3 comprises the sequence as set forth in SEQ ID NO: 29; (v) CDRH1 comprises the sequence as set forth in SEQ ID NO: 1, CDRH2 comprises the sequence as set forth in SEQ ID NO: 2, CDRH3 comprises the sequence as set forth in SEQ ID NO: 3, CDRL1 comprises the sequence as set forth in SEQ ID NO: 4, CDRL2 comprises the sequence as set forth in SEQ ID NO: 24, and CDRL3 comprises the sequence as set forth in SEQ ID NO: 30; (vi) CDRH1 comprises the sequence as set forth in SEQ ID NO: 1, CDRH2 comprises the sequence as set forth in SEQ ID NO: 2, CDRH3 comprises the sequence as set forth in SEQ ID NO: 3, CDRL1 comprises the sequence as set forth in SEQ ID NO: 4, CDRL2 comprises the sequence as set forth in SEQ ID NO: 24, and CDRL3 comprises the sequence as set forth in SEQ ID NO: 29; (vii) CDRH1 comprises the sequence as set forth in SEQ ID NO: 26, CDRH2 comprises the sequence as set forth in SEQ ID NO: 28, CDRH3 comprises the sequence as set forth in SEQ ID NO: 3, CDRL1 comprises the sequence as set forth in SEQ ID NO: 4, CDRL2 comprises the sequence as set forth in SEQ ID NO: 24, and CDRL3 comprises the sequence as set forth in SEQ ID NO: 6; or (viii) CDRH1 comprises the sequence as set forth in SEQ ID NO: 31, CDRH2 comprises the sequence as set forth in SEQ ID NO: 32, CDRH3 comprises the sequence as set forth in SEQ ID NO: 33, CDRL1 comprises the sequence as set forth in SEQ ID NO: 34, CDRL2 comprises the sequence as set forth in SEQ ID NO: 35, and CDRL3 comprises the sequence as set forth in SEQ ID NO: 36.

In some embodiments, the antibody or antigen binding fragment is chimeric.

In some embodiments, the antibody or antigen binding fragment is humanized.

In some embodiments, the antibody or antigen binding fragment is a single-chain variable fragment (scFv). In some embodiments, the scFv comprises the amino acid sequence set forth of any one of SEQ ID NOs: 9, 11, 14, 16, 17, 18, 20, or 23.

In some aspects, the present disclosure provides an isolated nucleic acid encoding the antibody or antigen binding fragment described herein.

In some embodiments, the isolated nucleic acid comprising the sequence set forth in any one of SEQ ID NOs: 49-64. In some embodiments, the isolated nucleic acid comprising the sequence set forth in any one of SEQ ID NOs: 65-72.

In some aspects, the present disclosure provides a vector comprising the isolated nucleic acid described herein. In some embodiments, the vector is a viral vector. In some embodiments, the viral vector is a lentiviral vector. In some embodiments, the lentiviral vector comprises the sequence set forth in any one of SEQ ID NOs: 73-80.

In some aspects, the present disclosure provides a host cell comprising the antibody or antigen binding fragment, the isolated nucleic acid, or the vector described herein. In some embodiments, the cell is a mammalian cell, bacterial cell, yeast cell, or insect cell. In some embodiments, the cell is a hybridoma cell.

In some aspects, the present disclosure provides a chimeric antigen receptor (CAR) comprising the anti-BCMA antigen binding fragment (e.g., anti-BCMA scFv) described herein.

In some embodiments, the CAR further comprises a hinge region. In some embodiments, the hinge region comprises an amino acid sequence at least 80% identical to SEQ ID NO: 82.

In some embodiments, the CAR further comprises a transmembrane domain. In some embodiments, the transmembrane domain comprises an amino acid sequence at least 80% identical to SEQ ID NO: 83.

In some embodiments, the CAR further comprises a cytoplasmic domain. In some embodiments, the cytoplasmic domain comprises an amino acid sequence at least 80% identical to SEQ ID NO: 84.

In some embodiments, the CAR further comprises a co-stimulatory domain. In some embodiments, the co-stimulatory domain comprises an amino acid sequence at least 80% identical to SEQ ID NO: 85.

In some embodiments, the CAR comprises the amino acid sequence of any one of SEQ ID NOs: 86-93.

In some aspects, the present disclosure provides an immune cell comprising the ant-BCMA CAR described herein. In some embodiments, the immune cell is a natural killer (NK) cell or a T cell. In some embodiments, the immune cell is an invariant natural killer T (iNKT) cell.

In some embodiments, the immune cell is engineered to express one or more immunoregulatory gene products. In some embodiments, the one or more immunoregulatory gene products comprises IL-15, IL-12, CD40L, or 4-1BB.

In some aspects, the present disclosure provides a pharmaceutical composition comprising the anti-BCMA antibody or antigen binding fragment, or the immune cell described herein, and a pharmaceutically acceptable excipient.

In some aspects, the present disclosure provides a method for killing a cell, the method comprising contacting the cell with the anti-BCMA antibody or antigen binding fragment, the immune cell, or the pharmaceutical composition described herein.

In some aspects, the present disclosure provides a method for treating cancer in a subject, the method comprising administering to a subject having or suspected of having cancer, the immune cell, or the pharmaceutical composition described herein.

In some aspects, the present disclosure provides a method for reducing tumor growth, the method comprising contacting the tumor in a subject with the anti-BCMA antibody or antigen binding fragment, the immune cell, or the pharmaceutical composition described herein.

In some embodiments, the cell is a mammalian cell. In some embodiments, the cell is a human cell. In some embodiments, the cell is a cancer cell. In some embodiments, the cancer is a blood cancer. In some embodiments, the blood cancer is a leukemia or lymphoma. In some embodiments, the cancer is multiple myeloma.

In some embodiments, the administration is via injection.

In some embodiments, the subject does not undergo lymphodepletion prior to administration of the iNKT cells.

### BRIEF DESCRIPTION OF THE DRAWINGS

The accompanying drawings, which are incorporated in and constitute a part of this specification, illustrate certain embodiments, and together with the written description, serve to provide non-limiting examples of certain aspects of the compositions and methods disclosed herein.

FIG. 1 shows a schematic depicting iNKT cells targeting tumor cells via direct or indirect mechanisms.

FIG. 2 shows a schematic of an exemplary anti-BCMA-CAR-iNKT cell. The anti-BCMA-CAR iNKT cell shown is also engineered to secrete IL-15.

FIG. 3 shows a schematic of an overview of a screening process of anti-BCMA CAR iNKT cells.

FIG. 4 shows a schematic of a traditional phage campaign to select BCMA binders.

FIG. 5 shows a schematic of a mammalian screening strategy for identification of highly functional BCMA CARs.

FIG. 6 shows representative data for binding affinity of exemplary anti-BCMA scFvs F6 and F1.

FIGS. 7A-7B show representative data for epitope mapping of BCMA CAR candidates. FIG. 7A shows the loss of binding to BCMA mutants (in which accessible amino acids were mutated to alanine for mapping the epitope of the anti-BCMA CAR candidates) in comparison to wild type BCMA by Biacore. FIG. 7B shows the 3D structure demonstrating the binding site of F6G-Var2 to BCMA based on FIG. 7A (Asp15, Leu17, Leu18 on the top, Arg27, Thr32 and Leu35 at the bottom).

FIGS. 8A-8I shows representative data of anti-BCMA-CAR iNKT cell-mediated cytotoxicity assays. FIG. 8A shows a schematic of cytotoxicity assays mediated by iNKT cells transiently transfected with BCMA-CARs. FIGS. 8B-8H show killing of target cells by iNKT cells expressing different BCMA-CAR candidates or a clinical benchmark bb2121. Legend on FIG. 8B applies to FIGS. 8B-8H. FIG. 8I shows the level of endogenous BCMA expression for each tumor line as assessed by flow cytometry (Bottom right). K562 cells wild type or stably transduced with BCMA were also included in the assay.

FIGS. 9A-9C show an example of an Accelerated Drug Discovery (ADD) approach to affinity mature F6G. FIG. 9A shows a schematic representation of the CDR positions undergone mutagenesis. FIG. 9B shows the affinity KD (M) values of mutant scFvs binding to BCMA antigen. Sequences shown are GYYMH (SEQ ID NO: 1), NPNSGN (SEQ ID NO: 105), QWEPL (SEQ ID NO: 106), DALPKKYTY (SEQ ID NO: 107), EDTKRPS (SEQ ID NO: 5), and LSTDPTGQ (SEQ ID NO: 108). FIG. 9C shows individual Biacore sensorgrams for E to Y substitution in position 49 of CDRL2.

FIGS. 10A-10D show representative data for affinity maturation of F6G by phage libraries. FIG. 10A shows a schematic representation of the phage display affinity maturation libraries strategy. The CDR amino acids marked as “x” were mutated to any other amino acid and to form the library. FIG. 10B shows that the libraries were used for phage panning followed by mammalian display. FIG. 10C shows representative data for target cell killing by iNKT cells expressing different BCMA-CAR candidates or a clinical benchmark (bb2121). FIG. 10D shows representative data indicating cytotoxicity towards BCMA negative K562 cells was negligible and similar to bb2121, and activation was negligible and similar to bb2121 for most tested candidates.

FIG. 11 shows representative data for the binding kinetics of F6 variants. Affinity matured clones had 10-fold improved affinity in comparison to the original F6 clone and almost 50-fold improved affinity in comparison to the germline F6G.

FIG. 12 shows representative data for kinetics of other known anti BCMA CARs compared to F6G-Var2.

FIGS. 13A-13G show representative data for anti-BCMA-CAR iNKT cell-mediated killing of tumor cells. FIGS. 13A-13C show flow cytometry analysis of RFP expression to show CAR expression and antigen binding with BCMA-APC in BCMA-CAR iNKTs from two iNKT donors. FIG. 13D shows the level of BCMA expression on each tumor cell line as assessed by flow cytometry. The left peak shows isotype staining and right peak shows BCMA expression.

FIG. 13E shows the percent of dead target cells after iNKT cells from FIG. 13A were co-cultured with tumor cell lines endogenously expressing BCMA or K562 for 24 hrs at 3 different effector-to-target ratios. FIG. 13F shows the percentage of CD69/CD25 co-expressing iNKT cells after co-culturing with BCMA expressing target cells or K562 cells. FIG. 13G shows the percentage of 4-1BB expressing iNKT cells after co-culturing with BCMA expressing target cells or K562 cells.

FIG. 14 shows a sequence alignment of F6G variants' heavy and light variable regions. Heavy chain sequences shown: consensus (SEQ ID NO: 7); F6G-Var5 (SEQ ID NO: 7); F6G-Var3 (SEQ ID NO: 12); F6G-Var2 (SEQ ID NO: 12); F6G-Var6 (SEQ ID NO: 19); F6G-Var4 (SEQ ID NO: 7); and F6G-Var1 (SEQ ID NO: 7). Light chain sequences shown: consensus (SEQ ID NO: 109); F6G-Var6 (SEQ ID NO: 109); F6G-Var1 (SEQ ID NO: 109); F6G-Var5 (SEQ ID NO: 110); F6G-Var3 (SEQ ID NO: 110); F6G-Var2 (SEQ ID NO: 111); and F6G-Var4 (SEQ ID NO: 111)

FIG. 15 shows representative data for predicted immunogenicity of F6G variants.

FIG. 16 shows an overview of stable CAR-iNKT generation process (left) and flow cytometry assessment of purity and number of BCMA CAR-iNKT cells (right).

FIG. 17 shows flow cytometry analysis of anti-BCMA CAR IL-15 iNKT cell generated by lentiviral transduction and antigen mediated CAR enrichment.

FIG. 18A-18L show representative data for in vitro cytotoxicity assays of BCMA-CAR iNKT with or without IL-15 armoring challenged with multiple myeloma tumor lines expressing BCMA. bb2121 IL-15 iNKT cells and anti-CD19 CAR IL-15 iNKT cells, and non-transduced iNKT cells were used as controls. FIG. 18A-18F show cytotoxicity assay results of iNKT cells from donor 1. FIGS. 18G-18L show representative cytotoxicity assay data of iNKT cells from donor 2. Sample legends are shown between FIG. 18A and FIG. 18B.

FIG. 19A-19D show representative data for antigen specific activation of BCMA-CAR-IL-15 iNKT cells. FIGS. 19A-19B show antigen specific activation of BCMA-CAR-IL-15 iNKT cells from donor 1. FIGS. 19C-19D show antigen specific activation of BCMA-CAR-IL-15 iNKT cells from donor 2.

FIGS. 20A-20B show representative data for increased IFN-γ production by BCMA-CAR-IL-15 iNKT cells when challenged with BCMA-expressing tumor lines. FIG. 20A shows IFN-γ production by BCMA-CAR-IL-15 iNKT cells from donor 1 when challenged with BCMA expressing tumor lines. FIG. 20B shows IFN-γ production by BCMA-CAR-IL-15 iNKT cells from donor 1 when challenged with BCMA expressing tumor lines.

FIGS. 21A-21E show representative data indicating IL-15 production increases in vivo persistence of anti-BCMA CAR iNKT cells in blood (FIG. 21A), bone marrow (FIG. 21B), liver (FIG. 21C), spleen (FIG. 21D), and lungs (FIG. 21E).

FIG. 22A-22H show representative data indicating adoptive cell transfer of BCMA-CAR-IL-15 iNKT cells delayed engraftment of multiple myeloma MM1.s tumors in xenograft mice. FIGS. 22A-22B show that BCMA-CAR-IL-15 iNKT cells showed delayed tumor engraftment for 3 weeks after adoptive transfer. FIG. 22C shows Kaplan-Meier Survival graph showed significant survival benefit in mice treated with BCMA-CAR-IL-15 iNKT cells compared to CD19-IL-15 CAR iNKT cells and untreated mice cohorts. FIG. 22D shows that BCMA-CAR-IL-15 iNKT cells treated mice showed no multiple myeloma associated or IL-15 associated (F6G-Var2 IL-15 iNKT cells vs F6G-Var2 iNKT cells cohort) toxicity or body weight lost. FIGS. 22E-22F show that flow-cytometric analysis and quantification of bone marrow tissues from all mice showed that at both harvest time-points, samples showed little/no traces of GFP-positive MM1.s indicating significant tumor control. FIGS. 22G-22H show that flow-cytometric analysis of bone marrow indicating persistence of BCMA-CAR-IL-15 at both harvest time-points. IL-15 as expressed in F6G-Var2 IL-15 compared to its non-IL-15 counterpart F6G-Var2 indicates benefit of IL-15 expression in iNKT persistence at early time-point.

### DETAILED DESCRIPTION

The present disclosure, at least in part, is based on novel BCMA antibodies or antigen binding fragments thereof. In some aspects, the present disclosure also relates to chimeric antigen receptors (CAR) comprising the anti-BCMA antibodies or antigen binding fragments described herein, and immune cells (e.g., iNKT cells, T cells, NK cells etc.) expressing such CARs. In some embodiments, the immune cells (e.g., iNKT cells, T cells, NK cells, etc.) are engineered to secrete IL-15, which was observed to promote persistence of the immune cells described herein in subjects. Also provided are the use of the anti-BCMA antibodies or antigen binding fragments, and/or the immune cells expressing the anti-BCMA antigen binding fragments for killing cancer cells (e.g., cancer cells expressing BCMA), and/or treating cancer (e.g., BCMA positive cancer). As described further in the Examples, anti-BCMA antibodies, and/or the cells expressing a CAR comprising an anti-BCMA antibody described herein demonstrate improved properties, including increased binding to BCMA, killing of BCMA-expressing cancer cells in vitro and in vivo; and enhanced persistence in subjects receiving the compositions (e.g., relative to subjects receiving other CAR T cell therapies or anti-BCMA antibody therapies).

The foregoing and other aspects, implementations, acts, functionalities, features and embodiments of the present teachings can be more fully understood from the following description in conjunction with the accompanying drawings.

### I. Definitions

Section headings have been provided for convenience but shall not be interpreted to limit disclosure. For instance, terms are defined throughout the specification and not solely in the section titled “Definitions.”

Administering: As used herein, the terms “administering” or “administration” means to provide a therapeutic agent or composition thereof to a subject in a manner that is physiologically and/or pharmacologically useful (e.g., to treat a condition in the subject).

Affinity Matured Antibody: “Affinity Matured Antibody” is used herein to refer to an antibody with one or more alterations in one or more CDRs, which result in an improvement in the affinity (i.e., KD, kd or ka) of the antibody for a target antigen compared to a parent antibody, which does not possess the alteration(s). In some embodiments, affinity matured antibodies will have nanomolar or even picomolar affinities for the target antigen. A variety of procedures for producing affinity matured antibodies are known in the art, including the screening of a combinatory antibody library that has been prepared using bio-display. For example, Marks et al., BioTechnology, 10:779-783 (1992) describes affinity maturation by VH and VL domain shuffling. Random mutagenesis of CDR and/or framework residues is described by Barbas et al., Proc. Nat. Acad. Sci. USA, 91:3809-3813 (1994); Schier et al., Gene, 169:147-155 (1995); Yelton et al., J. Immunol., 155:1994-2004 (1995); Jackson et al., J. Immunol., 154 (7): 3310-3319 (1995); and Hawkins et al, J. Mol. Biol., 226:889-896 (1992). Selective mutation at selective mutagenesis positions and at contact or hypermutation positions with an activity-enhancing amino acid residue is described in U.S. Pat. No. 6,914,128 B1.

Antibody: As used herein, the term “antibody” or “antibodies” refers to a polypeptide that includes at least one immunoglobulin variable domain or at least one antigenic determinant, e.g., paratope that specifically binds to an antigen. Examples of antibodies include monoclonal antibodies, recombinantly produced antibodies, monospecific antibodies, multi-specific antibodies (including bispecific antibodies), human antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, heteroconjugate antibodies, antibody-drug conjugates, single domain antibodies, monovalent antibodies, single chain antibodies or single-chain Fvs (scFv), camelized antibodies, affybodies, Fab fragments, F(ab′)2 fragments, disulfide-linked Fvs (sdFv), anti-idiotypic (anti-Id) antibodies (including, e.g., anti-anti-Id antibodies), and antigen-binding fragments of any of the above. In some embodiments, an antibody is a full-length antibody which further comprises the constant region of an immunoglobulin heavy chain and light chain. In some embodiments, an antibody is a chimeric antibody. In some embodiments, an antibody is a humanized antibody. However, in some embodiments, an antibody is a Fab fragment, a F(ab′)2 fragment, a Fv fragment or a scFv fragment. In some embodiments, an antibody is a nanobody derived from a camelid antibody or a nanobody derived from shark antibody. In some embodiments, an antibody is a diabody. In some embodiments, an antibody comprises a framework having germline sequence from a species (e.g., a human germline sequence). In some embodiments, an antibody comprises a heavy (H) chain variable region (abbreviated herein as VH), and/or a light (L) chain variable region (abbreviated herein as VL). In some embodiments, the VH comprises an amino acid sequence that is at least 70%, 75%, 80%, 85%, 90%, 95%, 98%, or at least 99% identical to any of the heavy chain variable domain provided herein. In some embodiments, the VL comprises an amino acid sequence that is at least 70%, 75%, 80%, 85%, 90%, 95%, 98%, or at least 99% identical to any of the light chain variable domain provided herein. In some embodiments, an antibody comprises a constant domain, e.g., an Fc region. An immunoglobulin constant domain refers to a heavy or light chain constant domain. Human IgG heavy chain and light chain constant domain amino acid sequences and their functional variations are known. In another embodiment, an antibody comprises a heavy chain constant domain selected from the group consisting of IgG, IgG1, IgG2, IgG2A, IgG2B, IgG2C, IgG3, IgG4, IgA1, IgA2, IgD, IgM, and IgE constant domains. With respect to the heavy chain, in some embodiments, the heavy chain of an antibody described herein can be an alpha (α), delta (Δ), epsilon (ε), gamma (γ) or mu (μ) heavy chain. In some embodiments, the heavy chain of an antibody described herein can comprise a human alpha (α), delta (Δ), epsilon (ε), gamma (γ) or mu (μ) heavy chain. In a particular embodiment, an antibody described herein comprises a human gamma 1 CH1, CH2, and/or CH3 domain. In some embodiments, the amino acid sequence of the VH domain comprises the amino acid sequence of a human gamma (γ) heavy chain constant region, such as any known in the art. Non-limiting examples of human constant region sequences have been described in the art, e.g., see U.S. Pat. No. 5,693,780 and Kabat E A et al., (1991) supra. In some embodiments, an antibody is modified, e.g., modified via glycosylation, phosphorylation, sumoylation, and/or methylation. In some embodiments, an antibody is a glycosylated antibody, which is conjugated to one or more sugar or carbohydrate molecules. In some embodiments, the one or more sugar or carbohydrate molecule are conjugated to the antibody via N-glycosylation, O-glycosylation, C-glycosylation, glypiation (GPI anchor attachment), and/or phosphoglycosylation. In some embodiments, the one or more sugar or carbohydrate molecule are monosaccharides, disaccharides, oligosaccharides, or glycans. In some embodiments, the one or more sugar or carbohydrate molecule is a branched oligosaccharide or a branched glycan. In some embodiments, the one or more sugar or carbohydrate molecule includes a mannose unit, a glucose unit, an N-acetylglucosamine unit, or a phospholipid unit.

Approximately: As used herein, the term “approximately” or “about,” as applied to one or more values of interest, refers to a value that is similar to a stated reference value. In certain embodiments, the term “approximately” or “about” refers to a range of values that fall within 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

B-cell maturation antigen (BCMA): Human BCMA is a 184 amino acid protein encoded by the TNFRSF17 gene, which is a member of the TNF-receptor superfamily. BCMA is expressed in mature B lymphocytes, and may be important for B cell development and autoimmune response. This receptor has been shown to specifically bind to the tumor necrosis factor (ligand) superfamily, member 13b (TNFSF13B/TALL-1/BAFF), and to lead to NF-kappaB and MAPK8/JNK activation. This receptor also binds to various TRAF family members, and thus may transduce signals for cell survival and proliferation.

Binding affinity: As used herein, the term “binding affinity” generally refers to the strength of the sum total of non-covalent interactions between a single binding site of a molecule (e.g., an antibody) and its binding partner (e.g., an epitope). Unless indicated otherwise, as used herein, “binding affinity” refers to intrinsic binding affinity which reflects a 1:1 interaction between members of a binding pair. The affinity of a molecule X for its partner Y can generally be represented by the dissociation constant (K_{D}). Affinity can be measured and/or expressed in a number of ways known in the art, including, but not limited to, equilibrium dissociation constant (K_{D}) and equilibrium association constant (K_{A}). The K_{D} is calculated from the quotient of k_{off}/kₒₙ, whereas K_{A} is calculated from the quotient of kₒₙ/k_{off}. kₒₙ refers to the association rate constant of, e.g., an antibody to an epitope, and k_{off} refers to the dissociation rate constant of, e.g., an antibody to an epitope.

Binding affinity (or binding specificity) can be determined by a variety of methods including equilibrium dialysis, equilibrium binding, gel filtration, ELISA, surface plasmon resonance (SPR), florescent activated cell sorting (FACS) or spectroscopy (e.g., using a fluorescence assay). Exemplary conditions for evaluating binding affinity are in HBS-P buffer (10 mM HEPES pH7.4, 150 mM NaCl, 0.005% (v/v) surfactant P20) and PBS buffer (10 mM PO4-3, 137 mM NaCl, and 2.7 mM KCl). These techniques can be used to measure the concentration of bound proteins as a function of target protein concentration. The concentration of bound protein ([Bound]) is generally related to the concentration of free target protein ([Free]) by the following equation:

[Bound]=[Free]/(Kd+[Free])

It is not always necessary to make an exact determination of K_{A}, though, since sometimes it is sufficient to obtain a quantitative measurement of affinity, e.g., determined using a method such as ELISA or FACS analysis, is proportional to K_{A}, and thus can be used for comparisons, such as determining whether a higher affinity is, e.g., 2-fold higher, to obtain a qualitative measurement of affinity, or to obtain an inference of affinity, e.g., by activity in a functional assay, e.g., an in vitro or in vivo assay.

CDR: As used herein, the term “CDR” refers to the complementarity determining region within antibody variable sequences. A typical antibody molecule comprises a heavy chain variable region (VH) and a light chain variable region (VL), which are usually involved in antigen binding. The VH and VL regions can be further subdivided into regions of hypervariability, also known as “complementarity determining regions” (“CDR”), interspersed with regions that are more conserved, which are known as “framework regions” (“FR”). Each VH and VL is typically composed of three CDRs and four FRs, arranged from amino-terminus to carboxy-terminus in the following order: FR1, CDR1, FR2, CDR2, FR3, CDR3, FR4. The extent of the framework region and CDRs can be precisely identified using methodology known in the art, for example, by the Kabat definition, the IMGT definition, the Chothia definition, the AbM definition, and/or the contact definition, all of which are well known in the art. See, e.g., Kabat, E. A., et al. (1991) Sequences of Proteins of Immunological Interest, Fifth Edition, U.S. Department of Health and Human Services, NIH Publication No. 91-3242; IMGT®, the international ImMunoGeneTics information system® http://www.imgt.org, Lefranc, M.-P. et al., Nucleic Acids Res., 27:209-212 (1999); Ruiz, M. et al., Nucleic Acids Res., 28:219-221 (2000); Lefranc, M.-P., Nucleic Acids Res., 29:207-209 (2001); Lefranc, M.-P., Nucleic Acids Res., 31:307-310 (2003); Lefranc, M.-P. et al., In Silico Biol., 5, 0006 (2004) [Epub], 5:45-60 (2005); Lefranc, M.-P. et al., Nucleic Acids Res., 33:D593-597 (2005); Lefranc, M.-P. et al., Nucleic Acids Res., 37:D1006-1012 (2009); Lefranc, M.-P. et al., Nucleic Acids Res., 43:D413-422 (2015); Chothia et al., (1989) Nature 342:877; Chothia, C. et al. (1987) J. Mol. Biol. 196:901-917, Al-lazikani et al (1997) J. Molec. Biol. 273:927-948; and Almagro, J. Mol. Recognit. 17:132-143 (2004). ee also hgmp.mrc.ac.uk and bioinf.org.uk/abs. As used herein, a CDR may refer to the CDR defined by any method known in the art. Two antibodies having the same CDR means that the two antibodies have the same amino acid sequence of that CDR as determined by the same method, for example, the Kabat definition.

Generally, there are three CDRs in each of the variable regions of the heavy chain and the light chain, which are designated CDR1, CDR2 and CDR3, for each of the variable regions. The term “CDR set” as used herein refers to a group of three CDRs that occur in a single variable region capable of binding the antigen. The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Sub-portions of CDRs may be designated as L1, L2 and L3 or H1, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan (FASEB J. 9:133-139 (1995)) and MacCallum (J Mol Biol 262 (5): 732-45 (1996)). Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems.

The CDRs of an antibody may have different amino acid sequences when different definition systems are used (e.g., the IMGT definition, the Kabat definition, or the Chothia definition). A definition system annotates each amino acid in a given antibody sequence (e.g., VH or VL sequence of any of the anti-BCMA antibody described in Table 3) with a number, and numbers corresponding to the heavy chain and light chain CDRs are provided in Table 1. The CDRs of the anti-BCMA antibodies provided herein (e.g., CDRs of any of the anti-BCMA antibodies listed in Table 3) are defined in accordance with the Kabat definition. One skilled in the art is able to derive the CDR sequences using the different numbering systems for the anti-BCMA antibodies provided in Table 3.

TABLE 1
CDR Definitions

| | IMGT¹ | Kabat² | Chothia³ |
|---|---|---|---|
| CDRH1 | 27-38 | 31-35 | 26-32 |
| CDRH2 | 56-65 | 50-65 | 53-55 |
| CDRH3 | 105-116/117 | 95-102 | 96-101 |
| CDRL1 | 27-38 | 24-34 | 26-32 |
| CDRL2 | 56-65 | 50-56 | 50-52 |
| CDRL3 | 105-116/117 | 89-97 | 91-96 |

¹IMGT ®, the international ImMunoGeneTics information system ®, imgt.org, Lefranc, M.-P. et al., Nucleic Acids Res., 27: 209-212 (1999)
²Kabat et al. (1991) Sequences of Proteins of Immunological Interest, Fifth Edition, U.S. Department of Health and Human Services, NIH Publication No. 91-3242
³Chothia et al., J. Mol. Biol. 196: 901-917 (1987))

Chimeric antigen receptor: As used herein, a “chimeric antigen receptor” or “CAR” refers to an engineered receptor that binds specificity for an antigen (e.g., BCMA) or other ligand or molecule onto an immune effector cell (e.g., a T cell, NK cell, a NKT cell, an iNKT cell). A chimeric antigen receptor typically comprises at least an extracellular ligand-binding domain or moiety capable of specifically binding an antigen and an intracellular domain that comprises one or more signaling domains and/or co-stimulatory domains.

In some embodiments, extracellular ligand-binding domain of a CAR is in the form of a binding protein, small molecule, a peptide, a targeting agent, an agonist, or an antagonist. In some embodiments, the binding protein is an antibody, an antigen-binding fragment of an antibody (e.g., a scFv), a ligand, a cytokine, or a receptor. In some embodiments, the antigen binding fragment is a scFv (e.g., a scFv targeting BCMA).

In some embodiments, the extracellular ligand-binding domain is in the form of a single-chain variable fragment (scFv) derived from an antibody (e.g., a monoclonal antibody), which provides specificity for a particular epitope or antigen (e.g., an epitope or antigen preferentially present on the surface of a cell, such as a cancer cell or other disease-causing cell or particle).

In some embodiments, the CAR comprises an intracellular signaling domain. Intracellular signaling domains are cytoplasmic domains that transmit an activation signal to the cell following binding of the extracellular domain. An intracellular signaling domain can be any intracellular signaling domain of interest that is known in the art. Such cytoplasmic signaling domains can include, without limitation, CD3ζ. In some embodiments, the intracellular domain also includes one or more intracellular co-stimulatory domains, such as those described herein, which transmit a co-stimulatory signal which promotes cell proliferation, cell survival, and/or cytokine secretion after binding of the extracellular domain. Such intracellular co-stimulatory domains can include, without limitation, any co-stimulatory domain disclosed herein or those domains known in the art, such as, CD27, CD28, CD8, 4-1BB (CD137), OX40, CD30, CD40, CD127, PD-1, ICOS, lymphocyte function-associated antigen-1 (LFA-1), CD2, CD7, LIGHT, NKG2C, B7-H3, and a ligand that specifically binds with CD83, N1, N6, or any combination thereof. Additional suitable co-stimulatory domains are described in PCT International Application No. PCT/US2017/055133, which is incorporated by reference herein in its entirety. In some embodiments, the co-stimulatory domain is 4-1BB (CD137). In some embodiments, a chimeric antigen receptor further includes additional structural elements, including a transmembrane domain which is attached to the extracellular ligand-binding domain via a hinge or junction sequence. The transmembrane domain can be derived from any membrane-bound or transmembrane protein. For example, the transmembrane polypeptide can be a subunit of the T-cell receptor (i.e., an α, β, γ or ζ, polypeptide constituting CD3 complex), IL2 receptor p55 (α chain), p75 (β chain) or γ chain, subunit chain of Fc receptors (e.g., Fcy receptor III) or CD proteins such as the CD8 alpha chain. Alternatively, the transmembrane domain can be synthetic and can comprise predominantly hydrophobic residues such as leucine and valine. In some embodiments, the CAR comprises a CD8 transmembrane domain. The hinge region refers to any oligo- or polypeptide that functions to link the transmembrane domain to the extracellular ligand-binding domain. For example, a hinge region may comprise up to 300 amino acids, 10 to 100 amino acids and 25 to 50 amino acids. Hinge regions may be derived from all or part of naturally occurring molecules, such as from all or part of the extracellular region of CD8, CD4 or CD28, or from all or part of an antibody constant region. Alternatively, the hinge region may be a synthetic sequence that corresponds to a naturally occurring hinge sequence or may be an entirely synthetic hinge sequence. In some embodiments, a hinge domain can comprise a part of a human CD8 alpha chain, FcγRllla receptor or IgG1. In some embodiments, the CAR comprises a CD8 hinge region.

CDR-grafted antibody: The term “CDR-grafted antibody” refers to antibodies which comprise heavy and light chain variable region sequences from one species but in which the sequences of one or more of the CDR regions of VH and/or VL are replaced with CDR sequences of another species, such as antibodies having murine heavy and light chain variable regions in which one or more of the murine CDRs (e.g., CDR3) has been replaced with human CDR sequences.

Chimeric antibody: The term “chimeric antibody” refers to antibodies which comprise heavy and light chain variable region sequences from one species and constant region sequences from another species, such as antibodies having murine heavy and light chain variable regions linked to human constant regions.

Complementary: As used herein, the term “complementary” refers to the capacity for precise pairing between two nucleotides or two sets of nucleotides. In particular, complementary is a term that characterizes an extent of hydrogen bond pairing that brings about binding between two nucleotides or two sets of nucleotides. For example, if a base at one position of an oligonucleotide is capable of hydrogen bonding with a base at the corresponding position of a target nucleic acid (e.g., an mRNA), then the bases are considered to be complementary to each other at that position. Base pairings may include both canonical Watson-Crick base pairing and non-Watson-Crick base pairing (e.g., Wobble base pairing and Hoogsteen base pairing). For example, in some embodiments, for complementary base pairings, adenosine-type bases (A) are complementary to thymidine-type bases (T) or uracil-type bases (U), that cytosine-type bases (C) are complementary to guanosine-type bases (G), and that universal bases such as 3-nitropyrrole or 5-nitroindole can hybridize to and are considered complementary to any A, C, U, or T. Inosine (I) has also been considered in the art to be a universal base and is considered complementary to any A, C, U or T.

Conservative amino acid substitution: As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g. Molecular Cloning: A Laboratory Manual, J. Sambrook, et al., eds., Fourth Edition, Cold Spring Harbor Laboratory Press, Cold Spring Harbor, New York, 2012, or Current Protocols in Molecular Biology, F. M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

Co-stimulatory domain: As used herein, a “co-stimulatory domain” refers to a polypeptide domain which transmits an intracellular proliferative and/or cell-survival signal upon activation. Activation of a co-stimulatory domain may occur following homodimerization of two co-stimulatory domain polypeptides. Activation may also occur, for example, following activation of a construct comprising the co-stimulatory domain (e.g., a chimeric antigen receptor or an inducible regulatory construct). Generally, a co-stimulatory domain can be derived from a transmembrane co-stimulatory receptor, particularly from an intracellular portion of a co-stimulatory receptor. Non-limiting examples of co-stimulatory polypeptides include, but are not limited to, 4-1BB, CD28, ICOS, OX-40, and CD27 and any other co-stimulatory domain described further herein. In some embodiments, the CAR described herein comprises a 4-1BB co-stimulatory domain.

Co-stimulatory signal: As used herein, a “co-stimulatory signal” refers to an intracellular signal induced by a co-stimulatory domain that promotes cell proliferation, expansion of a cell population in vitro and/or in vivo, promotes cell survival, modulates (e.g., upregulates or downregulates) the secretion of cytokines, and/or modulates the production and/or secretion of other immunomodulatory molecules. In some embodiments, a co-stimulatory signal is induced following homodimerization of two co-stimulatory domain polypeptides. In some embodiments, a co-stimulatory signal is induced following activation of a construct comprising the co-stimulatory domain (e.g. a chimeric antigen receptor or an inducible regulatory construct).

Cross-reactive: As used herein and in the context of a targeting agent (e.g., antibody), the term “cross-reactive,” refers to a property of the agent being capable of specifically binding to more than one antigen of a similar type or class (e.g., antigens of multiple homologs, paralogs, or orthologs) with similar affinity or avidity. For example, in some embodiments, an antibody that is cross-reactive against human and non-human primate antigens of a similar type or class (e.g., a human BCMA and non-human primate BCMA) is capable of binding to the human antigen and non-human primate antigens with a similar affinity or avidity. In some embodiments, an antibody is cross-reactive against a human antigen and a rodent antigen of a similar type or class. In some embodiments, an antibody is cross-reactive against a rodent antigen and a non-human primate antigen of a similar type or class. In some embodiments, an antibody is cross-reactive against a human antigen, a non-human primate antigen, and a rodent antigen of a similar type or class.

Effective Amount: As used herein, “an effective amount” refers to the amount of each active agent (e.g., anti-BCMA antibody) required to confer therapeutic effect on the subject, either alone or in combination with one or more other active agents. In some embodiments, the therapeutic effect includes but is not limited to reducing tumor size, eradicating tumor, or alleviating symptoms associated with tumor.

Epitope: As used herein, an “epitope” is a term in the art and refers to a localized region of an antigen (e.g., a peptide or a peptide-MHC complex) to which an antibody or a chimeric antigen receptor can bind. In certain embodiments, the epitope to which an antibody or a chimeric antigen receptor binds can be determined by, e.g., NMR spectroscopy, X-ray diffraction crystallography studies, ELISA assays, hydrogen/deuterium exchange coupled with mass spectrometry (e.g., liquid chromatography electrospray mass spectrometry), flow cytometry analysis, mutagenesis mapping (e.g., site-directed mutagenesis mapping), and/or structural modeling. For X-ray crystallography, crystallization may be accomplished using any of the known methods in the art (e.g., Giegé R et al., (1994) Acta Crystallogr D Biol Crystallogr 50 (Pt 4): 339-350; McPherson A (1990) Eur J Biochem 189:1-23; Chayen NE (1997) Structure 5:1269-1274; McPherson A (1976) J Biol Chem 251:6300-6303, each of which is herein incorporated by reference in its entirety). Antibody: antigen crystals may be studied using well-known X-ray diffraction techniques and may be refined using computer software such as X-PLOR (Yale University, 1992, distributed by Molecular Simulations, Inc.; see, e.g., Meth Enzymol (1985) volumes 114 & 115, eds Wyckoff H W et al., U.S. 2004/0014194), and BUSTER (Bricogne G (1993) Acta Crystallogr D Biol Crystallogr 49 (Pt 1): 37-60; Bricogne G (1997) Meth Enzymol 276A: 361-423, ed Carter C W; Roversi P et al., (2000) Acta Crystallogr D Biol Crystallogr 56 (Pt 10): 1316-1323), each of which is herein incorporated by reference in its entirety. Mutagenesis mapping studies may be accomplished using any method known to one of skill in the art. See, e.g., Champe M et al., (1995) J Biol Chem 270:1388-1394 and Cunningham B C & Wells J A (1989) Science 244:1081-1085, each of which is herein incorporated by reference in its entirety, for a description of mutagenesis techniques, including alanine scanning mutagenesis techniques. In a specific embodiment, the epitope of an antigen is determined using alanine scanning mutagenesis studies. In a specific embodiment, the epitope of an antigen is determined using hydrogen/deuterium exchange coupled with mass spectrometry.

Framework: As used herein, the term “framework” or “framework sequence” refers to the remaining sequences of a variable region minus the CDRs. Because the exact definition of a CDR sequence can be determined by different systems, the meaning of a framework sequence is subject to correspondingly different interpretations. The six CDRs (CDR-L1, CDR-L2, and CDR-L3 of light chain and CDR-H1, CDR-H2, and CDR-H3 of heavy chain) also divide the framework regions on the light chain and the heavy chain into four sub-regions (FR1, FR2, FR3 and FR4) on each chain, in which CDR1 is positioned between FR1 and FR2, CDR2 between FR2 and FR3, and CDR3 between FR3 and FR4. Without specifying the particular sub-regions as FR1, FR2, FR3 or FR4, a framework region, as referred by others, represents the combined FRs within the variable region of a single, naturally occurring immunoglobulin chain. As used herein, a FR represents one of the four sub-regions, and FRs represents two or more of the four sub-regions constituting a framework region. Human heavy chain and light chain acceptor sequences are known in the art. In one embodiment, the acceptor sequences known in the art may be used in the antibodies disclosed herein.

Human antibody: The term “human antibody”, as used herein, is intended to include antibodies having variable and constant regions derived from human germline immunoglobulin sequences. The human antibodies of the disclosure may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences.

Humanized antibody: The term “humanized antibody” refers to antibodies which comprise heavy and light chain variable region sequences from a non-human species (e.g., a mouse) but in which at least a portion of the VH and/or VL sequence has been altered to be more “human-like”, i.e., more similar to human germline variable sequences. One type of humanized antibody is a CDR-grafted antibody, in which human CDR sequences are introduced into non-human VH and VL sequences to replace the corresponding nonhuman CDR sequences. In one embodiment, humanized anti-BCMA antibodies and antigen binding fragments thereof are provided. Such antibodies may be generated by obtaining murine anti-BCMA monoclonal antibodies using traditional hybridoma technology followed by humanization using in vitro genetic engineering, such as those disclosed in Kasaian et al PCT publication No. WO 2005/123126 A2. In some embodiments, a humanized antibody comprises human immunoglobulins (recipient antibody) in which residues from a complementary determining region (CDR) of the recipient are replaced by residues from a CDR of a non-human species (donor antibody) such as mouse, rat, or rabbit having the desired specificity, affinity, and capacity. In some embodiments, Fv framework region (FR) residues of the human immunoglobulin are replaced by corresponding non-human residues. Furthermore, the humanized antibody may comprise residues that are found neither in the recipient antibody nor in the imported CDR or framework sequences, but are included to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin. Antibodies may have Fc regions modified as described in WO 99/58572. Other forms of humanized antibodies have one or more CDRs (one, two, three, four, five, six) which are altered with respect to the original antibody, which are also termed one or more CDRs derived from one or more CDRs from the original antibody. Humanized antibodies may also involve affinity maturation.

Invariant natural killer cells (iNKT): The term “invariant Natural Killer T cells”, or “invariant NKT cells”, “iNKT cells”, or “Type I NKT cell), as used herein, refer to a population of T lymphocytes expressing a conserved semi-invariant TCR specific for lipid antigens (Ags) restricted for the monomorphic MHC class I-related molecule CD1d. Natural killer T cells (NKT cells) were originally characterized in mice as T cells that express both a TCR and NK1.1 (NKR-Pla-c or CD161), a C-type lectin NK receptor. Invariant NKT (iNKT) cells express a semi-invariant aß TCR (e.g., formed by an invariant TRAV11-TRAJ18 (4) rearrangement in mice, or the homologous invariant TRAV10-TRAJ18 chain in humans), paired with a limited set of diverse Vβ chains, predominantly TRBV1, TRBV29, or TRBV13 in mice (6) and TRBV25 in humans (see., e.g., Dellabona et al., An invariant V alpha 24-J alpha Q/V beta 11 T cell receptor is expressed in all individuals by clonally expanded CD4-8-T cells. J Exp Med. (1994) 180:1171-6. 10.1084). In some embodiments, the semi-invariant TCR recognizes exogenous and endogenous lipid Ags presented by the monomorphic MHC class I-related molecule CD1d (see., e.g., Brennan et al., Invariant natural killer T cells: an innate activation scheme linked to diverse effector functions. Nat Rev Immunol. (2013) 13:101-17. 10.1038). In some embodiments, exogenous lipid Ags include the prototypical α-Galactosylceramide (α-GalCer) (Kawano et al., CD1d-restricted and TCR-mediated activation of valpha14 NKT cells by glycosylceramides. Science. (1997) 278:1626-9. 10.1126) and a number of bacterial-derived Ags can activate iNKT cells.

iNKT cells undergo a distinct developmental pathway compared to T cells, leading to the acquisition of innate effector functions already in the thymus. Thymic iNKT cells indeed express markers usually upregulated by peripheral effector/memory T cells, such as CD44 and CD69, together with distinctive NK differentiation markers, such as NK1.1 (in some mouse genetic backgrounds, CD161 in humans), CD122 (the IL-2R/IL-15R β-chain), CD94/NKG2 and Ly49 (A-J), and a broad spectrum of TH1/2/17 effector cytokines. Once migrated in the periphery, in some embodiments, iNKT cells form a tissue resident population that survey the cellular integrity and rapidly respond to local damage and inflammation, jump starting the reaction by cells of the innate and adaptive immune response. In some embodiments, the anti-BCMA CAR iNKT cell is a Th1 iNKT cell, Th2 iNKT cell, a Th17 iNKT cell, or a combination thereof.

In some aspects, because iNKT cells can rapidly produce IFNγ, IL-4, or both, they have been found to play a role in various diseases by establishing a Th1- or Th2-based immune response. In bacterial and viral infections, iNKT cells typically help in early control of the pathogen by establishing a productive Th1 response. In both mouse and human studies, roles for iNKT cells have been described in diseases associated with excessive Th1 responses like type 1 diabetes and chronic obstructive pulmonary disease. Roles have also been described for iNKT cells helping to suppress Th1 responses and drive tolerogenic responses to grafts. As an example, following hematopoietic stem cell transfer, the presence of iNKT cells is predictive for survival with a reduction in graft versus host disease (GvHD) in patients and preclinical models.

In some aspects, iNKT cells infiltrate tumors and play an important role in the immune surveillance against tumors (e.g., solid and/or hematological malignancies) (see, e.g., Wolf et al., Novel Approaches to Exploiting Invariant NKT Cells in Cancer Immunotherapy, Front Immunol. 2018 Mar. 2; 9:384). In some instances, iNKT cells can be attractive platforms for adoptive cells immunotherapy of cancer compared to conventional T cells. In some embodiments, iNKT cells can directly kill cancer cells (e.g., CD1d-expressing cancer cells). In some embodiments, iNKT cells can restrict immunosuppressive myelomonocytic populations in the tumor microenvironment (TME) (e.g., via CD1d-cognate recognition), promoting anti-tumor responses (e.g., irrespective of the CD1d expression by cancer cells). In some embodiments, iNKT cells can be adoptively transferred across MHC barriers without risk of alloreaction because CD1d molecules are identical in all individuals, in addition to their ability to suppress graft vs. host disease (GvHD) without impairing the anti-tumor responses. In other embodiments, iNKT cells can be engineered to acquire a second antigen-specificity by expressing recombinant TCRs and/or Chimeric Antigen Receptor (CAR) specific for tumor-associated antigens, enabling the direct targeting of antigen-expressing cancer cells, while maintaining their CD1d-dependent functions. In some embodiments, iNKT cells can be used for donor unrestricted, and off the shelf, adoptive cell therapies enabling the concurrent targeting of cancer cells and suppressive microenvironment. Compared to conventional αβ T cells, iNKT cell adoptive immunotherapy include but are not limited to following advantages: (i) Control the tumor microenvironment (TME); (ii) Can be redirected against cancer cells by engineering with a tumor-specific CAR and/or TCR while maintaining their intrinsic control of the TME; (iii) Are devoid of alloreactivity, being restricted for the monomorphic CD1d molecule, allowing their possible use off the shelf in a donor-unrestricted manner.

Isolated antibody: An “isolated antibody”, as used herein, is intended to refer to an antibody that is substantially free of other antibodies having different antigenic specificities (e.g., an isolated antibody that specifically binds BCMA is substantially free of antibodies that specifically bind antigens other than BCMA). Moreover, an isolated antibody may be substantially free of other cellular material and/or chemicals.

Percent identity: The determination of “percent identity” or “percent identical” between two sequences (e.g., amino acid sequences or nucleic acid sequences) can be accomplished using a mathematical algorithm. A specific, non-limiting example of a mathematical algorithm utilized for the comparison of two sequences is the algorithm of Karlin S & Altschul S F (1990) PNAS 87:2264-2268, modified as in Karlin S & Altschul S F (1993) PNAS 90:5873-5877, each of which is herein incorporated by reference in its entirety. Such an algorithm is incorporated into the NBLAST and XBLAST programs of Altschul S F et al., (1990) J Mol Biol 215:403, which is herein incorporated by reference in its entirety. BLAST nucleotide searches can be performed with the NBLAST nucleotide program parameters set, e.g., at score=100, word length=12 to obtain nucleotide sequences homologous to a nucleic acid molecule described herein. BLAST protein searches can be performed with the XBLAST program parameters set, e.g., at score=50, word length=3 to obtain amino acid sequences homologous to a protein molecule described herein. To obtain gapped alignments for comparison purposes, Gapped BLAST can be utilized as described in Altschul S F et al., (1997) Nuc Acids Res 25:3389-3402, which is herein incorporated by reference in its entirety. Alternatively, PSI BLAST can be used to perform an iterated search which detects distant relationships between molecules. Id. When utilizing BLAST, Gapped BLAST, and PSI Blast programs, the default parameters of the respective programs (e.g., of XBLAST and NBLAST) can be used (see, e.g., National Center for Biotechnology Information (NCBI) on the worldwide web, ncbi.nlm.nih.gov). Another specific, non-limiting example of a mathematical algorithm utilized for the comparison of sequences is the algorithm of Myers and Miller, 1988, CABIOS 4:11-17, which is herein incorporated by reference in its entirety. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

Recombinant antibody: The term “recombinant human antibody”, as used herein, is intended to include all human antibodies that are prepared, expressed, created or isolated by recombinant means, such as antibodies expressed using a recombinant expression vector transfected into a host cell (described in more details in this disclosure), antibodies isolated from a recombinant, combinatorial human antibody library (Hoogenboom H. R., (1997) TIB Tech. 15:62-70; Azzazy H., and Highsmith W. E., (2002) Clin. Biochem. 35:425-445; Gavilondo J. V., and Larrick J. W. (2002) BioTechniques 29:128-145; Hoogenboom H., and Chames P. (2000) Immunology Today 21:371-378), antibodies isolated from an animal (e.g., a mouse) that is transgenic for human immunoglobulin genes (see e.g., Taylor, L. D., et al. (1992) Nucl. Acids Res. 20:6287-6295; Kellermann S-A., and Green L. L. (2002) Current Opinion in Biotechnology 13:593-597; Little M. et al (2000) Immunology Today 21:364-370) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo. One embodiment of the disclosure provides fully human antibodies capable of binding human BCMA which can be generated using techniques well known in the art, such as, but not limited to, using human Ig phage libraries such as those disclosed in Jermutus et al., PCT publication No. WO 2005/007699 A2.

Single-chain variable fragment (scFv): As used herein, the term “Single-chain variable fragment (scFv)”, refers to a fusion protein of the variable regions of the heavy (VH) and light chains (VL) of immunoglobulins, connected with a short linker peptide. A linker refers to a peptide or a short oligopeptide sequence used to join two subunits into a single polypeptide. A linker may have a sequence that is found in natural proteins, or may be an artificial sequence that is not found in any natural protein. A linker may be flexible and lacking in secondary structure or may have a propensity to form a specific three-dimensional structure under physiological conditions. In some embodiments, the linker is a glycine rich linker. In some embodiments, the linker is rich in serine or threonine. In some embodiments, a scFv is a fusion protein in which the N-terminus of the VH is linked with the C-terminus of the VL. In some embodiments, a scFv is a fusion protein in which the N-terminus of the VL is linked with the C-terminus of the VH. A scFv retains the specificity of the original immunoglobulin, despite removal of the constant regions and the introduction of the linker. In some embodiments, a scFv can be created to facilitate phage display. In some embodiments, a scFv can be created directly from subcloned heavy and light chains derived from a hybridoma. ScFvs have many uses, e.g., flow cytometry, immunohistochemistry, and as antigen-binding domains of a chimeric antigen receptor.

Specifically binds: As used herein, the term “specifically binds” or “specific binding” refers to the ability of a molecule to bind to a binding partner with a degree of affinity or avidity that enables the molecule to be used to distinguish the binding partner from an appropriate control in a binding assay or other binding context. With respect to an antibody, the term, “specifically binds”, refers to the ability of the antibody to bind to a specific antigen with a degree of affinity or avidity, compared with an appropriate reference antigen or antigens, that enables the antibody to be used to distinguish the specific antigen from others, e.g., to an extent that permits preferential targeting to certain cells, e.g., cancer cells, through binding to the antigen, as described herein. In some embodiments, an antibody specifically binds to a target if the antibody has a K_{D} for binding the target of at least about 104 M, 10⁻⁵ M, 10⁻⁶ M, 10⁻⁷ M, 10⁻⁸ M, 10⁻⁹ M, 10-10 M, 10-11 M, 10-12 M, 10-13 M, or less. In some embodiments, an antibody specifically binds to BCMA.

Subject: As used herein, the term “subject” refers to a mammal. In some embodiments, a subject is non-human primate, or rodent. In some embodiments, a subject is a human. In some embodiments, a subject is a patient, e.g., a human patient that has or is suspected of having a disease. In some embodiments, the subject is a human patient who has or is suspected of having cancer (e.g., BCMA-expressing cancer) or other disorders associated with aberrant BCMA expression or activity.

Treatment: As used herein, the term “treating” or “treatment” refers to the application or administration of a composition including one or more active agents (e.g., anti-BCMA antibodies) to a subject, who has a target disease or disorder (e.g., cancer), a symptom of the disease/disorder (e.g., tumor growth, tumor metastasis, fatigue, weight changes including unintended loss or gain, pain, fever, sores that don't heal, persistent cough or hoarseness, unusual bleeding, or anemia), or a predisposition toward the disease/disorder (e.g., cancer), with the purpose to cure, heal, alleviate, relieve, alter, remedy, ameliorate, improve, or affect the disorder (e.g., cancer), the symptom of the disease (e.g., tumor growth, tumor metastasis, fatigue, weight changes including unintended loss or gain, pain, fever, sores that don't heal, persistent cough or hoarseness, unusual bleeding, or anemia), or the predisposition toward the disease or disorder. Alleviating a target disease/disorder includes delaying or preventing the development or progression of the disease, or reducing disease severity.

### II. Anti-BCMA Antibodies or Antigen Binding Fragments Thereof

In some aspects, the present disclosure provides antibodies or antigen binding fragments specific for B-cell maturation antigen (BCMA). In some embodiments, the anti-BCMA antibodies or antigen binding fragments thereof provided herein are antibodies that bind to BCMA with high specificity and affinity. In some embodiments, the anti-BCMA antibody antigen binding fragments thereof described herein specifically binds to an extracellular epitope of BCMA or an epitope that becomes exposed to an antibody. In some embodiments, anti-BCMA antibodies antigen binding fragments thereof provided herein bind specifically to BCMA from human, non-human primates, mouse, rat, etc. In some embodiments, anti-BCMA antibodies antigen binding fragments thereof provided herein bind to human BCMA. In some embodiments, an anti-BCMA antibody or antigen binding fragment thereof described herein specifically binds to an epitope on human BCMA (e.g., human BCMA as set forth in SEQ ID NOs: 103 or 104). In some embodiments, an anti-BCMA antibody or antigen binding fragment thereof described binds to a fragment of a human BCMA (e.g., human BCMA as set forth in SEQ ID NOs: 103 or 104). In some embodiments, an anti-BCMA antibody or antigen binding fragment thereof described herein binds to a fragment of BCMA (e.g., human BCMA as set forth in SEQ ID NOs: 103 or 104) between about 5 and about 184 amino acids, between about 10 and about 184 amino acids, between about 20 and about 184 amino acids, between about 30 and about 150 amino acids, between about 30 and about 120 amino acids, between about 30 and about 100 amino acids, between about 30 and about 90 amino acids, between about 30 and about 80 amino acids, between about 30 and about 60 amino acids, between about 30 and about 50 amino acids, between about 40 and about 80 amino acids, or between about 40 and about 60 amino acids in length. In some embodiments, an anti-BCMA antibody or antigen binding fragment thereof described herein binds to a fragment comprising a contiguous number of amino acids from human BCMA protein (e.g., human BCMA as set forth in SEQ ID NOs: 103 or 104). In some embodiments, an anti-BCMA antibody or antigen binding fragment thereof described herein binds to a fragment comprising at least 5, at least 10, at least 20, at least 30, at least 40, at least 50, at least 60, at least 70, at least 80, at least 90, at least 100, at least 110, at least 120, at least 130, at least 140, at least 150, at least 160, at least 170, or at least 180 contiguous amino acids of human BCMA protein (e.g., human BCMA as set forth in SEQ ID NOs: 103 or 104). In some embodiments, an anti-BCMA antibody or antigen binding fragment thereof described herein binds to a fragment having an amino acid sequence having up to five amino acids, up to four amino acids, up to three amino acids, up to two amino acids, or up to one amino acid different from a fragment of human BCMA protein (e.g., human BCMA as set forth in SEQ ID NOs: 103 or 104). Exemplary human BCMA amino acid sequences are set forth in NP_001183 (SEQ ID NO: 103 or BAB60895 (SEQ ID NO: 104).

Exemplary BCMA amino acid sequence set forth in SEQ ID NO: 103 (NP_001183):

MLQMAGQCSQNEYFDSLLHACIPCQLRCSSNTPPLTCQRYCNASVTNSV
KGTNAILWTCLGLSLIISLAVFVLMFLLRKINSEPLKDEFKNTGSGLLG
MANIDLEKSRTGDEIILPRGLEYTVEECTCEDCIKSKPKVDSDHCFPLP
AMEEGATILVTTKTNDYCKSLPAALSATEIEKSISAR

Exemplary BCMA amino acid sequence set forth in SEQ ID NO: 104 (BAB60895):

MLQMAGQCSQNEYFDSLLHACIPCQLRCSSNTPPLTCQRYCNASVTNSV
KGTNAILWTCLGLSLIISLAVFVLMFLLRKISSEPLKDEFKNTGSGLLG
MANIDLEKSRTGDEIILPRGLEYTVEECTCEDCIKSKPKVDSDHCFPLP
AMEEGATILVTTKTNDYCKSLPAALSATEIEKSISAR

Exemplary BCMA epitopes are set forth in Table 2. In some embodiments, an anti-BCMA antibody or antigen binding fragment thereof described herein binds to an epitope having an amino acid sequence at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to any one of SEQ ID NOs: 38-48. In some embodiments, an anti-BCMA antibody or antigen binding fragment thereof described herein binds to an epitope having an amino acid sequence up to one amino acid, up to two amino acids, up to three amino acids, up to four amino acids, up to five amino acids, up to six amino acids, up to seven amino acids, up to eight amino acids, up to nine amino acids, or up to ten amino acids different from any one of SEQ ID NOs: 38-48.

TABLE 2
BCMA epitopes

| BCMA | | |
|---|---|---|
| Epitope | Epitope Sequence | SEQ ID NO |
| Epitope 1 | MLQMAGQCSQNEYFASALHACIPCQLRCSSNTPPL | 38 |
| | TCQRYCNASVTNSVKGTNA | |
| Epitope 2 | MLQMAGACSANEYFDSLLHACIPCQLRCSSNTPPLT | 39 |
| | CORYCNASVTNSVKGTNA | |
| Epitope 3 | MLQMAGQCSQNEYFAALLHACIPCQLRCSSNTPPL | 40 |
| | TCQRYCNASVTNSVKGTNA | |
| Epitope 4 | MLQMAGQCSQNEYFDSAAHACIPCQLRCSSNTPPL | 41 |
| | TCQRYCNASVTNSVKGTNA | |
| Epitope 5 | MLQMAGQCSQNEYFDSLLHACIPCQAACSSNTPPL | 42 |
| | TCQRYCNASVTNSVKGTNA | |
| Epitope 6 | MLQMAGQCSQNEYFDSLLHACIPCQLRCSAATPPL | 43 |
| | TCQRYCNASVTNSVKGTNA | |
| Epitope 7 | MLQMAGQCSQNEYFDSLLHACIPCQLRCSSNAPPA | 44 |
| | TCQRYCNASVTNSVKGTNA | |
| Epitope 8 | MLQMAGQCSQNEYFDSLLHACIPCQLRCSSNTPPL | 45 |
| | ACARYCNASVTNSVKGTNA | |
| Epitope 9 | MLQMAGQCSQNEYFDSLLHACIPCQLRCSSNTPPLT | 46 |
| | CQAYCAASVTNSVKGTNA | |
| Epitope 10 | MLQMAGQCSQNEYFDSLLHACIPCQLRCSSNTPPLT | 47 |
| | CQRYCN | |
| Epitope 11 | MLQMAGQCSQNEYFDSLLHACIPCQLRCSSNTPPLT | 48 |
| (wild type) | CQRYCNASVTNSVKGTNA | |

In some embodiments, an anti BCMA antibody or antigen binding fragment thereof specifically binds BCMA with binding affinity (e.g., as indicated by K_{D}) of at least about 104 M, 105 M, 10⁻⁶ M, 10⁻⁷ M, 10⁻⁸ M, 10⁻⁹ M, 10-10 M, 10-11 M, 10-12 M, 10-13 M, or less. For example, an anti BCMA antibody or antigen binding fragment thereof of the present disclosure can bind to a BCMA protein (e.g., human BCMA epitope as set forth in any one of SEQ ID NOs: 38-48) with an affinity between 5 pM and 500 nM, between 10 pM and 450 nM, between 20 pM and 400 nM, between 30 pM and 350 nM, between 40 pM and 300 nM, between 50 pM and 250 nM, between 60 pM and 200 nM, between 70 pM and 150 nM, between 80 pM and 100 nM, between 80 pM and 90 nM, between 90 pM and 80 nM, between 100 pM and 70 nM, between 200 pM and 60 nM, between 300 pM and 50 nM, between 400 pM and 40 nM, between 500 pM and 30 nM, between 600 pM and 20 nM, between 700 pM and 10 nM, between 800 pM and 5 nM, or between 900 pM and 2 nM.

The disclosure also includes antibodies that compete with any of the antibodies described herein for binding to a BCMA protein (e.g., human BCMA epitope as set forth in any one of SEQ ID NOs: 38-48) and that have an affinity of 100 nM or lower (e.g., 80 nM or lower, 50 nM or lower, 20 nM or lower, 10 nM or lower, 500 pM or lower, 50 pM or lower, or 5 pM or lower). The affinity and binding kinetics of the anti-BCMA antibodies or antigen binding fragment thereof can be tested using any suitable method including but not limited to biosensor technology (e.g., OCTET or BIACORE). In some embodiments, the anti-BCMA antibodies or antigen binding fragment thereof described herein binds to BCMA with a K_{D} of sub-nanomolar range.

Non-limiting examples of anti-BCMA antibodies are provided in Table 3.

TABLE 3
Exemplary anti-BCMA antibodies (CDRs according to the Kabat definition; CDR
sequences in the VH/VL/ScFv sequences bolded; VH/VL sequences in the scFv underlined)

| Anti-BCMA antibodies | | Sequences | SEQ ID NO |
|---|---|---|---|
| F6G | CDRH1 | GYYMH | 1 |
| (IGHV1-8\| | CDRH2 | WMNPNSGNTGYAQKFQG | 2 |
| IGHJ4*02) | CDRH3 | AQWEPLDY | 3 |
| | CDRL1 | SGDALPKKYT | 4 |
| | CDRL2 | EDTKRPS | 5 |
| | CDRL3 | LSTDPTGQESV | 6 |
| | VH | QVQLVQSGAEVKKPGASVKVSCKASGYTFTGYYMHWVRQA | 7 |
| | | PGQGLEWMGWMNPNSGNTGYAQKFQGRVTMTRNTSISTAY | |
| | | MELSSLRSEDTAVYYCARAQWEPLDYWGQGTLVTVSS | |
| | VL | SYELTQPPSVSVSPGQTARITCSGDALPKKYTYWYQQKSG | 8 |
| | | QAPVLVIYEDTKRPSGIPERFSGSSSGTMATLTISGAQVE | |
| | | DEADYYCLSTDPTGQESVFGGGTKLTVLGQP | |
| | scFv | QVQLVQSGAEVKKPGASVKVSCKASGYTFTGYYMHWVRQA | 9 |
| | | PGQGLEWMGWMNPNSGNTGYAQKFQGRVTMTRNTSISTAY | |
| | | MELSSLRSEDTAVYYCARAQWEPLDYWGQGTLVTVSSGGG | |
| | | GSGGGGSGGGASSYELTQPPSVSVSPGQTARITCSGDALP | |
| | | KKYTYWYQQKSGQAPVLVIYEDTKRPSGIPERFSGSSSGT | |
| | | MATLTISGAQVEDEADYYCLSTDPTGQESVFGGGTKLTVL | |
| | | GQP | |
| F6G-Var1 | CDRH1 | GYYMH | 1 |
| | CDRH2 | WMNPNSGNTGYAQKFQG | 2 |
| | CDRH3 | AQWEPLDY | 3 |
| | CDRL1 | SGDALPKKYT | 4 |
| | CDRL2 | YDTKRPS | 24 |
| | CDRL3 | LSTDPTGQESV | 6 |
| | VH | QVQLVQSGAEVKKPGASVKVSCKASGYTFTGYYMHWVRQA | 7 |
| | | PGQGLEWMGWMNPNSGNTGYAQKFQGRVTMTRNTSISTAY | |
| | | MELSSLRSEDTAVYYCARAQWEPLDYWGQGTLVTVSS | |
| | VL | SYELTQPPSVSVSPGQTARITCSGDALPKKYTYWYQQKSG | 10 |
| | | QAPVLVIYYDTKRPSGIPERFSGSSSGTMATLTISGAQVE | |
| | | DEADYYCLSTDPTGQESVFGGGTKLTVLGQP | |
| | scFv | QVQLVQSGAEVKKPGASVKVSCKASGYTFTGYYMHWVRQA | 11 |
| | | PGQGLEWMGWMNPNSGNTGYAQKFQGRVTMTRNTSISTAY | |
| | | MELSSLRSEDTAVYYCARAQWEPLDYWGQGTLVTVSSGGG | |
| | | GSGGGGSGGGASSYELTQPPSVSVSPGQTARITCSGDALP | |
| | | KKYTYWYQQKSGQAPVLVIYYDTKRPSGIPERFSGSSSGT | |
| | | MATLTISGAQVEDEADYYCLSTDPTGQESVFGGGTKLTVL | |
| | | GQP | |
| F6G-Var2 | CDRH1 | GYYMQ | 25 |
| | CDRH2 | WMNPISGNTGYAQKFQG | 27 |
| | CDRH3 | AQWEPLDY | 3 |
| | CDRL1 | SGDALPKKYT | 4 |
| | CDRL2 | YDTKRPS | 24 |
| | CDRL3 | LSTHPAGQESV | 30 |
| | VH | QVQLVQSGAEVKKPGASVKVSCKASGYTFTGYYMQWVRQA | 12 |
| | | PGQGLEWMGWMNPISGNTGYAQKFQGRVTMTRNTSISTAY | |
| | | MELSSLRSEDTAVYYCARAQWEPLDYWGQGTLVTVSS | |
| | VL | SYELTQPPSVSVSPGQTARITCSGDALPKKYTYWYQQKSG | 13 |
| | | QAPVLVIYYDTKRPSGIPERFSGSSSGTMATLTISGAQVE | |
| | | DEADYYCLSTHPAGQESVFGGGTKLTVLGQP | |
| | scFv | QVQLVQSGAEVKKPGASVKVSCKASGYTFTGYYMQWVRQA | 14 |
| | | PGQGLEWMGWMNPISGNTGYAQKFQGRVTMTRNTSISTAY | |
| | | MELSSLRSEDTAVYYCARAQWEPLDYWGQGTLVTVSSGGG | |
| | | GSGGGGSGGGASSYELTQPPSVSVSPGQTARITCSGDALP | |
| | | KKYTYWYQQKSGQAPVLVIYYDTKRPSGIPERFSGSSSGT | |
| | | MATLTISGAQVEDEADYYCLSTHPAGQESVFGGGTKLTVL | |
| | | GQP | |
| F6G-Var3 | CDRH1 | GYYMQ | 25 |
| | CDRH2 | WMNPISGNTGYAQKFQG | 27 |
| | CDRH3 | AQWEPLDY | 3 |
| | CDRL1 | SGDALPKKYT | 4 |
| | CDRL2 | YDTKRPS | 24 |
| | CDRL3 | LSTNPTGHESV | 29 |
| | VH | QVQLVQSGAEVKKPGASVKVSCKASGYTFTGYYMQWVRQA | 12 |
| | | PGQGLEWMGWMNPISGNTGYAQKFQGRVTMTRNTSISTAY | |
| | | MELSSLRSEDTAVYYCARAQWEPLDYWGQGTLVTVSS | |
| | VL | SYELTQPPSVSVSPGQTARITCSGDALPKKYTYWYQQKSG | 15 |
| | | QAPVLVIYYDTKRPSGIPERFSGSSSGTMATLTISGAQVE | |
| | | DEADYYCLSTNPTGHESVFGGGTKLTVLGQP | |
| | scFv | QVQLVQSGAEVKKPGASVKVSCKASGYTFTGYYMQWVRQA | 16 |
| | | PGQGLEWMGWMNPISGNTGYAQKFQGRVTMTRNTSISTAY | |
| | | MELSSLRSEDTAVYYCARAQWEPLDYWGQGTLVTVSSGGG | |
| | | GSGGGGSGGGASSYELTQPPSVSVSPGQTARITCSGDALP | |
| | | KKYTYWYQQKSGQAPVLVIYYDTKRPSGIPERFSGSSSGT | |
| | | MATLTISGAQVEDEADYYCLSTNPTGHESVFGGGTKLTVL | |
| | | GQP | |
| F6G-Var4 | CDRH1 | GYYMH | 1 |
| | CDRH2 | WMNPNSGNTGYAQKFQG | 2 |
| | CDRH3 | AQWEPLDY | 3 |
| | CDRL1 | SGDALPKKYT | 4 |
| | CDRL2 | YDTKRPS | 24 |
| | CDRL3 | LSTHPAGQESV | 30 |
| | VH | QVQLVQSGAEVKKPGASVKVSCKASGYTFTGYYMHWVRQA | 7 |
| | | PGQGLEWMGWMNPNSGNTGYAQKFQGRVTMTRNTSISTAY | |
| | | MELSSLRSEDTAVYYCARAQWEPLDYWGQGTLVTVSS | |
| | VL | SYELTQPPSVSVSPGQTARITCSGDALPKKYTYWYQQKSG | 13 |
| | | QAPVLVIYYDTKRPSGIPERFSGSSSGTMATLTISGAQVE | |
| | | DEADYYCLSTHPAGQESVFGGGTKLTVLGQP | |
| | scFv | QVQLVQSGAEVKKPGASVKVSCKASGYTFTGYYMHWVRQA | 17 |
| | | PGQGLEWMGWMNPNSGNTGYAQKFQGRVTMTRNTSISTAY | |
| | | MELSSLRSEDTAVYYCARAQWEPLDYWGQGTLVTVSSGGG | |
| | | GSGGGGSGGGASSYELTQPPSVSVSPGQTARITCSGDALP | |
| | | KKYTYWYQQKSGQAPVLVIYYDTKRPSGIPERFSGSSSGT | |
| | | MATLTISGAQVEDEADYYCLSTHPAGQESVFGGGTKLTVL | |
| | | GQP | |
| F6G-Var5 | CDRH1 | GYYMH | 1 |
| | CDRH2 | WMNPNSGNTGYAQKFQG | 2 |
| | CDRH3 | AQWEPLDY | 3 |
| | CDRL1 | SGDALPKKYT | 4 |
| | CDRL2 | YDTKRPS | 24 |
| | CDRL3 | LSTNPTGHESV | 29 |
| | VH | QVQLVQSGAEVKKPGASVKVSCKASGYTFTGYYMHWVRQA | 7 |
| | | PGQGLEWMGWMNPNSGNTGYAQKFQGRVTMTRNTSISTAY | |
| | | MELSSLRSEDTAVYYCARAQWEPLDYWGQGTLVTVSS | |
| | VL | SYELTQPPSVSVSPGQTARITCSGDALPKKYTYWYQQKSG | 15 |
| | | QAPVLVIYYDTKRPSGIPERFSGSSSGTMATLTISGAQVE | |
| | | DEADYYCLSTNPTGHESVFGGGTKLTVLGQP | |
| | scFv | QVQLVQSGAEVKKPGASVKVSCKASGYTFTGYYMHWVRQA | 18 |
| | | PGQGLEWMGWMNPNSGNTGYAQKFQGRVTMTRNTSISTAY | |
| | | MELSSLRSEDTAVYYCARAQWEPLDYWGQGTLVTVSSGGG | |
| | | GSGGGGSGGGASSYELTQPPSVSVSPGQTARITCSGDALP | |
| | | KKYTYWYQQKSGQAPVLVIYYDTKRPSGIPERFSGSSSGT | |
| | | MATLTISGAQVEDEADYYCLSTNPTGHESVFGGGTKLTVL | |
| | | GQP | |
| F6G-Var6 | CDRH1 | EYYMK | 26 |
| | CDRH2 | WMNPNTGKTGYAQKFQG | 28 |
| | CDRH3 | AQWEPLDY | 3 |
| | CDRL1 | SGDALPKKYT | 4 |
| | CDRL2 | YDTKRPS | 24 |
| | CDRL3 | LSTDPTGQESV | 6 |
| | VH | QVQLVQSGAEVKKPGASVKVSCKASGYTFTEYYMKWVRQA | 19 |
| | | PGQGLEWMGWMNPNTGKTGYAQKFQGRVTMTRNTSISTAY | |
| | | MELSSLRSEDTAVYYCARAQWEPLDYWGQGTLVTVSS | |
| | VL | SYELTQPPSVSVSPGQTARITCSGDALPKKYTYWYQQKSG | 10 |
| | | QAPVLVIYYDTKRPSGIPERFSGSSSGTMATLTISGAQVE | |
| | | DEADYYCLSTDPTGQESVFGGGTKLTVLGQP | |
| | scFv | QVQLVQSGAEVKKPGASVKVSCKASGYTFTEYYMKWVRQA | 20 |
| | | PGQGLEWMGWMNPNTGKTGYAQKFQGRVTMTRNTSISTAY | |
| | | MELSSLRSEDTAVYYCARAQWEPLDYWGQGTLVTVSSGGG | |
| | | GSGGGGSGGGASSYELTQPPSVSVSPGQTARITCSGDALP | |
| | | KKYTYWYQQKSGQAPVLVIYYDTKRPSGIPERFSGSSSGT | |
| | | MATLTISGAQVEDEADYYCLSTDPTGQESVFGGGTKLTVL | |
| | | GQP | |
| F1G- | CDRH1 | SYGIS | 31 |
| HFW3 | CDRH2 | WISAYNGNTYYAEDLQG | 32 |
| (IGHV1-18\| | CDRH3 | EGSYYDSSGYYLPY | 33 |
| IGHJ4*02- | CDRL1 | TGTSSDVGAYNYVS | 34 |
| IGLV2-11\| | CDRL2 | DVTERPS | 35 |
| IGLJ3*02) | CDRL3 | SSYTSSSIA | 36 |
| | VH | QVQLVQSGAEVKKPGASVKVSCKASGYTFTSYGISWVRQA | 21 |
| | | PGQGLEWMGWISAYNGNTYYAEDLQGRLTMTTDTSTSTAY | |
| | | MELRSLRSDDTAVYYCAREGSYYDSSGYYLPYWGQGTLVT | |
| | | VSS | |
| | VL | QSALTQPRSVSGSPGQSVTISCTGTSSDVGAYNYVSWYQQ | 22 |
| | | HPGKAPKLMIYDVTERPSGVPDRFSGSKSGNTASLTISGL | |
| | | QAEDEADYYCSSYTSSSIAFGGGTKLTVLGQP | |
| | scFv | QVQLVQSGAEVKKPGASVKVSCKASGYTFTSYGISWVRQA | 23 |
| | | PGQGLEWMGWISAYNGNTYYAEDLQGRLTMTTDTSTSTAY | |
| | | MELRSLRSDDTAVYYCAREGSYYDSSGYYLPYWGQGTLVT | |
| | | VSSGGGGSGGGGSGGGASQSALTQPRSVSGSPGQSVTISC | |
| | | TGTSSDVGAYNYVSWYQQHPGKAPKLMIYDVTERPSGVPD | |
| | | RFSGSKSGNTASLTISGLQAEDEADYYCSSYTSSSIAFGG | |
| | | GTKLTVLGQP | |

In some embodiments, an anti-BCMA antibody or antigen binding fragment thereof of the present disclosure comprises one or more of the heavy chain CDRs (e.g., CDRH1, CDRH2, or CDRH3) amino acid sequences from any one of the anti-BCMA antibodies selected from Table 3. In some embodiments, an anti-BCMA antibody or antigen binding fragment thereof of the present disclosure comprise the CDRH1, CDRH2, and CDRH3 as provided for any one of the antibodies elected from Table 3. In some embodiments, an anti-BCMA antibody or antigen binding fragment thereof of the present disclosure comprises one or more of the light chain CDRs (e.g., CDRL1, CDRL2, or CDRL3) amino acid sequences from any one of the anti-BCMA antibodies selected from Table 3. In some embodiments, an anti-BCMA antibody or antigen binding fragment thereof of the present disclosure comprise the CDRL1, CDRL2, and CDRL3 as provided for any one of the anti-BCMA antibodies selected from Table 3.

In some embodiments, an anti-BCMA antibody or antigen binding fragment thereof comprises the CDRH1, CDRH2, CDRH3, CDRL1, CDRL2, and CDRL3 as provided for any one of the anti-BCMA antibodies selected from Table 3. In some embodiments, antibody heavy and light chain CDR3 domains may play a particularly important role in the binding specificity/affinity of an antibody for an antigen. Accordingly, an anti-BCMA antibody or antigen binding fragment thereof may include at least the heavy and/or light chain CDR3s of any one of the anti-BCMA antibodies selected from Table 3.

Also within the scope of the present disclosure are functional variants of any of the exemplary anti-BCMA antibody or antigen binding fragment thereof as disclosed herein. A functional variant may contain one or more amino acid residue variations in the VH and/or VL, or in one or more of the heavy chain CDRs and/or one or more of the light chain CDRs as relative to the reference antibody, while retaining substantially similar binding and biological activities (e.g., substantially similar binding affinity, binding specificity, inhibitory activity, anti-inflammatory activity, or a combination thereof) as the reference antibody.

In some embodiments, any of the anti-BCMA antibody or antigen binding fragment thereof of the disclosure have one or more CDRs (e.g., heavy chain CDR or light chain CDR) sequences substantially similar to any of the CDRH1, CDRH2, CDRH3, CDRL1, CDRL2, and/or CDRL3 sequences from one of the anti-BCMA antibodies selected from Table 3. In some embodiments, the position of one or more CDRs along the VH (e.g., CDRH1, CDRH2, or CDRH3) and/or VL (e.g., CDRL1, CDRL2, or CDRL3) region of an antibody described herein can vary by one, two, three, four, five, or six amino acid positions so long as specific binding to BCMA (e.g., human BCMA) is maintained (e.g., substantially maintained, for example, at least 50%, at least 60%, at least 70%, at least 80%, at least 90%, at least 95% of the binding of the original antibody from which it is derived). For example, in some embodiments, the position defining a CDR of any antibody described herein can vary by shifting the N-terminal and/or C-terminal boundary of the CDR by one, two, three, four, five, or six amino acids, relative to the CDR position of any one of the antibodies described herein, so long as specific binding to BCMA (e.g., human BCMA) is maintained (e.g., substantially maintained, for example, at least 50%, at least 60%, at least 70%, at least 80%, at least 90%, at least 95% of the binding of the original antibody from which it is derived). In another embodiment, the length of one or more CDRs along the VH (e.g., CDRH1, CDRH2, or CDRH3) and/or VL (e.g., CDRL1, CDRL2, or CDRL3) region of an antibody described herein can vary (e.g., be shorter or longer) by one, two, three, four, five, or more amino acids, so long as specific binding to BCMA (e.g., human BCMA) is maintained (e.g., substantially maintained, for example, at least 50%, at least 60%, at least 70%, at least 80%, at least 90%, at least 95% of the binding of the original antibody from which it is derived).

Accordingly, in some embodiments, a CDRH1, CDRH2, CDRH3, CDRL1, CDRL2, and/or CDRL3 of an anti-BCMA antibody or antigen binding fragment thereof may be one, two, three, four, five or more amino acids shorter than one or more of the CDRs described herein (e.g., CDRs from any of the anti-BCMA antibodies selected from Table 3) so long as specific binding to BCMA (e.g., human BCMA) is maintained (e.g., substantially maintained, for example, at least 50%, at least 60%, at least 70%, at least 80%, at least 90%, at least 95% relative to the binding of the original antibody from which it is derived). In some embodiments, a CDRH1, CDRH2, CDRH3, CDRL1, CDRL2, and/or CDRL3 of an anti-BCMA antibody or antigen binding fragment thereof may be one, two, three, four, five or more amino acids longer than one or more of the CDRs described herein (e.g., CDRs from any of the anti-BCMA antibodies selected from Table 3) so long as specific binding to BCMA (e.g., human BCMA) is maintained (e.g., substantially maintained, for example, at least 50%, at least 60%, at least 70%, at least 80%, at least 90%, at least 95% relative to the binding of the original antibody from which it is derived). In some embodiments, the amino portion of a CDRH1, CDRH2, CDRH3, CDRL1, CDRL2, and/or CDRL3 of an anti-BCMA antibody or antigen binding fragment thereof can be extended by one, two, three, four, five or more amino acids compared to one or more of the CDRs described herein (e.g., CDRs from any of the anti-BCMA antibodies selected from Table 3) so long as specific binding to BCMA (e.g., human BCMA) is maintained (e.g., substantially maintained, for example, at least 50%, at least 60%, at least 70%, at least 80%, at least 90%, at least 95% relative to the binding of the original antibody from which it is derived). In some embodiments, the carboxy portion of a CDRH1, CDRH2, CDRH3, CDRL1, CDRL2, and/or CDRL3 of an anti-BCMA antibody or antigen binding fragment thereof can be extended by one, two, three, four, five or more amino acids compared to one or more of the CDRs described herein (e.g., CDRs from any of the anti-BCMA antibodies selected from Table 3) so long as specific binding to BCMA (e.g., human BCMA) is maintained (e.g., substantially maintained, for example, at least 50%, at least 60%, at least 70%, at least 80%, at least 90%, at least 95% relative to the binding of the original antibody from which it is derived). In some embodiments, the amino portion of a CDRH1, CDRH2, CDRH3, CDRL1, CDRL2, and/or CDRL3 of an anti-BCMA antibody or antigen binding fragment thereof can be shortened by one, two, three, four, five or more amino acids compared to one or more of the CDRs described herein (e.g., CDRs from any of the anti-BCMA antibodies selected from Table 3) so long as specific binding to BCMA (e.g., human BCMA) is maintained (e.g., substantially maintained, for example, at least 50%, at least 60%, at least 70%, at least 80%, at least 90%, at least 95% relative to the binding of the original antibody from which it is derived). In some embodiments, the carboxy portion of a CDRH1, CDRH2, CDRH3, CDRL1, CDRL2, and/or CDRL3 of an anti-BCMA antibody or antigen binding fragment thereof can be shortened by one, two, three, four, five or more amino acids compared to one or more of the CDRs described herein (e.g., CDRs from any of the anti-BCMA antibodies selected from Table 3) so long as specific binding to BCMA (e.g., human BCMA) is maintained (e.g., substantially maintained, for example, at least 50%, at least 60%, at least 70%, at least 80%, at least 90%, at least 95% relative to the binding of the original antibody from which it is derived). Any method can be used to ascertain whether specific binding to BCMA (e.g., human BCMA) is maintained, for example, using binding assays and conditions described in the art.

In some examples, an anti-BCMA antibody or antigen binding fragment thereof comprises one or more CDR (e.g., heavy chain CDRs or light chain CDR) sequences substantially similar to any one of the anti-BCMA antibodies selected from Table 3. For example, the antibodies may include one or more CDR sequence(s) from any of the anti-BCMA antibodies selected from Table 3 containing up to 5, 4, 3, 2, or 1 amino acid residue variations as compared to the corresponding CDR region in any one of the CDRs provided herein (e.g., CDRs from any of the anti-BCMA antibodies selected from Table 3) so long as specific binding to BCMA (e.g., human BCMA) is maintained (e.g., substantially maintained, for example, at least 50%, at least 60%, at least 70%, at least 80%, at least 90%, at least 95% relative to the binding of the original antibody from which it is derived). In some embodiments, any of the amino acid variations in any of the CDRs provided herein may be conservative variations. Conservative variations can be introduced into the CDRs at positions where the residues are not likely to be involved in interacting with a BCMA protein (e.g., human BCMA), for example, as determined based on a crystal structure.

Some aspects of the disclosure provide anti-BCMA antibodies that comprise one or more of the heavy chain variable (VH) and/or light chain variable (VL) domains provided herein. In some embodiments, any of the VH domains provided herein include one or more of the heavy chain CDR sequences (e.g., CDRH1, CDRH2, and CDRH3) provided herein, for example, any of the heavy chain CDR sequences provided in any one of the anti-BCMA antibodies selected from Table 3. In some embodiments, any of the VL domains provided herein include one or more of the CDR-L sequences (e.g., CDRL1, CDRL2, and CDRL3) provided herein, for example, any of the light chain CDR sequences provided in any one of the anti-BCMA antibodies selected from Table 3.

In some embodiments, an anti-BCMA antibody or antigen binding fragment thereof includes any antibody that comprises a heavy chain variable domain and/or a light chain variable domain of any one of the anti-BCMA antibodies selected from Table 3, and variants thereof. In some embodiments, an anti-BCMA antibody or antigen binding fragment thereof includes any antibody that comprises the heavy chain variable and light chain variable pairs of any anti-BCMA antibodies selected from Table 3.

Aspects of the disclosure provide anti-BCMA antibodies or antigen binding fragments thereof comprising a heavy chain variable (VH) and/or a light chain variable (VL) domain amino acid sequence homologous to any of those described herein. In some embodiments, an anti-BCMA antibody or antigen binding fragment thereof comprises a VH or a VL that is at least 75% (e.g., at least 80%, at least 85%, at least 90%, at least 91%, at least 92%, at least 93%, at least 94%, at least 95%, at least 96%, at least 97%, at least 98%, at least 99%, or 100%) identical to the VH and/or any VL of any one of the anti-BCMA antibodies selected from Table 3. In some embodiments, the homologous VH and/or a VL amino acid sequences do not vary within any of the CDR sequences provided herein. For example, in some embodiments, the degree of sequence variation (e.g., at least 80%, at least 85%, at least 90%, at least 91%, at least 92%, at least 93%, at least 94%, at least 95%, at least 96%, at least 97%, at least 98%, at least 99%, or 100%) may occur within a VH and/or a VL sequence excluding any of the CDR sequences provided herein. In some embodiments, any of the anti-BCMA antibodies or antigen binding fragments thereof provided herein comprise a VH sequence and a VL sequence that comprises a framework sequence that is at least 75%, 80%, 85%, 90%, 95%, 98%, or 99% identical to the framework sequence of any anti-BCMA antibodies selected from Table 3. In some embodiments, an anti-BCMA antibody or antigen binding fragment thereof comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH of any of the anti-BCMA antibodies listed in Table 3. Alternatively or in addition, an anti-BCMA antibody or antigen binding fragment thereof comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL of any one of the anti-BCMA antibodies listed in Table 3.

In some embodiments, an anti-BCMA antibody or antigen binding fragment thereof is a humanized antibody (e.g., a humanized variant containing one or more CDRs of Table 3). In some embodiments, an anti-BCMA antibody or antigen binding fragment thereof comprises a CDRH1, a CDRH2, a CDRH3, a CDRL1, a CDRL2, and a CDRL3 that are the same as the CDRH1, CDRH2, CDRH3, CDRL1, CDRL2, and CDRL3 shown in Table 3, and comprises a humanized VH and/or a humanized VL. In some embodiments, an anti-BCMA antibody or antigen binding fragment thereof is a humanized variant comprising one or more amino acid substitutions (e.g., in the VH framework region) as compared with any one of the VHs listed in Table 3, and/or one or more amino acid substitutions (e.g., in the VL framework region) as compared with any one of the VLs listed in Table 3.

In some embodiments, an anti-BCMA antibody or antigen binding fragment thereof comprises a CDRH1, a CDRH2 and a CDRH3 of a heavy chain variable domain (VH) having the amino acid sequence of SEQ ID NO: 7. Alternatively or in addition, an anti-BCMA antibody or antigen binding fragment thereof comprises a CDRL1, a CDRL2 and a CDRL3 of a light chain variable domain (VL) having the amino acid sequence of SEQ ID NO: 8.

In some embodiments, according to the Kabat definition system, an anti-BCMA antibody or antigen binding fragment thereof comprises a CDRH3 having the amino acid sequence of SEQ ID NO: 3. In some embodiments, according to the Kabat definition system, an anti-BCMA antibody or antigen binding fragment thereof comprises a CDRH1 having the amino acid sequence of SEQ ID NO: 1, a CDRH2 having the amino acid sequence of SEQ ID NO: 2, and a CDRH3 having the amino acid sequence of SEQ ID NO: 3. In some embodiments, according to the Kabat definition system, an anti-BCMA antibody or antigen binding fragment thereof comprises a CDRH1 having the amino acid sequence of SEQ ID NO: 1, a CDRH2 having the amino acid sequence of SEQ ID NO: 2, a CDRH3 having the amino acid sequence of SEQ ID NO: 3, a CDRL1 having the amino acid sequence of SEQ ID NO: 4, a CDRL2 having the amino acid sequence of SEQ ID NO: 5, and a CDRL3 having the amino acid sequence of SEQ ID NO: 6.

In some embodiments, an anti-BCMA antibody or antigen binding fragment thereof comprises a CDRH1, a CDRH2, and a CDRH3, which collectively contains no more than 5 amino acid variations (e.g., no more than 5, 4, 3, 2, or 1 amino acid variation) as compared with the CDRH1 having the amino acid sequence of SEQ ID NO: 1, CDRH2 having the amino acid sequence of SEQ ID NO: 2, and CDRH3 having the amino acid sequence of SEQ ID NO: 3. “Collectively,” as used anywhere in the present disclosure, means that the total number of amino acid variations in all of the three heavy chain CDRs is within the defined range. Alternatively or in addition, an anti-BCMA antibody or antigen binding fragment thereof comprises a CDRL1, a CDRL2, and a CDRL3, which collectively contains no more than 5 amino acid variations (e.g., no more than 5, 4, 3, 2 or 1 amino acid variation) as compared with the CDRL1 having the amino acid sequence of SEQ ID NO: 4, CDRL2 having the amino acid sequence of SEQ ID NO: 5, and CDRL3 having the amino acid sequence of SEQ ID NO: 6.

In some embodiments, an anti-BCMA antibody or antigen binding fragment thereof comprises a CDRH1, a CDRH2, and a CDRH3 that collectively are at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the CDRH1 having the amino acid sequence of SEQ ID NO: 1, CDRH2 having the amino acid sequence of SEQ ID NO: 2, and CDRH3 having the amino acid sequence of SEQ ID NO: 3. Alternatively or in addition, an anti-BCMA antibody or antigen binding fragment thereof comprises a CDRL1, a CDRL2, and a CDRL3 that collectively are at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the to the CDRL1 having the amino acid sequence of SEQ ID NO: 4, CDRL2 having the amino acid sequence of SEQ ID NO: 5, and CDRL3 having the amino acid sequence of SEQ ID NO: 6.

In some embodiments, an anti-BCMA antibody or antigen binding fragment thereof comprises: a CDRH1 having no more than 3 amino acid variations (e.g., no more than 3, 2, or 1 amino acid variation) as compared with the CDRH1 having the amino acid sequence of SEQ ID NO: 1; a CDRH2 having no more than 3 amino acid variations (e.g., no more than 3, 2, or 1 amino acid variation) as compared with the CDRH2 having the amino acid sequence of SEQ ID NO: 2; and/or a CDRH3 having no more than 3 amino acid variations (e.g., no more than 3, 2, or 1 amino acid variation) as compared with the CDRH3 having the amino acid sequence of SEQ ID NO: 3. Alternatively or in addition, an anti-BCMA antibody or antigen binding fragment thereof comprises: a CDRL1 having no more than 3 amino acid variations (e.g., no more than 3, 2, or 1 amino acid variation) as compared with the CDRL1 having the amino acid sequence of SEQ ID NO: 4; a CDRL2 having no more than 3 amino acid variations (e.g., no more than 3, 2, or 1 amino acid variation) as compared with the CDRL2 having the amino acid sequence of SEQ ID NO: 5; and/or a CDRL3 having no more than 3 amino acid variations (e.g., no more than 3, 2, or 1 amino acid variation) as compared with the CDRL3 having the amino acid sequence of SEQ ID NO: 6.

In some embodiments, an anti-BCMA antibody or antigen binding fragment thereof comprises a VH comprising the amino acid sequence of SEQ ID NO: 7. Alternatively or in addition, an anti-BCMA antibody or antigen binding fragment thereof comprises a VL comprising the amino acid sequence of SEQ ID NO: 8.

In some embodiments, an anti-BCMA antibody or antigen binding fragment thereof comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 7. Alternatively or in addition, an anti-BCMA antibody or antigen binding fragment thereof comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 8.

In some embodiments, an anti-BCMA antibody or antigen binding fragment thereof comprises a VH comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VH as set forth in SEQ ID NO: 7. Alternatively or in addition, an anti-BCMA antibody or antigen binding fragment thereof comprises a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VL as set forth in SEQ ID NO: 8.

In some embodiments, an anti-BCMA antibody or antigen binding fragment thereof comprises a CDRH1, a CDRH2 and a CDRH3 of a heavy chain variable domain (VH) having the amino acid sequence of SEQ ID NO: 7. Alternatively or in addition, an anti-BCMA antibody or antigen binding fragment thereof comprises a CDRL1, a CDRL2 and a CDRL3 of a light chain variable domain (VL) having the amino acid sequence of SEQ ID NO: 10.

In some embodiments, according to the Kabat definition system, an anti-BCMA antibody or antigen binding fragment thereof comprises a CDRH3 having the amino acid sequence of SEQ ID NO: 3. In some embodiments, according to the Kabat definition system, an anti-BCMA antibody or antigen binding fragment thereof comprises a CDRH1 having the amino acid sequence of SEQ ID NO: 1, a CDRH2 having the amino acid sequence of SEQ ID NO: 2, and a CDRH3 having the amino acid sequence of SEQ ID NO: 3. In some embodiments, according to the Kabat definition system, an anti-BCMA antibody or antigen binding fragment thereof comprises a CDRH1 having the amino acid sequence of SEQ ID NO: 1, a CDRH2 having the amino acid sequence of SEQ ID NO: 2, a CDRH3 having the amino acid sequence of SEQ ID NO: 3, a CDRL1 having the amino acid sequence of SEQ ID NO: 4, a CDRL2 having the amino acid sequence of SEQ ID NO: 24, and a CDRL3 having the amino acid sequence of SEQ ID NO: 6.

In some embodiments, an anti-BCMA antibody or antigen binding fragment thereof comprises a CDRH1, a CDRH2, and a CDRH3, which collectively contains no more than 5 amino acid variations (e.g., no more than 5, 4, 3, 2, or 1 amino acid variation) as compared with the CDRH1 having the amino acid sequence of SEQ ID NO: 1, CDRH2 having the amino acid sequence of SEQ ID NO: 2, and CDRH3 having the amino acid sequence of SEQ ID NO: 3. Alternatively or in addition, an anti-BCMA antibody or antigen binding fragment thereof comprises a CDRL1, a CDRL2, and a CDRL3, which collectively contains no more than 5 amino acid variations (e.g., no more than 5, 4, 3, 2 or 1 amino acid variation) as compared with the CDRL1 having the amino acid sequence of SEQ ID NO: 4, CDRL2 having the amino acid sequence of SEQ ID NO: 24, and CDRL3 having the amino acid sequence of SEQ ID NO: 6.

In some embodiments, an anti-BCMA antibody or antigen binding fragment thereof comprises a CDRH1, a CDRH2, and a CDRH3 that collectively are at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the CDRH1 having the amino acid sequence of SEQ ID NO: 1, CDRH2 having the amino acid sequence of SEQ ID NO: 2, and CDRH3 having the amino acid sequence of SEQ ID NO: 3. Alternatively or in addition, an anti-BCMA antibody or antigen binding fragment thereof comprises a CDRL1, a CDRL2, and a CDRL3 that collectively are at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the to the CDRL1 having the amino acid sequence of SEQ ID NO: 4, CDRL2 having the amino acid sequence of SEQ ID NO: 24, and CDRL3 having the amino acid sequence of SEQ ID NO: 6.

In some embodiments, an anti-BCMA antibody or antigen binding fragment thereof comprises: a CDRH1 having no more than 3 amino acid variations (e.g., no more than 3, 2, or 1 amino acid variation) as compared with the CDRH1 having the amino acid sequence of SEQ ID NO: 1; a CDRH2 having no more than 3 amino acid variations (e.g., no more than 3, 2, or 1 amino acid variation) as compared with the CDRH2 having the amino acid sequence of SEQ ID NO: 2; and/or a CDRH3 having no more than 3 amino acid variations (e.g., no more than 3, 2, or 1 amino acid variation) as compared with the CDRH3 having the amino acid sequence of SEQ ID NO: 3. Alternatively or in addition, an anti-BCMA antibody or antigen binding fragment thereof comprises: a CDRL1 having no more than 3 amino acid variations (e.g., no more than 3, 2, or 1 amino acid variation) as compared with the CDRL1 having the amino acid sequence of SEQ ID NO: 4; a CDRL2 having no more than 3 amino acid variations (e.g., no more than 3, 2, or 1 amino acid variation) as compared with the CDRL2 having the amino acid sequence of SEQ ID NO: 24; and/or a CDRL3 having no more than 3 amino acid variations (e.g., no more than 3, 2, or 1 amino acid variation) as compared with the CDRL3 having the amino acid sequence of SEQ ID NO: 6.

In some embodiments, an anti-BCMA antibody or antigen binding fragment thereof comprises a VH comprising the amino acid sequence of SEQ ID NO: 7. Alternatively or in addition, an anti-BCMA antibody or antigen binding fragment thereof comprises a VL comprising the amino acid sequence of SEQ ID NO: 10.

In some embodiments, an anti-BCMA antibody or antigen binding fragment thereof comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 7. Alternatively or in addition, an anti-BCMA antibody or antigen binding fragment thereof comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 10.

In some embodiments, an anti-BCMA antibody or antigen binding fragment thereof comprises a VH comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VH as set forth in SEQ ID NO: 7. Alternatively or in addition, an anti-BCMA antibody or antigen binding fragment thereof comprises a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VL as set forth in SEQ ID NO: 10.

In some embodiments, an anti-BCMA antibody or antigen binding fragment thereof comprises a CDRH1, a CDRH2 and a CDRH3 of a heavy chain variable domain (VH) having the amino acid sequence of SEQ ID NO: 12. Alternatively or in addition, an anti-BCMA antibody or antigen binding fragment thereof comprises a CDRL1, a CDRL2 and a CDRL3 of a light chain variable domain (VL) having the amino acid sequence of SEQ ID NO: 13.

In some embodiments, according to the Kabat definition system, an anti-BCMA antibody or antigen binding fragment thereof comprises a CDRH3 having the amino acid sequence of SEQ ID NO: 3. In some embodiments, according to the Kabat definition system, an anti-BCMA antibody or antigen binding fragment thereof comprises a CDRH1 having the amino acid sequence of SEQ ID NO: 25, a CDRH2 having the amino acid sequence of SEQ ID NO: 27, and a CDRH3 having the amino acid sequence of SEQ ID NO: 3. In some embodiments, according to the Kabat definition system, an anti-BCMA antibody or antigen binding fragment thereof comprises a CDRH1 having the amino acid sequence of SEQ ID NO: 25, a CDRH2 having the amino acid sequence of SEQ ID NO: 27, a CDRH3 having the amino acid sequence of SEQ ID NO: 3, a CDRL1 having the amino acid sequence of SEQ ID NO: 4, a CDRL2 having the amino acid sequence of SEQ ID NO: 24, and a CDRL3 having the amino acid sequence of SEQ ID NO: 30.

In some embodiments, an anti-BCMA antibody or antigen binding fragment thereof comprises a CDRH1, a CDRH2, and a CDRH3, which collectively contains no more than 5 amino acid variations (e.g., no more than 5, 4, 3, 2, or 1 amino acid variation) as compared with the CDRH1 having the amino acid sequence of SEQ ID NO: 25, CDRH2 having the amino acid sequence of SEQ ID NO: 27, and CDRH3 having the amino acid sequence of SEQ ID NO: 3. Alternatively or in addition, an anti-BCMA antibody or antigen binding fragment thereof comprises a CDRL1, a CDRL2, and a CDRL3, which collectively contains no more than 5 amino acid variations (e.g., no more than 5, 4, 3, 2 or 1 amino acid variation) as compared with the CDRL1 having the amino acid sequence of SEQ ID NO: 4, CDRL2 having the amino acid sequence of SEQ ID NO: 24, and CDRL3 having the amino acid sequence of SEQ ID NO: 30.

In some embodiments, an anti-BCMA antibody or antigen binding fragment thereof comprises a CDRH1, a CDRH2, and a CDRH3 that collectively are at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the CDRH1 having the amino acid sequence of SEQ ID NO: 25, CDRH2 having the amino acid sequence of SEQ ID NO: 27, and CDRH3 having the amino acid sequence of SEQ ID NO: 3. Alternatively or in addition, an anti-BCMA antibody or antigen binding fragment thereof comprises a CDRL1, a CDRL2, and a CDRL3 that collectively are at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the to the CDRL1 having the amino acid sequence of SEQ ID NO: 4, CDRL2 having the amino acid sequence of SEQ ID NO: 24, and CDRL3 having the amino acid sequence of SEQ ID NO: 30.

In some embodiments, an anti-BCMA antibody or antigen binding fragment thereof comprises: a CDRH1 having no more than 3 amino acid variations (e.g., no more than 3, 2, or 1 amino acid variation) as compared with the CDRH1 having the amino acid sequence of SEQ ID NO: 25; a CDRH2 having no more than 3 amino acid variations (e.g., no more than 3, 2, or 1 amino acid variation) as compared with the CDRH2 having the amino acid sequence of SEQ ID NO: 27; and/or a CDRH3 having no more than 3 amino acid variations (e.g., no more than 3, 2, or 1 amino acid variation) as compared with the CDRH3 having the amino acid sequence of SEQ ID NO: 3. Alternatively or in addition, an anti-BCMA antibody or antigen binding fragment thereof comprises: a CDRL1 having no more than 3 amino acid variations (e.g., no more than 3, 2, or 1 amino acid variation) as compared with the CDRL1 having the amino acid sequence of SEQ ID NO: 4; a CDRL2 having no more than 3 amino acid variations (e.g., no more than 3, 2, or 1 amino acid variation) as compared with the CDRL2 having the amino acid sequence of SEQ ID NO: 24; and/or a CDRL3 having no more than 3 amino acid variations (e.g., no more than 3, 2, or 1 amino acid variation) as compared with the CDRL3 having the amino acid sequence of SEQ ID NO: 30.

In some embodiments, an anti-BCMA antibody or antigen binding fragment thereof comprises a VH comprising the amino acid sequence of SEQ ID NO: 12. Alternatively or in addition, an anti-BCMA antibody or antigen binding fragment thereof comprises a VL comprising the amino acid sequence of SEQ ID NO: 13.

In some embodiments, an anti-BCMA antibody or antigen binding fragment thereof comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 12. Alternatively or in addition, an anti-BCMA antibody or antigen binding fragment thereof comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 13.

In some embodiments, an anti-BCMA antibody or antigen binding fragment thereof comprises a VH comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VH as set forth in SEQ ID NO: 12. Alternatively or in addition, an anti-BCMA antibody or antigen binding fragment thereof comprises a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VL as set forth in SEQ ID NO: 13.

In some embodiments, an anti-BCMA antibody or antigen binding fragment thereof comprises a CDRH1, a CDRH2 and a CDRH3 of a heavy chain variable domain (VH) having the amino acid sequence of SEQ ID NO: 12. Alternatively or in addition, an anti-BCMA antibody or antigen binding fragment thereof comprises a CDRL1, a CDRL2 and a CDRL3 of a light chain variable domain (VL) having the amino acid sequence of SEQ ID NO: 15.

In some embodiments, according to the Kabat definition system, an anti-BCMA antibody or antigen binding fragment thereof comprises a CDRH3 having the amino acid sequence of SEQ ID NO: 3. In some embodiments, according to the Kabat definition system, an anti-BCMA antibody or antigen binding fragment thereof comprises a CDRH1 having the amino acid sequence of SEQ ID NO: 25, a CDRH2 having the amino acid sequence of SEQ ID NO: 27, and a CDRH3 having the amino acid sequence of SEQ ID NO: 3. In some embodiments, according to the Kabat definition system, an anti-BCMA antibody or antigen binding fragment thereof comprises a CDRH1 having the amino acid sequence of SEQ ID NO: 25, a CDRH2 having the amino acid sequence of SEQ ID NO: 27, a CDRH3 having the amino acid sequence of SEQ ID NO: 3, a CDRL1 having the amino acid sequence of SEQ ID NO: 4, a CDRL2 having the amino acid sequence of SEQ ID NO: 24, and a CDRL3 having the amino acid sequence of SEQ ID NO: 29.

In some embodiments, an anti-BCMA antibody or antigen binding fragment thereof comprises a CDRH1, a CDRH2, and a CDRH3, which collectively contains no more than 5 amino acid variations (e.g., no more than 5, 4, 3, 2, or 1 amino acid variation) as compared with the CDRH1 having the amino acid sequence of SEQ ID NO: 25, CDRH2 having the amino acid sequence of SEQ ID NO: 27, and CDRH3 having the amino acid sequence of SEQ ID NO: 3. Alternatively or in addition, an anti-BCMA antibody or antigen binding fragment thereof comprises a CDRL1, a CDRL2, and a CDRL3, which collectively contains no more than 5 amino acid variations (e.g., no more than 5, 4, 3, 2 or 1 amino acid variation) as compared with the CDRL1 having the amino acid sequence of SEQ ID NO: 4, CDRL2 having the amino acid sequence of SEQ ID NO: 24, and CDRL3 having the amino acid sequence of SEQ ID NO: 29.

In some embodiments, an anti-BCMA antibody or antigen binding fragment thereof comprises a CDRH1, a CDRH2, and a CDRH3 that collectively are at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the CDRH1 having the amino acid sequence of SEQ ID NO: 25, CDRH2 having the amino acid sequence of SEQ ID NO: 27, and CDRH3 having the amino acid sequence of SEQ ID NO: 3. Alternatively or in addition, an anti-BCMA antibody or antigen binding fragment thereof comprises a CDRL1, a CDRL2, and a CDRL3 that collectively are at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the to the CDRL1 having the amino acid sequence of SEQ ID NO: 4, CDRL2 having the amino acid sequence of SEQ ID NO: 24, and CDRL3 having the amino acid sequence of SEQ ID NO: 29.

In some embodiments, an anti-BCMA antibody or antigen binding fragment thereof comprises: a CDRH1 having no more than 3 amino acid variations (e.g., no more than 3, 2, or 1 amino acid variation) as compared with the CDRH1 having the amino acid sequence of SEQ ID NO: 25; a CDRH2 having no more than 3 amino acid variations (e.g., no more than 3, 2, or 1 amino acid variation) as compared with the CDRH2 having the amino acid sequence of SEQ ID NO: 27; and/or a CDRH3 having no more than 3 amino acid variations (e.g., no more than 3, 2, or 1 amino acid variation) as compared with the CDRH3 having the amino acid sequence of SEQ ID NO: 3. Alternatively or in addition, an anti-BCMA antibody or antigen binding fragment thereof comprises: a CDRL1 having no more than 3 amino acid variations (e.g., no more than 3, 2, or 1 amino acid variation) as compared with the CDRL1 having the amino acid sequence of SEQ ID NO: 4; a CDRL2 having no more than 3 amino acid variations (e.g., no more than 3, 2, or 1 amino acid variation) as compared with the CDRL2 having the amino acid sequence of SEQ ID NO: 24; and/or a CDRL3 having no more than 3 amino acid variations (e.g., no more than 3, 2, or 1 amino acid variation) as compared with the CDRL3 having the amino acid sequence of SEQ ID NO: 29.

In some embodiments, an anti-BCMA antibody or antigen binding fragment thereof comprises a VH comprising the amino acid sequence of SEQ ID NO: 12. Alternatively or in addition, an anti-BCMA antibody or antigen binding fragment thereof comprises a VL comprising the amino acid sequence of SEQ ID NO: 15.

In some embodiments, an anti-BCMA antibody or antigen binding fragment thereof comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 12. Alternatively or in addition, an anti-BCMA antibody or antigen binding fragment thereof comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 15.

In some embodiments, an anti-BCMA antibody or antigen binding fragment thereof comprises a VH comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VH as set forth in SEQ ID NO: 12. Alternatively or in addition, an anti-BCMA antibody or antigen binding fragment thereof comprises a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VL as set forth in SEQ ID NO: 15.

In some embodiments, an anti-BCMA antibody or antigen binding fragment thereof comprises a CDRH1, a CDRH2 and a CDRH3 of a heavy chain variable domain (VH) having the amino acid sequence of SEQ ID NO: 7. Alternatively or in addition, an anti-BCMA antibody or antigen binding fragment thereof comprises a CDRL1, a CDRL2 and a CDRL3 of a light chain variable domain (VL) having the amino acid sequence of SEQ ID NO: 13.

In some embodiments, according to the Kabat definition system, an anti-BCMA antibody or antigen binding fragment thereof comprises a CDRH3 having the amino acid sequence of SEQ ID NO: 3. In some embodiments, according to the Kabat definition system, an anti-BCMA antibody or antigen binding fragment thereof comprises a CDRH1 having the amino acid sequence of SEQ ID NO: 1, a CDRH2 having the amino acid sequence of SEQ ID NO: 2, and a CDRH3 having the amino acid sequence of SEQ ID NO: 3. In some embodiments, according to the Kabat definition system, an anti-BCMA antibody or antigen binding fragment thereof comprises a CDRH1 having the amino acid sequence of SEQ ID NO: 1, a CDRH2 having the amino acid sequence of SEQ ID NO: 2, a CDRH3 having the amino acid sequence of SEQ ID NO: 3, a CDRL1 having the amino acid sequence of SEQ ID NO: 4, a CDRL2 having the amino acid sequence of SEQ ID NO: 24, and a CDRL3 having the amino acid sequence of SEQ ID NO: 30.

In some embodiments, an anti-BCMA antibody or antigen binding fragment thereof comprises a CDRH1, a CDRH2, and a CDRH3, which collectively contains no more than 5 amino acid variations (e.g., no more than 5, 4, 3, 2, or 1 amino acid variation) as compared with the CDRH1 having the amino acid sequence of SEQ ID NO: 1, CDRH2 having the amino acid sequence of SEQ ID NO: 2, and CDRH3 having the amino acid sequence of SEQ ID NO: 3. Alternatively or in addition, an anti-BCMA antibody or antigen binding fragment thereof comprises a CDRL1, a CDRL2, and a CDRL3, which collectively contains no more than 5 amino acid variations (e.g., no more than 5, 4, 3, 2 or 1 amino acid variation) as compared with the CDRL1 having the amino acid sequence of SEQ ID NO: 4, CDRL2 having the amino acid sequence of SEQ ID NO: 24, and CDRL3 having the amino acid sequence of SEQ ID NO: 30.

In some embodiments, an anti-BCMA antibody or antigen binding fragment thereof comprises a CDRH1, a CDRH2, and a CDRH3 that collectively are at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the CDRH1 having the amino acid sequence of SEQ ID NO: 1, CDRH2 having the amino acid sequence of SEQ ID NO: 2, and CDRH3 having the amino acid sequence of SEQ ID NO: 3. Alternatively or in addition, an anti-BCMA antibody or antigen binding fragment thereof comprises a CDRL1, a CDRL2, and a CDRL3 that collectively are at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the to the CDRL1 having the amino acid sequence of SEQ ID NO: 4, CDRL2 having the amino acid sequence of SEQ ID NO: 24, and CDRL3 having the amino acid sequence of SEQ ID NO: 30.

In some embodiments, an anti-BCMA antibody or antigen binding fragment thereof comprises: a CDRH1 having no more than 3 amino acid variations (e.g., no more than 3, 2, or 1 amino acid variation) as compared with the CDRH1 having the amino acid sequence of SEQ ID NO: 1; a CDRH2 having no more than 3 amino acid variations (e.g., no more than 3, 2, or 1 amino acid variation) as compared with the CDRH2 having the amino acid sequence of SEQ ID NO: 2; and/or a CDRH3 having no more than 3 amino acid variations (e.g., no more than 3, 2, or 1 amino acid variation) as compared with the CDRH3 having the amino acid sequence of SEQ ID NO: 3. Alternatively or in addition, an anti-BCMA antibody or antigen binding fragment thereof comprises: a CDRL1 having no more than 3 amino acid variations (e.g., no more than 3, 2, or 1 amino acid variation) as compared with the CDRL1 having the amino acid sequence of SEQ ID NO: 4; a CDRL2 having no more than 3 amino acid variations (e.g., no more than 3, 2, or 1 amino acid variation) as compared with the CDRL2 having the amino acid sequence of SEQ ID NO: 24; and/or a CDRL3 having no more than 3 amino acid variations (e.g., no more than 3, 2, or 1 amino acid variation) as compared with the CDRL3 having the amino acid sequence of SEQ ID NO: 30.

In some embodiments, an anti-BCMA antibody or antigen binding fragment thereof comprises a VH comprising the amino acid sequence of SEQ ID NO: 7. Alternatively or in addition, an anti-BCMA antibody or antigen binding fragment thereof comprises a VL comprising the amino acid sequence of SEQ ID NO: 13.

In some embodiments, an anti-BCMA antibody or antigen binding fragment thereof comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 7. Alternatively or in addition, an anti-BCMA antibody or antigen binding fragment thereof comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 13.

In some embodiments, an anti-BCMA antibody or antigen binding fragment thereof comprises a VH comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VH as set forth in SEQ ID NO: 7. Alternatively or in addition, an anti-BCMA antibody or antigen binding fragment thereof comprises a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VL as set forth in SEQ ID NO: 13.

In some embodiments, an anti-BCMA antibody or antigen binding fragment thereof comprises a CDRH1, a CDRH2 and a CDRH3 of a heavy chain variable domain (VH) having the amino acid sequence of SEQ ID NO: 7. Alternatively or in addition, an anti-BCMA antibody or antigen binding fragment thereof comprises a CDRL1, a CDRL2 and a CDRL3 of a light chain variable domain (VL) having the amino acid sequence of SEQ ID NO: 15.

In some embodiments, according to the Kabat definition system, an anti-BCMA antibody or antigen binding fragment thereof comprises a CDRH3 having the amino acid sequence of SEQ ID NO: 3. In some embodiments, according to the Kabat definition system, an anti-BCMA antibody or antigen binding fragment thereof comprises a CDRH1 having the amino acid sequence of SEQ ID NO: 1, a CDRH2 having the amino acid sequence of SEQ ID NO: 2, and a CDRH3 having the amino acid sequence of SEQ ID NO: 3. In some embodiments, according to the Kabat definition system, an anti-BCMA antibody or antigen binding fragment thereof comprises a CDRH1 having the amino acid sequence of SEQ ID NO: 1, a CDRH2 having the amino acid sequence of SEQ ID NO: 2, a CDRH3 having the amino acid sequence of SEQ ID NO: 3, a CDRL1 having the amino acid sequence of SEQ ID NO: 4, a CDRL2 having the amino acid sequence of SEQ ID NO: 24, and a CDRL3 having the amino acid sequence of SEQ ID NO: 29.

In some embodiments, an anti-BCMA antibody or antigen binding fragment thereof comprises a CDRH1, a CDRH2, and a CDRH3, which collectively contains no more than 5 amino acid variations (e.g., no more than 5, 4, 3, 2, or 1 amino acid variation) as compared with the CDRH1 having the amino acid sequence of SEQ ID NO: 1, CDRH2 having the amino acid sequence of SEQ ID NO: 2, and CDRH3 having the amino acid sequence of SEQ ID NO: 3. Alternatively or in addition, an anti-BCMA antibody or antigen binding fragment thereof comprises a CDRL1, a CDRL2, and a CDRL3, which collectively contains no more than 5 amino acid variations (e.g., no more than 5, 4, 3, 2 or 1 amino acid variation) as compared with the CDRL1 having the amino acid sequence of SEQ ID NO: 4, CDRL2 having the amino acid sequence of SEQ ID NO: 24, and CDRL3 having the amino acid sequence of SEQ ID NO: 29.

In some embodiments, an anti-BCMA antibody or antigen binding fragment thereof comprises a CDRH1, a CDRH2, and a CDRH3 that collectively are at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the CDRH1 having the amino acid sequence of SEQ ID NO: 1, CDRH2 having the amino acid sequence of SEQ ID NO: 2, and CDRH3 having the amino acid sequence of SEQ ID NO: 3. Alternatively or in addition, an anti-BCMA antibody or antigen binding fragment thereof comprises a CDRL1, a CDRL2, and a CDRL3 that collectively are at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the to the CDRL1 having the amino acid sequence of SEQ ID NO: 4, CDRL2 having the amino acid sequence of SEQ ID NO: 24, and CDRL3 having the amino acid sequence of SEQ ID NO: 29.

In some embodiments, an anti-BCMA antibody or antigen binding fragment thereof comprises: a CDRH1 having no more than 3 amino acid variations (e.g., no more than 3, 2, or 1 amino acid variation) as compared with the CDRH1 having the amino acid sequence of SEQ ID NO: 1; a CDRH2 having no more than 3 amino acid variations (e.g., no more than 3, 2, or 1 amino acid variation) as compared with the CDRH2 having the amino acid sequence of SEQ ID NO: 2; and/or a CDRH3 having no more than 3 amino acid variations (e.g., no more than 3, 2, or 1 amino acid variation) as compared with the CDRH3 having the amino acid sequence of SEQ ID NO: 3. Alternatively or in addition, an anti-BCMA antibody or antigen binding fragment thereof comprises: a CDRL1 having no more than 3 amino acid variations (e.g., no more than 3, 2, or 1 amino acid variation) as compared with the CDRL1 having the amino acid sequence of SEQ ID NO: 4; a CDRL2 having no more than 3 amino acid variations (e.g., no more than 3, 2, or 1 amino acid variation) as compared with the CDRL2 having the amino acid sequence of SEQ ID NO: 24; and/or a CDRL3 having no more than 3 amino acid variations (e.g., no more than 3, 2, or 1 amino acid variation) as compared with the CDRL3 having the amino acid sequence of SEQ ID NO: 29.

In some embodiments, an anti-BCMA antibody or antigen binding fragment thereof comprises a VH comprising the amino acid sequence of SEQ ID NO: 7. Alternatively or in addition, an anti-BCMA antibody or antigen binding fragment thereof comprises a VL comprising the amino acid sequence of SEQ ID NO: 15.

In some embodiments, an anti-BCMA antibody or antigen binding fragment thereof comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 7. Alternatively or in addition, an anti-BCMA antibody or antigen binding fragment thereof comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 15.

In some embodiments, an anti-BCMA antibody or antigen binding fragment thereof comprises a VH comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VH as set forth in SEQ ID NO: 7. Alternatively or in addition, an anti-BCMA antibody or antigen binding fragment thereof comprises a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VL as set forth in SEQ ID NO: 15.

In some embodiments, an anti-BCMA antibody or antigen binding fragment thereof comprises a CDRH1, a CDRH2 and a CDRH3 of a heavy chain variable domain (VH) having the amino acid sequence of SEQ ID NO: 19. Alternatively or in addition, an anti-BCMA antibody or antigen binding fragment thereof comprises a CDRL1, a CDRL2 and a CDRL3 of a light chain variable domain (VL) having the amino acid sequence of SEQ ID NO: 10.

In some embodiments, according to the Kabat definition system, an anti-BCMA antibody or antigen binding fragment thereof comprises a CDRH3 having the amino acid sequence of SEQ ID NO: 3. In some embodiments, according to the Kabat definition system, an anti-BCMA antibody or antigen binding fragment thereof comprises a CDRH1 having the amino acid sequence of SEQ ID NO: 26, a CDRH2 having the amino acid sequence of SEQ ID NO: 28, and a CDRH3 having the amino acid sequence of SEQ ID NO: 3. In some embodiments, according to the Kabat definition system, an anti-BCMA antibody or antigen binding fragment thereof comprises a CDRH1 having the amino acid sequence of SEQ ID NO: 26, a CDRH2 having the amino acid sequence of SEQ ID NO: 28, a CDRH3 having the amino acid sequence of SEQ ID NO: 3, a CDRL1 having the amino acid sequence of SEQ ID NO: 4, a CDRL2 having the amino acid sequence of SEQ ID NO: 24, and a CDRL3 having the amino acid sequence of SEQ ID NO: 6.

In some embodiments, an anti-BCMA antibody or antigen binding fragment thereof comprises a CDRH1, a CDRH2, and a CDRH3, which collectively contains no more than 5 amino acid variations (e.g., no more than 5, 4, 3, 2, or 1 amino acid variation) as compared with the CDRH1 having the amino acid sequence of SEQ ID NO: 26, CDRH2 having the amino acid sequence of SEQ ID NO: 28, and CDRH3 having the amino acid sequence of SEQ ID NO: 3. Alternatively or in addition, an anti-BCMA antibody or antigen binding fragment thereof comprises a CDRL1, a CDRL2, and a CDRL3, which collectively contains no more than 5 amino acid variations (e.g., no more than 5, 4, 3, 2 or 1 amino acid variation) as compared with the CDRL1 having the amino acid sequence of SEQ ID NO: 4, CDRL2 having the amino acid sequence of SEQ ID NO: 24, and CDRL3 having the amino acid sequence of SEQ ID NO: 6.

In some embodiments, an anti-BCMA antibody or antigen binding fragment thereof comprises a CDRH1, a CDRH2, and a CDRH3 that collectively are at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the CDRH1 having the amino acid sequence of SEQ ID NO: 26, CDRH2 having the amino acid sequence of SEQ ID NO: 28, and CDRH3 having the amino acid sequence of SEQ ID NO: 3. Alternatively or in addition, an anti-BCMA antibody or antigen binding fragment thereof comprises a CDRL1, a CDRL2, and a CDRL3 that collectively are at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the to the CDRL1 having the amino acid sequence of SEQ ID NO: 4, CDRL2 having the amino acid sequence of SEQ ID NO: 24, and CDRL3 having the amino acid sequence of SEQ ID NO: 6.

In some embodiments, an anti-BCMA antibody or antigen binding fragment thereof comprises: a CDRH1 having no more than 3 amino acid variations (e.g., no more than 3, 2, or 1 amino acid variation) as compared with the CDRH1 having the amino acid sequence of SEQ ID NO: 26; a CDRH2 having no more than 3 amino acid variations (e.g., no more than 3, 2, or 1 amino acid variation) as compared with the CDRH2 having the amino acid sequence of SEQ ID NO: 28; and/or a CDRH3 having no more than 3 amino acid variations (e.g., no more than 3, 2, or 1 amino acid variation) as compared with the CDRH3 having the amino acid sequence of SEQ ID NO: 3. Alternatively or in addition, an anti-BCMA antibody or antigen binding fragment thereof comprises: a CDRL1 having no more than 3 amino acid variations (e.g., no more than 3, 2, or 1 amino acid variation) as compared with the CDRL1 having the amino acid sequence of SEQ ID NO: 4; a CDRL2 having no more than 3 amino acid variations (e.g., no more than 3, 2, or 1 amino acid variation) as compared with the CDRL2 having the amino acid sequence of SEQ ID NO: 24; and/or a CDRL3 having no more than 3 amino acid variations (e.g., no more than 3, 2, or 1 amino acid variation) as compared with the CDRL3 having the amino acid sequence of SEQ ID NO: 6.

In some embodiments, an anti-BCMA antibody or antigen binding fragment thereof comprises a VH comprising the amino acid sequence of SEQ ID NO: 19. Alternatively or in addition, an anti-BCMA antibody or antigen binding fragment thereof comprises a VL comprising the amino acid sequence of SEQ ID NO: 10.

In some embodiments, an anti-BCMA antibody or antigen binding fragment thereof comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 19. Alternatively or in addition, an anti-BCMA antibody or antigen binding fragment thereof comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 10.

In some embodiments, an anti-BCMA antibody or antigen binding fragment thereof comprises a VH comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VH as set forth in SEQ ID NO: 19. Alternatively or in addition, an anti-BCMA antibody or antigen binding fragment thereof comprises a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VL as set forth in SEQ ID NO: 10.

In some embodiments, an anti-BCMA antibody or antigen binding fragment thereof comprises a CDRH1, a CDRH2 and a CDRH3 of a heavy chain variable domain (VH) having the amino acid sequence of SEQ ID NO: 21. Alternatively or in addition, an anti-BCMA antibody or antigen binding fragment thereof comprises a CDRL1, a CDRL2 and a CDRL3 of a light chain variable domain (VL) having the amino acid sequence of SEQ ID NO: 22.

In some embodiments, according to the Kabat definition system, an anti-BCMA antibody or antigen binding fragment thereof comprises a CDRH3 having the amino acid sequence of SEQ ID NO: 33. In some embodiments, according to the Kabat definition system, an anti-BCMA antibody or antigen binding fragment thereof comprises a CDRH1 having the amino acid sequence of SEQ ID NO: 31, a CDRH2 having the amino acid sequence of SEQ ID NO: 32, and a CDRH3 having the amino acid sequence of SEQ ID NO: 33. In some embodiments, according to the Kabat definition system, an anti-BCMA antibody or antigen binding fragment thereof comprises a CDRH1 having the amino acid sequence of SEQ ID NO: 31, a CDRH2 having the amino acid sequence of SEQ ID NO: 32, a CDRH3 having the amino acid sequence of SEQ ID NO: 33, a CDRL1 having the amino acid sequence of SEQ ID NO: 34, a CDRL2 having the amino acid sequence of SEQ ID NO: 35, and a CDRL3 having the amino acid sequence of SEQ ID NO: 36.

In some embodiments, an anti-BCMA antibody or antigen binding fragment thereof comprises a CDRH1, a CDRH2, and a CDRH3, which collectively contains no more than 5 amino acid variations (e.g., no more than 5, 4, 3, 2, or 1 amino acid variation) as compared with the CDRH1 having the amino acid sequence of SEQ ID NO: 31, CDRH2 having the amino acid sequence of SEQ ID NO: 32, and CDRH3 having the amino acid sequence of SEQ ID NO: 33. Alternatively or in addition, an anti-BCMA antibody or antigen binding fragment thereof comprises a CDRL1, a CDRL2, and a CDRL3, which collectively contains no more than 5 amino acid variations (e.g., no more than 5, 4, 3, 2 or 1 amino acid variation) as compared with the CDRL1 having the amino acid sequence of SEQ ID NO: 34, CDRL2 having the amino acid sequence of SEQ ID NO: 35, and CDRL3 having the amino acid sequence of SEQ ID NO: 36.

In some embodiments, an anti-BCMA antibody or antigen binding fragment thereof comprises a CDRH1, a CDRH2, and a CDRH3 that collectively are at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the CDRH1 having the amino acid sequence of SEQ ID NO: 31, CDRH2 having the amino acid sequence of SEQ ID NO: 32, and CDRH3 having the amino acid sequence of SEQ ID NO: 33. Alternatively or in addition, an anti-BCMA antibody or antigen binding fragment thereof comprises a CDRL1, a CDRL2, and a CDRL3 that collectively are at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the to the CDRL1 having the amino acid sequence of SEQ ID NO: 34, CDRL2 having the amino acid sequence of SEQ ID NO: 35, and CDRL3 having the amino acid sequence of SEQ ID NO: 36.

In some embodiments, an anti-BCMA antibody or antigen binding fragment thereof comprises: a CDRH1 having no more than 3 amino acid variations (e.g., no more than 3, 2, or 1 amino acid variation) as compared with the CDRH1 having the amino acid sequence of SEQ ID NO: 31; a CDRH2 having no more than 3 amino acid variations (e.g., no more than 3, 2, or 1 amino acid variation) as compared with the CDRH2 having the amino acid sequence of SEQ ID NO: 32; and/or a CDRH3 having no more than 3 amino acid variations (e.g., no more than 3, 2, or 1 amino acid variation) as compared with the CDRH3 having the amino acid sequence of SEQ ID NO: 33. Alternatively or in addition, an anti-BCMA antibody or antigen binding fragment thereof comprises: a CDRL1 having no more than 3 amino acid variations (e.g., no more than 3, 2, or 1 amino acid variation) as compared with the CDRL1 having the amino acid sequence of SEQ ID NO: 34; a CDRL2 having no more than 3 amino acid variations (e.g., no more than 3, 2, or 1 amino acid variation) as compared with the CDRL2 having the amino acid sequence of SEQ ID NO: 35; and/or a CDRL3 having no more than 3 amino acid variations (e.g., no more than 3, 2, or 1 amino acid variation) as compared with the CDRL3 having the amino acid sequence of SEQ ID NO: 36.

In some embodiments, an anti-BCMA antibody or antigen binding fragment thereof comprises a VH comprising the amino acid sequence of SEQ ID NO: 21. Alternatively or in addition, an anti-BCMA antibody or antigen binding fragment thereof comprises a VL comprising the amino acid sequence of SEQ ID NO: 22.

In some embodiments, an anti-BCMA antibody or antigen binding fragment thereof comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 21. Alternatively or in addition, an anti-BCMA antibody or antigen binding fragment thereof comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 22.

In some embodiments, an anti-BCMA antibody or antigen binding fragment thereof comprises a VH comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VH as set forth in SEQ ID NO: 21. Alternatively or in addition, an anti-BCMA antibody or antigen binding fragment thereof comprises a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VL as set forth in SEQ ID NO: 22.

An anti-BCMA antibody or antigen binding fragment thereof described herein can be in any antibody form, including, but not limited to, intact (i.e., full-length) antibodies, antigen-binding fragments thereof (such as Fab, F(ab′), F(ab′)2, Fv), single chain antibodies (e.g., scFv), bi-specific antibodies, or nanobodies. In some embodiments, an anti-BCMA antibody or antigen binding fragment thereof described herein is a single-chain variable fragment (scFv). In some embodiments, an anti-BCMA antibody or antigen binding fragment thereof described herein is a scFv-Fab (e.g., scFv fused to a portion of a constant region).

In some embodiments, an anti-BCMA antibody or antigen binding fragment thereof comprises a VL domain and/or VH domain of any one of the anti-BCMA antibodies selected from Table 3, and comprises a constant region comprising the amino acid sequences of the constant regions of an IgG, IgE, IgM, IgD, IgA or IgY immunoglobulin molecule, any class (e.g., IgG1, IgG2, IgG3, IgG4, IgA1 and IgA2), or any subclass (e.g., IgG2a and IgG2b) of immunoglobulin molecule. Non-limiting examples of human constant regions are described in the art, e.g., see Kabat E A et al., (1991) supra. Other antibody heavy and light chain constant regions are well known in the art, e.g., those provided in the IMGT database (imgt.org) or at vbase2.org/vbstat.php., both of which are incorporated by reference herein.

In some embodiments, an anti-BCMA antibody or antigen binding fragment thereof is a single-chain variable fragment (scFv). In some embodiments, an anti-BCMA scFv comprises a VH and a VL of any one of the anti-BCMA antibodies selected from Table 3. In some embodiments, the VH and the VL of an anti-BCMA scFv are joined together by a linker. In some embodiments, a linker may have a length of about 2 to 10 amino acids, 5 to 20 amino acids, 10 to 30 amino acids, 20-50 amino acids, 40 to 60 amino acids, 60 to 80 amino acids, or more than 80 amino acids. In some embodiment, a linker may include a sequence that substantially comprises glycine and serine. An exemplary linker sequence is GGGGSGGGGSGGGAS (SEQ ID NO: 81). In some embodiments, a linker may include, without limitation, any of those encompassed by U.S. Pat. Nos. 8,445,251 and 9,434,931. In some embodiments, an anti-BCMA comprises a linker between the VH and the VL, and the linker comprises an amino acid sequence at least 80%, at least 85%, at least 90%, at least 92%, at least 95%, at least 98%, at least 99%, or 100% identical to SEQ ID NO: 81.

In some embodiments, an anti-BCMA scFv comprises a VH and a VL, and the C-terminus of the VH is joined with the N terminus of the VL via a linker (e.g., the linker as set forth in SEQ ID NO: 81). In some embodiments, an anti-BCMA scFv comprises a VH and a VL, and the C-terminus of the VL is joined with the N terminus of the VH via a linker (e.g., the linker as set forth in SEQ ID NO: 81).

In some embodiments, an anti-BCMA scFv comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH of any of the anti-BCMA antibodies listed in Table 3. Alternatively or in addition, an anti-BCMA scFv comprises a VL containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH of any of the anti-BCMA antibodies listed in Table 3. In some embodiments, an anti-BCMA scFv comprises a VH comprising an amino acid sequence that is at least 80% (e.g., 80%, 85%, 90%, 95%, 98%, or 99%) identical to the VH of any of the anti-BCMA antibodies listed in Table 3. Alternatively or in addition, an anti-BCMA scFv comprises a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VH of any of the anti-BCMA antibodies listed in Table 3.

In some embodiments, an anti-BCMA scFv comprises an amino acid sequence containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the any of the anti-BCMA scFv listed in Table 3. In some embodiments, an anti-BCMA scFv comprises an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to any of the anti-BCMA scFv listed in Table 3.

In some embodiments, an anti-BCMA scFv comprises a VH comprising the amino acid sequence of SEQ ID NO: 7, and/or a VL comprising the amino acid sequence of SEQ ID NO: 8. In some embodiments, an anti-BCMA scFv comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 7, and/or a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 8. In some embodiments, an anti-BCMA scFv comprises a VH comprising an amino acid sequence that is at least 80% (e.g., 80%, 85%, 90%, 95%, 98%, or 99%) identical to the VH as set forth in SEQ ID NO: 7, and/or a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VL as set forth in SEQ ID NO: 8.

In some embodiments, an anti-BCMA scFv comprises an amino acid sequence containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the scFv amino acid sequence as set forth in SEQ ID NO: 9. In some embodiments, an anti-BCMA scFv comprises an amino acid sequence that is at least 80% ((e.g., at least 80%, at least 85%, at least 90%, at least 91%, at least 92%, at least 93%, at least 94%, at least 95%, at least 96%, at least 97%, at least 98%, at least 99%, or 100%) identical to the scFv amino acid sequence as set forth in SEQ ID NO: 9. In some embodiments, an anti-BCMA scFv comprises an amino acid sequence of SEQ ID NO: 9.

In some embodiments, an anti-BCMA scFv comprises a VH comprising the amino acid sequence of SEQ ID NO: 7, and/or a VL comprising the amino acid sequence of SEQ ID NO: 10. In some embodiments, an anti-BCMA scFv comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 7, and/or a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 10. In some embodiments, an anti-BCMA scFv comprises a VH comprising an amino acid sequence that is at least 80% (e.g., 80%, 85%, 90%, 95%, 98%, or 99%) identical to the VH as set forth in SEQ ID NO: 7, and/or a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VL as set forth in SEQ ID NO: 10.

In some embodiments, an anti-BCMA scFv comprises an amino acid sequence containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the scFv amino acid sequence as set forth in SEQ ID NO: 11. In some embodiments, an anti-BCMA scFv comprises an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the scFv amino acid sequence as set forth in SEQ ID NO: 11. In some embodiments, an anti-BCMA scFv comprises an amino acid sequence of SEQ ID NO: 11.

In some embodiments, an anti-BCMA scFv comprises a VH comprising the amino acid sequence of SEQ ID NO: 12, and/or a VL comprising the amino acid sequence of SEQ ID NO: 13. In some embodiments, an anti-BCMA scFv comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 12, and/or a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 13. In some embodiments, an anti-BCMA scFv comprises a VH comprising an amino acid sequence that is at least 80% (e.g., 80%, 85%, 90%, 95%, 98%, or 99%) identical to the VH as set forth in SEQ ID NO: 12, and/or a VL comprising an amino acid sequence that is at least 80 (e.g., at least 80%, at least 85%, at least 90%, at least 91%, at least 92%, at least 93%, at least 94%, at least 95%, at least 96%, at least 97%, at least 98%, at least 99%, or 100%) identical to the VL as set forth in SEQ ID NO: 13.

In some embodiments, an anti-BCMA scFv comprises an amino acid sequence containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the scFv amino acid sequence as set forth in SEQ ID NO: 14. In some embodiments, an anti-BCMA scFv comprises an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the scFv amino acid sequence as set forth in SEQ ID NO: 14. In some embodiments, an anti-BCMA scFv comprises an amino acid sequence of SEQ ID NO: 14.

In some embodiments, an anti-BCMA scFv comprises a VH comprising the amino acid sequence of SEQ ID NO: 12, and/or a VL comprising the amino acid sequence of SEQ ID NO: 15. In some embodiments, an anti-BCMA scFv comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 12, and/or a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 15. In some embodiments, an anti-BCMA scFv comprises a VH comprising an amino acid sequence that is at least 80% (e.g., 80%, 85%, 90%, 95%, 98%, or 99%) identical to the VH as set forth in SEQ ID NO: 12, and/or a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VL as set forth in SEQ ID NO: 15.

In some embodiments, an anti-BCMA scFv comprises an amino acid sequence containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the scFv amino acid sequence as set forth in SEQ ID NO: 16. In some embodiments, an anti-BCMA scFv comprises an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the scFv amino acid sequence as set forth in SEQ ID NO: 16. In some embodiments, an anti-BCMA scFv comprises an amino acid sequence of SEQ ID NO: 16.

In some embodiments, an anti-BCMA scFv comprises a VH comprising the amino acid sequence of SEQ ID NO: 7, and/or a VL comprising the amino acid sequence of SEQ ID NO: 13. In some embodiments, an anti-BCMA scFv comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 7, and/or a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 13. In some embodiments, an anti-BCMA scFv comprises a VH comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VH as set forth in SEQ ID NO: 7, and/or a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VL as set forth in SEQ ID NO: 13.

In some embodiments, an anti-BCMA scFv comprises an amino acid sequence containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the scFv amino acid sequence as set forth in SEQ ID NO: 17. In some embodiments, an anti-BCMA scFv comprises an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the scFv amino acid sequence as set forth in SEQ ID NO: 17. In some embodiments, an anti-BCMA scFv comprises an amino acid sequence of SEQ ID NO: 17.

In some embodiments, an anti-BCMA scFv comprises a VH comprising the amino acid sequence of SEQ ID NO: 7, and/or a VL comprising the amino acid sequence of SEQ ID NO: 15. In some embodiments, an anti-BCMA scFv comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 7, and/or a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 15. In some embodiments, an anti-BCMA scFv comprises a VH comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VH as set forth in SEQ ID NO: 7, and/or a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VL as set forth in SEQ ID NO: 15.

In some embodiments, an anti-BCMA scFv comprises an amino acid sequence containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the scFv amino acid sequence as set forth in SEQ ID NO: 18. In some embodiments, an anti-BCMA scFv comprises an amino acid sequence that is at least 80 (e.g., at least 80%, at least 85%, at least 90%, at least 91%, at least 92%, at least 93%, at least 94%, at least 95%, at least 96%, at least 97%, at least 98%, at least 99%, or 100%) identical to the scFv amino acid sequence as set forth in SEQ ID NO: 18. In some embodiments, an anti-BCMA scFv comprises an amino acid sequence of SEQ ID NO: 18.

In some embodiments, an anti-BCMA scFv comprises a VH comprising the amino acid sequence of SEQ ID NO: 19, and/or a VL comprising the amino acid sequence of SEQ ID NO: 10. In some embodiments, an anti-BCMA scFv comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 19, and/or a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 10. In some embodiments, an anti-BCMA scFv comprises a VH comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VH as set forth in SEQ ID NO: 19, and/or a VL comprising an amino acid sequence that is at least 80% (e.g., 80%, 85%, 90%, 95%, 98%, or 99%) identical to the VL as set forth in SEQ ID NO: 10.

In some embodiments, an anti-BCMA scFv comprises an amino acid sequence containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the scFv amino acid sequence as set forth in SEQ ID NO: 20. In some embodiments, an anti-BCMA scFv comprises an amino acid sequence that is at least 80% (e.g., 80%, 85%, 90%, 95%, 98%, or 99%) identical to the scFv amino acid sequence as set forth in SEQ ID NO: 20. In some embodiments, an anti-BCMA scFv comprises an amino acid sequence of SEQ ID NO: 20.

In some embodiments, an anti-BCMA scFv comprises a VH comprising the amino acid sequence of SEQ ID NO: 21, and/or a VL comprising the amino acid sequence of SEQ ID NO: 22. In some embodiments, an anti-BCMA scFv comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 21, and/or a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 22. In some embodiments, an anti-BCMA scFv comprises a VH comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VH as set forth in SEQ ID NO: 21, and/or a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VL as set forth in SEQ ID NO: 22.

In some embodiments, an anti-BCMA scFv comprises an amino acid sequence containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the scFv amino acid sequence as set forth in SEQ ID NO: 23. In some embodiments, an anti-BCMA scFv comprises an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the scFv amino acid sequence as set forth in SEQ ID NO: 23. In some embodiments, an anti-BCMA scFv comprises an amino acid sequence of SEQ ID NO: 23.

In some embodiments, any of the anti-BCMA antibody or antigen binding fragment described herein is modified, e.g., modified via glycosylation, phosphorylation, sumoylation, and/or methylation. In some embodiments, the anti-BCMA antibody or antigen binding fragment is a glycosylated antibody, which is conjugated to one or more sugar or carbohydrate molecules. In some embodiments, the one or more sugar or carbohydrate molecule are conjugated to the antibody via N-glycosylation, O-glycosylation, C-glycosylation, glypiation (GPI anchor attachment), and/or phosphoglycosylation. In some embodiments, the one or more sugar or carbohydrate molecules are monosaccharides, disaccharides, oligosaccharides, or glycans. In some embodiments, the one or more sugar or carbohydrate molecule is a branched oligosaccharide or a branched glycan. In some embodiments, the one or more sugar or carbohydrate molecule includes a mannose unit, a glucose unit, an N-acetylglucosamine unit, an N-acetylgalactosamine unit, a galactose unit, a fucose unit, or a phospholipid unit. In some embodiments, there are about 1-10, about 1-5, about 5-10, about 1-4, about 1-3, or about 2 sugar molecules. In some embodiments, a glycosylated antibody is fully or partially glycosylated. In some embodiments, an antibody is glycosylated by chemical reactions or by enzymatic means. In some embodiments, an antibody is glycosylated in vitro or inside a cell, which may optionally be deficient in an enzyme in the N- or O-glycosylation pathway, e.g. a glycosyltransferase. In some embodiments, an antibody is functionalized with sugar or carbohydrate molecules as described in International Patent Application Publication WO2014065661, published on May 1, 2014, entitled, “Modified antibody, antibody-conjugate and process for the preparation thereof”.

In some embodiments, conservative mutations can be introduced into antibody sequences (e.g., CDRs or framework sequences) at positions where the residues are not likely to be involved in interacting with a target antigen (e.g., BCMA), for example, as determined based on a crystal structure.

In some embodiments, any one of the anti-BCMA antibody or antigen binding fragment described herein may comprise a signal peptide in the heavy and/or light chain sequence (e.g., a N-terminal signal peptide). In some embodiments, the anti-BCMA antibody or antigen binding fragment described herein comprises any one of the VH and VL sequences, any one of the IgG heavy chain and light chain sequences, or any one of the scFv sequences described herein (e.g., any one of the scFv as set forth in SEQ ID NOs: 9, 11, 14, 16, 17, 18, 20, or 23) and further comprises a signal peptide (e.g., a N-terminal signal peptide).

### II. Anti-BCMA Chimeric Antigen Receptor

The present disclosure, at least in part, also provides chimeric antigen receptors (CARs) comprising extracellular ligand-binding domain. In some embodiments, the choice of ligand-binding domain depends upon the type and number of ligands that define the surface of a target cell. For example, the ligand-binding domain may be chosen to recognize one or more ligands that act as a cell surface marker on target cells associated with a particular disease state. Thus, examples of cell surface markers that may act as ligands for the ligand-binding domain in the CAR of the present disclosure can include those associated with viral, bacterial and parasitic infections, autoimmune disease, and, more preferably, cancer cells. In some embodiments, a CAR of the present disclosure is engineered to target one or more tumor antigens of interest by way of engineering a desired ligand-binding moiety that specifically binds to one or more antigens on a tumor cell. In the context of the present disclosure, “tumor antigen” refers to an antigen that is common to or characterizes specific hyperproliferative disorders such as cancer. Generally, a CAR (e.g., anti-BCMA CAR) of the present disclosure will comprise at least an extracellular domain and an intracellular domain. In some embodiments, the extracellular domain comprises a target-specific binding element (e.g., a scFv that specifically binds to BCMA) otherwise referred to herein as a ligand-binding domain (also referred to herein as an antigen-binding domain). In some embodiments, the extracellular domain is an antigen-binding domain or a portion thereof. In some embodiments, the extracellular ligand-binding domain is a Fab. In some embodiments, the extracellular ligand-binding domain is a scFv. In some embodiments, an extracellular ligand-binding domain of a CAR described herein comprises an antigen binding fragment that specifically binds to BCMA (e.g., human BCMA). In some embodiments, an extracellular ligand-binding domain of a CAR described herein comprises any one of the BCMA antibodies or antigen binding fragments thereof (e.g., anti-BCMA scFv).

In some embodiments, an anti-BCMA CAR of the present disclosure comprises an extracellular ligand-binding domain specifically binds BCMA with binding affinity (e.g., as indicated by K_{D}) of at least about 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, 10⁻¹² M, 10⁻¹³ M, or less. For example, an anti-BCMA CAR of the present disclosure can bind to a BCMA protein (e.g., human BCMA epitope as set forth in any one of SEQ ID NOs: 38-48) with an affinity between 5 pM and 500 nM, between 10 pM and 450 nM, between 20 pM and 400 nM, between 30 pM and 350 nM, between 40 pM and 300 nM, between 50 pM and 250 nM, between 60 pM and 200 nM, between 70 pM and 150 nM, between 80 pM and 100 nM, between 80 pM and 90 nM, between 90 pM and 80 nM, between 100 pM and 70 nM, between 200 pM and 60 nM, between 300 pM and 50 nM, between 400 pM and 40 nM, between 500 pM and 30 nM, between 600 pM and 20 nM, between 700 pM and 10 nM, between 800 pM and 5 nM, or between 900 pM and 2 nM.

The disclosure also includes CARs that compete with any of the CARs described herein for binding to a BCMA protein (e.g., human cell surface BCMA epitope as set forth in any one of SEQ ID NOs: 38-48) and that have an affinity of 100 nM or lower (e.g., 80 nM or lower, 50 nM or lower, 20 nM or lower, 10 nM or lower, 500 pM or lower, 50 pM or lower, or 5 pM or lower). The affinity and binding kinetics of the anti-BCMA CARs can be tested using any suitable method including but not limited to biosensor technology (e.g., OCTET or BIACORE). In some embodiments, the anti-BCMA CARs described herein binds to BCMA with a K_{D} of sub-nanomolar range.

In some embodiments, an anti-BCMA CAR of the present disclosure comprises one or more of the heavy chain CDRs (e.g., CDRH1, CDRH2, or CDRH3) amino acid sequences from any one of the anti-BCMA antibodies selected from Table 3. In some embodiments, an anti-BCMA CAR of the present disclosure comprise the CDRH1, CDRH2, and CDRH3 as provided for any one of the antibodies elected from Table 3. In some embodiments, an anti-BCMA CAR of the present disclosure comprises one or more of the light chain CDRs (e.g., CDRL1, CDRL2, or CDRL3) amino acid sequences from any one of the anti-BCMA antibodies selected from Table 3. In some embodiments, an anti-BCMA CAR of the present disclosure comprise the CDRL1, CDRL2, and CDRL3 as provided for any one of the anti-BCMA antibodies selected from Table 3.

In some embodiments, an anti-BCMA CAR comprises the CDRH1, CDRH2, CDRH3, CDRL1, CDRL2, and CDRL3 as provided for any one of the anti-BCMA antibodies selected from Table 3. In some embodiments, antibody heavy and light chain CDR3 domains may play a particularly important role in the binding specificity/affinity of an antibody for an antigen. Accordingly, an anti-BCMA CAR may include at least the heavy and/or light chain CDR3s of any one of the anti-BCMA antibodies selected from Table 3.

In some embodiments, any of the anti-BCMA CAR of the disclosure have one or more CDRs (e.g., heavy chain CDR or light chain CDR) sequences substantially similar to any of the CDRH1, CDRH2, CDRH3, CDRL1, CDRL2, and/or CDRL3 sequences from one of the anti-BCMA antibodies selected from Table 3. In some embodiments, the position of one or more CDRs along the VH (e.g., CDRH1, CDRH2, or CDRH3) and/or VL (e.g., CDRL1, CDRL2, or CDRL3) region of a chimeric antigen receptor described herein can vary by one, two, three, four, five, or six amino acid positions so long as specific binding to BCMA (e.g., human BCMA) is maintained (e.g., substantially maintained, for example, at least 50%, at least 60%, at least 70%, at least 80%, at least 90%, at least 95% of the binding of the original antibody from which it is derived).

In some examples, an anti-BCMA CAR comprises one or more CDR (e.g., heavy chain CDRs or light chain CDR) sequences substantially similar to any one of the anti-BCMA antibodies selected from Table 3. For example, the anti-BCMA CAR may include one or more CDR sequence(s) from any of the anti-BCMA antibodies selected from Table 3 containing up to 5, 4, 3, 2, or 1 amino acid residue variations as compared to the corresponding CDR region in any one of the CDRs provided herein (e.g., CDRs from any of the anti-BCMA antibodies selected from Table 3) so long as specific binding to BCMA (e.g., human BCMA) is maintained (e.g., substantially maintained, for example, at least 50%, at least 60%, at least 70%, at least 80%, at least 90%, at least 95% relative to the binding of the original antibody from which it is derived).

Some aspects of the disclosure provide anti-BCMA CARs that comprise one or more of the heavy chain variable (VH) and/or light chain variable (VL) domains provided herein. In some embodiments, any of the VH domains provided herein include one or more of the heavy chain CDR sequences (e.g., CDRH1, CDRH2, and CDRH3) provided herein, for example, any of the heavy chain CDR sequences provided in any one of the anti-BCMA antibodies selected from Table 3. In some embodiments, any of the VL domains provided herein include one or more of the CDR-L sequences (e.g., CDRL1, CDRL2, and CDRL3) provided herein, for example, any of the light chain CDR sequences provided in any one of the anti-BCMA antibodies selected from Table 3.

In some embodiments, an anti-BCMA CAR comprises a heavy chain variable domain and/or a light chain variable domain of any one of the anti-BCMA antibodies selected from Table 3, and variants thereof. In some embodiments, an anti-BCMA CAR comprises the heavy chain variable and light chain variable pairs of any anti-BCMA antibodies selected from Table 3.

Aspects of the disclosure provide anti-BCMA CAR comprising a heavy chain variable (VH) and/or a light chain variable (VL) domain amino acid sequence homologous to any of those described herein. In some embodiments, an anti-BCMA CAR comprises a VH or a VL that is at least 75% (e.g., at least 80%, at least 85%, at least 90%, at least 91%, at least 92%, at least 93%, at least 94%, at least 95%, at least 96%, at least 97%, at least 98%, at least 99%, or 100%) identical to the VH and/or any VL of any one of the anti-BCMA antibodies selected from Table 3. In some embodiments, the homologous VH and/or a VL amino acid sequences of the anti-BCMA CAR do not vary within any of the CDR sequences provided herein. For example, in some embodiments, the degree of sequence variation (e.g., 75%, 80%, 85%, 90%, 95%, 98%, or 99%) may occur within a VH and/or a VL sequence of an anti-BCMA CAR excluding any of the CDR sequences provided herein. In some embodiments, any of the anti-BCMA CAR provided herein comprise a VH sequence and a VL sequence that comprises a framework sequence that is at least 75%, 80%, 85%, 90%, 95%, 98%, or 99% identical to the framework sequence of any anti-BCMA antibodies selected from Table 3. In some embodiments, an anti-BCMA CAR comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH of any of the anti-BCMA antibodies listed in Table 3. Alternatively or in addition, an anti-BCMA CAR comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL of any one of the anti-BCMA antibodies listed in Table 3.

In some embodiments, an anti-BCMA CAR comprises a CDRH1, a CDRH2, a CDRH3, a CDRL1, a CDRL2, and a CDRL3 that are the same as the CDRH1, CDRH2, CDRH3, CDRL1, CDRL2, and CDRL3 shown in Table 3, and comprises a humanized VH and/or a humanized VL. In some embodiments, an anti-BCMA CAR is a humanized variant comprising one or more amino acid substitutions (e.g., in the VH framework region) as compared with any one of the VHs listed in Table 3, and/or one or more amino acid substitutions (e.g., in the VL framework region) as compared with any one of the VLs listed in Table 3.

In some embodiments, an anti-BCMA CAR comprises a CDRH1, a CDRH2 and a CDRH3 of a heavy chain variable domain (VH) having the amino acid sequence of SEQ ID NO: 7. Alternatively or in addition, an anti-BCMA CAR comprises a CDRL1, a CDRL2 and a CDRL3 of a light chain variable domain (VL) having the amino acid sequence of SEQ ID NO: 8.

In some embodiments, according to the Kabat definition system, an anti-BCMA CAR comprises a CDRH3 having the amino acid sequence of SEQ ID NO: 3. In some embodiments, according to the Kabat definition system, an anti-BCMA CAR comprises a CDRH1 having the amino acid sequence of SEQ ID NO: 1, a CDRH2 having the amino acid sequence of SEQ ID NO: 2, and a CDRH3 having the amino acid sequence of SEQ ID NO: 3. In some embodiments, according to the Kabat definition system, an anti-BCMA CAR comprises a CDRH1 having the amino acid sequence of SEQ ID NO: 1, a CDRH2 having the amino acid sequence of SEQ ID NO: 2, a CDRH3 having the amino acid sequence of SEQ ID NO: 3, a CDRL1 having the amino acid sequence of SEQ ID NO: 4, a CDRL2 having the amino acid sequence of SEQ ID NO: 5, and a CDRL3 having the amino acid sequence of SEQ ID NO: 6.

In some embodiments, an anti-BCMA CAR comprises a CDRH1, a CDRH2, and a CDRH3, which collectively contains no more than 5 amino acid variations (e.g., no more than 5, 4, 3, 2, or 1 amino acid variation) as compared with the CDRH1 having the amino acid sequence of SEQ ID NO: 1, CDRH2 having the amino acid sequence of SEQ ID NO: 2, and CDRH3 having the amino acid sequence of SEQ ID NO: 3. Alternatively or in addition, an anti-BCMA CAR comprises a CDRL1, a CDRL2, and a CDRL3, which collectively contains no more than 5 amino acid variations (e.g., no more than 5, 4, 3, 2 or 1 amino acid variation) as compared with the CDRL1 having the amino acid sequence of SEQ ID NO: 4, CDRL2 having the amino acid sequence of SEQ ID NO: 5, and CDRL3 having the amino acid sequence of SEQ ID NO: 6.

In some embodiments, an anti-BCMA CAR comprises a CDRH1, a CDRH2, and a CDRH3 that collectively are at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the CDRH1 having the amino acid sequence of SEQ ID NO: 1, CDRH2 having the amino acid sequence of SEQ ID NO: 2, and CDRH3 having the amino acid sequence of SEQ ID NO: 3. Alternatively or in addition, an anti-BCMA CAR comprises a CDRL1, a CDRL2, and a CDRL3 that collectively are at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the to the CDRL1 having the amino acid sequence of SEQ ID NO: 4, CDRL2 having the amino acid sequence of SEQ ID NO: 5, and CDRL3 having the amino acid sequence of SEQ ID NO: 6.

In some embodiments, an anti-BCMA CAR comprises: a CDRH1 having no more than 3 amino acid variations (e.g., no more than 3, 2, or 1 amino acid variation) as compared with the CDRH1 having the amino acid sequence of SEQ ID NO: 1; a CDRH2 having no more than 3 amino acid variations (e.g., no more than 3, 2, or 1 amino acid variation) as compared with the CDRH2 having the amino acid sequence of SEQ ID NO: 2; and/or a CDRH3 having no more than 3 amino acid variations (e.g., no more than 3, 2, or 1 amino acid variation) as compared with the CDRH3 having the amino acid sequence of SEQ ID NO: 3. Alternatively or in addition, an anti-BCMA CAR thereof comprises: a CDRL1 having no more than 3 amino acid variations (e.g., no more than 3, 2, or 1 amino acid variation) as compared with the CDRL1 having the amino acid sequence of SEQ ID NO: 4; a CDRL2 having no more than 3 amino acid variations (e.g., no more than 3, 2, or 1 amino acid variation) as compared with the CDRL2 having the amino acid sequence of SEQ ID NO: 5; and/or a CDRL3 having no more than 3 amino acid variations (e.g., no more than 3, 2, or 1 amino acid variation) as compared with the CDRL3 having the amino acid sequence of SEQ ID NO: 6.

In some embodiments, an anti-BCMA CAR comprises a VH comprising the amino acid sequence of SEQ ID NO: 7. Alternatively or in addition, an anti-BCMA CAR comprises a VL comprising the amino acid sequence of SEQ ID NO: 8.

In some embodiments, an anti-BCMA CAR comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 7. Alternatively or in addition, an anti-BCMA CAR comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 8.

In some embodiments, an anti-BCMA CAR comprises a VH comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VH as set forth in SEQ ID NO: 7. Alternatively or in addition, an anti-BCMA CAR comprises a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VL as set forth in SEQ ID NO: 8.

In some embodiments, an anti-BCMA CAR comprises a CDRH1, a CDRH2 and a CDRH3 of a heavy chain variable domain (VH) having the amino acid sequence of SEQ ID NO: 7. Alternatively or in addition, an anti-BCMA CAR comprises a CDRL1, a CDRL2 and a CDRL3 of a light chain variable domain (VL) having the amino acid sequence of SEQ ID NO: 10.

In some embodiments, according to the Kabat definition system, an anti-BCMA CAR comprises a CDRH3 having the amino acid sequence of SEQ ID NO: 3. In some embodiments, according to the Kabat definition system, an anti-BCMA CAR comprises a CDRH1 having the amino acid sequence of SEQ ID NO: 1, a CDRH2 having the amino acid sequence of SEQ ID NO: 2, and a CDRH3 having the amino acid sequence of SEQ ID NO: 3. In some embodiments, according to the Kabat definition system, an anti-BCMA CAR comprises a CDRH1 having the amino acid sequence of SEQ ID NO: 1, a CDRH2 having the amino acid sequence of SEQ ID NO: 2, a CDRH3 having the amino acid sequence of SEQ ID NO: 3, a CDRL1 having the amino acid sequence of SEQ ID NO: 4, a CDRL2 having the amino acid sequence of SEQ ID NO: 24, and a CDRL3 having the amino acid sequence of SEQ ID NO: 6.

In some embodiments, an anti-BCMA CAR comprises a CDRH1, a CDRH2, and a CDRH3, which collectively contains no more than 5 amino acid variations (e.g., no more than 5, 4, 3, 2, or 1 amino acid variation) as compared with the CDRH1 having the amino acid sequence of SEQ ID NO: 1, CDRH2 having the amino acid sequence of SEQ ID NO: 2, and CDRH3 having the amino acid sequence of SEQ ID NO: 3. Alternatively or in addition, an anti-BCMA CAR comprises a CDRL1, a CDRL2, and a CDRL3, which collectively contains no more than 5 amino acid variations (e.g., no more than 5, 4, 3, 2 or 1 amino acid variation) as compared with the CDRL1 having the amino acid sequence of SEQ ID NO: 4, CDRL2 having the amino acid sequence of SEQ ID NO: 24, and CDRL3 having the amino acid sequence of SEQ ID NO: 6.

In some embodiments, an anti-BCMA CAR comprises a CDRH1, a CDRH2, and a CDRH3 that collectively are at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the CDRH1 having the amino acid sequence of SEQ ID NO: 1, CDRH2 having the amino acid sequence of SEQ ID NO: 2, and CDRH3 having the amino acid sequence of SEQ ID NO: 3. Alternatively or in addition, an anti-BCMA CAR comprises a CDRL1, a CDRL2, and a CDRL3 that collectively are at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the to the CDRL1 having the amino acid sequence of SEQ ID NO: 4, CDRL2 having the amino acid sequence of SEQ ID NO: 24, and CDRL3 having the amino acid sequence of SEQ ID NO: 6.

In some embodiments, an anti-BCMA CAR comprises: a CDRH1 having no more than 3 amino acid variations (e.g., no more than 3, 2, or 1 amino acid variation) as compared with the CDRH1 having the amino acid sequence of SEQ ID NO: 1; a CDRH2 having no more than 3 amino acid variations (e.g., no more than 3, 2, or 1 amino acid variation) as compared with the CDRH2 having the amino acid sequence of SEQ ID NO: 2; and/or a CDRH3 having no more than 3 amino acid variations (e.g., no more than 3, 2, or 1 amino acid variation) as compared with the CDRH3 having the amino acid sequence of SEQ ID NO: 3. Alternatively or in addition, an anti-BCMA CAR comprises: a CDRL1 having no more than 3 amino acid variations (e.g., no more than 3, 2, or 1 amino acid variation) as compared with the CDRL1 having the amino acid sequence of SEQ ID NO: 4; a CDRL2 having no more than 3 amino acid variations (e.g., no more than 3, 2, or 1 amino acid variation) as compared with the CDRL2 having the amino acid sequence of SEQ ID NO: 24; and/or a CDRL3 having no more than 3 amino acid variations (e.g., no more than 3, 2, or 1 amino acid variation) as compared with the CDRL3 having the amino acid sequence of SEQ ID NO: 6.

In some embodiments, an anti-BCMA CAR comprises a VH comprising the amino acid sequence of SEQ ID NO: 7. Alternatively or in addition, an anti-BCMA CAR comprises a VL comprising the amino acid sequence of SEQ ID NO: 10.

In some embodiments, an anti-BCMA CAR comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 7. Alternatively or in addition, an anti-BCMA CAR comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 10.

In some embodiments, an anti-BCMA CAR comprises a VH comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VH as set forth in SEQ ID NO: 7. Alternatively or in addition, an anti-BCMA CAR comprises a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VL as set forth in SEQ ID NO: 10.

In some embodiments, an anti-BCMA CAR comprises a CDRH1, a CDRH2 and a CDRH3 of a heavy chain variable domain (VH) having the amino acid sequence of SEQ ID NO: 12. Alternatively or in addition, an anti-BCMA CAR comprises a CDRL1, a CDRL2 and a CDRL3 of a light chain variable domain (VL) having the amino acid sequence of SEQ ID NO: 13.

In some embodiments, according to the Kabat definition system, an anti-BCMA CAR comprises a CDRH3 having the amino acid sequence of SEQ ID NO: 3. In some embodiments, according to the Kabat definition system, an anti-BCMA CAR comprises a CDRH1 having the amino acid sequence of SEQ ID NO: 25, a CDRH2 having the amino acid sequence of SEQ ID NO: 27, and a CDRH3 having the amino acid sequence of SEQ ID NO: 3. In some embodiments, according to the Kabat definition system, an anti-BCMA CAR comprises a CDRH1 having the amino acid sequence of SEQ ID NO: 25, a CDRH2 having the amino acid sequence of SEQ ID NO: 27, a CDRH3 having the amino acid sequence of SEQ ID NO: 3, a CDRL1 having the amino acid sequence of SEQ ID NO: 4, a CDRL2 having the amino acid sequence of SEQ ID NO: 24, and a CDRL3 having the amino acid sequence of SEQ ID NO: 30.

In some embodiments, an anti-BCMA CAR comprises a CDRH1, a CDRH2, and a CDRH3, which collectively contains no more than 5 amino acid variations (e.g., no more than 5, 4, 3, 2, or 1 amino acid variation) as compared with the CDRH1 having the amino acid sequence of SEQ ID NO: 25, CDRH2 having the amino acid sequence of SEQ ID NO: 27, and CDRH3 having the amino acid sequence of SEQ ID NO: 3. Alternatively or in addition, an anti-BCMA CAR comprises a CDRL1, a CDRL2, and a CDRL3, which collectively contains no more than 5 amino acid variations (e.g., no more than 5, 4, 3, 2 or 1 amino acid variation) as compared with the CDRL1 having the amino acid sequence of SEQ ID NO: 4, CDRL2 having the amino acid sequence of SEQ ID NO: 24, and CDRL3 having the amino acid sequence of SEQ ID NO: 30.

In some embodiments, an anti-BCMA CAR comprises a CDRH1, a CDRH2, and a CDRH3 that collectively are at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the CDRH1 having the amino acid sequence of SEQ ID NO: 25, CDRH2 having the amino acid sequence of SEQ ID NO: 27, and CDRH3 having the amino acid sequence of SEQ ID NO: 3. Alternatively or in addition, an anti-BCMA CAR comprises a CDRL1, a CDRL2, and a CDRL3 that collectively are at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the to the CDRL1 having the amino acid sequence of SEQ ID NO: 4, CDRL2 having the amino acid sequence of SEQ ID NO: 24, and CDRL3 having the amino acid sequence of SEQ ID NO: 30.

In some embodiments, an anti-BCMA CAR comprises: a CDRH1 having no more than 3 amino acid variations (e.g., no more than 3, 2, or 1 amino acid variation) as compared with the CDRH1 having the amino acid sequence of SEQ ID NO: 25; a CDRH2 having no more than 3 amino acid variations (e.g., no more than 3, 2, or 1 amino acid variation) as compared with the CDRH2 having the amino acid sequence of SEQ ID NO: 27; and/or a CDRH3 having no more than 3 amino acid variations (e.g., no more than 3, 2, or 1 amino acid variation) as compared with the CDRH3 having the amino acid sequence of SEQ ID NO: 3. Alternatively or in addition, an anti-BCMA CAR comprises: a CDRL1 having no more than 3 amino acid variations (e.g., no more than 3, 2, or 1 amino acid variation) as compared with the CDRL1 having the amino acid sequence of SEQ ID NO: 4; a CDRL2 having no more than 3 amino acid variations (e.g., no more than 3, 2, or 1 amino acid variation) as compared with the CDRL2 having the amino acid sequence of SEQ ID NO: 24; and/or a CDRL3 having no more than 3 amino acid variations (e.g., no more than 3, 2, or 1 amino acid variation) as compared with the CDRL3 having the amino acid sequence of SEQ ID NO: 30.

In some embodiments, an anti-BCMA CAR comprises a VH comprising the amino acid sequence of SEQ ID NO: 12. Alternatively or in addition, an anti-BCMA CAR comprises a VL comprising the amino acid sequence of SEQ ID NO: 13.

In some embodiments, an anti-BCMA CAR comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 12. Alternatively or in addition, an anti-BCMA CAR comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 13.

In some embodiments, an anti-BCMA CAR comprises a VH comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VH as set forth in SEQ ID NO: 12. Alternatively or in addition, an anti-BCMA CAR comprises a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VL as set forth in SEQ ID NO: 13.

In some embodiments, an anti-BCMA CAR comprises a CDRH1, a CDRH2 and a CDRH3 of a heavy chain variable domain (VH) having the amino acid sequence of SEQ ID NO: 12. Alternatively or in addition, an anti-BCMA CAR comprises a CDRL1, a CDRL2 and a CDRL3 of a light chain variable domain (VL) having the amino acid sequence of SEQ ID NO: 15.

In some embodiments, according to the Kabat definition system, an anti-BCMA CAR comprises a CDRH3 having the amino acid sequence of SEQ ID NO: 3. In some embodiments, according to the Kabat definition system, an anti-BCMA CAR comprises a CDRH1 having the amino acid sequence of SEQ ID NO: 25, a CDRH2 having the amino acid sequence of SEQ ID NO: 27, and a CDRH3 having the amino acid sequence of SEQ ID NO: 3. In some embodiments, according to the Kabat definition system, an anti-BCMA CAR comprises a CDRH1 having the amino acid sequence of SEQ ID NO: 25, a CDRH2 having the amino acid sequence of SEQ ID NO: 27, a CDRH3 having the amino acid sequence of SEQ ID NO: 3, a CDRL1 having the amino acid sequence of SEQ ID NO: 4, a CDRL2 having the amino acid sequence of SEQ ID NO: 24, and a CDRL3 having the amino acid sequence of SEQ ID NO: 29.

In some embodiments, an anti-BCMA CAR comprises a CDRH1, a CDRH2, and a CDRH3, which collectively contains no more than 5 amino acid variations (e.g., no more than 5, 4, 3, 2, or 1 amino acid variation) as compared with the CDRH1 having the amino acid sequence of SEQ ID NO: 25, CDRH2 having the amino acid sequence of SEQ ID NO: 27, and CDRH3 having the amino acid sequence of SEQ ID NO: 3. Alternatively or in addition, an anti-BCMA CAR comprises a CDRL1, a CDRL2, and a CDRL3, which collectively contains no more than 5 amino acid variations (e.g., no more than 5, 4, 3, 2 or 1 amino acid variation) as compared with the CDRL1 having the amino acid sequence of SEQ ID NO: 4, CDRL2 having the amino acid sequence of SEQ ID NO: 24, and CDRL3 having the amino acid sequence of SEQ ID NO: 29.

In some embodiments, an anti-BCMA CAR comprises a CDRH1, a CDRH2, and a CDRH3 that collectively are at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the CDRH1 having the amino acid sequence of SEQ ID NO: 25, CDRH2 having the amino acid sequence of SEQ ID NO: 27, and CDRH3 having the amino acid sequence of SEQ ID NO: 3. Alternatively or in addition, an anti-BCMA CAR comprises a CDRL1, a CDRL2, and a CDRL3 that collectively are at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the to the CDRL1 having the amino acid sequence of SEQ ID NO: 4, CDRL2 having the amino acid sequence of SEQ ID NO: 24, and CDRL3 having the amino acid sequence of SEQ ID NO: 29.

In some embodiments, an anti-BCMA CAR comprises: a CDRH1 having no more than 3 amino acid variations (e.g., no more than 3, 2, or 1 amino acid variation) as compared with the CDRH1 having the amino acid sequence of SEQ ID NO: 25; a CDRH2 having no more than 3 amino acid variations (e.g., no more than 3, 2, or 1 amino acid variation) as compared with the CDRH2 having the amino acid sequence of SEQ ID NO: 27; and/or a CDRH3 having no more than 3 amino acid variations (e.g., no more than 3, 2, or 1 amino acid variation) as compared with the CDRH3 having the amino acid sequence of SEQ ID NO: 3. Alternatively or in addition, an anti-BCMA CAR comprises: a CDRL1 having no more than 3 amino acid variations (e.g., no more than 3, 2, or 1 amino acid variation) as compared with the CDRL1 having the amino acid sequence of SEQ ID NO: 4; a CDRL2 having no more than 3 amino acid variations (e.g., no more than 3, 2, or 1 amino acid variation) as compared with the CDRL2 having the amino acid sequence of SEQ ID NO: 24; and/or a CDRL3 having no more than 3 amino acid variations (e.g., no more than 3, 2, or 1 amino acid variation) as compared with the CDRL3 having the amino acid sequence of SEQ ID NO: 29.

In some embodiments, an anti-BCMA CAR comprises a VH comprising the amino acid sequence of SEQ ID NO: 12. Alternatively or in addition, an anti-BCMA CAR comprises a VL comprising the amino acid sequence of SEQ ID NO: 15.

In some embodiments, an anti-BCMA CAR comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 12. Alternatively or in addition, an anti-BCMA CAR comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 15.

In some embodiments, an anti-BCMA CAR comprises a VH comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VH as set forth in SEQ ID NO: 12. Alternatively or in addition, an anti-BCMA CAR comprises a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VL as set forth in SEQ ID NO: 15.

In some embodiments, an anti-BCMA CAR comprises a CDRH1, a CDRH2 and a CDRH3 of a heavy chain variable domain (VH) having the amino acid sequence of SEQ ID NO: 7. Alternatively or in addition, an anti-BCMA CAR comprises a CDRL1, a CDRL2 and a CDRL3 of a light chain variable domain (VL) having the amino acid sequence of SEQ ID NO: 13.

In some embodiments, according to the Kabat definition system, an anti-BCMA CAR comprises a CDRH3 having the amino acid sequence of SEQ ID NO: 3. In some embodiments, according to the Kabat definition system, an anti-BCMA CAR comprises a CDRH1 having the amino acid sequence of SEQ ID NO: 1, a CDRH2 having the amino acid sequence of SEQ ID NO: 2, and a CDRH3 having the amino acid sequence of SEQ ID NO: 3. In some embodiments, according to the Kabat definition system, an anti-BCMA CAR comprises a CDRH1 having the amino acid sequence of SEQ ID NO: 1, a CDRH2 having the amino acid sequence of SEQ ID NO: 2, a CDRH3 having the amino acid sequence of SEQ ID NO: 3, a CDRL1 having the amino acid sequence of SEQ ID NO: 4, a CDRL2 having the amino acid sequence of SEQ ID NO: 24, and a CDRL3 having the amino acid sequence of SEQ ID NO: 30.

In some embodiments, an anti-BCMA CAR comprises a CDRH1, a CDRH2, and a CDRH3, which collectively contains no more than 5 amino acid variations (e.g., no more than 5, 4, 3, 2, or 1 amino acid variation) as compared with the CDRH1 having the amino acid sequence of SEQ ID NO: 1, CDRH2 having the amino acid sequence of SEQ ID NO: 2, and CDRH3 having the amino acid sequence of SEQ ID NO: 3. Alternatively or in addition, an anti-BCMA CAR comprises a CDRL1, a CDRL2, and a CDRL3, which collectively contains no more than 5 amino acid variations (e.g., no more than 5, 4, 3, 2 or 1 amino acid variation) as compared with the CDRL1 having the amino acid sequence of SEQ ID NO: 4, CDRL2 having the amino acid sequence of SEQ ID NO: 24, and CDRL3 having the amino acid sequence of SEQ ID NO: 30.

In some embodiments, an anti-BCMA CAR comprises a CDRH1, a CDRH2, and a CDRH3 that collectively are at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the CDRH1 having the amino acid sequence of SEQ ID NO: 1, CDRH2 having the amino acid sequence of SEQ ID NO: 2, and CDRH3 having the amino acid sequence of SEQ ID NO: 3. Alternatively or in addition, an anti-BCMA CAR comprises a CDRL1, a CDRL2, and a CDRL3 that collectively are at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the to the CDRL1 having the amino acid sequence of SEQ ID NO: 4, CDRL2 having the amino acid sequence of SEQ ID NO: 24, and CDRL3 having the amino acid sequence of SEQ ID NO: 30.

In some embodiments, an anti-BCMA CAR comprises: a CDRH1 having no more than 3 amino acid variations (e.g., no more than 3, 2, or 1 amino acid variation) as compared with the CDRH1 having the amino acid sequence of SEQ ID NO: 1; a CDRH2 having no more than 3 amino acid variations (e.g., no more than 3, 2, or 1 amino acid variation) as compared with the CDRH2 having the amino acid sequence of SEQ ID NO: 2; and/or a CDRH3 having no more than 3 amino acid variations (e.g., no more than 3, 2, or 1 amino acid variation) as compared with the CDRH3 having the amino acid sequence of SEQ ID NO: 3. Alternatively or in addition, an anti-BCMA CAR comprises: a CDRL1 having no more than 3 amino acid variations (e.g., no more than 3, 2, or 1 amino acid variation) as compared with the CDRL1 having the amino acid sequence of SEQ ID NO: 4; a CDRL2 having no more than 3 amino acid variations (e.g., no more than 3, 2, or 1 amino acid variation) as compared with the CDRL2 having the amino acid sequence of SEQ ID NO: 24; and/or a CDRL3 having no more than 3 amino acid variations (e.g., no more than 3, 2, or 1 amino acid variation) as compared with the CDRL3 having the amino acid sequence of SEQ ID NO: 30.

In some embodiments, an anti-BCMA CAR comprises a VH comprising the amino acid sequence of SEQ ID NO: 7. Alternatively or in addition, an anti-BCMA CAR comprises a VL comprising the amino acid sequence of SEQ ID NO: 13.

In some embodiments, an anti-BCMA CAR comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 7. Alternatively or in addition, an anti-BCMA CAR comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 13.

In some embodiments, an anti-BCMA CAR comprises a VH comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VH as set forth in SEQ ID NO: 7. Alternatively or in addition, an anti-BCMA CAR comprises a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VL as set forth in SEQ ID NO: 13.

In some embodiments, an anti-BCMA CAR comprises a CDRH1, a CDRH2 and a CDRH3 of a heavy chain variable domain (VH) having the amino acid sequence of SEQ ID NO: 7. Alternatively or in addition, an anti-BCMA CAR comprises a CDRL1, a CDRL2 and a CDRL3 of a light chain variable domain (VL) having the amino acid sequence of SEQ ID NO: 15.

In some embodiments, according to the Kabat definition system, an anti-BCMA CAR comprises a CDRH3 having the amino acid sequence of SEQ ID NO: 3. In some embodiments, according to the Kabat definition system, an anti-BCMA CAR comprises a CDRH1 having the amino acid sequence of SEQ ID NO: 1, a CDRH2 having the amino acid sequence of SEQ ID NO: 2, and a CDRH3 having the amino acid sequence of SEQ ID NO: 3. In some embodiments, according to the Kabat definition system, an anti-BCMA CAR comprises a CDRH1 having the amino acid sequence of SEQ ID NO: 1, a CDRH2 having the amino acid sequence of SEQ ID NO: 2, a CDRH3 having the amino acid sequence of SEQ ID NO: 3, a CDRL1 having the amino acid sequence of SEQ ID NO: 4, a CDRL2 having the amino acid sequence of SEQ ID NO: 24, and a CDRL3 having the amino acid sequence of SEQ ID NO: 29.

In some embodiments, an anti-BCMA CAR comprises a CDRH1, a CDRH2, and a CDRH3, which collectively contains no more than 5 amino acid variations (e.g., no more than 5, 4, 3, 2, or 1 amino acid variation) as compared with the CDRH1 having the amino acid sequence of SEQ ID NO: 1, CDRH2 having the amino acid sequence of SEQ ID NO: 2, and CDRH3 having the amino acid sequence of SEQ ID NO: 3. Alternatively or in addition, an anti-BCMA CAR comprises a CDRL1, a CDRL2, and a CDRL3, which collectively contains no more than 5 amino acid variations (e.g., no more than 5, 4, 3, 2 or 1 amino acid variation) as compared with the CDRL1 having the amino acid sequence of SEQ ID NO: 4, CDRL2 having the amino acid sequence of SEQ ID NO: 24, and CDRL3 having the amino acid sequence of SEQ ID NO: 29.

In some embodiments, an anti-BCMA CAR comprises a CDRH1, a CDRH2, and a CDRH3 that collectively are at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the CDRH1 having the amino acid sequence of SEQ ID NO: 1, CDRH2 having the amino acid sequence of SEQ ID NO: 2, and CDRH3 having the amino acid sequence of SEQ ID NO: 3. Alternatively or in addition, an anti-BCMA CAR comprises a CDRL1, a CDRL2, and a CDRL3 that collectively are at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the to the CDRL1 having the amino acid sequence of SEQ ID NO: 4, CDRL2 having the amino acid sequence of SEQ ID NO: 24, and CDRL3 having the amino acid sequence of SEQ ID NO: 29.

In some embodiments, an anti-BCMA CAR comprises: a CDRH1 having no more than 3 amino acid variations (e.g., no more than 3, 2, or 1 amino acid variation) as compared with the CDRH1 having the amino acid sequence of SEQ ID NO: 1; a CDRH2 having no more than 3 amino acid variations (e.g., no more than 3, 2, or 1 amino acid variation) as compared with the CDRH2 having the amino acid sequence of SEQ ID NO: 2; and/or a CDRH3 having no more than 3 amino acid variations (e.g., no more than 3, 2, or 1 amino acid variation) as compared with the CDRH3 having the amino acid sequence of SEQ ID NO: 3. Alternatively or in addition, an anti-BCMA CAR comprises: a CDRL1 having no more than 3 amino acid variations (e.g., no more than 3, 2, or 1 amino acid variation) as compared with the CDRL1 having the amino acid sequence of SEQ ID NO: 4; a CDRL2 having no more than 3 amino acid variations (e.g., no more than 3, 2, or 1 amino acid variation) as compared with the CDRL2 having the amino acid sequence of SEQ ID NO: 24; and/or a CDRL3 having no more than 3 amino acid variations (e.g., no more than 3, 2, or 1 amino acid variation) as compared with the CDRL3 having the amino acid sequence of SEQ ID NO: 29.

In some embodiments, an anti-BCMA CAR comprises a VH comprising the amino acid sequence of SEQ ID NO: 7. Alternatively or in addition, an anti-BCMA CAR comprises a VL comprising the amino acid sequence of SEQ ID NO: 15.

In some embodiments, an anti-BCMA CAR comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 7. Alternatively or in addition, an anti-BCMA CAR comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 15.

In some embodiments, an anti-BCMA CAR comprises a VH comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VH as set forth in SEQ ID NO: 7. Alternatively or in addition, an anti-BCMA CAR comprises a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VL as set forth in SEQ ID NO: 15.

In some embodiments, an anti-BCMA CAR comprises a CDRH1, a CDRH2 and a CDRH3 of a heavy chain variable domain (VH) having the amino acid sequence of SEQ ID NO: 19. Alternatively or in addition, an anti-BCMA CAR comprises a CDRL1, a CDRL2 and a CDRL3 of a light chain variable domain (VL) having the amino acid sequence of SEQ ID NO: 10.

In some embodiments, according to the Kabat definition system, an anti-BCMA CAR comprises a CDRH3 having the amino acid sequence of SEQ ID NO: 3. In some embodiments, according to the Kabat definition system, an anti-BCMA CAR comprises a CDRH1 having the amino acid sequence of SEQ ID NO: 26, a CDRH2 having the amino acid sequence of SEQ ID NO: 28, and a CDRH3 having the amino acid sequence of SEQ ID NO: 3. In some embodiments, according to the Kabat definition system, an anti-BCMA CAR comprises a CDRH1 having the amino acid sequence of SEQ ID NO: 26, a CDRH2 having the amino acid sequence of SEQ ID NO: 28, a CDRH3 having the amino acid sequence of SEQ ID NO: 3, a CDRL1 having the amino acid sequence of SEQ ID NO: 4, a CDRL2 having the amino acid sequence of SEQ ID NO: 24, and a CDRL3 having the amino acid sequence of SEQ ID NO: 6.

In some embodiments, an anti-BCMA CAR comprises a CDRH1, a CDRH2, and a CDRH3, which collectively contains no more than 5 amino acid variations (e.g., no more than 5, 4, 3, 2, or 1 amino acid variation) as compared with the CDRH1 having the amino acid sequence of SEQ ID NO: 26, CDRH2 having the amino acid sequence of SEQ ID NO: 28, and CDRH3 having the amino acid sequence of SEQ ID NO: 3. Alternatively or in addition, an anti-BCMA CAR comprises a CDRL1, a CDRL2, and a CDRL3, which collectively contains no more than 5 amino acid variations (e.g., no more than 5, 4, 3, 2 or 1 amino acid variation) as compared with the CDRL1 having the amino acid sequence of SEQ ID NO: 4, CDRL2 having the amino acid sequence of SEQ ID NO: 24, and CDRL3 having the amino acid sequence of SEQ ID NO: 6.

In some embodiments, an anti-BCMA CAR comprises a CDRH1, a CDRH2, and a CDRH3 that collectively are at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the CDRH1 having the amino acid sequence of SEQ ID NO: 26, CDRH2 having the amino acid sequence of SEQ ID NO: 28, and CDRH3 having the amino acid sequence of SEQ ID NO: 3. Alternatively or in addition, an anti-BCMA CAR comprises a CDRL1, a CDRL2, and a CDRL3 that collectively are at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the to the CDRL1 having the amino acid sequence of SEQ ID NO: 4, CDRL2 having the amino acid sequence of SEQ ID NO: 24, and CDRL3 having the amino acid sequence of SEQ ID NO: 6.

In some embodiments, an anti-BCMA CAR comprises: a CDRH1 having no more than 3 amino acid variations (e.g., no more than 3, 2, or 1 amino acid variation) as compared with the CDRH1 having the amino acid sequence of SEQ ID NO: 26; a CDRH2 having no more than 3 amino acid variations (e.g., no more than 3, 2, or 1 amino acid variation) as compared with the CDRH2 having the amino acid sequence of SEQ ID NO: 28; and/or a CDRH3 having no more than 3 amino acid variations (e.g., no more than 3, 2, or 1 amino acid variation) as compared with the CDRH3 having the amino acid sequence of SEQ ID NO: 3. Alternatively or in addition, an anti-BCMA CAR comprises: a CDRL1 having no more than 3 amino acid variations (e.g., no more than 3, 2, or 1 amino acid variation) as compared with the CDRL1 having the amino acid sequence of SEQ ID NO: 4; a CDRL2 having no more than 3 amino acid variations (e.g., no more than 3, 2, or 1 amino acid variation) as compared with the CDRL2 having the amino acid sequence of SEQ ID NO: 24; and/or a CDRL3 having no more than 3 amino acid variations (e.g., no more than 3, 2, or 1 amino acid variation) as compared with the CDRL3 having the amino acid sequence of SEQ ID NO: 6.

In some embodiments, an anti-BCMA CAR comprises a VH comprising the amino acid sequence of SEQ ID NO: 19. Alternatively or in addition, an anti-BCMA CAR comprises a VL comprising the amino acid sequence of SEQ ID NO: 10.

In some embodiments, an anti-BCMA CAR comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 19. Alternatively or in addition, an anti-BCMA CAR comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 10.

In some embodiments, an anti-BCMA CAR comprises a VH comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VH as set forth in SEQ ID NO: 19. Alternatively or in addition, an anti-BCMA CAR comprises a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VL as set forth in SEQ ID NO: 10.

In some embodiments, an anti-BCMA CAR comprises a CDRH1, a CDRH2 and a CDRH3 of a heavy chain variable domain (VH) having the amino acid sequence of SEQ ID NO: 21. Alternatively or in addition, an anti-BCMA CAR comprises a CDRL1, a CDRL2 and a CDRL3 of a light chain variable domain (VL) having the amino acid sequence of SEQ ID NO: 22.

In some embodiments, according to the Kabat definition system, an anti-BCMA CAR comprises a CDRH3 having the amino acid sequence of SEQ ID NO: 33. In some embodiments, according to the Kabat definition system, an anti-BCMA CAR comprises a CDRH1 having the amino acid sequence of SEQ ID NO: 31, a CDRH2 having the amino acid sequence of SEQ ID NO: 32, and a CDRH3 having the amino acid sequence of SEQ ID NO: 33. In some embodiments, according to the Kabat definition system, an anti-BCMA CAR comprises a CDRH1 having the amino acid sequence of SEQ ID NO: 31, a CDRH2 having the amino acid sequence of SEQ ID NO: 32, a CDRH3 having the amino acid sequence of SEQ ID NO: 33, a CDRL1 having the amino acid sequence of SEQ ID NO: 34, a CDRL2 having the amino acid sequence of SEQ ID NO: 35, and a CDRL3 having the amino acid sequence of SEQ ID NO: 36.

In some embodiments, an anti-BCMA CAR comprises a CDRH1, a CDRH2, and a CDRH3, which collectively contains no more than 5 amino acid variations (e.g., no more than 5, 4, 3, 2, or 1 amino acid variation) as compared with the CDRH1 having the amino acid sequence of SEQ ID NO: 31, CDRH2 having the amino acid sequence of SEQ ID NO: 32, and CDRH3 having the amino acid sequence of SEQ ID NO: 33. Alternatively or in addition, an anti-BCMA CAR comprises a CDRL1, a CDRL2, and a CDRL3, which collectively contains no more than 5 amino acid variations (e.g., no more than 5, 4, 3, 2 or 1 amino acid variation) as compared with the CDRL1 having the amino acid sequence of SEQ ID NO: 34, CDRL2 having the amino acid sequence of SEQ ID NO: 35, and CDRL3 having the amino acid sequence of SEQ ID NO: 36.

In some embodiments, an anti-BCMA CAR comprises a CDRH1, a CDRH2, and a CDRH3 that collectively are at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the CDRH1 having the amino acid sequence of SEQ ID NO: 31, CDRH2 having the amino acid sequence of SEQ ID NO: 32, and CDRH3 having the amino acid sequence of SEQ ID NO: 33. Alternatively or in addition, an anti-BCMA CAR comprises a CDRL1, a CDRL2, and a CDRL3 that collectively are at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the to the CDRL1 having the amino acid sequence of SEQ ID NO: 34, CDRL2 having the amino acid sequence of SEQ ID NO: 35, and CDRL3 having the amino acid sequence of SEQ ID NO: 36.

In some embodiments, an anti-BCMA CAR comprises: a CDRH1 having no more than 3 amino acid variations (e.g., no more than 3, 2, or 1 amino acid variation) as compared with the CDRH1 having the amino acid sequence of SEQ ID NO: 31; a CDRH2 having no more than 3 amino acid variations (e.g., no more than 3, 2, or 1 amino acid variation) as compared with the CDRH2 having the amino acid sequence of SEQ ID NO: 32; and/or a CDRH3 having no more than 3 amino acid variations (e.g., no more than 3, 2, or 1 amino acid variation) as compared with the CDRH3 having the amino acid sequence of SEQ ID NO: 33. Alternatively or in addition, an anti-BCMA CAR comprises: a CDRL1 having no more than 3 amino acid variations (e.g., no more than 3, 2, or 1 amino acid variation) as compared with the CDRL1 having the amino acid sequence of SEQ ID NO: 34; a CDRL2 having no more than 3 amino acid variations (e.g., no more than 3, 2, or 1 amino acid variation) as compared with the CDRL2 having the amino acid sequence of SEQ ID NO: 35; and/or a CDRL3 having no more than 3 amino acid variations (e.g., no more than 3, 2, or 1 amino acid variation) as compared with the CDRL3 having the amino acid sequence of SEQ ID NO: 36.

In some embodiments, an anti-BCMA CAR comprises a VH comprising the amino acid sequence of SEQ ID NO: 21. Alternatively or in addition, an anti-BCMA CAR comprises a VL comprising the amino acid sequence of SEQ ID NO: 22.

In some embodiments, an anti-BCMA CAR comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 21. Alternatively or in addition, an anti-BCMA CAR comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 22.

In some embodiments, an anti-BCMA CAR comprises a VH comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VH as set forth in SEQ ID NO: 21. Alternatively or in addition, an anti-BCMA CAR comprises a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VL as set forth in SEQ ID NO: 22.

An anti-BCMA antibody or antigen binding fragment thereof described herein can be grafted into a chimeric antigen receptor including, but not limited to, antigen-binding fragments thereof (such as Fab, F(ab′), F(ab′)2, Fv), single chain antibodies (e.g., scFv), bi-specific antibodies, or nanobodies. In some embodiments, an anti-BCMA CAR described herein comprises a single-chain variable fragment (scFv) as the extracellular ligand-binding domain.

In some embodiments, an anti-BCMA CAR comprises an extracellular ligand-binding domain that comprises a single-chain variable fragment (scFv). In some embodiments, an anti-BCMA CAR comprises an extracellular ligand-binding domain that comprises a VH and a VL of any one of the anti-BCMA scFv selected from Table 3. In some embodiments, the VH and the VL of an anti-BCMA CAR are joined together by a linker. In some embodiments, a linker may have a length of about 2 to 10 amino acids, 5 to 20 amino acids, 10 to 30 amino acids, 20-50 amino acids, 40 to 60 amino acids, 60 to 80 amino acids, or more than 80 amino acids. In some embodiment, a linker may include a sequence that substantially comprises glycine and serine. An exemplary linker sequence is GGGGSGGGGSGGGAS (SEQ ID NO: 81). In some embodiments, a linker may include, without limitation, any of those encompassed by U.S. Pat. Nos. 8,445,251 and 9,434,931. In some embodiments, an anti-BCMA CAR comprises a linker between the VH and the VL, and the linker comprises an amino acid sequence at least 80%, at least 85%, at least 90%, at least 92%, at least 95%, at least 98%, at least 99%, or 100% identical to SEQ ID NO: 81.

In some embodiments, an anti-BCMA CAR comprises an extracellular ligand-binding domain that comprises a scFv comprising a VH and a VL, and the C-terminus of the VH is joined with the N terminus of the VL via a linker (e.g., the linker as set forth in SEQ ID NO: 81). In some embodiments, an anti-BCMA CAR comprises an extracellular ligand-binding domain that comprises scFv comprising a VH and a VL, and the C-terminus of the VL is joined with the N terminus of the VH via a linker (e.g., the linker as set forth in SEQ ID NO: 81).

In some embodiments, an anti-BCMA CAR comprises an extracellular ligand-binding domain that comprises a scFv comprising a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH of any of the anti-BCMA antibodies listed in Table 3. Alternatively or in addition, an anti-BCMA CAR comprises an extracellular ligand-binding domain that comprises a scFv comprising a VL containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH of any of the anti-BCMA antibodies listed in Table 3. In some embodiments, an anti-BCMA CAR comprises an extracellular ligand-binding domain that comprises a scFv comprising a VH comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VH of any of the anti-BCMA antibodies listed in Table 3. Alternatively or in addition, an anti-BCMA CAR comprises an extracellular ligand-binding domain that comprises a scFv comprising a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the VH of any of the anti-BCMA antibodies listed in Table 3.

In some embodiments, an anti-BCMA CAR comprises an extracellular ligand-binding domain that comprises a scFv comprising an amino acid sequence containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the any of the anti-BCMA scFv listed in Table 3. In some embodiments, an anti-BCMA CAR comprises an extracellular ligand-binding domain that comprises a scFv comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to any of the anti-BCMA scFv listed in Table 3.

In some embodiments, an anti-BCMA CAR comprises an extracellular ligand-binding domain that comprises a scFv comprising an amino acid sequence containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the scFv amino acid sequence as set forth in SEQ ID NO: 9. In some embodiments, an anti-BCMA CAR comprises an extracellular ligand-binding domain that comprises a scFv comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the scFv amino acid sequence as set forth in SEQ ID NO: 9. In some embodiments, an anti-BCMA CAR comprises an extracellular ligand-binding domain that comprises a scFv comprising an amino acid sequence of SEQ ID NO: 9.

In some embodiments, an anti-BCMA CAR comprises an extracellular ligand-binding domain that comprises a scFv comprising an amino acid sequence containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the scFv amino acid sequence as set forth in SEQ ID NO: 11. In some embodiments, an anti-BCMA CAR comprises an extracellular ligand-binding domain that comprises a scFv comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the scFv amino acid sequence as set forth in SEQ ID NO: 11. In some embodiments, an anti-BCMA CAR comprises an extracellular ligand-binding domain that comprises a scFv comprising an amino acid sequence of SEQ ID NO: 11.

In some embodiments, an anti-BCMA scFv comprises an amino acid sequence containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the scFv amino acid sequence as set forth in SEQ ID NO: 14. In some embodiments, an anti-BCMA CAR comprises an extracellular ligand-binding domain that comprises a scFv comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the scFv amino acid sequence as set forth in SEQ ID NO: 14. In some embodiments, an anti-BCMA CAR comprises an extracellular ligand-binding domain that comprises a scFv comprising an amino acid sequence of SEQ ID NO: 14.

In some embodiments, an anti-BCMA CAR comprises an extracellular ligand-binding domain that comprises a scFv comprising an amino acid sequence containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the scFv amino acid sequence as set forth in SEQ ID NO: 16. In some embodiments, an anti-BCMA CAR comprises an extracellular ligand-binding domain that comprises a scFv comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the scFv amino acid sequence as set forth in SEQ ID NO: 16. In some embodiments, an anti-BCMA CAR comprises an extracellular ligand-binding domain that comprises a scFv comprising an amino acid sequence of SEQ ID NO: 16.

In some embodiments, an anti-BCMA CAR comprises an extracellular ligand-binding domain that comprises a scFv comprising an amino acid sequence containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the scFv amino acid sequence as set forth in SEQ ID NO: 17. In some embodiments, an anti-BCMA CAR comprises an extracellular ligand-binding domain that comprises a scFv comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the scFv amino acid sequence as set forth in SEQ ID NO: 17. In some embodiments, an anti-BCMA CAR comprises an extracellular ligand-binding domain that comprises a scFv comprising an amino acid sequence of SEQ ID NO: 17.

In some embodiments, an anti-BCMA CAR comprises an extracellular ligand-binding domain that comprises a scFv comprising an amino acid sequence containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the scFv amino acid sequence as set forth in SEQ ID NO: 18. In some embodiments, an anti-BCMA CAR comprises an extracellular ligand-binding domain that comprises a scFv comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the scFv amino acid sequence as set forth in SEQ ID NO: 18. In some embodiments, an anti-BCMA CAR comprises an extracellular ligand-binding domain that comprises a scFv comprising an amino acid sequence of SEQ ID NO: 18.

In some embodiments, an anti-BCMA CAR comprises an extracellular ligand-binding domain that comprises a scFv comprising an amino acid sequence containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the scFv amino acid sequence as set forth in SEQ ID NO: 20. In some embodiments, an anti-BCMA CAR comprises an extracellular ligand-binding domain that comprises a scFv comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the scFv amino acid sequence as set forth in SEQ ID NO: 20. In some embodiments, an anti-BCMA CAR comprises an extracellular ligand-binding domain that comprises a scFv comprising an amino acid sequence of SEQ ID NO: 20.

In some embodiments, an anti-BCMA CAR comprises an extracellular ligand-binding domain that comprises a scFv comprising an amino acid sequence containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the scFv amino acid sequence as set forth in SEQ ID NO: 23. In some embodiments, an anti-BCMA CAR comprises an extracellular ligand-binding domain that comprises a scFv comprising an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the scFv amino acid sequence as set forth in SEQ ID NO: 23. In some embodiments, an anti-BCMA CAR comprises an extracellular ligand-binding domain that comprises a scFv comprising an amino acid sequence of SEQ ID NO: 23.

In some embodiments, an anti-BCMA CAR further comprises a hinge region. In some embodiments, the hinge region is a CD8 hinge region. An exemplary amino acid sequence for a CD8 hinge region is set forth in SEQ ID NO: 82:

TTTPAPRPPTPAPTIASOPLSLRPEACRPAAGGAVHTRGLDFACD

In some embodiments, an anti-BCMA CAR comprises a CD8 hinge region having the amino acid sequence as set forth in SEQ ID NO: 82, or a variant thereof. In some embodiments, an anti-BCMA CAR comprises a hinge region comprising an amino acid sequence at least 70%, at least 80%, at least 85%, at least 90%, at least 92%, at least 95%, at least 98%, at least 99%, or 100% identical to SEQ ID NO: 82. In some embodiments, the hinge region can be any suitable hinge regions known in the art, e.g., the hinge regions described by Guedan et al., Engineering and Design of Chimeric Antigen Receptors, Mol Ther Methods Clin Dev. 2019 Mar. 15; 12:145-156, such as hinge regions derived from IgG1, IgG2, IgG4, CD28, CD8, or a hybrid thereof.

In some embodiments, an anti-BCMA CAR further comprises a transmembrane domain, which links the extracellular ligand-binding domain with the intracellular signaling and co-stimulatory domains. With respect to the transmembrane domain, the CAR can be designed to comprise a transmembrane domain that is fused to the extracellular domain (e.g., the antigen binding domain) of the CAR. Any transmembrane domain is contemplated for use herein as long as the domain is capable of anchoring a CAR comprising the domain to a cell membrane. In some embodiments, the transmembrane domain that naturally is associated with one of the domains in the CAR is used. In some instances, the transmembrane domain can be selected or modified by amino acid substitution to avoid binding of such domains to the transmembrane domains of the same or different surface membrane proteins to minimize interactions with other members of the receptor complex. One skilled in the art would appreciate that the full transmembrane domain, or portion thereof, is implemented with the cytoplasmic domain, or a portion thereof. The transmembrane domain may be derived either from a natural or from a synthetic source. Where the source is natural, the domain may be derived from any membrane-bound or transmembrane protein. In some embodiments, the transmembrane domain may be synthetic, in which case it will comprise predominantly hydrophobic residues such as leucine and valine. Preferably a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the cytoplasmic signaling domain of the CAR. A glycine-serine doublet provides a particularly suitable linker.

In some embodiments, the transmembrane domain is a CD8 transmembrane domain. An exemplary amino acid sequence for a CD8 transmembrane domain is set forth in SEQ ID NO: 83:

IYIWAPLAGTCGVLLLSLVITLYC

In some embodiments, an anti-BCMA CAR comprises a CD8 transmembrane domain having the amino acid sequence as set forth in SEQ ID NO: 83, or a variant thereof. In some embodiments, an anti-BCMA CAR comprises a transmembrane domain comprising an amino acid sequence at least 70%, at least 80%, at least 85%, at least 90%, at least 92%, at least 95%, at least 98%, at least 99%, or 100% identical to SEQ ID NO: 83. In some embodiments, the transmembrane domain can be any suitable transmembrane domain known in the art, e.g., transmembrane domain derived from TCRα, TCRβ, TCRζ, CD3ζ, CD3ε, CD3γ, CD3δ, CD4, CD5, CD8, CD9, CD16, CD22, CD28, CD32, CD33, CD34, CD37, CD45, CD64, CD80, CD86, CD134, CD137, CD154, or inducible T cell costimulator (ICOS). However, any transmembrane domain is contemplated for use herein as long as the domain is capable of anchoring a CAR comprising the extracellular domain to a cell membrane. Transmembrane domains can be identified using any method known in the art or described herein, e.g., by using the UniProt Database.

In some embodiments, an anti-BCMA CAR further comprises an intracellular (or cytoplasmic) domain. In some embodiments, the intracellular domain of the CAR is responsible for activation of at least one of the normal effector functions of the immune cell in which the CAR has been placed in. The term “effector function” refers to a specialized function of a cell (e.g., an iNKT cell). Effector function of a cell (e.g., an iNKT cell), for example, may be cytolytic activity or helper activity including the secretion of cytokines. Thus the term “intracellular signaling domain” refers to the portion of a protein which transduces the effector function signal and directs the cell to perform a specialized function. While usually the entire intracellular signaling domain can be employed, in many cases it is not necessary to use the entire domain. To the extent that a truncated portion of the intracellular signaling domain is used, such truncated portion may be used in place of the intact domain as long as it transduces the effector function signal. The term intracellular signaling domain is thus meant to include any truncated portion of the intracellular signaling domain sufficient to transduce the effector function signal. In certain embodiments, the intracellular (or cytoplasmic) domain of a chimeric antigen receptor as disclosed herein may include, but is not limited to, a 4-1BB intracellular domain, a OX40 intracellular domain, a CD30 intracellular domain, a CD40 intracellular domain, an ICOS intracellular domain, a LFA-1 intracellular domain, a CD2 intracellular domain, a CD3ζ intracellular domain, a CD3 γ intracellular domain, a CD3 δ intracellular domain, a CD3 ε intracellular domain, and a CD7 intracellular domain, and a CD22 intracellular domain. In some embodiments, an anti-BCMA CAR comprises a CD3 ζ intracellular domain. An exemplary amino acid sequence of a CD3 ζ intracellular domain is set forth in SEQ ID NO: 84:

RVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATK
DTYDALHMQALPPR

In some embodiments, an anti-BCMA CAR comprises a CD3 ζ intracellular domain having the amino acid sequence as set forth in SEQ ID NO: 84, or a variant thereof. In some embodiments, an anti-BCMA CAR comprises an intracellular domain comprising an amino acid sequence at least 70%, at least 80%, at least 85%, at least 90%, at least 92%, at least 95%, at least 98%, at least 99%, or 100% identical to SEQ ID NO: 84.

In some embodiments, the intracellular domain further comprises one or more intracellular co-stimulatory domains, such as those described herein, which transmit a co-stimulatory signal which promotes cell proliferation, cell survival, and/or cytokine secretion after binding of the extracellular domain. In some embodiments, such intracellular co-stimulatory domains include, without limitation, any co-stimulatory domain disclosed herein or those domains known in the art, including but not limited to CD28, ICOS, 4-1BB, OX40, or CD27. In some embodiments, an anti-BCMA CAR comprises a 4-1BB co-stimulatory domain. An exemplary amino acid sequence of a 4-1BB co-stimulatory domain is set forth in SEQ ID NO: 85:

KRGRKKLLYIFKQPFMRPVQTTQEEDGCSCRFPEEEEGGCEL

In some embodiments, an anti-BCMA CAR comprises a 4-1BB costimulatory domain having the amino acid sequence as set forth in SEQ ID NO: 85, or a variant thereof. In some embodiments, an anti-BCMA CAR comprises a 4-1BB co-stimulatory domain comprising an amino acid sequence at least 70%, at least 80%, at least 85%, at least 90%, at least 92%, at least 95%, at least 98%, at least 99%, or 100% identical to SEQ ID NO: 85.

The intracellular signaling domain of a chimeric antigen receptor of the present disclosure is responsible for activation of at least one of the normal effector functions of the cell in which the CAR has been placed and/or activation of proliferative and cell survival pathways.

It is to be understood that a chimeric antigen receptor as disclosed herein can include a domain (e.g., an extracellular domain, a transmembrane domain, an intracellular (cytoplasmic) domain, a co-stimulatory domain, a signaling domain, or any combination thereof) having a sequence as set forth herein, or a variant thereof, or a fragment thereof, of any one or more of the domains disclosed herein (e.g., a variant and/or fragment that retains the function required for the chimeric antigen receptor activity).

In some embodiments, exemplary anti-BCMA CAR amino acid sequences are set forth in Table 4.

TABLE 4
anti-BCMA CAR amino acid sequences

| Anti-BCMA | | |
|---|---|---|
| CAR | Sequences | SEQ ID NO |
| F6G CAR | QVQLVQSGAEVKKPGASVKVSCKASGYTFTGYYMHWVR | 86 |
| | QAPGQGLEWMGWMNPNSGNTGYAQKFQGRVTMTRNTSI | |
| | STAYMELSSLRSEDTAVYYCARAQWEPLDYWGQGTLVT | |
| | VSSGGGGSGGGGSGGGASSYELTQPPSVSVSPGQTARI | |
| | TCSGDALPKKYTYWYQQKSGQAPVLVIYEDTKRPSGIP | |
| | ERFSGSSSGTMATLTISGAQVEDEADYYCLSTDPTGQE | |
| | SVFGGGTKLTVLGQPAAATTTPAPRPPTPAPTIASQPL | |
| | SLRPEACRPAAGGAVHTRGLDFACDIYIWAPLAGTCGV | |
| | LLLSLVITLYCKRGRKKLLYIFKQPFMRPVQTTQEEDG | |
| | CSCRFPEEEEGGCELRVKFSRSADAPAYQQGQNQLYNE | |
| | LNLGRREEYDVLDKRRGRDPEMGGKPRRKNPQEGLYNE | |
| | LQKDKMAEAYSEIGMKGERRRGKGHDGLYQGLSTATKD | |
| | TYDALHMQALPPR | |
| F6G-Var1 CAR | QVQLVQSGAEVKKPGASVKVSCKASGYTFTGYYMHWVR | 87 |
| | QAPGQGLEWMGWMNPNSGNTGYAQKFQGRVTMTRNTSI | |
| | STAYMELSSLRSEDTAVYYCARAQWEPLDYWGQGTLVT | |
| | VSSGGGGSGGGGSGGGASSYELTQPPSVSVSPGQTARI | |
| | TCSGDALPKKYTYWYQQKSGQAPVLVIYYDTKRPSGIP | |
| | ERFSGSSSGTMATLTISGAQVEDEADYYCLSTDPTGQE | |
| | SVFGGGTKLTVLGQPAAATTTPAPRPPTPAPTIASQPL | |
| | SLRPEACRPAAGGAVHTRGLDFACDIYIWAPLAGTCGV | |
| | LLLSLVITLYCKRGRKKLLYIFKQPFMRPVQTTQEEDG | |
| | CSCRFPEEEEGGCELRVKFSRSADAPAYQQGQNQLYNE | |
| | LNLGRREEYDVLDKRRGRDPEMGGKPRRKNPQEGLYNE | |
| | LQKDKMAEAYSEIGMKGERRRGKGHDGLYQGLSTATKD | |
| | TYDALHMQALPPR | |
| F6G-Var2 CAR | QVQLVQSGAEVKKPGASVKVSCKASGYTFTGYYMQWVR | 88 |
| | QAPGQGLEWMGWMNPISGNTGYAQKFQGRVTMTRNTSI | |
| | STAYMELSSLRSEDTAVYYCARAQWEPLDYWGQGTLVT | |
| | VSSGGGGSGGGGSGGGASSYELTQPPSVSVSPGQTARI | |
| | TCSGDALPKKYTYWYQQKSGQAPVLVIYYDTKRPSGIP | |
| | ERFSGSSSGTMATLTISGAQVEDEADYYCLSTHPAGQE | |
| | SVFGGGTKLTVLGQPAAATTTPAPRPPTPAPTIASQPL | |
| | SLRPEACRPAAGGAVHTRGLDFACDIYIWAPLAGTCGV | |
| | LLLSLVITLYCKRGRKKLLYIFKQPFMRPVQTTQEEDG | |
| | CSCRFPEEEEGGCELRVKFSRSADAPAYQQGQNQLYNE | |
| | LNLGRREEYDVLDKRRGRDPEMGGKPRRKNPQEGLYNE | |
| | LQKDKMAEAYSEIGMKGERRRGKGHDGLYQGLSTATKD | |
| | TYDALHMQALPPR | |
| F6G-Var3 CAR | QVQLVQSGAEVKKPGASVKVSCKASGYTFTGYYMQWVR | 89 |
| | QAPGQGLEWMGWMNPISGNTGYAQKFQGRVTMTRNTSI | |
| | STAYMELSSLRSEDTAVYYCARAQWEPLDYWGQGTLVT | |
| | VSSGGGGSGGGGSGGGASSYELTQPPSVSVSPGQTARI | |
| | TCSGDALPKKYTYWYQQKSGQAPVLVIYYDTKRPSGIP | |
| | ERFSGSSSGTMATLTISGAQVEDEADYYCLSTNPTGHE | |
| | SVFGGGTKLTVLGQPAAATTTPAPRPPTPAPTIASQPL | |
| | SLRPEACRPAAGGAVHTRGLDFACDIYIWAPLAGTCGV | |
| | LLLSLVITLYCKRGRKKLLYIFKQPFMRPVQTTQEEDG | |
| | CSCRFPEEEEGGCELRVKFSRSADAPAYQQGQNQLYNE | |
| | LNLGRREEYDVLDKRRGRDPEMGGKPRRKNPQEGLYNE | |
| | LQKDKMAEAYSEIGMKGERRRGKGHDGLYQGLSTATKD | |
| | TYDALHMQALPPR | |
| F6G-Var4 CAR | QVQLVQSGAEVKKPGASVKVSCKASGYTFTGYYMHWVR | 90 |
| | QAPGQGLEWMGWMNPNSGNTGYAQKFQGRVTMTRNTSI | |
| | STAYMELSSLRSEDTAVYYCARAQWEPLDYWGQGTLVT | |
| | VSSGGGGSGGGGSGGGASSYELTQPPSVSVSPGQTARI | |
| | TCSGDALPKKYTYWYQQKSGQAPVLVIYYDTKRPSGIP | |
| | ERFSGSSSGTMATLTISGAQVEDEADYYCLSTHPAGQE | |
| | SVFGGGTKLTVLGQPAAATTTPAPRPPTPAPTIASQPL | |
| | SLRPEACRPAAGGAVHTRGLDFACDIYIWAPLAGTCGV | |
| | LLLSLVITLYCKRGRKKLLYIFKQPFMRPVQTTQEEDG | |
| | CSCRFPEEEEGGCELRVKFSRSADAPAYQQGQNQLYNE | |
| | LNLGRREEYDVLDKRRGRDPEMGGKPRRKNPQEGLYNE | |
| | LQKDKMAEAYSEIGMKGERRRGKGHDGLYQGLSTATKD | |
| | TYDALHMQALPPR | |
| F6G-Var5 CAR | QVQLVQSGAEVKKPGASVKVSCKASGYTFTGYYMHWVR | 91 |
| | QAPGQGLEWMGWMNPNSGNTGYAQKFQGRVTMTRNTSI | |
| | STAYMELSSLRSEDTAVYYCARAQWEPLDYWGQGTLVT | |
| | VSSGGGGSGGGGSGGGASSYELTQPPSVSVSPGQTARI | |
| | TCSGDALPKKYTYWYQQKSGQAPVLVIYYDTKRPSGIP | |
| | ERFSGSSSGTMATLTISGAQVEDEADYYCLSTNPTGHE | |
| | SVFGGGTKLTVLGQPAAATTTPAPRPPTPAPTIASQPL | |
| | SLRPEACRPAAGGAVHTRGLDFACDIYIWAPLAGTCGV | |
| | LLLSLVITLYCKRGRKKLLYIFKQPFMRPVQTTQEEDG | |
| | CSCRFPEEEEGGCELRVKFSRSADAPAYQQGQNQLYNE | |
| | LNLGRREEYDVLDKRRGRDPEMGGKPRRKNPQEGLYNE | |
| | LQKDKMAEAYSEIGMKGERRRGKGHDGLYQGLSTATKD | |
| | TYDALHMQALPPR | |
| F6G-Var6 CAR | QVQLVQSGAEVKKPGASVKVSCKASGYTFTEYYMKWVR | 92 |
| | QAPGQGLEWMGWMNPNTGKTGYAQKFQGRVTMTRNTSI | |
| | STAYMELSSLRSEDTAVYYCARAQWEPLDYWGQGTLVT | |
| | VSSGGGGSGGGGSGGGASSYELTQPPSVSVSPGQTARI | |
| | TCSGDALPKKYTYWYQQKSGQAPVLVIYYDTKRPSGIP | |
| | ERFSGSSSGTMATLTISGAQVEDEADYYCLSTDPTGQE | |
| | SVFGGGTKLTVLGQPAAATTTPAPRPPTPAPTIASQPL | |
| | SLRPEACRPAAGGAVHTRGLDFACDIYIWAPLAGTCGV | |
| | LLLSLVITLYCKRGRKKLLYIFKQPFMRPVQTTQEEDG | |
| | CSCRFPEEEEGGCELRVKESRSADAPAYQQGQNQLYNE | |
| | LNLGRREEYDVLDKRRGRDPEMGGKPRRKNPQEGLYNE | |
| | LQKDKMAEAYSEIGMKGERRRGKGHDGLYQGLSTATKD | |
| | TYDALHMQALPPR | |
| F1G scFv CAR | QVQLVQSGAEVKKPGASVKVSCKASGYTFTSYGISWVR | 93 |
| | QAPGQGLEWMGWISAYNGNTYYAEDLQGRLTMTTDTST | |
| | STAYMELRSLRSDDTAVYYCAREGSYYDSSGYYLPYWG | |
| | QGTLVTVSSGGGGSGGGGSGGGASQSALTQPRSVSGSP | |
| | GQSVTISCTGTSSDVGAYNYVSWYQQHPGKAPKLMIYD | |
| | VTERPSGVPDRESGSKSGNTASLTISGLQAEDEADYYC | |
| | SSYTSSSIAFGGGTKLTVLGQPAAATTTPAPRPPTPAP | |
| | TIASQPLSLRPEACRPAAGGAVHTRGLDFACDIYIWAP | |
| | LAGTCGVLLLSLVITLYCKRGRKKLLYIFKQPFMRPVQ | |
| | TTQEEDGCSCRFPEEEEGGCELRVKFSRSADAPAYQQG | |
| | QNQLYNELNLGRREEYDVLDKRRGRDPEMGGKPRRKNP | |
| | QEGLYNELQKDKMAEAYSEIGMKGERRRGKGHDGLYQG | |
| | LSTATKDTYDALHMQALPPR | |

In some embodiments, an anti-BCMA CAR comprises an amino acid sequence containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the anti-BCMA CAR amino acid sequence as set forth in SEQ ID NO: 86. In some embodiments, an anti-BCMA CAR comprises an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the anti-BCMA CAR amino acid sequence as set forth in SEQ ID NO: 86. In some embodiments, an anti-BCMA CAR comprises an amino acid sequence of SEQ ID NO: 86.

In some embodiments, an anti-BCMA CAR comprises an amino acid sequence containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the anti-BCMA CAR amino acid sequence as set forth in SEQ ID NO: 87. In some embodiments, an anti-BCMA CAR comprises an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the anti-BCMA CAR amino acid sequence as set forth in SEQ ID NO: 87. In some embodiments, an anti-BCMA CAR comprises an amino acid sequence of SEQ ID NO: 87.

In some embodiments, an anti-BCMA CAR comprises an amino acid sequence containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the anti-BCMA CAR amino acid sequence as set forth in SEQ ID NO: 88. In some embodiments, an anti-BCMA CAR comprises an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the anti-BCMA CAR amino acid sequence as set forth in SEQ ID NO: 88. In some embodiments, an anti-BCMA CAR comprises an amino acid sequence of SEQ ID NO: 88.

In some embodiments, an anti-BCMA CAR comprises an amino acid sequence containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the anti-BCMA CAR amino acid sequence as set forth in SEQ ID NO: 89. In some embodiments, an anti-BCMA CAR comprises an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the anti-BCMA CAR amino acid sequence as set forth in SEQ ID NO: 89. In some embodiments, an anti-BCMA CAR comprises an amino acid sequence of SEQ ID NO: 89.

In some embodiments, an anti-BCMA CAR comprises an amino acid sequence containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the anti-BCMA CAR amino acid sequence as set forth in SEQ ID NO: 90. In some embodiments, an anti-BCMA CAR comprises an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the anti-BCMA CAR amino acid sequence as set forth in SEQ ID NO: 90. In some embodiments, an anti-BCMA CAR comprises an amino acid sequence of SEQ ID NO: 90.

In some embodiments, an anti-BCMA CAR comprises an amino acid sequence containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the anti-BCMA CAR amino acid sequence as set forth in SEQ ID NO: 91. In some embodiments, an anti-BCMA CAR comprises an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the anti-BCMA CAR amino acid sequence as set forth in SEQ ID NO: 91. In some embodiments, an anti-BCMA CAR comprises an amino acid sequence of SEQ ID NO: 91.

In some embodiments, an anti-BCMA CAR comprises an amino acid sequence containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the anti-BCMA CAR amino acid sequence as set forth in SEQ ID NO: 92. In some embodiments, an anti-BCMA CAR comprises an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the anti-BCMA CAR amino acid sequence as set forth in SEQ ID NO: 92. In some embodiments, an anti-BCMA CAR comprises an amino acid sequence of SEQ ID NO: 92.

In some embodiments, an anti-BCMA CAR comprises an amino acid sequence containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the anti-BCMA CAR amino acid sequence as set forth in SEQ ID NO: 93. In some embodiments, an anti-BCMA CAR comprises an amino acid sequence that is at least 80% (e.g., at least 80%, at least 85%, at least 90%, at least 91%, at least 92%, at least 93%, at least 94%, at least 95%, at least 96%, at least 97%, at least 98%, at least 99%, or 100%) identical to the anti-BCMA CAR amino acid sequence as set forth in SEQ ID NO: 93. In some embodiments, an anti-BCMA CAR comprises an amino acid sequence of SEQ ID NO: 93.

### III. Genetically Modified Cells Expressing the Anti-BCMA Chimeric Antigen Receptor and Method for Producing the Same

Also provided by the present disclosure are genetically modified cells that comprises at least one of the anti-BCMA antibodies or antigen binding fragments thereof, or at least one of the anti-BCMA CAR as disclosed herein.

In some embodiments, a cell can be genetically modified to express any one of the chimeric antigen receptors described herein. In some embodiments, the genetically modified cells described herein are eukaryotic cells. In some embodiments, the genetically modified cells comprising the CARs (e.g., anti-BCMA CAR) as described herein are human cells. In some embodiments, the genetically modified cells comprising the CARs (e.g., anti-BCMA CAR) as described herein are immune cells. In some embodiments, the genetically-modified cells the CARs (e.g., anti-BCMA CAR) are immune cells (e.g., T cells such as cytotoxic T lymphocytes, regulatory T cells, NK cells, NKT cells such as invariant NKT cells, macrophages, monocytes, neutrophils, eosinophils, or any combination thereof). In some embodiments, the genetically modified immune cells are iNKT cells modified to express one or more of the anti-BCMA CARs as described herein.

In some embodiments, the present disclosure provides a population of genetically modified immune cells (e.g., iNKT cells) comprising an anti-BCMA CAR described herein. In some embodiments, the present disclosure provides a population of genetically modified immune cells (e.g., iNKT cells) comprising an anti-BCMA CAR which comprises a CDRH1, a CDRH2 and a CDRH3 of a heavy chain variable domain (VH) having the amino acid sequence of any one of SEQ ID NOs: 7, 12, 19, or 21, and/or a CDRL1, a CDRL2 and a CDRL3 of a light chain variable domain (VL) having the amino acid sequence of any one of SEQ ID NOs: 8, 10, 13, 15, or 22.

In some embodiments, the present disclosure provides a population of genetically modified immune cells (e.g., iNKT cells) comprising: (i) an anti-BCMA CAR comprises a CDRH1 having the amino acid sequence of SEQ ID NO: 1, a CDRH2 having the amino acid sequence of SEQ ID NO: 2, a CDRH3 having the amino acid sequence of SEQ ID NO: 3, a CDRL1 having the amino acid sequence of SEQ ID NO: 4, a CDRL2 having the amino acid sequence of SEQ ID NO: 5, and a CDRL3 having the amino acid sequence of SEQ ID NO: 6; (ii) an anti-BCMA CAR comprises a CDRH1 having the amino acid sequence of SEQ ID NO: 1, a CDRH2 having the amino acid sequence of SEQ ID NO: 2, a CDRH3 having the amino acid sequence of SEQ ID NO: 3, a CDRL1 having the amino acid sequence of SEQ ID NO: 4, a CDRL2 having the amino acid sequence of SEQ ID NO: 24, and a CDRL3 having the amino acid sequence of SEQ ID NO: 6; (iii) an anti-BCMA CAR comprises a CDRH1 having the amino acid sequence of SEQ ID NO: 25, a CDRH2 having the amino acid sequence of SEQ ID NO: 27, a CDRH3 having the amino acid sequence of SEQ ID NO: 3, a CDRL1 having the amino acid sequence of SEQ ID NO: 4, a CDRL2 having the amino acid sequence of SEQ ID NO: 24, and a CDRL3 having the amino acid sequence of SEQ ID NO: 30; (iv) an anti-BCMA CAR comprises a CDRH1 having the amino acid sequence of SEQ ID NO: 25, a CDRH2 having the amino acid sequence of SEQ ID NO: 27, a CDRH3 having the amino acid sequence of SEQ ID NO: 3, a CDRL1 having the amino acid sequence of SEQ ID NO: 4, a CDRL2 having the amino acid sequence of SEQ ID NO: 24, and a CDRL3 having the amino acid sequence of SEQ ID NO: 29; (v) an anti-BCMA CAR comprises a CDRH1 having the amino acid sequence of SEQ ID NO: 1, a CDRH2 having the amino acid sequence of SEQ ID NO: 2, a CDRH3 having the amino acid sequence of SEQ ID NO: 3, a CDRL1 having the amino acid sequence of SEQ ID NO: 4, a CDRL2 having the amino acid sequence of SEQ ID NO: 24, and a CDRL3 having the amino acid sequence of SEQ ID NO: 30; (vi) an anti-BCMA CAR comprises a CDRH1 having the amino acid sequence of SEQ ID NO: 1, a CDRH2 having the amino acid sequence of SEQ ID NO: 2, a CDRH3 having the amino acid sequence of SEQ ID NO: 3, a CDRL1 having the amino acid sequence of SEQ ID NO: 4, a CDRL2 having the amino acid sequence of SEQ ID NO: 24, and a CDRL3 having the amino acid sequence of SEQ ID NO: 29; (vii) an anti-BCMA CAR comprises a CDRH1 having the amino acid sequence of SEQ ID NO: 26, a CDRH2 having the amino acid sequence of SEQ ID NO: 28, a CDRH3 having the amino acid sequence of SEQ ID NO: 3, a CDRL1 having the amino acid sequence of SEQ ID NO: 4, a CDRL2 having the amino acid sequence of SEQ ID NO: 24, and a CDRL3 having the amino acid sequence of SEQ ID NO: 6; or (viii) an anti-BCMA CAR comprises a CDRH1 having the amino acid sequence of SEQ ID NO: 31, a CDRH2 having the amino acid sequence of SEQ ID NO: 32, a CDRH3 having the amino acid sequence of SEQ ID NO: 33, a CDRL1 having the amino acid sequence of SEQ ID NO: 34, a CDRL2 having the amino acid sequence of SEQ ID NO: 35, and a CDRL3 having the amino acid sequence of SEQ ID NO: 36.

In some embodiments, the present disclosure provides a population of genetically modified immune cells (e.g., iNKT cells) comprising: (i) an anti-BCMA CAR comprises a VH comprising the amino acid sequence of SEQ ID NO: 7, and/or a VL comprising the amino acid sequence of SEQ ID NO: 8; (ii) an anti-BCMA CAR comprises a VH comprising the amino acid sequence of SEQ ID NO: 7, and/or a VL comprising the amino acid sequence of SEQ ID NO: 10; (iii) an anti-BCMA CAR comprises a VH comprising the amino acid sequence of SEQ ID NO: 12, and/or a VL comprising the amino acid sequence of SEQ ID NO: 13; (iv) an anti-BCMA CAR comprises a VH comprising the amino acid sequence of SEQ ID NO: 12, and/or a VL comprising the amino acid sequence of SEQ ID NO: 15; (v) an anti-BCMA CAR comprises a VH comprising the amino acid sequence of SEQ ID NO: 7, and/or a VL comprising the amino acid sequence of SEQ ID NO: 13; (vi) an anti-BCMA CAR comprises a VH comprising the amino acid sequence of SEQ ID NO: 7, and/or a VL comprising the amino acid sequence of SEQ ID NO: 15; (vii) an anti-BCMA CAR comprises a VH comprising the amino acid sequence of SEQ ID NO: 19, and/or a VL comprising the amino acid sequence of SEQ ID NO: 10; or (viii) an anti-BCMA CAR comprises a VH comprising the amino acid sequence of SEQ ID NO: 21, and/or a VL comprising the amino acid sequence of SEQ ID NO: 22.

In some embodiments, the present disclosure provides a population of genetically modified immune cells (e.g., iNKT cells) comprising an anti-BCMA CAR, which comprises a scFv as the extracellular ligand-binding domain comprising an amino acid sequence of any one of SEQ ID NOs: 9, 11, 14, 16, 17, 18, 20, or 23.

In some embodiments, the present disclosure provides a population of genetically modified immune cells (e.g., iNKT cells) comprising an anti-BCMA CAR, which comprises a scFv as the extracellular ligand-binding domain comprising an amino acid sequence of any one of SEQ ID NOs: 9, 11, 14, 16, 17, 18, 20, or 23.

In some embodiments, the present disclosure provides a population of genetically modified immune cells (e.g., iNKT cells) comprising an anti-BCMA CAR comprising an amino acid sequence of any one of SEQ ID NOs: 86-93.

In some embodiments, the genetically modified cells (e.g., iNKT cells) are armored CAR expressing cells engineered to express another molecule (e.g., a cytokine or ligand) capable of enhancing one or more properties (e.g., survival/persistence, immune interaction with other cells such as macrophages and/or dendritic cells, disrupt immunosuppressive tumor microenvironment) of the genetically modified cells. The genetically modified cells (e.g., iNKT cells) can be engineered to express any suitable armoring molecule known in the art, e.g., armoring molecule described by Yeku et al., Armored CAR T-cells: utilizing cytokines and pro-inflammatory ligands to enhance CAR T-cell anti-tumour efficacy, Biochem Soc Trans. 2016 Apr. 15; 44 (2): 412-418; Hawkins et al., Armored CAR T-Cells: The Next Chapter in T-Cell Cancer Immunotherapy, Biologics. 2021; 15:95-105). Non-limiting examples of armoring molecules include IL-15, IL-2, IL-12, CD40L, 4-1BBL, IL-18, IL-7, IL-33, dominant-negative TGF-β receptor II (dnTGF-BRII), constitutively active Akt (caAkt), hybrid IL-4/IL-7 receptor, checkpoint Inhibitors such as anti-PD1 antibodies, nanobodies targeting CD47, or bispecific T-cell engagers (BiTEs).

In some embodiments, the genetically modified cells expressing the CAR described herein (e.g., an anti-BCMA CAR) are also engineered to express IL-15. In some embodiments, the genetically modified cells expressing the CAR described herein (e.g., an anti-BCMA CAR) are also engineered to express secreted IL-15 (sIL-15). The role of IL-15 in enhancing the expansion and function of T cells and natural killer T cells (e.g., iNKT cells) have been previously described (see, e.g., Lin et al., Interleukin-15 enhances the expansion and function of natural killer T cells from adult peripheral and umbilical cord blood, Cytokine. 2015 December; 76 (2): 348-355; Battram et al., IL-15 Enhances the Persistence and Function of BCMA-Targeting CAR-T Cells Compared to IL-2 or IL-15/IL-7 by Limiting CAR-T Cell Dysfunction and Differentiation; Cancers (Basel). 2021 July; 13 (14): 3534). There is evidence that numerous cell types are responsible for the production of IL-15, including macrophages and DCs, and once released, IL-15 stimulates CD8+ T-cells and NK cells which increases their proliferation and cytotoxic capacity. Administration of IL-15 to mice has been shown to enhance anti-tumor activity of adoptively transferred CD8+ tumor-reactive T-cells, which suggests IL-15 could also enhance anti-tumor activity of CAR T-cell therapy. CAR T-cells further engineered to secrete IL-15 have been shown to augment tumor cytotoxicity compared to using CAR alone T-cells in vitro and in vivo, and increase T-cell expansion on antigen recognition in vitro. Further, IL-15 can increase antigen-independent T-cell proliferation, while enabling T-cell persistence after tumor clearance. It was also known that the secretion of IL-15 provided greater protection against tumor rechallenge in vivo than CAR T-cells not secreting IL-15. Certain previous studies used a form of IL-15 tethered to the membrane and found that this promoted the CAR T-cells to develop a memory phenotype. These data indicate that IL-15 could provide long-term CAR T-cell-mediated immunity toward the cancer antigen along with enhancing CAR T-cell function within the tumor microenvironment.

In some embodiments, the present disclosure, at least in part, is based on the discovery using secreted IL-15 as an “armor molecule” for the genetically modified immune cells (e.g., iNKT cells) expressing an anti-BCMA CAR. In some embodiments, secreted IL-15 enhances the persistence of the genetically modified immune cells (e.g., iNKT cells) in the tumor microenvironment (e.g., relative to persistence of other modified cells not producing an armoring molecule such as IL-15).

In some embodiments, a nucleic acid encoding a CAR (e.g., anti-BCMA CAR) is a multicistronic vector encoding a secreted IL-15 in addition to the CAR. An exemplary IL-15 coding sequence is set forth in SEQ ID NO: 94:

ATGCGGATCTCCAAGCCCCACCTCCGAAGCATCAGTATCCAGTGTTATC
TGTGCCTTCTTCTGAATTCCCATTTTTTGACAGAGGCAGGAATTCATGT
ATTCATATTGGGTTGTTTCTCCGCGGGGCTTCCTAAAACCGAAGCTAAC
TGGGTTAATGTCATTTCAGATCTGAAGAAAATTGAAGATCTTATTCAAT
CAATGCATATCGACGCGACCTTGTACACGGAGAGTGATGTCCACCCGAG
CTGCAAGGTAACGGCCATGAAATGCTTCTTGCTGGAATTGCAGGTAATT
AGTCTTGAGAGTGGAGATGCGTCTATACACGACACCGTAGAGAATCTCA
TTATTTTGGCGAACAATTCTCTCAGCAGTAACGGCAATGTTACTGAATC
AGGATGTAAGGAGTGTGAAGAGCTGGAAGAAAAGAACATTAAGGAATTT
TTGCAGAGCTTCGTACATATCGTCCAAATGTTCATAAATACAAGC

In some embodiments, the nucleic acid encoding the CAR (e.g., anti-BCMA CAR) further comprises a nucleic acid sequence at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the nucleic acid sequence of SEQ ID NO: 94.

In some embodiments, a multicistronic vector comprises two or more expression cassettes encoding one or more anti-BCMA CAR, and a sIL-15.

In some embodiments, multicistronic expression constructs are comprise expression cassettes that are positioned in different ways. For example, in some embodiments, a multicistronic expression construct is provided in which a first expression cassette (e.g., an expression cassette encoding an anti-BCMA CAR, or portion thereof) is positioned adjacent to a second expression cassette (e.g., an expression cassette encoding sIL-15). In some embodiments, the first expression cassette (e.g., an expression cassette encoding an anti-BCMA CAR, or portion thereof) is positioned 5′ to the second expression cassette (e.g., an expression cassette encoding sIL-15). In some embodiments, the first expression cassette (e.g., an expression cassette encoding an anti-BCMA CAR, or portion thereof) is positioned 3′ to the second expression cassette (e.g., an expression cassette encoding sIL-15). Multicistronic vectors have been previously described, for example, by Shaimardanova et al., Production and Application of Multicistronic Constructs for Various Human Disease Therapies, Pharmaceutics 2019, 11, 580. In some embodiments, the multicistronic vector comprises a nucleic acid sequence encoding a self-cleaving 2A peptide between the first expression cassette (e.g., an expression cassette encoding an anti-BCMA CAR, or portion thereof) and the second expression cassette (e.g., an expression cassette encoding sIL-15). In some embodiments, the self-cleaving 2A peptide is a P2A peptide. In some embodiments, the self-cleaving 2A peptide is a T2A peptide. In some embodiments, the multicistronic vector comprises a nucleic acid sequence encoding an internal ribosome entry site (IRES) between the first expression cassette (e.g., an expression cassette encoding an anti-BCMA CAR, or portion thereof) and the second expression cassette (e.g., an expression cassette encoding sIL-15).

In some embodiments, the nucleic acid sequence encoding sIL-15 (e.g., sIL-15 coding sequence as set forth in SEQ ID NO: 94) are delivered to the cell in a separate construct from the construct encoding the CAR. In some embodiments, the cell already express secreted IL-15 (e.g., engineered to express sIL-15) prior to receiving the nucleic acid construct encoding the CAR (e.g., anti-BCMA CAR).

Also provided herein are cells (e.g., iNKT cells) comprising a nucleic acid molecule encoding a chimeric antigen receptor as disclosed herein. In some embodiments, a nucleic acid molecule encoding a CAR (e.g., anti-BCMA CAR) as described herein is present (i.e., integrated) within the genome of the genetically-modified cell or, alternatively, is not integrated into the genome of the cell. In some embodiments, where the nucleic acid molecule encoding a CAR (e.g., anti-BCMA CAR) is not integrated into the genome, the nucleic acid molecule is present in the genetically-modified cell in a recombinant DNA construct, in an mRNA, in a viral genome, or in another nucleic acid which is not integrated into the genome of the cell.

In one embodiment, genetically-modified cells (e.g., iNKT cells) contain a nucleic acid molecule encoding a chimeric antigen receptor (e.g., an anti-BCMA CAR) as disclosed herein, positioned within the genome of a cell. In other embodiments, genetically-modified cells (e.g., an iNKT cell) contain a nucleic acid molecule encoding a chimeric antigen receptor (e.g., an anti-BCMA CAR) as disclosed herein, positioned within the endogenous T cell receptor alpha gene of the cell.

In some embodiments, the present invention encompasses an isolated nucleic acid comprising sequences of a CAR (e.g., an anti-BCMA CAR), wherein the sequence comprises the nucleic acid sequence of an extracellular ligand-binding domain operably linked to the nucleic acid sequence of transmembrane domain and a cytoplasmic domain. The nucleic acid sequences coding for the desired molecules can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned. The present invention also provides vectors in which a DNA of the present invention is inserted. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity. In another embodiment, the desired CAR can be expressed in the cells by way of transposons.

In brief summary, the expression of natural or synthetic nucleic acids encoding CARs is typically achieved by operably linking a nucleic acid encoding the CAR polypeptide (e.g., an anti-BCMA CAR) or portions thereof to a promoter, and incorporating the construct into an expression vector (e.g., a lentiviral vector). The vectors can be suitable for replication and integration into eukaryotes. Typical cloning vectors contain transcription and translation terminators, initiation sequences, and promoters useful for regulation of the expression of the desired nucleic acid sequence. The expression constructs of the present invention may also be used for nucleic acid immunization and gene therapy, using standard gene delivery protocols. Methods for gene delivery are known in the art. See, e.g., U.S. Pat. Nos. 5,399,346, 5,580,859, 5,589,466, incorporated by reference herein in their entireties.

The nucleic acid can be cloned into a number of types of vectors. For example, the nucleic acid can be cloned into a vector including, but not limited to a plasmid, a viral vector a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

A number of viral based systems have been developed for gene transfer into mammalian cells. For example, lentiviruses provide a convenient platform for gene delivery systems. A selected gene can be inserted into a vector and packaged in lentiviral particles using techniques known in the art. The recombinant virus can then be isolated and delivered to cells of the subject either in vivo or ex vivo. A number of lentiviral systems are known in the art.

In some embodiments, the vector described herein further comprises a promoter operably linked to the nucleic acid encoding the CAR described herein (e.g., an anti-BCMA CAR). Non-limiting examples of suitable promoters include cytomegalovirus (CMV) promoter, Elongation Factor-1α (EF-1α) promoter, simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. In some embodiments, the promoter is a EF-1α promoter. Further, the present disclosure also contemplate the use of inducible promoters. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

In order to assess the expression of a CAR polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as kanamycin resistant gene and the like, and fluorescent genes such as GFP, YFP, RFP and the like. In some embodiments, reporter genes or selectable marker genes are excluded from a CAR polypeptide used in a therapy as described herein.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means. Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York). A preferred method for the introduction of a polynucleotide into a host cell is calcium phosphate transfection. Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle). In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Regardless of the method used to introduce exogenous nucleic acids into a host cell or otherwise expose a cell to the inhibitor of the present invention, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

In some embodiments, the nucleic acid sequences encoding the anti-BCMA antibodies or antigen binding fragments thereof, the BCMA CAR, and the lentiviral vector sequences are set forth in Table 5.

TABLE 5
Nucleic acid sequences encoding the anti-BCMA antibodies, the
anti-BCMA CAR, and the lentiviral vector sequences.

| | | | SEQ |
|---|---|---|---|
| | | | ID |
| | | Coding Sequences | NO |
| F6G | VH | CAAGTTCAACTGGTTCAGTCTGGCGCCGAAGTGAAGAAACCTGGCGCCTCTGTG | 49 |
| | | AAGGTGTCCTGCAAGGCCAGCGGCTACACCTTTACCGGCTACTACATGCACTGG | |
| | | GTCCGACAGGCTCCAGGCCAAGGACTTGAATGGATGGGCTGGATGAACCCCAAC | |
| | | AGCGGCAATACCGGCTATGCCCAGAAATTCCAGGGCAGAGTGACCATGACCAGA | |
| | | AACACCAGCATCAGCACCGCCTACATGGAACTGAGCAGCCTGAGAAGCGAGGAC | |
| | | ACCGCCGTGTACTACTGTGCTAGAGCCCAGTGGGAGCCCCTGGATTATTGGGGA | |
| | | CAGGGCACACTGGTCACAGTCTCGAG | |
| | VL | TCTTATGAACTGACCCAGCCTCCAAGCGTGTCCGTGTCTCCTGGACAGACCGCC | 57 |
| | | AGAATCACATGTAGCGGAGATGCCCTGCCTAAGAAGTACACCTACTGGTATCAG | |
| | | CAGAAGTCCGGACAGGCCCCTGTGCTGGTCATCTACGAGGATACCAAACGGCCC | |
| | | AGCGGCATCCCCGAGAGATTTTCTGGAAGCAGCAGCGGCACAATGGCCACACTG | |
| | | ACAATTAGCGGAGCCCAGGTGGAAGATGAGGCCGACTACTACTGCCTGAGCACC | |
| | | GATCCTACAGGCCAAGAGTCCGTTTTTGGCGGAGGCACCAAGCTGACAGTGCTG | |
| | | GGACAACCT | |
| | scFv | CAAGTTCAACTGGTTCAGTCTGGCGCCGAAGTGAAGAAACCTGGCGCCTCTGTG | 65 |
| | | AAGGTGTCCTGCAAGGCCAGCGGCTACACCTTTACCGGCTACTACATGCACTGG | |
| | | GTCCGACAGGCTCCAGGCCAAGGACTTGAATGGATGGGCTGGATGAACCCCAAC | |
| | | AGCGGCAATACCGGCTATGCCCAGAAATTCCAGGGCAGAGTGACCATGACCAGA | |
| | | AACACCAGCATCAGCACCGCCTACATGGAACTGAGCAGCCTGAGAAGCGAGGAC | |
| | | ACCGCCGTGTACTACTGTGCTAGAGCCCAGTGGGAGCCCCTGGATTATTGGGGA | |
| | | CAGGGCACACTGGTCACAGTCTCGAGTGGTGGAGGCGGTTCAGGCGGAGGTGGC | |
| | | TCTGGCGGTGGCGCTAGCTCTTATGAACTGACCCAGCCTCCAAGCGTGTCCGTG | |
| | | TCTCCTGGACAGACCGCCAGAATCACATGTAGCGGAGATGCCCTGCCTAAGAAG | |
| | | TACACCTACTGGTATCAGCAGAAGTCCGGACAGGCCCCTGTGCTGGTCATCTAC | |
| | | GAGGATACCAAACGGCCCAGCGGCATCCCCGAGAGATTTTCTGGAAGCAGCAGC | |
| | | GGCACAATGGCCACACTGACAATTAGCGGAGCCCAGGTGGAAGATGAGGCCGAC | |
| | | TACTACTGCCTGAGCACCGATCCTACAGGCCAAGAGTCCGTTTTTGGCGGAGGC | |
| | | ACCAAGCTGACAGTGCTGGGACAACCT | |
| | CAR | CAAGTTCAACTGGTTCAGTCTGGCGCCGAAGTGAAGAAACCTGGCGCCTCTGTG | 95 |
| | | AAGGTGTCCTGCAAGGCCAGCGGCTACACCTTTACCGGCTACTACATGCACTGG | |
| | | GTCCGACAGGCTCCAGGCCAAGGACTTGAATGGATGGGCTGGATGAACCCCAAC | |
| | | AGCGGCAATACCGGCTATGCCCAGAAATTCCAGGGCAGAGTGACCATGACCAGA | |
| | | AACACCAGCATCAGCACCGCCTACATGGAACTGAGCAGCCTGAGAAGCGAGGAC | |
| | | ACCGCCGTGTACTACTGTGCTAGAGCCCAGTGGGAGCCCCTGGATTATTGGGGA | |
| | | CAGGGCACACTGGTCACAGTCTCGAGTGGTGGAGGCGGTTCAGGCGGAGGTGGC | |
| | | TCTGGCGGTGGCGCTAGCTCTTATGAACTGACCCAGCCTCCAAGCGTGTCCGTG | |
| | | TCTCCTGGACAGACCGCCAGAATCACATGTAGCGGAGATGCCCTGCCTAAGAAG | |
| | | TACACCTACTGGTATCAGCAGAAGTCCGGACAGGCCCCTGTGCTGGTCATCTAC | |
| | | GAGGATACCAAACGGCCCAGCGGCATCCCCGAGAGATTTTCTGGAAGCAGCAGC | |
| | | GGCACAATGGCCACACTGACAATTAGCGGAGCCCAGGTGGAAGATGAGGCCGAC | |
| | | TACTACTGCCTGAGCACCGATCCTACAGGCCAAGAGTCCGTTTTTGGCGGAGGC | |
| | | ACCAAGCTGACAGTGCTGGGACAACCTGCGGCCGCAACCACAACCCCTGCTCCA | |
| | | AGGCCCCCCACACCCGCTCCAACTATAGCCAGCCAACCATTGAGCCTCAGACCT | |
| | | GAAGCTTGCAGGCCCGCAGCAGGAGGCGCCGTCCATACGCGAGGCCTGGACTTC | |
| | | GCGTGTGATATTTATATTTGGGCCCCTTTGGCCGGAACATGTGGGGTGTTGCTT | |
| | | CTCTCCCTTGTGATCACTCTGTATTGTAAGCGCGGGAGAAAGAAGCTCCTGTAC | |
| | | ATCTTCAAGCAGCCTTTTATGCGACCTGTGCAAACCACTCAGGAAGAAGATGGG | |
| | | TGTTCATGCCGCTTCCCCGAGGAGGAAGAAGGAGGGTGTGAACTGAGGGTGAAA | |
| | | TTTTCTAGAAGCGCCGATGCTCCCGCATATCAGCAGGGTCAGAATCAGCTCTAC | |
| | | AATGAATTGAATCTCGGCAGGCGAGAAGAGTACGATGTTCTGGACAAGAGACGG | |
| | | GGCAGGGATCCCGAGATGGGGGGAAAGCCCCGGAGAAAAAATCCTCAGGAGGGG | |
| | | TTGTACAATGAGCTGCAGAAGGACAAGATGGCTGAAGCCTATAGCGAGATCGGA | |
| | | ATGAAAGGCGAAAGACGCAGAGGCAAGGGGCATGACGGTCTGTACCAGGGTCTC | |
| | | TCTACAGCCACCAAGGACACTTATGATGCGTTGCATATGCAAGCCTTGCCACCC | |
| | | CGC | |
| | Lentivira | cgttcagcccgaccgctgcgccttatccggtaactatcgtcttgagtccaacCC | 73 |
| | 1 vector | GGTAAGACACGACTTATCGCCACTGGCAGCAGCCACTGGTAACAGGATTAGCAG | |
| | | AGCGAGGTATGTAGGCGGTGCTACAGAGTTCTTGAAGTGGTGGCCTAACTACGG | |
| | | CTACACTAGAAGAACAGTATTTGGTATCTGCGCTCTGCTGAAGCCAGTTACCTT | |
| | | CGGAAAAAGAGTTGGTAGCTCTTGATCCGGCAAACAAACCACCGCTGGTAGCGG | |
| | | TGGTTTTTTTGTTTGCAAGCAGCAGATTACGCGCAGAAAAAAAGGATCTCAAGA | |
| | | AGATCCTTTGATCTTTTCTACGGGGTCTGACGCTCAGTGGAACGAAAACTCACG | |
| | | TTAAGGGATTTTGGTCATGAGATTATCAAAAAGGATCTTCACCTAGATCCTTTT | |
| | | AAATTAAAAATGAAGTTTTAAATCAATCTAAAGTATATATGAGTAAACTTGGTC | |
| | | TGACAGTTAGAAAAACTCATCGAGCATCAAATGAAACTGCAATTTATTCATATC | |
| | | AGGATTATCAATACCATATTTTTGAAAAAGCCGTTTCTGTAATGAAGGAGAAAA | |
| | | CTCACCGAGGCAGTTCCATAGGATGGCAAGATCCTGGTATCGGTCTGCGATTCC | |
| | | GACTCGTCCAACATCAATACAACCTATTAATTTCCCCTCGTCAAAAATAAGGTT | |
| | | ATCAAGTGAGAAATCACCATGAGTGACGACTGAATCCGGTGAGAATGGCAAAAG | |
| | | TTTATGCATTTCTTTCCAGACTTGTTCAACAGGCCAGCCATTACGCTCGTCATC | |
| | | AAAATCACTCGCATCAACCAAACCGTTATTCATTCGTGATTGCGCCTGAGCGAG | |
| | | ACGAAATACGCGATCGCTGTTAAAAGGACAATTACAAACAGGAATCGAATGCAA | |
| | | CCGGCGCAGGAACACTGCCAGCGCATCAACAATATTTTCACCTGAATCAGGATA | |
| | | TTCTTCTAATACCTGGAATGCTGTTTTTCCGGGGATCGCAGTGGTGAGTAACCA | |
| | | TGCATCATCAGGAGTACGGATAAAATGCTTGATGGTCGGAAGAGGCATAAATTC | |
| | | CGTCAGCCAGTTTAGTCTGACCATCTCATCTGTAACATCATTGGCAACGCTACC | |
| | | TTTGCCATGTTTCAGAAACAACTCTGGCGCATCGGGCTTCCCATACAAGCGATA | |
| | | GATTGTCGCACCTGATTGCCCGACATTATCGCGAGCCCATTTATACCCATATAA | |
| | | ATCAGCATCCATGTTGGAATTTAATCGCGGCCTCGACGTTTCCCGTTGAATATG | |
| | | GCTCATACTCTTCCTTTTTCAATATTATTGAAGCATTTATCAGGGTTATTGTCT | |
| | | CATGAGCGGATACATATTTGAATGTATTTAGAAAAATAAACAAATAGGGGTTCC | |
| | | GCGCACATTTCCCCGAAAAGTGCCACCTGACGGATCGGGAGATCTCCCGATCCC | |
| | | CTATGGTGCACTCTCAGTACAATCTGCTCTGATGCCGCATAGTTAAGCCAGTAT | |
| | | CTGCTCCCTGCTTGTGTGTTGGAGGTCGCTGAGTAGTGCGCGAGCAAAATTTAA | |
| | | GCTACAACAAGGCAAGGCTTGACCGACAATTGCATGAAGAATCTGCTTAGGGTT | |
| | | AGGCGTTTTGCGCTGCTTCGCGATGTACGGGCCAGATATACCCGTTGACATTGA | |
| | | TTATTGACTAGTTATTAATAGTAATCAATTACGGGGTCATTAGTTCATAGCCCA | |
| | | TATATGGAGTTCCGCGTTACATAACTTACGGTAAATGGCCCGCCTGGCTGACCG | |
| | | CCCAACGACCCCCGCCCATTGACGTCAATAATGACGTATGTTCCCATAGTAACG | |
| | | CCAATAGGGACTTTCCATTGACGTCAATGGGTGGAGTATTTACGGTAAACTGCC | |
| | | CACTTGGCAGTACATCAAGTGTATCATATGCCAAGTACGCCCCCTATTGACGTC | |
| | | AATGACGGTAAATGGCCCGCCTGGCATTATGCCCAGTACATGACCTTATGGGAC | |
| | | TTTCCTACTTGGCAGTACATCTACGTATTAGTCATCGCTATTACCATGGTGATG | |
| | | CGGTTTTGGCAGTACATCAATGGGCGTGGATAGCGGTTTGACTCACGGGGATTT | |
| | | CCAAGTCTCCACCCCATTGACGTCAATGGGAGTTTGTTTTGGCACCAAAATCAA | |
| | | CGGGACTTTCCAAAATGTCGTAACAACTCCGCCCCATTGACGCAAATGGGCGGT | |
| | | AGGCGTGTACGGTGGGAGGTCTATATAAGCAGCGCGTTTTGCCTGTACTGGGTC | |
| | | TCTCTGGTTAGACCAGATCTGAGCCTGGGAGCTCTCTGGCTAACTAGGGAACCC | |
| | | ACTGCTTAAGCCTCAATAAAGCTTGCCTTGAGTGCTTCAAGTAGTGTGTGCCCG | |
| | | TCTGTTGTGTGACTCTGGTAACTAGAGATCCCTCAGACCCTTTTAGTCAGTGTG | |
| | | GAAAATCTCTAGCAGTGGCGCCCGAACAGGGACTTGAAAGCGAAAGGGAAACCA | |
| | | GAGGAGCTCTCTCGACGCAGGACTCGGCTTGCTGAAGCGCGCACGGCAAGAGGC | |
| | | GAGGGGCGGCGACTGGTGAGTACGCCAAAAATTTTGACTAGCGGAGGCTAGAAG | |
| | | GAGAGAGATGGGTGCGAGAGCGTCAGTATTAAGCGGGGGAGAATTAGATCGCGA | |
| | | TGGGAAAAAATTCGGTTAAGGCCAGGGGGAAAGAAAAAATATAAATTAAAACAT | |
| | | ATAGTATGGGCAAGCAGGGAGCTAGAACGATTCGCAGTTAATCCTGGCCTGTTA | |
| | | GAAACATCAGAAGGCTGTAGACAAATACTGGGACAGCTACAACCATCCCTTCAG | |
| | | ACAGGATCAGAAGAACTTAGATCATTATATAATACAGTAGCAACCCTCTATTGT | |
| | | GTGCATCAAAGGATAGAGATAAAAGACACCAAGGAAGCTTTAGACAAGATAGAG | |
| | | GAAGAGCAAAACAAAAGTAAGACCACCGCACAGCAAGCGGCCGCTGATCTTCAG | |
| | | ACCTGGAGGAGGAGATATGAGGGACAATTGGAGAAGTGAATTATATAAATATAA | |
| | | AGTAGTAAAAATTGAACCATTAGGAGTAGCACCCACCAAGGCAAAGAGAAGAGT | |
| | | GGTGCAGAGAGAAAAAAGAGCAGTGGGAATAGGAGCTTTGTTCCTTGGGTTCTT | |
| | | GGGAGCAGCAGGAAGCACTATGGGCGCAGCGTCAATGACGCTGACGGTACAGGC | |
| | | CAGACAATTATTGTCTGGTATAGTGCAGCAGCAGAACAATTTGCTGAGGGCTAT | |
| | | TGAGGCGCAACAGCATCTGTTGCAACTCACAGTCTGGGGCATCAAGCAGCTCCA | |
| | | GGCAAGAATCCTGGCTGTGGAAAGATACCTAAAGGATCAACAGCTCCTGGGGAT | |
| | | TTGGGGTTGCTCTGGAAAACTCATTTGCACCACTGCTGTGCCTTGGAATGCTAG | |
| | | TTGGAGTAATAAATCTCTGGAACAGATTTGGAATCACACGACCTGGATGGAGTG | |
| | | GGACAGAGAAATTAACAATTACACAAGCTTAATACACTCCTTAATTGAAGAATC | |
| | | GCAAAACCAGCAAGAAAAGAATGAACAAGAATTATTGGAATTAGATAAATGGGC | |
| | | AAGTTTGTGGAATTGGTTTAACATAACAAATTGGCTGTGGTATATAAAATTATT | |
| | | CATAATGATAGTAGGAGGCTTGGTAGGTTTAAGAATAGTTTTTGCTGTACTTTC | |
| | | TATAGTGAATAGAGTTAGGCAGGGATATTCACCATTATCGTTTCAGACCCACCT | |
| | | CCCAACCCCGAGGGGACCCGACAGGCCCGAAGGAATAGAAGAAGAAGGTGGAGA | |
| | | GAGAGACAGAGACAGATCCATTCGATTAGTGAACGGATCTCGACGGTATCGGTT | |
| | | AACTTTTAAAAGAAAAGGGGGGATTGGGGGGTACAGTGCAGGGGAAAGAATAGT | |
| | | AGACATAATAGCAACAGACATACAAACTAAAGAATTACAAAAACAAATTACAAA | |
| | | ATTCAAAATTTTATCGATACTAGTATTATGCCCAGTACATGACCTTATGGGACT | |
| | | TTCCTACTTGGCAGTACATCTACAAACCCCGGGTGCAAAGATGGATAAAGTTTT | |
| | | AAACAGAGAGGAATCTTTGCAGCTAATGGACCTTCTAGGTCTTGAAAGGAGTGG | |
| | | GAATTGGCTCCGGTGCCCGTCAGTGGGCAGAGCGCACATCGCCCACAGTCCCCG | |
| | | AGAAGTTGGGGGGAGGGGTCGGCAATTGATCCGGTGCCTAGAGAAGGTGGCGCG | |
| | | GGGTAAACTGGGAAAGTGATGTCGTGTACTGGCTCCGCCTTTTTCCCGAGGGTG | |
| | | GGGGAGAACCGTATATAAGTGCAGTAGTCGCCGTGAACGTTCTTTTTCGCAACG | |
| | | GGTTTGCCGCCAGAACACAGGTAAGTGCCGTGTGTGGTTCCCGCGGGCCTGGCC | |
| | | TCTTTACGGGTTATGGCCCTTGCGTGCCTTGAATTACTTCCACTGGCTGCAGTA | |
| | | CGTGATTCTTGATCCCGAGCTTCGGGTTGGAAGTGGGTGGGAGAGTTCGAGGCC | |
| | | TTGCGCTTAAGGAGCCCCTTCGCCTCGTGCTTGAGTTGAGGCCTGGCCTGGGCG | |
| | | CTGGGGCCGCCGCGTGCGAATCTGGTGGCACCTTCGCGCCTGTCTCGCTGCTTT | |
| | | CGATAAGTCTCTAGCCATTTAAAATTTTTGATGACCTGCTGCGACGCTTTTTTT | |
| | | CTGGCAAGATAGTCTTGTAAATGCGGGCCAAGATCTGCACACTGGTATTTCGGT | |
| | | TTTTGGGGCCGCGGGCGGCGACGGGGCCCGTGCGTCCCAGCGCACATGTTCGGC | |
| | | GAGGCGGGGCCTGCGAGCGCGGCCACCGAGAATCGGACGGGGGTAGTCTCAAGC | |
| | | TGGCCGGCCTGCTCTGGTGCCTGGCCTCGCGCCGCCGTGTATCGCCCCGCCCTG | |
| | | GGCGGCAAGGCTGGCCCGGTCGGCACCAGTTGCGTGAGCGGAAAGATGGCCGCT | |
| | | TCCCGGCCCTGCTGCAGGGAGCTCAAAATGGAGGACGCGGCGCTCGGGAGAGCG | |
| | | GGCGGGTGAGTCACCCACACAAAGGAAAAGGGCCTTTCCGTCCTCAGCCGTCGC | |
| | | TTCATGTGACTCCACGGAGTACCGGGCGCCGTCCAGGCACCTCGATTAGTTCTC | |
| | | GAGCTTTTGGAGTACGTCGTCTTTAGGTTGGGGGGAGGGGTTTTATGCGATGGA | |
| | | GTTTCCCCACACTGAGTGGGTGGAGACTGAAGTTAGGCCAGCTTGGCACTTGAT | |
| | | GTAATTCTCCTTGGAATTTGCCCTTTTTGAGTTTGGATCTTGGTTCATTCTCAA | |
| | | GCCTCAGACAGTGGTTCAAAGTTTTTTTCTTCCATTTCAGGTGTCGTGACGTAG | |
| | | CGGCCCTAATACGACTCACTATAGGGCCCGCGTACGTCTAGACCTCCAGGCATC | |
| | | GATTCCACCATGGCACTCCCCGTCACCGCCCTTCTCTTGCCCCTCGCCCTGCTG | |
| | | CTGCATGCTGCCAGGCCCCAAGTTCAACTGGTTCAGTCTGGCGCCGAAGTGAAG | |
| | | AAACCTGGCGCCTCTGTGAAGGTGTCCTGCAAGGCCAGCGGCTACACCTTTACC | |
| | | GGCTACTACATGCACTGGGTCCGACAGGCTCCAGGCCAAGGACTTGAATGGATG | |
| | | GGCTGGATGAACCCCAACAGCGGCAATACCGGCTATGCCCAGAAATTCCAGGGC | |
| | | AGAGTGACCATGACCAGAAACACCAGCATCAGCACCGCCTACATGGAACTGAGC | |
| | | AGCCTGAGAAGCGAGGACACCGCCGTGTACTACTGTGCTAGAGCCCAGTGGGAG | |
| | | CCCCTGGATTATTGGGGACAGGGCACACTGGTCACAGTCTCGAGTGGTGGAGGC | |
| | | GGTTCAGGCGGAGGTGGCTCTGGCGGTGGCGCTAGCTCTTATGAACTGACCCAG | |
| | | CCTCCAAGCGTGTCCGTGTCTCCTGGACAGACCGCCAGAATCACATGTAGCGGA | |
| | | GATGCCCTGCCTAAGAAGTACACCTACTGGTATCAGCAGAAGTCCGGACAGGCC | |
| | | CCTGTGCTGGTCATCTACGAGGATACCAAACGGCCCAGCGGCATCCCCGAGAGA | |
| | | TTTTCTGGAAGCAGCAGCGGCACAATGGCCACACTGACAATTAGCGGAGCCCAG | |
| | | GTGGAAGATGAGGCCGACTACTACTGCCTGAGCACCGATCCTACAGGCCAAGAG | |
| | | TCCGTTTTTGGCGGAGGCACCAAGCTGACAGTGCTGGGACAACCTGCGGCCGCA | |
| | | ACCACAACCCCTGCTCCAAGGCCCCCCACACCCGCTCCAACTATAGCCAGCCAA | |
| | | CCATTGAGCCTCAGACCTGAAGCTTGCAGGCCCGCAGCAGGAGGCGCCGTCCAT | |
| | | ACGCGAGGCCTGGACTTCGCGTGTGATATTTATATTTGGGCCCCTTTGGCCGGA | |
| | | ACATGTGGGGTGTTGCTTCTCTCCCTTGTGATCACTCTGTATTGTAAGCGCGGG | |
| | | AGAAAGAAGCTCCTGTACATCTTCAAGCAGCCTTTTATGCGACCTGTGCAAACC | |
| | | ACTCAGGAAGAAGATGGGTGTTCATGCCGCTTCCCCGAGGAGGAAGAAGGAGGG | |
| | | TGTGAACTGAGGGTGAAATTTTCTAGAAGCGCCGATGCTCCCGCATATCAGCAG | |
| | | GGTCAGAATCAGCTCTACAATGAATTGAATCTCGGCAGGCGAGAAGAGTACGAT | |
| | | GTTCTGGACAAGAGACGGGGCAGGGATCCCGAGATGGGGGGAAAGCCCCGGAGA | |
| | | AAAAATCCTCAGGAGGGGTTGTACAATGAGCTGCAGAAGGACAAGATGGCTGAA | |
| | | GCCTATAGCGAGATCGGAATGAAAGGCGAAAGACGCAGAGGCAAGGGGCATGAC | |
| | | GGTCTGTACCAGGGTCTCTCTACAGCCACCAAGGACACTTATGATGCGTTGCAT | |
| | | ATGCAAGCCTTGCCACCCCGCGGATCAGGAGCGACTAACTTCTCTTTGTTGAAG | |
| | | CAAGCGGGTGACGTTGAAGAGAATCCCGGTCCATCCGGGGGATCCATGCGGATC | |
| | | TCCAAGCCCCACCTCCGAAGCATCAGTATCCAGTGTTATCTGTGCCTTCTTCTG | |
| | | AATTCCCATTTTTTGACAGAGGCAGGAATTCATGTATTCATATTGGGTTGTTTC | |
| | | TCCGCGGGGCTTCCTAAAACCGAAGCTAACTGGGTTAATGTCATTTCAGATCTG | |
| | | AAGAAAATTGAAGATCTTATTCAATCAATGCATATCGACGCGACCTTGTACACG | |
| | | GAGAGTGATGTCCACCCGAGCTGCAAGGTAACGGCCATGAAATGCTTCTTGCTG | |
| | | GAATTGCAGGTAATTAGTCTTGAGAGTGGAGATGCGTCTATACACGACACCGTA | |
| | | GAGAATCTCATTATTTTGGCGAACAATTCTCTCAGCAGTAACGGCAATGTTACT | |
| | | GAATCAGGATGTAAGGAGTGTGAAGAGCTGGAAGAAAAGAACATTAAGGAATTT | |
| | | TTGCAGAGCTTCGTACATATCGTCCAAATGTTCATAAATACAAGCTAAGTCGAC | |
| | | GGCCGGCCGCGAGCATCTTACCGCCATTTATACCCATATTTGTTCTGTTTTTCT | |
| | | TGATTTGGGTATACATTTAAATGTTAATAAAACAAAATGGTGGGGCAATCATTT | |
| | | ACATTTTATAGGATATGTAATTACTAGTTCAGGTGTATTGCCACAAGACAAACA | |
| | | TCTTAAGAAACTTTCCCGTTATTTACGCTCTGTTCCTGTTAATCAACCTCTGGA | |
| | | TTACAAAATTTGTGAAAGATTGACTGATATTCTTAACTATGTTGCTCCTTTTAC | |
| | | GCTGTGTGGATATGCTGCTTTAATACCTCTGTATCATACTATTGCTTCCTGTAC | |
| | | GGCTTTCGTTTTCTCCTCCTTGTATAAATCCTGGTTGCTGTCTCTTTATCAGGA | |
| | | GTTGTGGCCCGTTGTCCGTCAACGTGGCGTGGTGTGCTCTGTGTTTGCTGACGC | |
| | | AACCCCCACTGGCTGGGGCATTGCCACCACCTGTCAACTCCTTTCTGGGACTTT | |
| | | CGCTTTCCCCCTCCCGATCGCCACGGCAGAACTCATCGCCGCCTGCCTTGCCCG | |
| | | CTGCTGGACAGGGGCTAGGTTGCTGGGCACTGATAATTCCGTGGTGTTGTCCTG | |
| | | GTACCTTTAAGACCAATGACTTACAAGGCAGCTGTAGATCTTAGCCACTTTTTA | |
| | | AAAGAAAAGGGGGGACTGGAAGGGCTAATTCACTCCCAACGAAGATAAGATCTG | |
| | | CTTTTTGCTTGTACTGGGTCTCTCTGGTTAGACCAGATCTGAGCCTGGGAGCTC | |
| | | TCTGGCTAACTAGGGAACCCACTGCTTAAGCCTCAATAAAGCTTGCCTTGAGTG | |
| | | CTTCAAGTAGTGTGTGCCCGTCTGTTGTGTGACTCTGGTAACTAGAGATCCCTC | |
| | | AGACCCTTTTAGTCAGTGTGGAAAATCTCTAGCAGTAGTAGTTCATGTCATCTT | |
| | | ATTATTCAGTATTTATAACTTGCAAAGAAATGAATATCAGAGAGTGAGAGGAAC | |
| | | TTGTTTATTGCAGCTTATAATGGTTACAAATAAAGCAATAGCATCACAAATTTC | |
| | | ACAAATAAAGCATTTTTTTCACTGCATTCTAGTTGTGGTTTGTCCAAACTCATC | |
| | | AATGTATCTTATCATGTCTGGCTCTAGCTATCCCGCCCCTAACTCCGCCCATCC | |
| | | CGCCCCTAACTCCGCCCAGTTCCGCCCATTCTCCGCCCCATGGCTGACTAATTT | |
| | | TTTTTATTTATGCAGAGGCCGAGGCCGCCTCGGCCTCTGAGCTATTCCAGAAGT | |
| | | AGTGAGGAGGCTTTTTTGGAGGCCTAGACTTTTGCAGAGACCAAATTCGTAATC | |
| | | ATGTCATAGCTGTTTCCTGTGTGAAATTGTTATCCGCTCACAATTCCACACAAC | |
| | | ATACGAGCCGGAAGCATAAAGTGTAAAGCCTGGGGTGCCTAATGAGTGAGCTAA | |
| | | CTCACATTAATTGCGTTGCGCTCACTGCCCGCTTTCCAGTCGGGAAACCTGTCG | |
| | | TGCCAGCTGCATTAATGAATCGGCCAACGCGCGGGGAGAGGCGGTTTGCGTATT | |
| | | GGGCGCTCTTCCGCTTCCTCGCTCACTGACTCGCTGCGCTCGGTCGTTCGGCTG | |
| | | CGGCGAGCGGTATCAGCTCACTCAAAGGCGGTAATACGGTTATCCACAGAATCA | |
| | | GGGGATAACGCAGGAAAGAACATGTGAGCAAAAGGCCAGCAAAAGGCCAGGAAC | |
| | | CGTAAAAAGGCCGCGTTGCTGGCGTTTTTCCATAGGCTCCGCCCCCCTGACGAG | |
| | | CATCACAAAAATCGACGCTCAAGTCAGAGGTGGCGAAACCCGACAGGACTATAA | |
| | | AGATACCAGGCGTTTCCCCCTGGAAGCTCCCTCGTGCGCTCTCCTGTTCCGACC | |
| | | CTGCCGCTTACCGGATACCTGTCCGCCTTTCTCCCTTCGGGAAGCGTGGCGCTT | |
| | | TCTCATAGCTCACGCTGTAGGTATCTCAGTTCGGTGTAGGTCGTTCGCTCCAAG | |
| | | CTGGGCTGTGTGCACGAACCCCC | |
| F6G- | VH | CAAGTTCAACTGGTTCAGTCTGGCGCCGAAGTGAAGAAACCTGGCGCCTCTGTG | 50 |
| Var1 | | AAGGTGTCCTGCAAGGCCAGCGGCTACACCTTTACCGGCTACTACATGCACTGG | |
| | | GTCCGACAGGCtcCAGGCCAAGGACTTGAATGGATGGGCTGGATGAACCCCAAC | |
| | | AGCGGCAATACCGGCTATGCCCAGAAATTCCAGGGCAGAGTGACCATGACCAGA | |
| | | AACACCAGCATCAGCACCGCCTACATGGAACTGAGCAGCCTGAGAAGCGAGGAC | |
| | | ACCGCCGTGTACTACTGTGCTAGAGCCCAGTGGGAGCCCCTGGATTATTGGGGA | |
| | | CAGGGCACACTGGTCACAGTCTCGAG | |
| | VL | TCTTATGAACTGACCCAGCCTCCAAGCGTGTCCGTGTCTCCTGGACAGACCGCC | 58 |
| | | AGAATCACATGTAGCGGAGATGCCCTGCCTAAGAAGTACACCTACTGGTATCAG | |
| | | CAGAAGTCCGGACAGGCCCCTGTGCTGGTCATCTACTATGATACCAAACGGCCC | |
| | | AGCGGCATCCCCGAGAGATTTTCTGGAAGCAGCAGCGGCACAATGGCCACACTG | |
| | | ACAATTAGCGGAGCCCAGGTGGAAGATGAGGCCGACTACTACTGCCTGAGCACC | |
| | | GATCCTACAGGCCAAGAGTCCGTTTTTGGCGGAGGCACCAAGCTGACAGTGCTG | |
| | | GGACAACCT | |
| | scFv | CAAGTTCAACTGGTTCAGTCTGGCGCCGAAGTGAAGAAACCTGGCGCCTCTGTG | 66 |
| | | AAGGTGTCCTGCAAGGCCAGCGGCTACACCTTTACCGGCTACTACATGCACTGG | |
| | | GTCCGACAGGCtcCAGGCCAAGGACTTGAATGGATGGGCTGGATGAACCCCAAC | |
| | | AGCGGCAATACCGGCTATGCCCAGAAATTCCAGGGCAGAGTGACCATGACCAGA | |
| | | AACACCAGCATCAGCACCGCCTACATGGAACTGAGCAGCCTGAGAAGCGAGGAC | |
| | | ACCGCCGTGTACTACTGTGCTAGAGCCCAGTGGGAGCCCCTGGATTATTGGGGA | |
| | | CAGGGCACACTGGTCACAGTCTCGAGTGGTGGAGGCGGTTCAGGCGGAGGTGGC | |
| | | TCTGGCGGTGGCGCTAGCTCTTATGAACTGACCCAGCCTCCAAGCGTGTCCGTG | |
| | | TCTCCTGGACAGACCGCCAGAATCACATGTAGCGGAGATGCCCTGCCTAAGAAG | |
| | | TACACCTACTGGTATCAGCAGAAGTCCGGACAGGCCCCTGTGCTGGTCATCTAC | |
| | | TATGATACCAAACGGCCCAGCGGCATCCCCGAGAGATTTTCTGGAAGCAGCAGC | |
| | | GGCACAATGGCCACACTGACAATTAGCGGAGCCCAGGTGGAAGATGAGGCCGAC | |
| | | TACTACTGCCTGAGCACCGATCCTACAGGCCAAGAGTCCGTTTTTGGCGGAGGC | |
| | | ACCAAGCTGACAGTGCTGGGACAACCT | |
| | CAR | CAAGTTCAACTGGTTCAGTCTGGCGCCGAAGTGAAGAAACCTGGCGCCTCTGTG | 96 |
| | | AAGGTGTCCTGCAAGGCCAGCGGCTACACCTTTACCGGCTACTACATGCACTGG | |
| | | GTCCGACAGGCtcCAGGCCAAGGACTTGAATGGATGGGCTGGATGAACCCCAAC | |
| | | AGCGGCAATACCGGCTATGCCCAGAAATTCCAGGGCAGAGTGACCATGACCAGA | |
| | | AACACCAGCATCAGCACCGCCTACATGGAACTGAGCAGCCTGAGAAGCGAGGAC | |
| | | ACCGCCGTGTACTACTGTGCTAGAGCCCAGTGGGAGCCCCTGGATTATTGGGGA | |
| | | CAGGGCACACTGGTCACAGTCTCGAGTGGTGGAGGCGGTTCAGGCGGAGGTGGC | |
| | | TCTGGCGGTGGCGCTAGCTCTTATGAACTGACCCAGCCTCCAAGCGTGTCCGTG | |
| | | TCTCCTGGACAGACCGCCAGAATCACATGTAGCGGAGATGCCCTGCCTAAGAAG | |
| | | TACACCTACTGGTATCAGCAGAAGTCCGGACAGGCCCCTGTGCTGGTCATCTAC | |
| | | TATGATACCAAACGGCCCAGCGGCATCCCCGAGAGATTTTCTGGAAGCAGCAGC | |
| | | GGCACAATGGCCACACTGACAATTAGCGGAGCCCAGGTGGAAGATGAGGCCGAC | |
| | | TACTACTGCCTGAGCACCGATCCTACAGGCCAAGAGTCCGTTTTTGGCGGAGGC | |
| | | ACCAAGCTGACAGTGCTGGGACAACCTGCGGCCGCAACCACAACCCCTGCTCCA | |
| | | AGGCCCCCCACACCCGCTCCAACTATAGCCAGCCAACCATTGAGCCTCAGACCT | |
| | | GAAGCTTGCAGGCCCGCAGCAGGAGGCGCCGTCCATACGCGAGGCCTGGACTTC | |
| | | GCGTGTGATATTTATATTTGGGCCCCTTTGGCCGGAACATGTGGGGTGTTGCTT | |
| | | CTCTCCCTTGTGATCACTCTGTATTGTAAGCGCGGGAGAAAGAAGCTCCTGTAC | |
| | | ATCTTCAAGCAGCCTTTTATGCGACCTGTGCAAACCACTCAGGAAGAAGATGGG | |
| | | TGTTCATGCCGCTTCCCCGAGGAGGAAGAAGGAGGGTGTGAACTGAGGGTGAAA | |
| | | TTTTCTAGAAGCGCCGATGCTCCCGCATATCAGCAGGGTCAGAATCAGCTCTAC | |
| | | AATGAATTGAATCTCGGCAGGCGAGAAGAGTACGATGTTCTGGACAAGAGACGG | |
| | | GGCAGGGATCCCGAGATGGGGGGAAAGCCCCGGAGAAAAAATCCTCAGGAGGGG | |
| | | TTGTACAATGAGCTGCAGAAGGACAAGATGGCTGAAGCCTATAGCGAGATCGGA | |
| | | ATGAAAGGCGAAAGACGCAGAGGCAAGGGGCATGACGGTCTGTACCAGGGTCTC | |
| | | TCTACAGCCACCAAGGACACTTATGATGCGTTGCATATGCAAGCCTTGCCACCC | |
| | | CGC | |
| | Lentivira | cgttcagcccgaccgctgcgccttatccggtaactatcgtcttgagtccaacCC | 74 |
| | 1 vector | GGTAAGACACGACTTATCGCCACTGGCAGCAGCCACTGGTAACAGGATTAGCAG | |
| | | AGCGAGGTATGTAGGCGGTGCTACAGAGTTCTTGAAGTGGTGGCCTAACTACGG | |
| | | CTACACTAGAAGAACAGTATTTGGTATCTGCGCTCTGCTGAAGCCAGTTACCTT | |
| | | CGGAAAAAGAGTTGGTAGCTCTTGATCCGGCAAACAAACCACCGCTGGTAGCGG | |
| | | TGGTTTTTTTGTTTGCAAGCAGCAGATTACGCGCAGAAAAAAAGGATCTCAAGA | |
| | | AGATCCTTTGATCTTTTCTACGGGGTCTGACGCTCAGTGGAACGAAAACTCACG | |
| | | TTAAGGGATTTTGGTCATGAGATTATCAAAAAGGATCTTCACCTAGATCCTTTT | |
| | | AAATTAAAAATGAAGTTTTAAATCAATCTAAAGTATATATGAGTAAACTTGGTC | |
| | | TGACAGTTAGAAAAACTCATCGAGCATCAAATGAAACTGCAATTTATTCATATC | |
| | | AGGATTATCAATACCATATTTTTGAAAAAGCCGTTTCTGTAATGAAGGAGAAAA | |
| | | CTCACCGAGGCAGTTCCATAGGATGGCAAGATCCTGGTATCGGTCTGCGATTCC | |
| | | GACTCGTCCAACATCAATACAACCTATTAATTTCCCCTCGTCAAAAATAAGGTT | |
| | | ATCAAGTGAGAAATCACCATGAGTGACGACTGAATCCGGTGAGAATGGCAAAAG | |
| | | TTTATGCATTTCTTTCCAGACTTGTTCAACAGGCCAGCCATTACGCTCGTCATC | |
| | | AAAATCACTCGCATCAACCAAACCGTTATTCATTCGTGATTGCGCCTGAGCGAG | |
| | | ACGAAATACGCGATCGCTGTTAAAAGGACAATTACAAACAGGAATCGAATGCAA | |
| | | CCGGCGCAGGAACACTGCCAGCGCATCAACAATATTTTCACCTGAATCAGGATA | |
| | | TTCTTCTAATACCTGGAATGCTGTTTTTCCGGGGATCGCAGTGGTGAGTAACCA | |
| | | TGCATCATCAGGAGTACGGATAAAATGCTTGATGGTCGGAAGAGGCATAAATTC | |
| | | CGTCAGCCAGTTTAGTCTGACCATCTCATCTGTAACATCATTGGCAACGCTACC | |
| | | TTTGCCATGTTTCAGAAACAACTCTGGCGCATCGGGCTTCCCATACAAGCGATA | |
| | | GATTGTCGCACCTGATTGCCCGACATTATCGCGAGCCCATTTATACCCATATAA | |
| | | ATCAGCATCCATGTTGGAATTTAATCGCGGCCTCGACGTTTCCCGTTGAATATG | |
| | | GCTCATACTCTTCCTTTTTCAATATTATTGAAGCATTTATCAGGGTTATTGTCT | |
| | | CATGAGCGGATACATATTTGAATGTATTTAGAAAAATAAACAAATAGGGGTTCC | |
| | | GCGCACATTTCCCCGAAAAGTGCCACCTGACGGATCGGGAGATCTCCCGATCCC | |
| | | CTATGGTGCACTCTCAGTACAATCTGCTCTGATGCCGCATAGTTAAGCCAGTAT | |
| | | CTGCTCCCTGCTTGTGTGTTGGAGGTCGCTGAGTAGTGCGCGAGCAAAATTTAA | |
| | | GCTACAACAAGGCAAGGCTTGACCGACAATTGCATGAAGAATCTGCTTAGGGTT | |
| | | AGGCGTTTTGCGCTGCTTCGCGATGTACGGGCCAGATATACCCGTTGACATTGA | |
| | | TTATTGACTAGTTATTAATAGTAATCAATTACGGGGTCATTAGTTCATAGCCCA | |
| | | TATATGGAGTTCCGCGTTACATAACTTACGGTAAATGGCCCGCCTGGCTGACCG | |
| | | CCCAACGACCCCCGCCCATTGACGTCAATAATGACGTATGTTCCCATAGTAACG | |
| | | CCAATAGGGACTTTCCATTGACGTCAATGGGTGGAGTATTTACGGTAAACTGCC | |
| | | CACTTGGCAGTACATCAAGTGTATCATATGCCAAGTACGCCCCCTATTGACGTC | |
| | | AATGACGGTAAATGGCCCGCCTGGCATTATGCCCAGTACATGACCTTATGGGAC | |
| | | TTTCCTACTTGGCAGTACATCTACGTATTAGTCATCGCTATTACCATGGTGATG | |
| | | CGGTTTTGGCAGTACATCAATGGGCGTGGATAGCGGTTTGACTCACGGGGATTT | |
| | | CCAAGTCTCCACCCCATTGACGTCAATGGGAGTTTGTTTTGGCACCAAAATCAA | |
| | | CGGGACTTTCCAAAATGTCGTAACAACTCCGCCCCATTGACGCAAATGGGCGGT | |
| | | AGGCGTGTACGGTGGGAGGTCTATATAAGCAGCGCGTTTTGCCTGTACTGGGTC | |
| | | TCTCTGGTTAGACCAGATCTGAGCCTGGGAGCTCTCTGGCTAACTAGGGAACCC | |
| | | ACTGCTTAAGCCTCAATAAAGCTTGCCTTGAGTGCTTCAAGTAGTGTGTGCCCG | |
| | | TCTGTTGTGTGACTCTGGTAACTAGAGATCCCTCAGACCCTTTTAGTCAGTGTG | |
| | | GAAAATCTCTAGCAGTGGCGCCCGAACAGGGACTTGAAAGCGAAAGGGAAACCA | |
| | | GAGGAGCTCTCTCGACGCAGGACTCGGCTTGCTGAAGCGCGCACGGCAAGAGGC | |
| | | GAGGGGCGGCGACTGGTGAGTACGCCAAAAATTTTGACTAGCGGAGGCTAGAAG | |
| | | GAGAGAGATGGGTGCGAGAGCGTCAGTATTAAGCGGGGGAGAATTAGATCGCGA | |
| | | TGGGAAAAAATTCGGTTAAGGCCAGGGGGAAAGAAAAAATATAAATTAAAACAT | |
| | | ATAGTATGGGCAAGCAGGGAGCTAGAACGATTCGCAGTTAATCCTGGCCTGTTA | |
| | | GAAACATCAGAAGGCTGTAGACAAATACTGGGACAGCTACAACCATCCCTTCAG | |
| | | ACAGGATCAGAAGAACTTAGATCATTATATAATACAGTAGCAACCCTCTATTGT | |
| | | GTGCATCAAAGGATAGAGATAAAAGACACCAAGGAAGCTTTAGACAAGATAGAG | |
| | | GAAGAGCAAAACAAAAGTAAGACCACCGCACAGCAAGCGGCCGCTGATCTTCAG | |
| | | ACCTGGAGGAGGAGATATGAGGGACAATTGGAGAAGTGAATTATATAAATATAA | |
| | | AGTAGTAAAAATTGAACCATTAGGAGTAGCACCCACCAAGGCAAAGAGAAGAGT | |
| | | GGTGCAGAGAGAAAAAAGAGCAGTGGGAATAGGAGCTTTGTTCCTTGGGTTCTT | |
| | | GGGAGCAGCAGGAAGCACTATGGGCGCAGCGTCAATGACGCTGACGGTACAGGC | |
| | | CAGACAATTATTGTCTGGTATAGTGCAGCAGCAGAACAATTTGCTGAGGGCTAT | |
| | | TGAGGCGCAACAGCATCTGTTGCAACTCACAGTCTGGGGCATCAAGCAGCTCCA | |
| | | GGCAAGAATCCTGGCTGTGGAAAGATACCTAAAGGATCAACAGCTCCTGGGGAT | |
| | | TTGGGGTTGCTCTGGAAAACTCATTTGCACCACTGCTGTGCCTTGGAATGCTAG | |
| | | TTGGAGTAATAAATCTCTGGAACAGATTTGGAATCACACGACCTGGATGGAGTG | |
| | | GGACAGAGAAATTAACAATTACACAAGCTTAATACACTCCTTAATTGAAGAATC | |
| | | GCAAAACCAGCAAGAAAAGAATGAACAAGAATTATTGGAATTAGATAAATGGGC | |
| | | AAGTTTGTGGAATTGGTTTAACATAACAAATTGGCTGTGGTATATAAAATTATT | |
| | | CATAATGATAGTAGGAGGCTTGGTAGGTTTAAGAATAGTTTTTGCTGTACTTTC | |
| | | TATAGTGAATAGAGTTAGGCAGGGATATTCACCATTATCGTTTCAGACCCACCT | |
| | | CCCAACCCCGAGGGGACCCGACAGGCCCGAAGGAATAGAAGAAGAAGGTGGAGA | |
| | | GAGAGACAGAGACAGATCCATTCGATTAGTGAACGGATCTCGACGGTATCGGTT | |
| | | AACTTTTAAAAGAAAAGGGGGGATTGGGGGGTACAGTGCAGGGGAAAGAATAGT | |
| | | AGACATAATAGCAACAGACATACAAACTAAAGAATTACAAAAACAAATTACAAA | |
| | | ATTCAAAATTTTATCGATACTAGTATTATGCCCAGTACATGACCTTATGGGACT | |
| | | TTCCTACTTGGCAGTACATCTACAAACCCCGGGTGCAAAGATGGATAAAGTTTT | |
| | | AAACAGAGAGGAATCTTTGCAGCTAATGGACCTTCTAGGTCTTGAAAGGAGTGG | |
| | | GAATTGGCTCCGGTGCCCGTCAGTGGGCAGAGCGCACATCGCCCACAGTCCCCG | |
| | | AGAAGTTGGGGGGAGGGGTCGGCAATTGATCCGGTGCCTAGAGAAGGTGGCGCG | |
| | | GGGTAAACTGGGAAAGTGATGTCGTGTACTGGCTCCGCCTTTTTCCCGAGGGTG | |
| | | GGGGAGAACCGTATATAAGTGCAGTAGTCGCCGTGAACGTTCTTTTTCGCAACG | |
| | | GGTTTGCCGCCAGAACACAGGTAAGTGCCGTGTGTGGTTCCCGCGGGCCTGGCC | |
| | | TCTTTACGGGTTATGGCCCTTGCGTGCCTTGAATTACTTCCACTGGCTGCAGTA | |
| | | CGTGATTCTTGATCCCGAGCTTCGGGTTGGAAGTGGGTGGGAGAGTTCGAGGCC | |
| | | TTGCGCTTAAGGAGCCCCTTCGCCTCGTGCTTGAGTTGAGGCCTGGCCTGGGCG | |
| | | CTGGGGCCGCCGCGTGCGAATCTGGTGGCACCTTCGCGCCTGTCTCGCTGCTTT | |
| | | CGATAAGTCTCTAGCCATTTAAAATTTTTGATGACCTGCTGCGACGCTTTTTTT | |
| | | CTGGCAAGATAGTCTTGTAAATGCGGGCCAAGATCTGCACACTGGTATTTCGGT | |
| | | TTTTGGGGCCGCGGGCGGCGACGGGGCCCGTGCGTCCCAGCGCACATGTTCGGC | |
| | | GAGGCGGGGCCTGCGAGCGCGGCCACCGAGAATCGGACGGGGGTAGTCTCAAGC | |
| | | TGGCCGGCCTGCTCTGGTGCCTGGCCTCGCGCCGCCGTGTATCGCCCCGCCCTG | |
| | | GGCGGCAAGGCTGGCCCGGTCGGCACCAGTTGCGTGAGCGGAAAGATGGCCGCT | |
| | | TCCCGGCCCTGCTGCAGGGAGCTCAAAATGGAGGACGCGGCGCTCGGGAGAGCG | |
| | | GGCGGGTGAGTCACCCACACAAAGGAAAAGGGCCTTTCCGTCCTCAGCCGTCGC | |
| | | TTCATGTGACTCCACGGAGTACCGGGCGCCGTCCAGGCACCTCGATTAGTTCTC | |
| | | GAGCTTTTGGAGTACGTCGTCTTTAGGTTGGGGGGAGGGGTTTTATGCGATGGA | |
| | | GTTTCCCCACACTGAGTGGGTGGAGACTGAAGTTAGGCCAGCTTGGCACTTGAT | |
| | | GTAATTCTCCTTGGAATTTGCCCTTTTTGAGTTTGGATCTTGGTTCATTCTCAA | |
| | | GCCTCAGACAGTGGTTCAAAGTTTTTTTCTTCCATTTCAGGTGTCGTGACGTAG | |
| | | CGGCCCTAATACGACTCACTATAGGGCCCGCGTACGTCTAGACCTCCAGGCATC | |
| | | GATTCCACCATGGCACTCCCCGTCACCGCCCTTCTCTTGCCCCTCGCCCTGCTG | |
| | | CTGCATGCTGCCAGGCCCCAAGTTCAACTGGTTCAGTCTGGCGCCGAAGTGAAG | |
| | | AAACCTGGCGCCTCTGTGAAGGTGTCCTGCAAGGCCAGCGGCTACACCTTTACC | |
| | | GGCTACTACATGCACTGGGTCCGACAGGCtcCAGGCCAAGGACTTGAATGGATG | |
| | | GGCTGGATGAACCCCAACAGCGGCAATACCGGCTATGCCCAGAAATTCCAGGGC | |
| | | AGAGTGACCATGACCAGAAACACCAGCATCAGCACCGCCTACATGGAACTGAGC | |
| | | AGCCTGAGAAGCGAGGACACCGCCGTGTACTACTGTGCTAGAGCCCAGTGGGAG | |
| | | CCCCTGGATTATTGGGGACAGGGCACACTGGTCACAGTCTCGAGTGGTGGAGGC | |
| | | GGTTCAGGCGGAGGTGGCTCTGGCGGTGGCGCTAGCTCTTATGAACTGACCCAG | |
| | | CCTCCAAGCGTGTCCGTGTCTCCTGGACAGACCGCCAGAATCACATGTAGCGGA | |
| | | GATGCCCTGCCTAAGAAGTACACCTACTGGTATCAGCAGAAGTCCGGACAGGCC | |
| | | CCTGTGCTGGTCATCTACTATGATACCAAACGGCCCAGCGGCATCCCCGAGAGA | |
| | | TTTTCTGGAAGCAGCAGCGGCACAATGGCCACACTGACAATTAGCGGAGCCCAG | |
| | | GTGGAAGATGAGGCCGACTACTACTGCCTGAGCACCGATCCTACAGGCCAAGAG | |
| | | TCCGTTTTTGGCGGAGGCACCAAGCTGACAGTGCTGGGACAACCTGCGGCCGCA | |
| | | ACCACAACCCCTGCTCCAAGGCCCCCCACACCCGCTCCAACTATAGCCAGCCAA | |
| | | CCATTGAGCCTCAGACCTGAAGCTTGCAGGCCCGCAGCAGGAGGCGCCGTCCAT | |
| | | ACGCGAGGCCTGGACTTCGCGTGTGATATTTATATTTGGGCCCCTTTGGCCGGA | |
| | | ACATGTGGGGTGTTGCTTCTCTCCCTTGTGATCACTCTGTATTGTAAGCGCGGG | |
| | | AGAAAGAAGCTCCTGTACATCTTCAAGCAGCCTTTTATGCGACCTGTGCAAACC | |
| | | ACTCAGGAAGAAGATGGGTGTTCATGCCGCTTCCCCGAGGAGGAAGAAGGAGGG | |
| | | TGTGAACTGAGGGTGAAATTTTCTAGAAGCGCCGATGCTCCCGCATATCAGCAG | |
| | | GGTCAGAATCAGCTCTACAATGAATTGAATCTCGGCAGGCGAGAAGAGTACGAT | |
| | | GTTCTGGACAAGAGACGGGGCAGGGATCCCGAGATGGGGGGAAAGCCCCGGAGA | |
| | | AAAAATCCTCAGGAGGGGTTGTACAATGAGCTGCAGAAGGACAAGATGGCTGAA | |
| | | GCCTATAGCGAGATCGGAATGAAAGGCGAAAGACGCAGAGGCAAGGGGCATGAC | |
| | | GGTCTGTACCAGGGTCTCTCTACAGCCACCAAGGACACTTATGATGCGTTGCAT | |
| | | ATGCAAGCCTTGCCACCCCGCGGATCAGGAGCGACTAACTTCTCTTTGTTGAAG | |
| | | CAAGCGGGTGACGTTGAAGAGAATCCCGGTCCATCCGGGGGATCCATGCGGATC | |
| | | TCCAAGCCCCACCTCCGAAGCATCAGTATCCAGTGTTATCTGTGCCTTCTTCTG | |
| | | AATTCCCATTTTTTGACAGAGGCAGGAATTCATGTATTCATATTGGGTTGTTTC | |
| | | TCCGCGGGGCTTCCTAAAACCGAAGCTAACTGGGTTAATGTCATTTCAGATCTG | |
| | | AAGAAAATTGAAGATCTTATTCAATCAATGCATATCGACGCGACCTTGTACACG | |
| | | GAGAGTGATGTCCACCCGAGCTGCAAGGTAACGGCCATGAAATGCTTCTTGCTG | |
| | | GAATTGCAGGTAATTAGTCTTGAGAGTGGAGATGCGTCTATACACGACACCGTA | |
| | | GAGAATCTCATTATTTTGGCGAACAATTCTCTCAGCAGTAACGGCAATGTTACT | |
| | | GAATCAGGATGTAAGGAGTGTGAAGAGCTGGAAGAAAAGAACATTAAGGAATTT | |
| | | TTGCAGAGCTTCGTACATATCGTCCAAATGTTCATAAATACAAGCTAAGTCGAC | |
| | | GGCCGGCCGCGAGCATCTTACCGCCATTTATACCCATATTTGTTCTGTTTTTCT | |
| | | TGATTTGGGTATACATTTAAATGTTAATAAAACAAAATGGTGGGGCAATCATTT | |
| | | ACATTTTATAGGATATGTAATTACTAGTTCAGGTGTATTGCCACAAGACAAACA | |
| | | TCTTAAGAAACTTTCCCGTTATTTACGCTCTGTTCCTGTTAATCAACCTCTGGA | |
| | | TTACAAAATTTGTGAAAGATTGACTGATATTCTTAACTATGTTGCTCCTTTTAC | |
| | | GCTGTGTGGATATGCTGCTTTAATACCTCTGTATCATACTATTGCTTCCTGTAC | |
| | | GGCTTTCGTTTTCTCCTCCTTGTATAAATCCTGGTTGCTGTCTCTTTATCAGGA | |
| | | GTTGTGGCCCGTTGTCCGTCAACGTGGCGTGGTGTGCTCTGTGTTTGCTGACGC | |
| | | AACCCCCACTGGCTGGGGCATTGCCACCACCTGTCAACTCCTTTCTGGGACTTT | |
| | | CGCTTTCCCCCTCCCGATCGCCACGGCAGAACTCATCGCCGCCTGCCTTGCCCG | |
| | | CTGCTGGACAGGGGCTAGGTTGCTGGGCACTGATAATTCCGTGGTGTTGTCCTG | |
| | | GTACCTTTAAGACCAATGACTTACAAGGCAGCTGTAGATCTTAGCCACTTTTTA | |
| | | AAAGAAAAGGGGGGACTGGAAGGGCTAATTCACTCCCAACGAAGATAAGATCTG | |
| | | CTTTTTGCTTGTACTGGGTCTCTCTGGTTAGACCAGATCTGAGCCTGGGAGCTC | |
| | | TCTGGCTAACTAGGGAACCCACTGCTTAAGCCTCAATAAAGCTTGCCTTGAGTG | |
| | | CTTCAAGTAGTGTGTGCCCGTCTGTTGTGTGACTCTGGTAACTAGAGATCCCTC | |
| | | AGACCCTTTTAGTCAGTGTGGAAAATCTCTAGCAGTAGTAGTTCATGTCATCTT | |
| | | ATTATTCAGTATTTATAACTTGCAAAGAAATGAATATCAGAGAGTGAGAGGAAC | |
| | | TTGTTTATTGCAGCTTATAATGGTTACAAATAAAGCAATAGCATCACAAATTTC | |
| | | ACAAATAAAGCATTTTTTTCACTGCATTCTAGTTGTGGTTTGTCCAAACTCATC | |
| | | AATGTATCTTATCATGTCTGGCTCTAGCTATCCCGCCCCTAACTCCGCCCATCC | |
| | | CGCCCCTAACTCCGCCCAGTTCCGCCCATTCTCCGCCCCATGGCTGACTAATTT | |
| | | TTTTTATTTATGCAGAGGCCGAGGCCGCCTCGGCCTCTGAGCTATTCCAGAAGT | |
| | | AGTGAGGAGGCTTTTTTGGAGGCCTAGACTTTTGCAGAGACCAAATTCGTAATC | |
| | | ATGTCATAGCTGTTTCCTGTGTGAAATTGTTATCCGCTCACAATTCCACACAAC | |
| | | ATACGAGCCGGAAGCATAAAGTGTAAAGCCTGGGGTGCCTAATGAGTGAGCTAA | |
| | | CTCACATTAATTGCGTTGCGCTCACTGCCCGCTTTCCAGTCGGGAAACCTGTCG | |
| | | TGCCAGCTGCATTAATGAATCGGCCAACGCGCGGGGAGAGGCGGTTTGCGTATT | |
| | | GGGCGCTCTTCCGCTTCCTCGCTCACTGACTCGCTGCGCTCGGTCGTTCGGCTG | |
| | | CGGCGAGCGGTATCAGCTCACTCAAAGGCGGTAATACGGTTATCCACAGAATCA | |
| | | GGGGATAACGCAGGAAAGAACATGTGAGCAAAAGGCCAGCAAAAGGCCAGGAAC | |
| | | CGTAAAAAGGCCGCGTTGCTGGCGTTTTTCCATAGGCTCCGCCCCCCTGACGAG | |
| | | CATCACAAAAATCGACGCTCAAGTCAGAGGTGGCGAAACCCGACAGGACTATAA | |
| | | AGATACCAGGCGTTTCCCCCTGGAAGCTCCCTCGTGCGCTCTCCTGTTCCGACC | |
| | | CTGCCGCTTACCGGATACCTGTCCGCCTTTCTCCCTTCGGGAAGCGTGGCGCTT | |
| | | TCTCATAGCTCACGCTGTAGGTATCTCAGTTCGGTGTAGGTCGTTCGCTCCAAG | |
| | | CTGGGCTGTGTGCACGAACCCCC | |
| CAR | VH | CAGGTGCAATTGGTGCAATCAGGGGCCGAAGTCAAGAAACCGGGTGCTTCAGTC | 51 |
| F6G- | | AAGGTTTCTTGTAAAGCATCTGGCTATACGTTTACTGGGTACTACATGCAATGG | |
| Var2 | | GTGAGACAAGCTCCTGGCCAAGGGCTTGAATGGATGGGGTGGATGAATCCAATC | |
| | | TCTGGAAATACAGGCTACGCCCAAAAGTTCCAGGGAAGAGTTACCATGACACGG | |
| | | AATACGTCCATTTCCACGGCATACATGGAACTGTCCTCCCTTCGGAGCGAGGAC | |
| | | ACTGCAGTATATTACTGCGCGAGAGCACAATGGGAACCGCTTGATTATTGGGGT | |
| | | CAAGGTACCTTGGTTACTGTGTCCTC | |
| | VL | AGCTATGAACTGACTCAGCCACCGAGTGTATCTGTAAGTCCGGGTCAGACAGCG | 59 |
| | | CGGATCACTTGCTCAGGGGACGCCCTTCCCAAAAAATACACTTACTGGTATCAA | |
| | | CAGAAGAGTGGGCAGGCTCCTGTGTTGGTAATATACTATGACACGAAACGGCCT | |
| | | AGCGGGATCCCGGAGCGGTTCTCAGGTTCCTCCAGTGGGACGATGGCTACACTG | |
| | | ACGATCAGCGGGGCCCAAGTGGAGGACGAAGCTGATTATTATTGCCTTAGTACC | |
| | | CACCCCGCCGGACAGGAGTCTGTGTTTGGTGGCGGCACCAAGCTTACGGTACTC | |
| | | GGACAGCCT | |
| | scFv | CAGGTGCAATTGGTGCAATCAGGGGCCGAAGTCAAGAAACCGGGTGCTTCAGTC | 67 |
| | | AAGGTTTCTTGTAAAGCATCTGGCTATACGTTTACTGGGTACTACATGCAATGG | |
| | | GTGAGACAAGCTCCTGGCCAAGGGCTTGAATGGATGGGGTGGATGAATCCAATC | |
| | | TCTGGAAATACAGGCTACGCCCAAAAGTTCCAGGGAAGAGTTACCATGACACGG | |
| | | AATACGTCCATTTCCACGGCATACATGGAACTGTCCTCCCTTCGGAGCGAGGAC | |
| | | ACTGCAGTATATTACTGCGCGAGAGCACAATGGGAACCGCTTGATTATTGGGGT | |
| | | CAAGGTACCTTGGTTACTGTGTCCTCAGGTGGAGGCGGTTCAGGCGGAGGTGGC | |
| | | TCTGGCGGTGGCGCTAGCAGCTATGAACTGACTCAGCCACCGAGTGTATCTGTA | |
| | | AGTCCGGGTCAGACAGCGCGGATCACTTGCTCAGGGGACGCCCTTCCCAAAAAA | |
| | | TACACTTACTGGTATCAACAGAAGAGTGGGCAGGCTCCTGTGTTGGTAATATAC | |
| | | TATGACACGAAACGGCCTAGCGGGATCCCGGAGCGGTTCTCAGGTTCCTCCAGT | |
| | | GGGACGATGGCTACACTGACGATCAGCGGGGCCCAAGTGGAGGACGAAGCTGAT | |
| | | TATTATTGCCTTAGTACCCACCCCGCCGGACAGGAGTCTGTGTTTGGTGGCGGC | |
| | | ACCAAGCTTACGGTACTCGGACAGCCT | |
| | CAR | CAGGTGCAATTGGTGCAATCAGGGGCCGAAGTCAAGAAACCGGGTGCTTCAGTC | 97 |
| | | AAGGTTTCTTGTAAAGCATCTGGCTATACGTTTACTGGGTACTACATGCAATGG | |
| | | GTGAGACAAGCTCCTGGCCAAGGGCTTGAATGGATGGGGTGGATGAATCCAATC | |
| | | TCTGGAAATACAGGCTACGCCCAAAAGTTCCAGGGAAGAGTTACCATGACACGG | |
| | | AATACGTCCATTTCCACGGCATACATGGAACTGTCCTCCCTTCGGAGCGAGGAC | |
| | | ACTGCAGTATATTACTGCGCGAGAGCACAATGGGAACCGCTTGATTATTGGGGT | |
| | | CAAGGTACCTTGGTTACTGTGTCCTCAGGTGGAGGCGGTTCAGGCGGAGGTGGC | |
| | | TCTGGCGGTGGCGCTAGCAGCTATGAACTGACTCAGCCACCGAGTGTATCTGTA | |
| | | AGTCCGGGTCAGACAGCGCGGATCACTTGCTCAGGGGACGCCCTTCCCAAAAAA | |
| | | TACACTTACTGGTATCAACAGAAGAGTGGGCAGGCTCCTGTGTTGGTAATATAC | |
| | | TATGACACGAAACGGCCTAGCGGGATCCCGGAGCGGTTCTCAGGTTCCTCCAGT | |
| | | GGGACGATGGCTACACTGACGATCAGCGGGGCCCAAGTGGAGGACGAAGCTGAT | |
| | | TATTATTGCCTTAGTACCCACCCCGCCGGACAGGAGTCTGTGTTTGGTGGCGGC | |
| | | ACCAAGCTTACGGTACTCGGACAGCCTGCGGCCGCAACCACAACCCCTGCTCCA | |
| | | AGGCCCCCCACACCCGCTCCAACTATAGCCAGCCAACCATTGAGCCTCAGACCT | |
| | | GAAGCTTGCAGGCCCGCAGCAGGAGGCGCCGTCCATACGCGAGGCCTGGACTTC | |
| | | GCGTGTGATATTTATATTTGGGCCCCTTTGGCCGGAACATGTGGGGTGTTGCTT | |
| | | CTCTCCCTTGTGATCACTCTGTATTGTAAGCGCGGGAGAAAGAAGCTCCTGTAC | |
| | | ATCTTCAAGCAGCCTTTTATGCGACCTGTGCAAACCACTCAGGAAGAAGATGGG | |
| | | TGTTCATGCCGCTTCCCCGAGGAGGAAGAAGGAGGGTGTGAACTGAGGGTGAAA | |
| | | TTTTCTAGAAGCGCCGATGCTCCCGCATATCAGCAGGGTCAGAATCAGCTCTAC | |
| | | AATGAATTGAATCTCGGCAGGCGAGAAGAGTACGATGTTCTGGACAAGAGACGG | |
| | | GGCAGGGATCCCGAGATGGGGGGAAAGCCCCGGAGAAAAAATCCTCAGGAGGGG | |
| | | TTGTACAATGAGCTGCAGAAGGACAAGATGGCTGAAGCCTATAGCGAGATCGGA | |
| | | ATGAAAGGCGAAAGACGCAGAGGCAAGGGGCATGACGGTCTGTACCAGGGTCTC | |
| | | TCTACAGCCACCAAGGACACTTATGATGCGTTGCATATGCAAGCCTTGCCACCC | |
| | | CGCGGATCAGGAGCGACTAACTTCTCTTTGTTGAAGCAAGCGGGTGACGTTGAA | |
| | | GAGAATCCCGGTCCATCCGGGGGATCCATGCGGATCTCCAAGCCCCACCTCCGA | |
| | | AGCATCAGTATCCAGTGTTATCTGTGCCTTCTTCTGAATTCCCATTTTTTGACA | |
| | | GAGGCAGGAATTCATGTATTCATATTGGGTTGTTTCTCCGCGGGGCTTCCTAAA | |
| | | ACCGAAGCTAACTGGGTTAATGTCATTTCAGATCTGAAGAAAATTGAAGATCTT | |
| | | ATTCAATCAATGCATATCGACGCGACCTTGTACACGGAGAGTGATGTCCACCCG | |
| | | AGCTGCAAGGTAACGGCCATGAAATGCTTCTTGCTGGAATTGCAGGTAATTAGT | |
| | | CTTGAGAGTGGAGATGCGTCTATACACGACACCGTAGAGAATCTCATTATTTTG | |
| | | GCGAACAATTCTCTCAGCAGTAACGGCAATGTTACTGAATCAGGATGTAAGGAG | |
| | | TGTGAAGAGCTGGAAGAAAAGAACATTAAGGAATTTTTGCAGAGCTTCGTACAT | |
| | | ATCGTCCAAATGTTCATAAATACAAGCTAA | |
| | Lentivira | cgttcagcccgaccgctgcgccttatccggtaactatcgtcttgagtccaacCC | 75 |
| | 1 vector | GGTAAGACACGACTTATCGCCACTGGCAGCAGCCACTGGTAACAGGATTAGCAG | |
| | | AGCGAGGTATGTAGGCGGTGCTACAGAGTTCTTGAAGTGGTGGCCTAACTACGG | |
| | | CTACACTAGAAGAACAGTATTTGGTATCTGCGCTCTGCTGAAGCCAGTTACCTT | |
| | | CGGAAAAAGAGTTGGTAGCTCTTGATCCGGCAAACAAACCACCGCTGGTAGCGG | |
| | | TGGTTTTTTTGTTTGCAAGCAGCAGATTACGCGCAGAAAAAAAGGATCTCAAGA | |
| | | AGATCCTTTGATCTTTTCTACGGGGTCTGACGCTCAGTGGAACGAAAACTCACG | |
| | | TTAAGGGATTTTGGTCATGAGATTATCAAAAAGGATCTTCACCTAGATCCTTTT | |
| | | AAATTAAAAATGAAGTTTTAAATCAATCTAAAGTATATATGAGTAAACTTGGTC | |
| | | TGACAGTTAGAAAAACTCATCGAGCATCAAATGAAACTGCAATTTATTCATATC | |
| | | AGGATTATCAATACCATATTTTTGAAAAAGCCGTTTCTGTAATGAAGGAGAAAA | |
| | | CTCACCGAGGCAGTTCCATAGGATGGCAAGATCCTGGTATCGGTCTGCGATTCC | |
| | | GACTCGTCCAACATCAATACAACCTATTAATTTCCCCTCGTCAAAAATAAGGTT | |
| | | ATCAAGTGAGAAATCACCATGAGTGACGACTGAATCCGGTGAGAATGGCAAAAG | |
| | | TTTATGCATTTCTTTCCAGACTTGTTCAACAGGCCAGCCATTACGCTCGTCATC | |
| | | AAAATCACTCGCATCAACCAAACCGTTATTCATTCGTGATTGCGCCTGAGCGAG | |
| | | ACGAAATACGCGATCGCTGTTAAAAGGACAATTACAAACAGGAATCGAATGCAA | |
| | | CCGGCGCAGGAACACTGCCAGCGCATCAACAATATTTTCACCTGAATCAGGATA | |
| | | TTCTTCTAATACCTGGAATGCTGTTTTTCCGGGGATCGCAGTGGTGAGTAACCA | |
| | | TGCATCATCAGGAGTACGGATAAAATGCTTGATGGTCGGAAGAGGCATAAATTC | |
| | | CGTCAGCCAGTTTAGTCTGACCATCTCATCTGTAACATCATTGGCAACGCTACC | |
| | | TTTGCCATGTTTCAGAAACAACTCTGGCGCATCGGGCTTCCCATACAAGCGATA | |
| | | GATTGTCGCACCTGATTGCCCGACATTATCGCGAGCCCATTTATACCCATATAA | |
| | | ATCAGCATCCATGTTGGAATTTAATCGCGGCCTCGACGTTTCCCGTTGAATATG | |
| | | GCTCATACTCTTCCTTTTTCAATATTATTGAAGCATTTATCAGGGTTATTGTCT | |
| | | CATGAGCGGATACATATTTGAATGTATTTAGAAAAATAAACAAATAGGGGTTCC | |
| | | GCGCACATTTCCCCGAAAAGTGCCACCTGACGGATCGGGAGATCTCCCGATCCC | |
| | | CTATGGTGCACTCTCAGTACAATCTGCTCTGATGCCGCATAGTTAAGCCAGTAT | |
| | | CTGCTCCCTGCTTGTGTGTTGGAGGTCGCTGAGTAGTGCGCGAGCAAAATTTAA | |
| | | GCTACAACAAGGCAAGGCTTGACCGACAATTGCATGAAGAATCTGCTTAGGGTT | |
| | | AGGCGTTTTGCGCTGCTTCGCGATGTACGGGCCAGATATACCCGTTGACATTGA | |
| | | TTATTGACTAGTTATTAATAGTAATCAATTACGGGGTCATTAGTTCATAGCCCA | |
| | | TATATGGAGTTCCGCGTTACATAACTTACGGTAAATGGCCCGCCTGGCTGACCG | |
| | | CCCAACGACCCCCGCCCATTGACGTCAATAATGACGTATGTTCCCATAGTAACG | |
| | | CCAATAGGGACTTTCCATTGACGTCAATGGGTGGAGTATTTACGGTAAACTGCC | |
| | | CACTTGGCAGTACATCAAGTGTATCATATGCCAAGTACGCCCCCTATTGACGTC | |
| | | AATGACGGTAAATGGCCCGCCTGGCATTATGCCCAGTACATGACCTTATGGGAC | |
| | | TTTCCTACTTGGCAGTACATCTACGTATTAGTCATCGCTATTACCATGGTGATG | |
| | | CGGTTTTGGCAGTACATCAATGGGCGTGGATAGCGGTTTGACTCACGGGGATTT | |
| | | CCAAGTCTCCACCCCATTGACGTCAATGGGAGTTTGTTTTGGCACCAAAATCAA | |
| | | CGGGACTTTCCAAAATGTCGTAACAACTCCGCCCCATTGACGCAAATGGGCGGT | |
| | | AGGCGTGTACGGTGGGAGGTCTATATAAGCAGCGCGTTTTGCCTGTACTGGGTC | |
| | | TCTCTGGTTAGACCAGATCTGAGCCTGGGAGCTCTCTGGCTAACTAGGGAACCC | |
| | | ACTGCTTAAGCCTCAATAAAGCTTGCCTTGAGTGCTTCAAGTAGTGTGTGCCCG | |
| | | TCTGTTGTGTGACTCTGGTAACTAGAGATCCCTCAGACCCTTTTAGTCAGTGTG | |
| | | GAAAATCTCTAGCAGTGGCGCCCGAACAGGGACTTGAAAGCGAAAGGGAAACCA | |
| | | GAGGAGCTCTCTCGACGCAGGACTCGGCTTGCTGAAGCGCGCACGGCAAGAGGC | |
| | | GAGGGGCGGCGACTGGTGAGTACGCCAAAAATTTTGACTAGCGGAGGCTAGAAG | |
| | | GAGAGAGATGGGTGCGAGAGCGTCAGTATTAAGCGGGGGAGAATTAGATCGCGA | |
| | | TGGGAAAAAATTCGGTTAAGGCCAGGGGGAAAGAAAAAATATAAATTAAAACAT | |
| | | ATAGTATGGGCAAGCAGGGAGCTAGAACGATTCGCAGTTAATCCTGGCCTGTTA | |
| | | GAAACATCAGAAGGCTGTAGACAAATACTGGGACAGCTACAACCATCCCTTCAG | |
| | | ACAGGATCAGAAGAACTTAGATCATTATATAATACAGTAGCAACCCTCTATTGT | |
| | | GTGCATCAAAGGATAGAGATAAAAGACACCAAGGAAGCTTTAGACAAGATAGAG | |
| | | GAAGAGCAAAACAAAAGTAAGACCACCGCACAGCAAGCGGCCGCTGATCTTCAG | |
| | | ACCTGGAGGAGGAGATATGAGGGACAATTGGAGAAGTGAATTATATAAATATAA | |
| | | AGTAGTAAAAATTGAACCATTAGGAGTAGCACCCACCAAGGCAAAGAGAAGAGT | |
| | | GGTGCAGAGAGAAAAAAGAGCAGTGGGAATAGGAGCTTTGTTCCTTGGGTTCTT | |
| | | GGGAGCAGCAGGAAGCACTATGGGCGCAGCGTCAATGACGCTGACGGTACAGGC | |
| | | CAGACAATTATTGTCTGGTATAGTGCAGCAGCAGAACAATTTGCTGAGGGCTAT | |
| | | TGAGGCGCAACAGCATCTGTTGCAACTCACAGTCTGGGGCATCAAGCAGCTCCA | |
| | | GGCAAGAATCCTGGCTGTGGAAAGATACCTAAAGGATCAACAGCTCCTGGGGAT | |
| | | TTGGGGTTGCTCTGGAAAACTCATTTGCACCACTGCTGTGCCTTGGAATGCTAG | |
| | | TTGGAGTAATAAATCTCTGGAACAGATTTGGAATCACACGACCTGGATGGAGTG | |
| | | GGACAGAGAAATTAACAATTACACAAGCTTAATACACTCCTTAATTGAAGAATC | |
| | | GCAAAACCAGCAAGAAAAGAATGAACAAGAATTATTGGAATTAGATAAATGGGC | |
| | | AAGTTTGTGGAATTGGTTTAACATAACAAATTGGCTGTGGTATATAAAATTATT | |
| | | CATAATGATAGTAGGAGGCTTGGTAGGTTTAAGAATAGTTTTTGCTGTACTTTC | |
| | | TATAGTGAATAGAGTTAGGCAGGGATATTCACCATTATCGTTTCAGACCCACCT | |
| | | CCCAACCCCGAGGGGACCCGACAGGCCCGAAGGAATAGAAGAAGAAGGTGGAGA | |
| | | GAGAGACAGAGACAGATCCATTCGATTAGTGAACGGATCTCGACGGTATCGGTT | |
| | | AACTTTTAAAAGAAAAGGGGGGATTGGGGGGTACAGTGCAGGGGAAAGAATAGT | |
| | | AGACATAATAGCAACAGACATACAAACTAAAGAATTACAAAAACAAATTACAAA | |
| | | ATTCAAAATTTTATCGATACTAGTATTATGCCCAGTACATGACCTTATGGGACT | |
| | | TTCCTACTTGGCAGTACATCTACAAACCCCGGGTGCAAAGATGGATAAAGTTTT | |
| | | AAACAGAGAGGAATCTTTGCAGCTAATGGACCTTCTAGGTCTTGAAAGGAGTGG | |
| | | GAATTGGCTCCGGTGCCCGTCAGTGGGCAGAGCGCACATCGCCCACAGTCCCCG | |
| | | AGAAGTTGGGGGGAGGGGTCGGCAATTGATCCGGTGCCTAGAGAAGGTGGCGCG | |
| | | GGGTAAACTGGGAAAGTGATGTCGTGTACTGGCTCCGCCTTTTTCCCGAGGGTG | |
| | | GGGGAGAACCGTATATAAGTGCAGTAGTCGCCGTGAACGTTCTTTTTCGCAACG | |
| | | GGTTTGCCGCCAGAACACAGGTAAGTGCCGTGTGTGGTTCCCGCGGGCCTGGCC | |
| | | TCTTTACGGGTTATGGCCCTTGCGTGCCTTGAATTACTTCCACTGGCTGCAGTA | |
| | | CGTGATTCTTGATCCCGAGCTTCGGGTTGGAAGTGGGTGGGAGAGTTCGAGGCC | |
| | | TTGCGCTTAAGGAGCCCCTTCGCCTCGTGCTTGAGTTGAGGCCTGGCCTGGGCG | |
| | | CTGGGGCCGCCGCGTGCGAATCTGGTGGCACCTTCGCGCCTGTCTCGCTGCTTT | |
| | | CGATAAGTCTCTAGCCATTTAAAATTTTTGATGACCTGCTGCGACGCTTTTTTT | |
| | | CTGGCAAGATAGTCTTGTAAATGCGGGCCAAGATCTGCACACTGGTATTTCGGT | |
| | | TTTTGGGGCCGCGGGCGGCGACGGGGCCCGTGCGTCCCAGCGCACATGTTCGGC | |
| | | GAGGCGGGGCCTGCGAGCGCGGCCACCGAGAATCGGACGGGGGTAGTCTCAAGC | |
| | | TGGCCGGCCTGCTCTGGTGCCTGGCCTCGCGCCGCCGTGTATCGCCCCGCCCTG | |
| | | GGCGGCAAGGCTGGCCCGGTCGGCACCAGTTGCGTGAGCGGAAAGATGGCCGCT | |
| | | TCCCGGCCCTGCTGCAGGGAGCTCAAAATGGAGGACGCGGCGCTCGGGAGAGCG | |
| | | GGCGGGTGAGTCACCCACACAAAGGAAAAGGGCCTTTCCGTCCTCAGCCGTCGC | |
| | | TTCATGTGACTCCACGGAGTACCGGGCGCCGTCCAGGCACCTCGATTAGTTCTC | |
| | | GAGCTTTTGGAGTACGTCGTCTTTAGGTTGGGGGGAGGGGTTTTATGCGATGGA | |
| | | GTTTCCCCACACTGAGTGGGTGGAGACTGAAGTTAGGCCAGCTTGGCACTTGAT | |
| | | GTAATTCTCCTTGGAATTTGCCCTTTTTGAGTTTGGATCTTGGTTCATTCTCAA | |
| | | GCCTCAGACAGTGGTTCAAAGTTTTTTTCTTCCATTTCAGGTGTCGTGACGTAG | |
| | | CGGCCCTAATACGACTCACTATAGGGCCCGCGTACGTCTAGACCTCCAGGCATC | |
| | | GATTCCACCATGGCACTCCCCGTCACCGCCCTTCTCTTGCCCCTCGCCCTGCTG | |
| | | CTGCATGCTGCCAGGCCCCAGGTGCAATTGGTGCAATCAGGGGCCGAAGTCAAG | |
| | | AAACCGGGTGCTTCAGTCAAGGTTTCTTGTAAAGCATCTGGCTATACGTTTACT | |
| | | GGGTACTACATGCAATGGGTGAGACAAGCTCCTGGCCAAGGGCTTGAATGGATG | |
| | | GGGTGGATGAATCCAATCTCTGGAAATACAGGCTACGCCCAAAAGTTCCAGGGA | |
| | | AGAGTTACCATGACACGGAATACGTCCATTTCCACGGCATACATGGAACTGTCC | |
| | | TCCCTTCGGAGCGAGGACACTGCAGTATATTACTGCGCGAGAGCACAATGGGAA | |
| | | CCGCTTGATTATTGGGGTCAAGGTACCTTGGTTACTGTGTCCTCAGGTGGAGGC | |
| | | GGTTCAGGCGGAGGTGGCTCTGGCGGTGGCGCTAGCAGCTATGAACTGACTCAG | |
| | | CCACCGAGTGTATCTGTAAGTCCGGGTCAGACAGCGCGGATCACTTGCTCAGGG | |
| | | GACGCCCTTCCCAAAAAATACACTTACTGGTATCAACAGAAGAGTGGGCAGGCT | |
| | | CCTGTGTTGGTAATATACTATGACACGAAACGGCCTAGCGGGATCCCGGAGCGG | |
| | | TTCTCAGGTTCCTCCAGTGGGACGATGGCTACACTGACGATCAGCGGGGCCCAA | |
| | | GTGGAGGACGAAGCTGATTATTATTGCCTTAGTACCCACCCCGCCGGACAGGAG | |
| | | TCTGTGTTTGGTGGCGGCACCAAGCTTACGGTACTCGGACAGCCTGCGGCCGCA | |
| | | ACCACAACCCCTGCTCCAAGGCCCCCCACACCCGCTCCAACTATAGCCAGCCAA | |
| | | CCATTGAGCCTCAGACCTGAAGCTTGCAGGCCCGCAGCAGGAGGCGCCGTCCAT | |
| | | ACGCGAGGCCTGGACTTCGCGTGTGATATTTATATTTGGGCCCCTTTGGCCGGA | |
| | | ACATGTGGGGTGTTGCTTCTCTCCCTTGTGATCACTCTGTATTGTAAGCGCGGG | |
| | | AGAAAGAAGCTCCTGTACATCTTCAAGCAGCCTTTTATGCGACCTGTGCAAACC | |
| | | ACTCAGGAAGAAGATGGGTGTTCATGCCGCTTCCCCGAGGAGGAAGAAGGAGGG | |
| | | TGTGAACTGAGGGTGAAATTTTCTAGAAGCGCCGATGCTCCCGCATATCAGCAG | |
| | | GGTCAGAATCAGCTCTACAATGAATTGAATCTCGGCAGGCGAGAAGAGTACGAT | |
| | | GTTCTGGACAAGAGACGGGGCAGGGATCCCGAGATGGGGGGAAAGCCCCGGAGA | |
| | | AAAAATCCTCAGGAGGGGTTGTACAATGAGCTGCAGAAGGACAAGATGGCTGAA | |
| | | GCCTATAGCGAGATCGGAATGAAAGGCGAAAGACGCAGAGGCAAGGGGCATGAC | |
| | | GGTCTGTACCAGGGTCTCTCTACAGCCACCAAGGACACTTATGATGCGTTGCAT | |
| | | ATGCAAGCCTTGCCACCCCGCGGATCAGGAGCGACTAACTTCTCTTTGTTGAAG | |
| | | CAAGCGGGTGACGTTGAAGAGAATCCCGGTCCATCCGGGGGATCCATGCGGATC | |
| | | TCCAAGCCCCACCTCCGAAGCATCAGTATCCAGTGTTATCTGTGCCTTCTTCTG | |
| | | AATTCCCATTTTTTGACAGAGGCAGGAATTCATGTATTCATATTGGGTTGTTTC | |
| | | TCCGCGGGGCTTCCTAAAACCGAAGCTAACTGGGTTAATGTCATTTCAGATCTG | |
| | | AAGAAAATTGAAGATCTTATTCAATCAATGCATATCGACGCGACCTTGTACACG | |
| | | GAGAGTGATGTCCACCCGAGCTGCAAGGTAACGGCCATGAAATGCTTCTTGCTG | |
| | | GAATTGCAGGTAATTAGTCTTGAGAGTGGAGATGCGTCTATACACGACACCGTA | |
| | | GAGAATCTCATTATTTTGGCGAACAATTCTCTCAGCAGTAACGGCAATGTTACT | |
| | | GAATCAGGATGTAAGGAGTGTGAAGAGCTGGAAGAAAAGAACATTAAGGAATTT | |
| | | TTGCAGAGCTTCGTACATATCGTCCAAATGTTCATAAATACAAGCTAAGTCGAC | |
| | | GGCCGGCCGCGAGCATCTTACCGCCATTTATACCCATATTTGTTCTGTTTTTCT | |
| | | TGATTTGGGTATACATTTAAATGTTAATAAAACAAAATGGTGGGGCAATCATTT | |
| | | ACATTTTATAGGATATGTAATTACTAGTTCAGGTGTATTGCCACAAGACAAACA | |
| | | TCTTAAGAAACTTTCCCGTTATTTACGCTCTGTTCCTGTTAATCAACCTCTGGA | |
| | | TTACAAAATTTGTGAAAGATTGACTGATATTCTTAACTATGTTGCTCCTTTTAC | |
| | | GCTGTGTGGATATGCTGCTTTAATACCTCTGTATCATACTATTGCTTCCTGTAC | |
| | | GGCTTTCGTTTTCTCCTCCTTGTATAAATCCTGGTTGCTGTCTCTTTATCAGGA | |
| | | GTTGTGGCCCGTTGTCCGTCAACGTGGCGTGGTGTGCTCTGTGTTTGCTGACGC | |
| | | AACCCCCACTGGCTGGGGCATTGCCACCACCTGTCAACTCCTTTCTGGGACTTT | |
| | | CGCTTTCCCCCTCCCGATCGCCACGGCAGAACTCATCGCCGCCTGCCTTGCCCG | |
| | | CTGCTGGACAGGGGCTAGGTTGCTGGGCACTGATAATTCCGTGGTGTTGTCCTG | |
| | | GTACCTTTAAGACCAATGACTTACAAGGCAGCTGTAGATCTTAGCCACTTTTTA | |
| | | AAAGAAAAGGGGGGACTGGAAGGGCTAATTCACTCCCAACGAAGATAAGATCTG | |
| | | CTTTTTGCTTGTACTGGGTCTCTCTGGTTAGACCAGATCTGAGCCTGGGAGCTC | |
| | | TCTGGCTAACTAGGGAACCCACTGCTTAAGCCTCAATAAAGCTTGCCTTGAGTG | |
| | | CTTCAAGTAGTGTGTGCCCGTCTGTTGTGTGACTCTGGTAACTAGAGATCCCTC | |
| | | AGACCCTTTTAGTCAGTGTGGAAAATCTCTAGCAGTAGTAGTTCATGTCATCTT | |
| | | ATTATTCAGTATTTATAACTTGCAAAGAAATGAATATCAGAGAGTGAGAGGAAC | |
| | | TTGTTTATTGCAGCTTATAATGGTTACAAATAAAGCAATAGCATCACAAATTTC | |
| | | ACAAATAAAGCATTTTTTTCACTGCATTCTAGTTGTGGTTTGTCCAAACTCATC | |
| | | AATGTATCTTATCATGTCTGGCTCTAGCTATCCCGCCCCTAACTCCGCCCATCC | |
| | | CGCCCCTAACTCCGCCCAGTTCCGCCCATTCTCCGCCCCATGGCTGACTAATTT | |
| | | TTTTTATTTATGCAGAGGCCGAGGCCGCCTCGGCCTCTGAGCTATTCCAGAAGT | |
| | | AGTGAGGAGGCTTTTTTGGAGGCCTAGACTTTTGCAGAGACCAAATTCGTAATC | |
| | | ATGTCATAGCTGTTTCCTGTGTGAAATTGTTATCCGCTCACAATTCCACACAAC | |
| | | ATACGAGCCGGAAGCATAAAGTGTAAAGCCTGGGGTGCCTAATGAGTGAGCTAA | |
| | | CTCACATTAATTGCGTTGCGCTCACTGCCCGCTTTCCAGTCGGGAAACCTGTCG | |
| | | TGCCAGCTGCATTAATGAATCGGCCAACGCGCGGGGAGAGGCGGTTTGCGTATT | |
| | | GGGCGCTCTTCCGCTTCCTCGCTCACTGACTCGCTGCGCTCGGTCGTTCGGCTG | |
| | | CGGCGAGCGGTATCAGCTCACTCAAAGGCGGTAATACGGTTATCCACAGAATCA | |
| | | GGGGATAACGCAGGAAAGAACATGTGAGCAAAAGGCCAGCAAAAGGCCAGGAAC | |
| | | CGTAAAAAGGCCGCGTTGCTGGCGTTTTTCCATAGGCTCCGCCCCCCTGACGAG | |
| | | CATCACAAAAATCGACGCTCAAGTCAGAGGTGGCGAAACCCGACAGGACTATAA | |
| | | AGATACCAGGCGTTTCCCCCTGGAAGCTCCCTCGTGCGCTCTCCTGTTCCGACC | |
| | | CTGCCGCTTACCGGATACCTGTCCGCCTTTCTCCCTTCGGGAAGCGTGGCGCTT | |
| | | TCTCATAGCTCACGCTGTAGGTATCTCAGTTCGGTGTAGGTCGTTCGCTCCAAG | |
| | | CTGGGCTGTGTGCACGAACCCCC | |
| F6G- | VH | CAGGTCCAATTGGTCCAATCTGGCGCCGAGGTCAAGAAGCCTGGCGCATCCGTA | 52 |
| Var3 | | AAAGTGTCTTGTAAAGCCTCTGGTTATACGTTTACGGGGTATTATATGCAATGG | |
| | | GTTCGGCAAGCCCCTGGTCAAGGACTTGAATGGATGGGCTGGATGAACCCTATC | |
| | | TCTGGTAATACGGGCTACGCCCAAAAGTTCCAGGGTAGAGTCACAATGACGCGC | |
| | | AACACGTCAATATCAACCGCTTACATGGAGCTTAGTTCTTTGAGGTCAGAGGAC | |
| | | ACCGCGGTCTATTATTGTGCTCGCGCCCAATGGGAACCGCTTGATTACTGGGGG | |
| | | CAAGGTACCTTGGTTACCGTCTCATC | |
| | VL | AGCTACGAACTGACACAGCCGCCCTCTGTTAGCGTTAGTCCTGGACAGACCGCC | 60 |
| | | AGGATAACGTGCTCCGGTGACGCATTGCCGAAGAAATATACGTACTGGTACCAG | |
| | | CAGAAGTCCGGGCAAGCACCTGTACTCGTGATCTATTATGATACAAAACGGCCG | |
| | | AGCGGTATACCAGAACGCTTTAGCGGCTCAAGCAGCGGAACGATGGCCACGTTG | |
| | | ACGATTAGCGGCGCGCAGGTCGAGGACGAAGCTGACTACTATTGCCTCAGTACT | |
| | | AACCCAACAGGGCACGAATCAGTTTTTGGAGGGGGAACCAAGCTTACTGTACTT | |
| | | GGTCAGCCG | |
| | scFv | CAGGTCCAATTGGTCCAATCTGGCGCCGAGGTCAAGAAGCCTGGCGCATCCGTA | 68 |
| | | AAAGTGTCTTGTAAAGCCTCTGGTTATACGTTTACGGGGTATTATATGCAATGG | |
| | | GTTCGGCAAGCCCCTGGTCAAGGACTTGAATGGATGGGCTGGATGAACCCTATC | |
| | | TCTGGTAATACGGGCTACGCCCAAAAGTTCCAGGGTAGAGTCACAATGACGCGC | |
| | | AACACGTCAATATCAACCGCTTACATGGAGCTTAGTTCTTTGAGGTCAGAGGAC | |
| | | ACCGCGGTCTATTATTGTGCTCGCGCCCAATGGGAACCGCTTGATTACTGGGGG | |
| | | CAAGGTACCTTGGTTACCGTCTCATCAGGTGGAGGCGGTTCAGGCGGAGGTGGC | |
| | | TCTGGCGGTGGCGCTAGCAGCTACGAACTGACACAGCCGCCCTCTGTTAGCGTT | |
| | | AGTCCTGGACAGACCGCCAGGATAACGTGCTCCGGTGACGCATTGCCGAAGAAA | |
| | | TATACGTACTGGTACCAGCAGAAGTCCGGGCAAGCACCTGTACTCGTGATCTAT | |
| | | TATGATACAAAACGGCCGAGCGGTATACCAGAACGCTTTAGCGGCTCAAGCAGC | |
| | | GGAACGATGGCCACGTTGACGATTAGCGGCGCGCAGGTCGAGGACGAAGCTGAC | |
| | | TACTATTGCCTCAGTACTAACCCAACAGGGCACGAATCAGTTTTTGGAGGGGGA | |
| | | ACCAAGCTTACTGTACTTGGTCAGCCG | |
| | CAR | CAGGTCCAATTGGTCCAATCTGGCGCCGAGGTCAAGAAGCCTGGCGCATCCGTA | 98 |
| | | AAAGTGTCTTGTAAAGCCTCTGGTTATACGTTTACGGGGTATTATATGCAATGG | |
| | | GTTCGGCAAGCCCCTGGTCAAGGACTTGAATGGATGGGCTGGATGAACCCTATC | |
| | | TCTGGTAATACGGGCTACGCCCAAAAGTTCCAGGGTAGAGTCACAATGACGCGC | |
| | | AACACGTCAATATCAACCGCTTACATGGAGCTTAGTTCTTTGAGGTCAGAGGAC | |
| | | ACCGCGGTCTATTATTGTGCTCGCGCCCAATGGGAACCGCTTGATTACTGGGGG | |
| | | CAAGGTACCTTGGTTACCGTCTCATCAGGTGGAGGCGGTTCAGGCGGAGGTGGC | |
| | | TCTGGCGGTGGCGCTAGCAGCTACGAACTGACACAGCCGCCCTCTGTTAGCGTT | |
| | | AGTCCTGGACAGACCGCCAGGATAACGTGCTCCGGTGACGCATTGCCGAAGAAA | |
| | | TATACGTACTGGTACCAGCAGAAGTCCGGGCAAGCACCTGTACTCGTGATCTAT | |
| | | TATGATACAAAACGGCCGAGCGGTATACCAGAACGCTTTAGCGGCTCAAGCAGC | |
| | | GGAACGATGGCCACGTTGACGATTAGCGGCGCGCAGGTCGAGGACGAAGCTGAC | |
| | | TACTATTGCCTCAGTACTAACCCAACAGGGCACGAATCAGTTTTTGGAGGGGGA | |
| | | ACCAAGCTTACTGTACTTGGTCAGCCGGCGGCCGCAACCACAACCCCTGCTCCA | |
| | | AGGCCCCCCACACCCGCTCCAACTATAGCCAGCCAACCATTGAGCCTCAGACCT | |
| | | GAAGCTTGCAGGCCCGCAGCAGGAGGCGCCGTCCATACGCGAGGCCTGGACTTC | |
| | | GCGTGTGATATTTATATTTGGGCCCCTTTGGCCGGAACATGTGGGGTGTTGCTT | |
| | | CTCTCCCTTGTGATCACTCTGTATTGTAAGCGCGGGAGAAAGAAGCTCCTGTAC | |
| | | ATCTTCAAGCAGCCTTTTATGCGACCTGTGCAAACCACTCAGGAAGAAGATGGG | |
| | | TGTTCATGCCGCTTCCCCGAGGAGGAAGAAGGAGGGTGTGAACTGAGGGTGAAA | |
| | | TTTTCTAGAAGCGCCGATGCTCCCGCATATCAGCAGGGTCAGAATCAGCTCTAC | |
| | | AATGAATTGAATCTCGGCAGGCGAGAAGAGTACGATGTTCTGGACAAGAGACGG | |
| | | GGCAGGGATCCCGAGATGGGGGGAAAGCCCCGGAGAAAAAATCCTCAGGAGGGG | |
| | | TTGTACAATGAGCTGCAGAAGGACAAGATGGCTGAAGCCTATAGCGAGATCGGA | |
| | | ATGAAAGGCGAAAGACGCAGAGGCAAGGGGCATGACGGTCTGTACCAGGGTCTC | |
| | | TCTACAGCCACCAAGGACACTTATGATGCGTTGCATATGCAAGCCTTGCCACCC | |
| | | CGC | |
| | Lentivira | cgttcagcccgaccgctgcgccttatccggtaactatcgtcttgagtccaacCC | 76 |
| | 1 vector | GGTAAGACACGACTTATCGCCACTGGCAGCAGCCACTGGTAACAGGATTAGCAG | |
| | | AGCGAGGTATGTAGGCGGTGCTACAGAGTTCTTGAAGTGGTGGCCTAACTACGG | |
| | | CTACACTAGAAGAACAGTATTTGGTATCTGCGCTCTGCTGAAGCCAGTTACCTT | |
| | | CGGAAAAAGAGTTGGTAGCTCTTGATCCGGCAAACAAACCACCGCTGGTAGCGG | |
| | | TGGTTTTTTTGTTTGCAAGCAGCAGATTACGCGCAGAAAAAAAGGATCTCAAGA | |
| | | AGATCCTTTGATCTTTTCTACGGGGTCTGACGCTCAGTGGAACGAAAACTCACG | |
| | | TTAAGGGATTTTGGTCATGAGATTATCAAAAAGGATCTTCACCTAGATCCTTTT | |
| | | AAATTAAAAATGAAGTTTTAAATCAATCTAAAGTATATATGAGTAAACTTGGTC | |
| | | TGACAGTTAGAAAAACTCATCGAGCATCAAATGAAACTGCAATTTATTCATATC | |
| | | AGGATTATCAATACCATATTTTTGAAAAAGCCGTTTCTGTAATGAAGGAGAAAA | |
| | | CTCACCGAGGCAGTTCCATAGGATGGCAAGATCCTGGTATCGGTCTGCGATTCC | |
| | | GACTCGTCCAACATCAATACAACCTATTAATTTCCCCTCGTCAAAAATAAGGTT | |
| | | ATCAAGTGAGAAATCACCATGAGTGACGACTGAATCCGGTGAGAATGGCAAAAG | |
| | | TTTATGCATTTCTTTCCAGACTTGTTCAACAGGCCAGCCATTACGCTCGTCATC | |
| | | AAAATCACTCGCATCAACCAAACCGTTATTCATTCGTGATTGCGCCTGAGCGAG | |
| | | ACGAAATACGCGATCGCTGTTAAAAGGACAATTACAAACAGGAATCGAATGCAA | |
| | | CCGGCGCAGGAACACTGCCAGCGCATCAACAATATTTTCACCTGAATCAGGATA | |
| | | TTCTTCTAATACCTGGAATGCTGTTTTTCCGGGGATCGCAGTGGTGAGTAACCA | |
| | | TGCATCATCAGGAGTACGGATAAAATGCTTGATGGTCGGAAGAGGCATAAATTC | |
| | | CGTCAGCCAGTTTAGTCTGACCATCTCATCTGTAACATCATTGGCAACGCTACC | |
| | | TTTGCCATGTTTCAGAAACAACTCTGGCGCATCGGGCTTCCCATACAAGCGATA | |
| | | GATTGTCGCACCTGATTGCCCGACATTATCGCGAGCCCATTTATACCCATATAA | |
| | | ATCAGCATCCATGTTGGAATTTAATCGCGGCCTCGACGTTTCCCGTTGAATATG | |
| | | GCTCATACTCTTCCTTTTTCAATATTATTGAAGCATTTATCAGGGTTATTGTCT | |
| | | CATGAGCGGATACATATTTGAATGTATTTAGAAAAATAAACAAATAGGGGTTCC | |
| | | GCGCACATTTCCCCGAAAAGTGCCACCTGACGGATCGGGAGATCTCCCGATCCC | |
| | | CTATGGTGCACTCTCAGTACAATCTGCTCTGATGCCGCATAGTTAAGCCAGTAT | |
| | | CTGCTCCCTGCTTGTGTGTTGGAGGTCGCTGAGTAGTGCGCGAGCAAAATTTAA | |
| | | GCTACAACAAGGCAAGGCTTGACCGACAATTGCATGAAGAATCTGCTTAGGGTT | |
| | | AGGCGTTTTGCGCTGCTTCGCGATGTACGGGCCAGATATACCCGTTGACATTGA | |
| | | TTATTGACTAGTTATTAATAGTAATCAATTACGGGGTCATTAGTTCATAGCCCA | |
| | | TATATGGAGTTCCGCGTTACATAACTTACGGTAAATGGCCCGCCTGGCTGACCG | |
| | | CCCAACGACCCCCGCCCATTGACGTCAATAATGACGTATGTTCCCATAGTAACG | |
| | | CCAATAGGGACTTTCCATTGACGTCAATGGGTGGAGTATTTACGGTAAACTGCC | |
| | | CACTTGGCAGTACATCAAGTGTATCATATGCCAAGTACGCCCCCTATTGACGTC | |
| | | AATGACGGTAAATGGCCCGCCTGGCATTATGCCCAGTACATGACCTTATGGGAC | |
| | | TTTCCTACTTGGCAGTACATCTACGTATTAGTCATCGCTATTACCATGGTGATG | |
| | | CGGTTTTGGCAGTACATCAATGGGCGTGGATAGCGGTTTGACTCACGGGGATTT | |
| | | CCAAGTCTCCACCCCATTGACGTCAATGGGAGTTTGTTTTGGCACCAAAATCAA | |
| | | CGGGACTTTCCAAAATGTCGTAACAACTCCGCCCCATTGACGCAAATGGGCGGT | |
| | | AGGCGTGTACGGTGGGAGGTCTATATAAGCAGCGCGTTTTGCCTGTACTGGGTC | |
| | | TCTCTGGTTAGACCAGATCTGAGCCTGGGAGCTCTCTGGCTAACTAGGGAACCC | |
| | | ACTGCTTAAGCCTCAATAAAGCTTGCCTTGAGTGCTTCAAGTAGTGTGTGCCCG | |
| | | TCTGTTGTGTGACTCTGGTAACTAGAGATCCCTCAGACCCTTTTAGTCAGTGTG | |
| | | GAAAATCTCTAGCAGTGGCGCCCGAACAGGGACTTGAAAGCGAAAGGGAAACCA | |
| | | GAGGAGCTCTCTCGACGCAGGACTCGGCTTGCTGAAGCGCGCACGGCAAGAGGC | |
| | | GAGGGGCGGCGACTGGTGAGTACGCCAAAAATTTTGACTAGCGGAGGCTAGAAG | |
| | | GAGAGAGATGGGTGCGAGAGCGTCAGTATTAAGCGGGGGAGAATTAGATCGCGA | |
| | | TGGGAAAAAATTCGGTTAAGGCCAGGGGGAAAGAAAAAATATAAATTAAAACAT | |
| | | ATAGTATGGGCAAGCAGGGAGCTAGAACGATTCGCAGTTAATCCTGGCCTGTTA | |
| | | GAAACATCAGAAGGCTGTAGACAAATACTGGGACAGCTACAACCATCCCTTCAG | |
| | | ACAGGATCAGAAGAACTTAGATCATTATATAATACAGTAGCAACCCTCTATTGT | |
| | | GTGCATCAAAGGATAGAGATAAAAGACACCAAGGAAGCTTTAGACAAGATAGAG | |
| | | GAAGAGCAAAACAAAAGTAAGACCACCGCACAGCAAGCGGCCGCTGATCTTCAG | |
| | | ACCTGGAGGAGGAGATATGAGGGACAATTGGAGAAGTGAATTATATAAATATAA | |
| | | AGTAGTAAAAATTGAACCATTAGGAGTAGCACCCACCAAGGCAAAGAGAAGAGT | |
| | | GGTGCAGAGAGAAAAAAGAGCAGTGGGAATAGGAGCTTTGTTCCTTGGGTTCTT | |
| | | GGGAGCAGCAGGAAGCACTATGGGCGCAGCGTCAATGACGCTGACGGTACAGGC | |
| | | CAGACAATTATTGTCTGGTATAGTGCAGCAGCAGAACAATTTGCTGAGGGCTAT | |
| | | TGAGGCGCAACAGCATCTGTTGCAACTCACAGTCTGGGGCATCAAGCAGCTCCA | |
| | | GGCAAGAATCCTGGCTGTGGAAAGATACCTAAAGGATCAACAGCTCCTGGGGAT | |
| | | TTGGGGTTGCTCTGGAAAACTCATTTGCACCACTGCTGTGCCTTGGAATGCTAG | |
| | | TTGGAGTAATAAATCTCTGGAACAGATTTGGAATCACACGACCTGGATGGAGTG | |
| | | GGACAGAGAAATTAACAATTACACAAGCTTAATACACTCCTTAATTGAAGAATC | |
| | | GCAAAACCAGCAAGAAAAGAATGAACAAGAATTATTGGAATTAGATAAATGGGC | |
| | | AAGTTTGTGGAATTGGTTTAACATAACAAATTGGCTGTGGTATATAAAATTATT | |
| | | CATAATGATAGTAGGAGGCTTGGTAGGTTTAAGAATAGTTTTTGCTGTACTTTC | |
| | | TATAGTGAATAGAGTTAGGCAGGGATATTCACCATTATCGTTTCAGACCCACCT | |
| | | CCCAACCCCGAGGGGACCCGACAGGCCCGAAGGAATAGAAGAAGAAGGTGGAGA | |
| | | GAGAGACAGAGACAGATCCATTCGATTAGTGAACGGATCTCGACGGTATCGGTT | |
| | | AACTTTTAAAAGAAAAGGGGGGATTGGGGGGTACAGTGCAGGGGAAAGAATAGT | |
| | | AGACATAATAGCAACAGACATACAAACTAAAGAATTACAAAAACAAATTACAAA | |
| | | ATTCAAAATTTTATCGATACTAGTATTATGCCCAGTACATGACCTTATGGGACT | |
| | | TTCCTACTTGGCAGTACATCTACAAACCCCGGGTGCAAAGATGGATAAAGTTTT | |
| | | AAACAGAGAGGAATCTTTGCAGCTAATGGACCTTCTAGGTCTTGAAAGGAGTGG | |
| | | GAATTGGCTCCGGTGCCCGTCAGTGGGCAGAGCGCACATCGCCCACAGTCCCCG | |
| | | AGAAGTTGGGGGGAGGGGTCGGCAATTGATCCGGTGCCTAGAGAAGGTGGCGCG | |
| | | GGGTAAACTGGGAAAGTGATGTCGTGTACTGGCTCCGCCTTTTTCCCGAGGGTG | |
| | | GGGGAGAACCGTATATAAGTGCAGTAGTCGCCGTGAACGTTCTTTTTCGCAACG | |
| | | GGTTTGCCGCCAGAACACAGGTAAGTGCCGTGTGTGGTTCCCGCGGGCCTGGCC | |
| | | TCTTTACGGGTTATGGCCCTTGCGTGCCTTGAATTACTTCCACTGGCTGCAGTA | |
| | | CGTGATTCTTGATCCCGAGCTTCGGGTTGGAAGTGGGTGGGAGAGTTCGAGGCC | |
| | | TTGCGCTTAAGGAGCCCCTTCGCCTCGTGCTTGAGTTGAGGCCTGGCCTGGGCG | |
| | | CTGGGGCCGCCGCGTGCGAATCTGGTGGCACCTTCGCGCCTGTCTCGCTGCTTT | |
| | | CGATAAGTCTCTAGCCATTTAAAATTTTTGATGACCTGCTGCGACGCTTTTTTT | |
| | | CTGGCAAGATAGTCTTGTAAATGCGGGCCAAGATCTGCACACTGGTATTTCGGT | |
| | | TTTTGGGGCCGCGGGCGGCGACGGGGCCCGTGCGTCCCAGCGCACATGTTCGGC | |
| | | GAGGCGGGGCCTGCGAGCGCGGCCACCGAGAATCGGACGGGGGTAGTCTCAAGC | |
| | | TGGCCGGCCTGCTCTGGTGCCTGGCCTCGCGCCGCCGTGTATCGCCCCGCCCTG | |
| | | GGCGGCAAGGCTGGCCCGGTCGGCACCAGTTGCGTGAGCGGAAAGATGGCCGCT | |
| | | TCCCGGCCCTGCTGCAGGGAGCTCAAAATGGAGGACGCGGCGCTCGGGAGAGCG | |
| | | GGCGGGTGAGTCACCCACACAAAGGAAAAGGGCCTTTCCGTCCTCAGCCGTCGC | |
| | | TTCATGTGACTCCACGGAGTACCGGGCGCCGTCCAGGCACCTCGATTAGTTCTC | |
| | | GAGCTTTTGGAGTACGTCGTCTTTAGGTTGGGGGGAGGGGTTTTATGCGATGGA | |
| | | GTTTCCCCACACTGAGTGGGTGGAGACTGAAGTTAGGCCAGCTTGGCACTTGAT | |
| | | GTAATTCTCCTTGGAATTTGCCCTTTTTGAGTTTGGATCTTGGTTCATTCTCAA | |
| | | GCCTCAGACAGTGGTTCAAAGTTTTTTTCTTCCATTTCAGGTGTCGTGACGTAG | |
| | | CGGCCCTAATACGACTCACTATAGGGCCCGCGTACGTCTAGACCTCCAGGCATC | |
| | | GATTCCACCATGGCACTCCCCGTCACCGCCCTTCTCTTGCCCCTCGCCCTGCTG | |
| | | CTGCATGCTGCCAGGCCCCAGGTCCAATTGGTCCAATCTGGCGCCGAGGTCAAG | |
| | | AAGCCTGGCGCATCCGTAAAAGTGTCTTGTAAAGCCTCTGGTTATACGTTTACG | |
| | | GGGTATTATATGCAATGGGTTCGGCAAGCCCCTGGTCAAGGACTTGAATGGATG | |
| | | GGCTGGATGAACCCTATCTCTGGTAATACGGGCTACGCCCAAAAGTTCCAGGGT | |
| | | AGAGTCACAATGACGCGCAACACGTCAATATCAACCGCTTACATGGAGCTTAGT | |
| | | TCTTTGAGGTCAGAGGACACCGCGGTCTATTATTGTGCTCGCGCCCAATGGGAA | |
| | | CCGCTTGATTACTGGGGGCAAGGTACCTTGGTTACCGTCTCATCAGGTGGAGGC | |
| | | GGTTCAGGCGGAGGTGGCTCTGGCGGTGGCGCTAGCAGCTACGAACTGACACAG | |
| | | CCGCCCTCTGTTAGCGTTAGTCCTGGACAGACCGCCAGGATAACGTGCTCCGGT | |
| | | GACGCATTGCCGAAGAAATATACGTACTGGTACCAGCAGAAGTCCGGGCAAGCA | |
| | | CCTGTACTCGTGATCTATTATGATACAAAACGGCCGAGCGGTATACCAGAACGC | |
| | | TTTAGCGGCTCAAGCAGCGGAACGATGGCCACGTTGACGATTAGCGGCGCGCAG | |
| | | GTCGAGGACGAAGCTGACTACTATTGCCTCAGTACTAACCCAACAGGGCACGAA | |
| | | TCAGTTTTTGGAGGGGGAACCAAGCTTACTGTACTTGGTCAGCCGGCGGCCGCA | |
| | | ACCACAACCCCTGCTCCAAGGCCCCCCACACCCGCTCCAACTATAGCCAGCCAA | |
| | | CCATTGAGCCTCAGACCTGAAGCTTGCAGGCCCGCAGCAGGAGGCGCCGTCCAT | |
| | | ACGCGAGGCCTGGACTTCGCGTGTGATATTTATATTTGGGCCCCTTTGGCCGGA | |
| | | ACATGTGGGGTGTTGCTTCTCTCCCTTGTGATCACTCTGTATTGTAAGCGCGGG | |
| | | AGAAAGAAGCTCCTGTACATCTTCAAGCAGCCTTTTATGCGACCTGTGCAAACC | |
| | | ACTCAGGAAGAAGATGGGTGTTCATGCCGCTTCCCCGAGGAGGAAGAAGGAGGG | |
| | | TGTGAACTGAGGGTGAAATTTTCTAGAAGCGCCGATGCTCCCGCATATCAGCAG | |
| | | GGTCAGAATCAGCTCTACAATGAATTGAATCTCGGCAGGCGAGAAGAGTACGAT | |
| | | GTTCTGGACAAGAGACGGGGCAGGGATCCCGAGATGGGGGGAAAGCCCCGGAGA | |
| | | AAAAATCCTCAGGAGGGGTTGTACAATGAGCTGCAGAAGGACAAGATGGCTGAA | |
| | | GCCTATAGCGAGATCGGAATGAAAGGCGAAAGACGCAGAGGCAAGGGGCATGAC | |
| | | GGTCTGTACCAGGGTCTCTCTACAGCCACCAAGGACACTTATGATGCGTTGCAT | |
| | | ATGCAAGCCTTGCCACCCCGCGGATCAGGAGCGACTAACTTCTCTTTGTTGAAG | |
| | | CAAGCGGGTGACGTTGAAGAGAATCCCGGTCCATCCGGGGGATCCATGCGGATC | |
| | | TCCAAGCCCCACCTCCGAAGCATCAGTATCCAGTGTTATCTGTGCCTTCTTCTG | |
| | | AATTCCCATTTTTTGACAGAGGCAGGAATTCATGTATTCATATTGGGTTGTTTC | |
| | | TCCGCGGGGCTTCCTAAAACCGAAGCTAACTGGGTTAATGTCATTTCAGATCTG | |
| | | AAGAAAATTGAAGATCTTATTCAATCAATGCATATCGACGCGACCTTGTACACG | |
| | | GAGAGTGATGTCCACCCGAGCTGCAAGGTAACGGCCATGAAATGCTTCTTGCTG | |
| | | GAATTGCAGGTAATTAGTCTTGAGAGTGGAGATGCGTCTATACACGACACCGTA | |
| | | GAGAATCTCATTATTTTGGCGAACAATTCTCTCAGCAGTAACGGCAATGTTACT | |
| | | GAATCAGGATGTAAGGAGTGTGAAGAGCTGGAAGAAAAGAACATTAAGGAATTT | |
| | | TTGCAGAGCTTCGTACATATCGTCCAAATGTTCATAAATACAAGCTAAGTCGAC | |
| | | GGCCGGCCGCGAGCATCTTACCGCCATTTATACCCATATTTGTTCTGTTTTTCT | |
| | | TGATTTGGGTATACATTTAAATGTTAATAAAACAAAATGGTGGGGCAATCATTT | |
| | | ACATTTTATAGGATATGTAATTACTAGTTCAGGTGTATTGCCACAAGACAAACA | |
| | | TCTTAAGAAACTTTCCCGTTATTTACGCTCTGTTCCTGTTAATCAACCTCTGGA | |
| | | TTACAAAATTTGTGAAAGATTGACTGATATTCTTAACTATGTTGCTCCTTTTAC | |
| | | GCTGTGTGGATATGCTGCTTTAATACCTCTGTATCATACTATTGCTTCCTGTAC | |
| | | GGCTTTCGTTTTCTCCTCCTTGTATAAATCCTGGTTGCTGTCTCTTTATCAGGA | |
| | | GTTGTGGCCCGTTGTCCGTCAACGTGGCGTGGTGTGCTCTGTGTTTGCTGACGC | |
| | | AACCCCCACTGGCTGGGGCATTGCCACCACCTGTCAACTCCTTTCTGGGACTTT | |
| | | CGCTTTCCCCCTCCCGATCGCCACGGCAGAACTCATCGCCGCCTGCCTTGCCCG | |
| | | CTGCTGGACAGGGGCTAGGTTGCTGGGCACTGATAATTCCGTGGTGTTGTCCTG | |
| | | GTACCTTTAAGACCAATGACTTACAAGGCAGCTGTAGATCTTAGCCACTTTTTA | |
| | | AAAGAAAAGGGGGGACTGGAAGGGCTAATTCACTCCCAACGAAGATAAGATCTG | |
| | | CTTTTTGCTTGTACTGGGTCTCTCTGGTTAGACCAGATCTGAGCCTGGGAGCTC | |
| | | TCTGGCTAACTAGGGAACCCACTGCTTAAGCCTCAATAAAGCTTGCCTTGAGTG | |
| | | CTTCAAGTAGTGTGTGCCCGTCTGTTGTGTGACTCTGGTAACTAGAGATCCCTC | |
| | | AGACCCTTTTAGTCAGTGTGGAAAATCTCTAGCAGTAGTAGTTCATGTCATCTT | |
| | | ATTATTCAGTATTTATAACTTGCAAAGAAATGAATATCAGAGAGTGAGAGGAAC | |
| | | TTGTTTATTGCAGCTTATAATGGTTACAAATAAAGCAATAGCATCACAAATTTC | |
| | | ACAAATAAAGCATTTTTTTCACTGCATTCTAGTTGTGGTTTGTCCAAACTCATC | |
| | | AATGTATCTTATCATGTCTGGCTCTAGCTATCCCGCCCCTAACTCCGCCCATCC | |
| | | CGCCCCTAACTCCGCCCAGTTCCGCCCATTCTCCGCCCCATGGCTGACTAATTT | |
| | | TTTTTATTTATGCAGAGGCCGAGGCCGCCTCGGCCTCTGAGCTATTCCAGAAGT | |
| | | AGTGAGGAGGCTTTTTTGGAGGCCTAGACTTTTGCAGAGACCAAATTCGTAATC | |
| | | ATGTCATAGCTGTTTCCTGTGTGAAATTGTTATCCGCTCACAATTCCACACAAC | |
| | | ATACGAGCCGGAAGCATAAAGTGTAAAGCCTGGGGTGCCTAATGAGTGAGCTAA | |
| | | CTCACATTAATTGCGTTGCGCTCACTGCCCGCTTTCCAGTCGGGAAACCTGTCG | |
| | | TGCCAGCTGCATTAATGAATCGGCCAACGCGCGGGGAGAGGCGGTTTGCGTATT | |
| | | GGGCGCTCTTCCGCTTCCTCGCTCACTGACTCGCTGCGCTCGGTCGTTCGGCTG | |
| | | CGGCGAGCGGTATCAGCTCACTCAAAGGCGGTAATACGGTTATCCACAGAATCA | |
| | | GGGGATAACGCAGGAAAGAACATGTGAGCAAAAGGCCAGCAAAAGGCCAGGAAC | |
| | | CGTAAAAAGGCCGCGTTGCTGGCGTTTTTCCATAGGCTCCGCCCCCCTGACGAG | |
| | | CATCACAAAAATCGACGCTCAAGTCAGAGGTGGCGAAACCCGACAGGACTATAA | |
| | | AGATACCAGGCGTTTCCCCCTGGAAGCTCCCTCGTGCGCTCTCCTGTTCCGACC | |
| | | CTGCCGCTTACCGGATACCTGTCCGCCTTTCTCCCTTCGGGAAGCGTGGCGCTT | |
| | | TCTCATAGCTCACGCTGTAGGTATCTCAGTTCGGTGTAGGTCGTTCGCTCCAAG | |
| | | CTGGGCTGTGTGCACGAACCCCC | |
| F6G- | VH | CAGGTCCAGCTGGTACAGTCTGGCGCTGAGGTAAAGAAGCCTGGTGCTTCAGTG | 53 |
| Var4 | | AAGGTGTCTTGCAAAGCCTCTGGCTATACTTTCACAGGTTATTATATGCACTGG | |
| | | GTGCGCCAGGCCCCCGGTCAGGGCCTTGAATGGATGGGATGGATGAATCCAAAT | |
| | | TCAGGCAATACCGGGTATGCTCAAAAGTTTCAGGGCCGCGTAACAATGACCCGA | |
| | | AATACAAGTATTTCAACTGCTTATATGGAACTCAGTTCTCTCAGAAGTGAAGAT | |
| | | ACGGCCGTATATTATTGCGCGCGGGCGCAATGGGAACCACTTGACTATTGGGGA | |
| | | CAAGGCACACTGGTTACGGTGTCATC | |
| | VL | TCATATGAACTCACACAGCCGCCATCTGTTAGCGTTTCTCCAGGGCAGACTGCC | 61 |
| | | CGAATTACATGCTCAGGCGATGCCCTCCCGAAAAAATATACCTACTGGTACCAA | |
| | | CAAAAATCCGGCCAGGCACCTGTGTTGGTGATATATTATGATACAAAACGCCCT | |
| | | TCAGGGATCCCGGAGAGATTCAGTGGGTCAAGTAGCGGAACAATGGCAACACTT | |
| | | ACTATAAGCGGGGCACAGGTTGAGGACGAGGCTGACTACTACTGCCTGTCTACT | |
| | | CATCCGGCCGGACAGGAGAGTGTTTTCGGGGGTGGAACTAAACTCACAGTACTG | |
| | | GGACAGCCC | |
| | scFv | CAGGTCCAGCTGGTACAGTCTGGCGCTGAGGTAAAGAAGCCTGGTGCTTCAGTG | 69 |
| | | AAGGTGTCTTGCAAAGCCTCTGGCTATACTTTCACAGGTTATTATATGCACTGG | |
| | | GTGCGCCAGGCCCCCGGTCAGGGCCTTGAATGGATGGGATGGATGAATCCAAAT | |
| | | TCAGGCAATACCGGGTATGCTCAAAAGTTTCAGGGCCGCGTAACAATGACCCGA | |
| | | AATACAAGTATTTCAACTGCTTATATGGAACTCAGTTCTCTCAGAAGTGAAGAT | |
| | | ACGGCCGTATATTATTGCGCGCGGGCGCAATGGGAACCACTTGACTATTGGGGA | |
| | | CAAGGCACACTGGTTACGGTGTCATCTGGTGGAGGCGGTTCAGGCGGAGGTGGC | |
| | | TCTGGCGGTGGCGCTAGCTCATATGAACTCACACAGCCGCCATCTGTTAGCGTT | |
| | | TCTCCAGGGCAGACTGCCCGAATTACATGCTCAGGCGATGCCCTCCCGAAAAAA | |
| | | TATACCTACTGGTACCAACAAAAATCCGGCCAGGCACCTGTGTTGGTGATATAT | |
| | | TATGATACAAAACGCCCTTCAGGGATCCCGGAGAGATTCAGTGGGTCAAGTAGC | |
| | | GGAACAATGGCAACACTTACTATAAGCGGGGCACAGGTTGAGGACGAGGCTGAC | |
| | | TACTACTGCCTGTCTACTCATCCGGCCGGACAGGAGAGTGTTTTCGGGGGTGGA | |
| | | ACTAAACTCACAGTACTGGGACAGCCC | |
| | CAR | CAGGTCCAGCTGGTACAGTCTGGCGCTGAGGTAAAGAAGCCTGGTGCTTCAGTG | 99 |
| | | AAGGTGTCTTGCAAAGCCTCTGGCTATACTTTCACAGGTTATTATATGCACTGG | |
| | | GTGCGCCAGGCCCCCGGTCAGGGCCTTGAATGGATGGGATGGATGAATCCAAAT | |
| | | TCAGGCAATACCGGGTATGCTCAAAAGTTTCAGGGCCGCGTAACAATGACCCGA | |
| | | AATACAAGTATTTCAACTGCTTATATGGAACTCAGTTCTCTCAGAAGTGAAGAT | |
| | | ACGGCCGTATATTATTGCGCGCGGGCGCAATGGGAACCACTTGACTATTGGGGA | |
| | | CAAGGCACACTGGTTACGGTGTCATCTGGTGGAGGCGGTTCAGGCGGAGGTGGC | |
| | | TCTGGCGGTGGCGCTAGCTCATATGAACTCACACAGCCGCCATCTGTTAGCGTT | |
| | | TCTCCAGGGCAGACTGCCCGAATTACATGCTCAGGCGATGCCCTCCCGAAAAAA | |
| | | TATACCTACTGGTACCAACAAAAATCCGGCCAGGCACCTGTGTTGGTGATATAT | |
| | | TATGATACAAAACGCCCTTCAGGGATCCCGGAGAGATTCAGTGGGTCAAGTAGC | |
| | | GGAACAATGGCAACACTTACTATAAGCGGGGCACAGGTTGAGGACGAGGCTGAC | |
| | | TACTACTGCCTGTCTACTCATCCGGCCGGACAGGAGAGTGTTTTCGGGGGTGGA | |
| | | ACTAAACTCACAGTACTGGGACAGCCCGCGGCCGCAACCACAACCCCTGCTCCA | |
| | | AGGCCCCCCACACCCGCTCCAACTATAGCCAGCCAACCATTGAGCCTCAGACCT | |
| | | GAAGCTTGCAGGCCCGCAGCAGGAGGCGCCGTCCATACGCGAGGCCTGGACTTC | |
| | | GCGTGTGATATTTATATTTGGGCCCCTTTGGCCGGAACATGTGGGGTGTTGCTT | |
| | | CTCTCCCTTGTGATCACTCTGTATTGTAAGCGCGGGAGAAAGAAGCTCCTGTAC | |
| | | ATCTTCAAGCAGCCTTTTATGCGACCTGTGCAAACCACTCAGGAAGAAGATGGG | |
| | | TGTTCATGCCGCTTCCCCGAGGAGGAAGAAGGAGGGTGTGAACTGAGGGTGAAA | |
| | | TTTTCTAGAAGCGCCGATGCTCCCGCATATCAGCAGGGTCAGAATCAGCTCTAC | |
| | | AATGAATTGAATCTCGGCAGGCGAGAAGAGTACGATGTTCTGGACAAGAGACGG | |
| | | GGCAGGGATCCCGAGATGGGGGGAAAGCCCCGGAGAAAAAATCCTCAGGAGGGG | |
| | | TTGTACAATGAGCTGCAGAAGGACAAGATGGCTGAAGCCTATAGCGAGATCGGA | |
| | | ATGAAAGGCGAAAGACGCAGAGGCAAGGGGCATGACGGTCTGTACCAGGGTCTC | |
| | | TCTACAGCCACCAAGGACACTTATGATGCGTTGCATATGCAAGCCTTGCCACCC | |
| | | CGC | |
| | Lentivira | cgttcagcccgaccgctgcgccttatccggtaactatcgtcttgagtccaacCC | 77 |
| | 1 vector | GGTAAGACACGACTTATCGCCACTGGCAGCAGCCACTGGTAACAGGATTAGCAG | |
| | | AGCGAGGTATGTAGGCGGTGCTACAGAGTTCTTGAAGTGGTGGCCTAACTACGG | |
| | | CTACACTAGAAGAACAGTATTTGGTATCTGCGCTCTGCTGAAGCCAGTTACCTT | |
| | | CGGAAAAAGAGTTGGTAGCTCTTGATCCGGCAAACAAACCACCGCTGGTAGCGG | |
| | | TGGTTTTTTTGTTTGCAAGCAGCAGATTACGCGCAGAAAAAAAGGATCTCAAGA | |
| | | AGATCCTTTGATCTTTTCTACGGGGTCTGACGCTCAGTGGAACGAAAACTCACG | |
| | | TTAAGGGATTTTGGTCATGAGATTATCAAAAAGGATCTTCACCTAGATCCTTTT | |
| | | AAATTAAAAATGAAGTTTTAAATCAATCTAAAGTATATATGAGTAAACTTGGTC | |
| | | TGACAGTTAGAAAAACTCATCGAGCATCAAATGAAACTGCAATTTATTCATATC | |
| | | AGGATTATCAATACCATATTTTTGAAAAAGCCGTTTCTGTAATGAAGGAGAAAA | |
| | | CTCACCGAGGCAGTTCCATAGGATGGCAAGATCCTGGTATCGGTCTGCGATTCC | |
| | | GACTCGTCCAACATCAATACAACCTATTAATTTCCCCTCGTCAAAAATAAGGTT | |
| | | ATCAAGTGAGAAATCACCATGAGTGACGACTGAATCCGGTGAGAATGGCAAAAG | |
| | | TTTATGCATTTCTTTCCAGACTTGTTCAACAGGCCAGCCATTACGCTCGTCATC | |
| | | AAAATCACTCGCATCAACCAAACCGTTATTCATTCGTGATTGCGCCTGAGCGAG | |
| | | ACGAAATACGCGATCGCTGTTAAAAGGACAATTACAAACAGGAATCGAATGCAA | |
| | | CCGGCGCAGGAACACTGCCAGCGCATCAACAATATTTTCACCTGAATCAGGATA | |
| | | TTCTTCTAATACCTGGAATGCTGTTTTTCCGGGGATCGCAGTGGTGAGTAACCA | |
| | | TGCATCATCAGGAGTACGGATAAAATGCTTGATGGTCGGAAGAGGCATAAATTC | |
| | | CGTCAGCCAGTTTAGTCTGACCATCTCATCTGTAACATCATTGGCAACGCTACC | |
| | | TTTGCCATGTTTCAGAAACAACTCTGGCGCATCGGGCTTCCCATACAAGCGATA | |
| | | GATTGTCGCACCTGATTGCCCGACATTATCGCGAGCCCATTTATACCCATATAA | |
| | | ATCAGCATCCATGTTGGAATTTAATCGCGGCCTCGACGTTTCCCGTTGAATATG | |
| | | GCTCATACTCTTCCTTTTTCAATATTATTGAAGCATTTATCAGGGTTATTGTCT | |
| | | CATGAGCGGATACATATTTGAATGTATTTAGAAAAATAAACAAATAGGGGTTCC | |
| | | GCGCACATTTCCCCGAAAAGTGCCACCTGACGGATCGGGAGATCTCCCGATCCC | |
| | | CTATGGTGCACTCTCAGTACAATCTGCTCTGATGCCGCATAGTTAAGCCAGTAT | |
| | | CTGCTCCCTGCTTGTGTGTTGGAGGTCGCTGAGTAGTGCGCGAGCAAAATTTAA | |
| | | GCTACAACAAGGCAAGGCTTGACCGACAATTGCATGAAGAATCTGCTTAGGGTT | |
| | | AGGCGTTTTGCGCTGCTTCGCGATGTACGGGCCAGATATACCCGTTGACATTGA | |
| | | TTATTGACTAGTTATTAATAGTAATCAATTACGGGGTCATTAGTTCATAGCCCA | |
| | | TATATGGAGTTCCGCGTTACATAACTTACGGTAAATGGCCCGCCTGGCTGACCG | |
| | | CCCAACGACCCCCGCCCATTGACGTCAATAATGACGTATGTTCCCATAGTAACG | |
| | | CCAATAGGGACTTTCCATTGACGTCAATGGGTGGAGTATTTACGGTAAACTGCC | |
| | | CACTTGGCAGTACATCAAGTGTATCATATGCCAAGTACGCCCCCTATTGACGTC | |
| | | AATGACGGTAAATGGCCCGCCTGGCATTATGCCCAGTACATGACCTTATGGGAC | |
| | | TTTCCTACTTGGCAGTACATCTACGTATTAGTCATCGCTATTACCATGGTGATG | |
| | | CGGTTTTGGCAGTACATCAATGGGCGTGGATAGCGGTTTGACTCACGGGGATTT | |
| | | CCAAGTCTCCACCCCATTGACGTCAATGGGAGTTTGTTTTGGCACCAAAATCAA | |
| | | CGGGACTTTCCAAAATGTCGTAACAACTCCGCCCCATTGACGCAAATGGGCGGT | |
| | | AGGCGTGTACGGTGGGAGGTCTATATAAGCAGCGCGTTTTGCCTGTACTGGGTC | |
| | | TCTCTGGTTAGACCAGATCTGAGCCTGGGAGCTCTCTGGCTAACTAGGGAACCC | |
| | | ACTGCTTAAGCCTCAATAAAGCTTGCCTTGAGTGCTTCAAGTAGTGTGTGCCCG | |
| | | TCTGTTGTGTGACTCTGGTAACTAGAGATCCCTCAGACCCTTTTAGTCAGTGTG | |
| | | GAAAATCTCTAGCAGTGGCGCCCGAACAGGGACTTGAAAGCGAAAGGGAAACCA | |
| | | GAGGAGCTCTCTCGACGCAGGACTCGGCTTGCTGAAGCGCGCACGGCAAGAGGC | |
| | | GAGGGGCGGCGACTGGTGAGTACGCCAAAAATTTTGACTAGCGGAGGCTAGAAG | |
| | | GAGAGAGATGGGTGCGAGAGCGTCAGTATTAAGCGGGGGAGAATTAGATCGCGA | |
| | | TGGGAAAAAATTCGGTTAAGGCCAGGGGGAAAGAAAAAATATAAATTAAAACAT | |
| | | ATAGTATGGGCAAGCAGGGAGCTAGAACGATTCGCAGTTAATCCTGGCCTGTTA | |
| | | GAAACATCAGAAGGCTGTAGACAAATACTGGGACAGCTACAACCATCCCTTCAG | |
| | | ACAGGATCAGAAGAACTTAGATCATTATATAATACAGTAGCAACCCTCTATTGT | |
| | | GTGCATCAAAGGATAGAGATAAAAGACACCAAGGAAGCTTTAGACAAGATAGAG | |
| | | GAAGAGCAAAACAAAAGTAAGACCACCGCACAGCAAGCGGCCGCTGATCTTCAG | |
| | | ACCTGGAGGAGGAGATATGAGGGACAATTGGAGAAGTGAATTATATAAATATAA | |
| | | AGTAGTAAAAATTGAACCATTAGGAGTAGCACCCACCAAGGCAAAGAGAAGAGT | |
| | | GGTGCAGAGAGAAAAAAGAGCAGTGGGAATAGGAGCTTTGTTCCTTGGGTTCTT | |
| | | GGGAGCAGCAGGAAGCACTATGGGCGCAGCGTCAATGACGCTGACGGTACAGGC | |
| | | CAGACAATTATTGTCTGGTATAGTGCAGCAGCAGAACAATTTGCTGAGGGCTAT | |
| | | TGAGGCGCAACAGCATCTGTTGCAACTCACAGTCTGGGGCATCAAGCAGCTCCA | |
| | | GGCAAGAATCCTGGCTGTGGAAAGATACCTAAAGGATCAACAGCTCCTGGGGAT | |
| | | TTGGGGTTGCTCTGGAAAACTCATTTGCACCACTGCTGTGCCTTGGAATGCTAG | |
| | | TTGGAGTAATAAATCTCTGGAACAGATTTGGAATCACACGACCTGGATGGAGTG | |
| | | GGACAGAGAAATTAACAATTACACAAGCTTAATACACTCCTTAATTGAAGAATC | |
| | | GCAAAACCAGCAAGAAAAGAATGAACAAGAATTATTGGAATTAGATAAATGGGC | |
| | | AAGTTTGTGGAATTGGTTTAACATAACAAATTGGCTGTGGTATATAAAATTATT | |
| | | CATAATGATAGTAGGAGGCTTGGTAGGTTTAAGAATAGTTTTTGCTGTACTTTC | |
| | | TATAGTGAATAGAGTTAGGCAGGGATATTCACCATTATCGTTTCAGACCCACCT | |
| | | CCCAACCCCGAGGGGACCCGACAGGCCCGAAGGAATAGAAGAAGAAGGTGGAGA | |
| | | GAGAGACAGAGACAGATCCATTCGATTAGTGAACGGATCTCGACGGTATCGGTT | |
| | | AACTTTTAAAAGAAAAGGGGGGATTGGGGGGTACAGTGCAGGGGAAAGAATAGT | |
| | | AGACATAATAGCAACAGACATACAAACTAAAGAATTACAAAAACAAATTACAAA | |
| | | ATTCAAAATTTTATCGATACTAGTATTATGCCCAGTACATGACCTTATGGGACT | |
| | | TTCCTACTTGGCAGTACATCTACAAACCCCGGGTGCAAAGATGGATAAAGTTTT | |
| | | AAACAGAGAGGAATCTTTGCAGCTAATGGACCTTCTAGGTCTTGAAAGGAGTGG | |
| | | GAATTGGCTCCGGTGCCCGTCAGTGGGCAGAGCGCACATCGCCCACAGTCCCCG | |
| | | AGAAGTTGGGGGGAGGGGTCGGCAATTGATCCGGTGCCTAGAGAAGGTGGCGCG | |
| | | GGGTAAACTGGGAAAGTGATGTCGTGTACTGGCTCCGCCTTTTTCCCGAGGGTG | |
| | | GGGGAGAACCGTATATAAGTGCAGTAGTCGCCGTGAACGTTCTTTTTCGCAACG | |
| | | GGTTTGCCGCCAGAACACAGGTAAGTGCCGTGTGTGGTTCCCGCGGGCCTGGCC | |
| | | TCTTTACGGGTTATGGCCCTTGCGTGCCTTGAATTACTTCCACTGGCTGCAGTA | |
| | | CGTGATTCTTGATCCCGAGCTTCGGGTTGGAAGTGGGTGGGAGAGTTCGAGGCC | |
| | | TTGCGCTTAAGGAGCCCCTTCGCCTCGTGCTTGAGTTGAGGCCTGGCCTGGGCG | |
| | | CTGGGGCCGCCGCGTGCGAATCTGGTGGCACCTTCGCGCCTGTCTCGCTGCTTT | |
| | | CGATAAGTCTCTAGCCATTTAAAATTTTTGATGACCTGCTGCGACGCTTTTTTT | |
| | | CTGGCAAGATAGTCTTGTAAATGCGGGCCAAGATCTGCACACTGGTATTTCGGT | |
| | | TTTTGGGGCCGCGGGCGGCGACGGGGCCCGTGCGTCCCAGCGCACATGTTCGGC | |
| | | GAGGCGGGGCCTGCGAGCGCGGCCACCGAGAATCGGACGGGGGTAGTCTCAAGC | |
| | | TGGCCGGCCTGCTCTGGTGCCTGGCCTCGCGCCGCCGTGTATCGCCCCGCCCTG | |
| | | GGCGGCAAGGCTGGCCCGGTCGGCACCAGTTGCGTGAGCGGAAAGATGGCCGCT | |
| | | TCCCGGCCCTGCTGCAGGGAGCTCAAAATGGAGGACGCGGCGCTCGGGAGAGCG | |
| | | GGCGGGTGAGTCACCCACACAAAGGAAAAGGGCCTTTCCGTCCTCAGCCGTCGC | |
| | | TTCATGTGACTCCACGGAGTACCGGGCGCCGTCCAGGCACCTCGATTAGTTCTC | |
| | | GAGCTTTTGGAGTACGTCGTCTTTAGGTTGGGGGGAGGGGTTTTATGCGATGGA | |
| | | GTTTCCCCACACTGAGTGGGTGGAGACTGAAGTTAGGCCAGCTTGGCACTTGAT | |
| | | GTAATTCTCCTTGGAATTTGCCCTTTTTGAGTTTGGATCTTGGTTCATTCTCAA | |
| | | GCCTCAGACAGTGGTTCAAAGTTTTTTTCTTCCATTTCAGGTGTCGTGACGTAG | |
| | | CGGCCCTAATACGACTCACTATAGGGCCCGCGTACGTCTAGACCTCCAGGCATC | |
| | | GATTCCACCATGGCACTCCCCGTCACCGCCCTTCTCTTGCCCCTCGCCCTGCTG | |
| | | CTGCATGCTGCCAGGCCCCAGGTCCAGCTGGTACAGTCTGGCGCTGAGGTAAAG | |
| | | AAGCCTGGTGCTTCAGTGAAGGTGTCTTGCAAAGCCTCTGGCTATACTTTCACA | |
| | | GGTTATTATATGCACTGGGTGCGCCAGGCCCCCGGTCAGGGCCTTGAATGGATG | |
| | | GGATGGATGAATCCAAATTCAGGCAATACCGGGTATGCTCAAAAGTTTCAGGGC | |
| | | CGCGTAACAATGACCCGAAATACAAGTATTTCAACTGCTTATATGGAACTCAGT | |
| | | TCTCTCAGAAGTGAAGATACGGCCGTATATTATTGCGCGCGGGCGCAATGGGAA | |
| | | CCACTTGACTATTGGGGACAAGGCACACTGGTTACGGTGTCATCTGGTGGAGGC | |
| | | GGTTCAGGCGGAGGTGGCTCTGGCGGTGGCGCTAGCTCATATGAACTCACACAG | |
| | | CCGCCATCTGTTAGCGTTTCTCCAGGGCAGACTGCCCGAATTACATGCTCAGGC | |
| | | GATGCCCTCCCGAAAAAATATACCTACTGGTACCAACAAAAATCCGGCCAGGCA | |
| | | CCTGTGTTGGTGATATATTATGATACAAAACGCCCTTCAGGGATCCCGGAGAGA | |
| | | TTCAGTGGGTCAAGTAGCGGAACAATGGCAACACTTACTATAAGCGGGGCACAG | |
| | | GTTGAGGACGAGGCTGACTACTACTGCCTGTCTACTCATCCGGCCGGACAGGAG | |
| | | AGTGTTTTCGGGGGTGGAACTAAACTCACAGTACTGGGACAGCCCGCGGCCGCA | |
| | | ACCACAACCCCTGCTCCAAGGCCCCCCACACCCGCTCCAACTATAGCCAGCCAA | |
| | | CCATTGAGCCTCAGACCTGAAGCTTGCAGGCCCGCAGCAGGAGGCGCCGTCCAT | |
| | | ACGCGAGGCCTGGACTTCGCGTGTGATATTTATATTTGGGCCCCTTTGGCCGGA | |
| | | ACATGTGGGGTGTTGCTTCTCTCCCTTGTGATCACTCTGTATTGTAAGCGCGGG | |
| | | AGAAAGAAGCTCCTGTACATCTTCAAGCAGCCTTTTATGCGACCTGTGCAAACC | |
| | | ACTCAGGAAGAAGATGGGTGTTCATGCCGCTTCCCCGAGGAGGAAGAAGGAGGG | |
| | | TGTGAACTGAGGGTGAAATTTTCTAGAAGCGCCGATGCTCCCGCATATCAGCAG | |
| | | GGTCAGAATCAGCTCTACAATGAATTGAATCTCGGCAGGCGAGAAGAGTACGAT | |
| | | GTTCTGGACAAGAGACGGGGCAGGGATCCCGAGATGGGGGGAAAGCCCCGGAGA | |
| | | AAAAATCCTCAGGAGGGGTTGTACAATGAGCTGCAGAAGGACAAGATGGCTGAA | |
| | | GCCTATAGCGAGATCGGAATGAAAGGCGAAAGACGCAGAGGCAAGGGGCATGAC | |
| | | GGTCTGTACCAGGGTCTCTCTACAGCCACCAAGGACACTTATGATGCGTTGCAT | |
| | | ATGCAAGCCTTGCCACCCCGCGGATCAGGAGCGACTAACTTCTCTTTGTTGAAG | |
| | | CAAGCGGGTGACGTTGAAGAGAATCCCGGTCCATCCGGGGGATCCATGCGGATC | |
| | | TCCAAGCCCCACCTCCGAAGCATCAGTATCCAGTGTTATCTGTGCCTTCTTCTG | |
| | | AATTCCCATTTTTTGACAGAGGCAGGAATTCATGTATTCATATTGGGTTGTTTC | |
| | | TCCGCGGGGCTTCCTAAAACCGAAGCTAACTGGGTTAATGTCATTTCAGATCTG | |
| | | AAGAAAATTGAAGATCTTATTCAATCAATGCATATCGACGCGACCTTGTACACG | |
| | | GAGAGTGATGTCCACCCGAGCTGCAAGGTAACGGCCATGAAATGCTTCTTGCTG | |
| | | GAATTGCAGGTAATTAGTCTTGAGAGTGGAGATGCGTCTATACACGACACCGTA | |
| | | GAGAATCTCATTATTTTGGCGAACAATTCTCTCAGCAGTAACGGCAATGTTACT | |
| | | GAATCAGGATGTAAGGAGTGTGAAGAGCTGGAAGAAAAGAACATTAAGGAATTT | |
| | | TTGCAGAGCTTCGTACATATCGTCCAAATGTTCATAAATACAAGCTAAGTCGAC | |
| | | GGCCGGCCGCGAGCATCTTACCGCCATTTATACCCATATTTGTTCTGTTTTTCT | |
| | | TGATTTGGGTATACATTTAAATGTTAATAAAACAAAATGGTGGGGCAATCATTT | |
| | | ACATTTTATAGGATATGTAATTACTAGTTCAGGTGTATTGCCACAAGACAAACA | |
| | | TCTTAAGAAACTTTCCCGTTATTTACGCTCTGTTCCTGTTAATCAACCTCTGGA | |
| | | TTACAAAATTTGTGAAAGATTGACTGATATTCTTAACTATGTTGCTCCTTTTAC | |
| | | GCTGTGTGGATATGCTGCTTTAATACCTCTGTATCATACTATTGCTTCCTGTAC | |
| | | GGCTTTCGTTTTCTCCTCCTTGTATAAATCCTGGTTGCTGTCTCTTTATCAGGA | |
| | | GTTGTGGCCCGTTGTCCGTCAACGTGGCGTGGTGTGCTCTGTGTTTGCTGACGC | |
| | | AACCCCCACTGGCTGGGGCATTGCCACCACCTGTCAACTCCTTTCTGGGACTTT | |
| | | CGCTTTCCCCCTCCCGATCGCCACGGCAGAACTCATCGCCGCCTGCCTTGCCCG | |
| | | CTGCTGGACAGGGGCTAGGTTGCTGGGCACTGATAATTCCGTGGTGTTGTCCTG | |
| | | GTACCTTTAAGACCAATGACTTACAAGGCAGCTGTAGATCTTAGCCACTTTTTA | |
| | | AAAGAAAAGGGGGGACTGGAAGGGCTAATTCACTCCCAACGAAGATAAGATCTG | |
| | | CTTTTTGCTTGTACTGGGTCTCTCTGGTTAGACCAGATCTGAGCCTGGGAGCTC | |
| | | TCTGGCTAACTAGGGAACCCACTGCTTAAGCCTCAATAAAGCTTGCCTTGAGTG | |
| | | CTTCAAGTAGTGTGTGCCCGTCTGTTGTGTGACTCTGGTAACTAGAGATCCCTC | |
| | | AGACCCTTTTAGTCAGTGTGGAAAATCTCTAGCAGTAGTAGTTCATGTCATCTT | |
| | | ATTATTCAGTATTTATAACTTGCAAAGAAATGAATATCAGAGAGTGAGAGGAAC | |
| | | TTGTTTATTGCAGCTTATAATGGTTACAAATAAAGCAATAGCATCACAAATTTC | |
| | | ACAAATAAAGCATTTTTTTCACTGCATTCTAGTTGTGGTTTGTCCAAACTCATC | |
| | | AATGTATCTTATCATGTCTGGCTCTAGCTATCCCGCCCCTAACTCCGCCCATCC | |
| | | CGCCCCTAACTCCGCCCAGTTCCGCCCATTCTCCGCCCCATGGCTGACTAATTT | |
| | | TTTTTATTTATGCAGAGGCCGAGGCCGCCTCGGCCTCTGAGCTATTCCAGAAGT | |
| | | AGTGAGGAGGCTTTTTTGGAGGCCTAGACTTTTGCAGAGACCAAATTCGTAATC | |
| | | ATGTCATAGCTGTTTCCTGTGTGAAATTGTTATCCGCTCACAATTCCACACAAC | |
| | | ATACGAGCCGGAAGCATAAAGTGTAAAGCCTGGGGTGCCTAATGAGTGAGCTAA | |
| | | CTCACATTAATTGCGTTGCGCTCACTGCCCGCTTTCCAGTCGGGAAACCTGTCG | |
| | | TGCCAGCTGCATTAATGAATCGGCCAACGCGCGGGGAGAGGCGGTTTGCGTATT | |
| | | GGGCGCTCTTCCGCTTCCTCGCTCACTGACTCGCTGCGCTCGGTCGTTCGGCTG | |
| | | CGGCGAGCGGTATCAGCTCACTCAAAGGCGGTAATACGGTTATCCACAGAATCA | |
| | | GGGGATAACGCAGGAAAGAACATGTGAGCAAAAGGCCAGCAAAAGGCCAGGAAC | |
| | | CGTAAAAAGGCCGCGTTGCTGGCGTTTTTCCATAGGCTCCGCCCCCCTGACGAG | |
| | | CATCACAAAAATCGACGCTCAAGTCAGAGGTGGCGAAACCCGACAGGACTATAA | |
| | | AGATACCAGGCGTTTCCCCCTGGAAGCTCCCTCGTGCGCTCTCCTGTTCCGACC | |
| | | CTGCCGCTTACCGGATACCTGTCCGCCTTTCTCCCTTCGGGAAGCGTGGCGCTT | |
| | | TCTCATAGCTCACGCTGTAGGTATCTCAGTTCGGTGTAGGTCGTTCGCTCCAAG | |
| | | CTGGGCTGTGTGCACGAACCCCC | |
| F6G- | VH | CAGGTCCAACTGGTGCAATCTGGCGCTGAAGTGAAAAAGCCCGGCGCATCAGTA | 54 |
| Var5 | | AAGGTGAGTTGCAAAGCATCTGGGTACACTTTCACTGGCTATTATATGCATTGG | |
| | | GTAAGGCAAGCCCCCGGCCAAGGCCTGGAATGGATGGGATGGATGAATCCGAAC | |
| | | TCCGGTAATACAGGGTACGCTCAAAAATTTCAAGGACGGGTCACAATGACGCGG | |
| | | AACACCAGTATATCTACTGCATATATGGAGCTCAGTTCACTGCGCTCCGAGGAT | |
| | | ACAGCTGTATACTATTGCGCCAGGGCTCAATGGGAACCACTGGACTATTGGGGT | |
| | | CAAGGGACACTTGTAACGGTAAGCTC | |
| | VL | TCCTATGAGCTTACTCAGCCACCCTCTGTATCTGTATCTCCTGGGCAGACAGCA | 62 |
| | | CGCATCACATGTAGTGGCGATGCCCTCCCGAAAAAATACACGTATTGGTATCAA | |
| | | CAGAAATCCGGACAGGCACCGGTTTTGGTGATATACTATGATACGAAAAGACCT | |
| | | AGTGGAATACCTGAGCGGTTTTCTGGAAGTTCTTCTGGTACCATGGCAACTTTG | |
| | | ACAATCTCAGGTGCCCAGGTCGAGGACGAGGCCGACTATTATTGTTTGTCTACA | |
| | | AATCCGACAGGTCATGAATCCGTGTTCGGCGGAGGGACAAAGCTTACCGTCCTT | |
| | | GGGCAGCCT | |
| | scFv | CAGGTCCAACTGGTGCAATCTGGCGCTGAAGTGAAAAAGCCCGGCGCATCAGTA | 70 |
| | | AAGGTGAGTTGCAAAGCATCTGGGTACACTTTCACTGGCTATTATATGCATTGG | |
| | | GTAAGGCAAGCCCCCGGCCAAGGCCTGGAATGGATGGGATGGATGAATCCGAAC | |
| | | TCCGGTAATACAGGGTACGCTCAAAAATTTCAAGGACGGGTCACAATGACGCGG | |
| | | AACACCAGTATATCTACTGCATATATGGAGCTCAGTTCACTGCGCTCCGAGGAT | |
| | | ACAGCTGTATACTATTGCGCCAGGGCTCAATGGGAACCACTGGACTATTGGGGT | |
| | | CAAGGGACACTTGTAACGGTAAGCTCAGGTGGAGGCGGTTCAGGCGGAGGTGGC | |
| | | TCTGGCGGTGGCGCTAGCTCCTATGAGCTTACTCAGCCACCCTCTGTATCTGTA | |
| | | TCTCCTGGGCAGACAGCACGCATCACATGTAGTGGCGATGCCCTCCCGAAAAAA | |
| | | TACACGTATTGGTATCAACAGAAATCCGGACAGGCACCGGTTTTGGTGATATAC | |
| | | TATGATACGAAAAGACCTAGTGGAATACCTGAGCGGTTTTCTGGAAGTTCTTCT | |
| | | GGTACCATGGCAACTTTGACAATCTCAGGTGCCCAGGTCGAGGACGAGGCCGAC | |
| | | TATTATTGTTTGTCTACAAATCCGACAGGTCATGAATCCGTGTTCGGCGGAGGG | |
| | | ACAAAGCTTACCGTCCTTGGGCAGCCT | |
| | CAR | CAGGTCCAACTGGTGCAATCTGGCGCTGAAGTGAAAAAGCCCGGCGCATCAGTA | 100 |
| | | AAGGTGAGTTGCAAAGCATCTGGGTACACTTTCACTGGCTATTATATGCATTGG | |
| | | GTAAGGCAAGCCCCCGGCCAAGGCCTGGAATGGATGGGATGGATGAATCCGAAC | |
| | | TCCGGTAATACAGGGTACGCTCAAAAATTTCAAGGACGGGTCACAATGACGCGG | |
| | | AACACCAGTATATCTACTGCATATATGGAGCTCAGTTCACTGCGCTCCGAGGAT | |
| | | ACAGCTGTATACTATTGCGCCAGGGCTCAATGGGAACCACTGGACTATTGGGGT | |
| | | CAAGGGACACTTGTAACGGTAAGCTCAGGTGGAGGCGGTTCAGGCGGAGGTGGC | |
| | | TCTGGCGGTGGCGCTAGCTCCTATGAGCTTACTCAGCCACCCTCTGTATCTGTA | |
| | | TCTCCTGGGCAGACAGCACGCATCACATGTAGTGGCGATGCCCTCCCGAAAAAA | |
| | | TACACGTATTGGTATCAACAGAAATCCGGACAGGCACCGGTTTTGGTGATATAC | |
| | | TATGATACGAAAAGACCTAGTGGAATACCTGAGCGGTTTTCTGGAAGTTCTTCT | |
| | | GGTACCATGGCAACTTTGACAATCTCAGGTGCCCAGGTCGAGGACGAGGCCGAC | |
| | | TATTATTGTTTGTCTACAAATCCGACAGGTCATGAATCCGTGTTCGGCGGAGGG | |
| | | ACAAAGCTTACCGTCCTTGGGCAGCCTGCGGCCGCAACCACAACCCCTGCTCCA | |
| | | AGGCCCCCCACACCCGCTCCAACTATAGCCAGCCAACCATTGAGCCTCAGACCT | |
| | | GAAGCTTGCAGGCCCGCAGCAGGAGGCGCCGTCCATACGCGAGGCCTGGACTTC | |
| | | GCGTGTGATATTTATATTTGGGCCCCTTTGGCCGGAACATGTGGGGTGTTGCTT | |
| | | CTCTCCCTTGTGATCACTCTGTATTGTAAGCGCGGGAGAAAGAAGCTCCTGTAC | |
| | | ATCTTCAAGCAGCCTTTTATGCGACCTGTGCAAACCACTCAGGAAGAAGATGGG | |
| | | TGTTCATGCCGCTTCCCCGAGGAGGAAGAAGGAGGGTGTGAACTGAGGGTGAAA | |
| | | TTTTCTAGAAGCGCCGATGCTCCCGCATATCAGCAGGGTCAGAATCAGCTCTAC | |
| | | AATGAATTGAATCTCGGCAGGCGAGAAGAGTACGATGTTCTGGACAAGAGACGG | |
| | | GGCAGGGATCCCGAGATGGGGGGAAAGCCCCGGAGAAAAAATCCTCAGGAGGGG | |
| | | TTGTACAATGAGCTGCAGAAGGACAAGATGGCTGAAGCCTATAGCGAGATCGGA | |
| | | ATGAAAGGCGAAAGACGCAGAGGCAAGGGGCATGACGGTCTGTACCAGGGTCTC | |
| | | TCTACAGCCACCAAGGACACTTATGATGCGTTGCATATGCAAGCCTTGCCACCC | |
| | | CGC | |
| | Lentivira | cgttcagcccgaccgctgcgccttatccggtaactatcgtcttgagtccaacCC | 78 |
| | 1 vector | GGTAAGACACGACTTATCGCCACTGGCAGCAGCCACTGGTAACAGGATTAGCAG | |
| | | AGCGAGGTATGTAGGCGGTGCTACAGAGTTCTTGAAGTGGTGGCCTAACTACGG | |
| | | CTACACTAGAAGAACAGTATTTGGTATCTGCGCTCTGCTGAAGCCAGTTACCTT | |
| | | CGGAAAAAGAGTTGGTAGCTCTTGATCCGGCAAACAAACCACCGCTGGTAGCGG | |
| | | TGGTTTTTTTGTTTGCAAGCAGCAGATTACGCGCAGAAAAAAAGGATCTCAAGA | |
| | | AGATCCTTTGATCTTTTCTACGGGGTCTGACGCTCAGTGGAACGAAAACTCACG | |
| | | TTAAGGGATTTTGGTCATGAGATTATCAAAAAGGATCTTCACCTAGATCCTTTT | |
| | | AAATTAAAAATGAAGTTTTAAATCAATCTAAAGTATATATGAGTAAACTTGGTC | |
| | | TGACAGTTAGAAAAACTCATCGAGCATCAAATGAAACTGCAATTTATTCATATC | |
| | | AGGATTATCAATACCATATTTTTGAAAAAGCCGTTTCTGTAATGAAGGAGAAAA | |
| | | CTCACCGAGGCAGTTCCATAGGATGGCAAGATCCTGGTATCGGTCTGCGATTCC | |
| | | GACTCGTCCAACATCAATACAACCTATTAATTTCCCCTCGTCAAAAATAAGGTT | |
| | | ATCAAGTGAGAAATCACCATGAGTGACGACTGAATCCGGTGAGAATGGCAAAAG | |
| | | TTTATGCATTTCTTTCCAGACTTGTTCAACAGGCCAGCCATTACGCTCGTCATC | |
| | | AAAATCACTCGCATCAACCAAACCGTTATTCATTCGTGATTGCGCCTGAGCGAG | |
| | | ACGAAATACGCGATCGCTGTTAAAAGGACAATTACAAACAGGAATCGAATGCAA | |
| | | CCGGCGCAGGAACACTGCCAGCGCATCAACAATATTTTCACCTGAATCAGGATA | |
| | | TTCTTCTAATACCTGGAATGCTGTTTTTCCGGGGATCGCAGTGGTGAGTAACCA | |
| | | TGCATCATCAGGAGTACGGATAAAATGCTTGATGGTCGGAAGAGGCATAAATTC | |
| | | CGTCAGCCAGTTTAGTCTGACCATCTCATCTGTAACATCATTGGCAACGCTACC | |
| | | TTTGCCATGTTTCAGAAACAACTCTGGCGCATCGGGCTTCCCATACAAGCGATA | |
| | | GATTGTCGCACCTGATTGCCCGACATTATCGCGAGCCCATTTATACCCATATAA | |
| | | ATCAGCATCCATGTTGGAATTTAATCGCGGCCTCGACGTTTCCCGTTGAATATG | |
| | | GCTCATACTCTTCCTTTTTCAATATTATTGAAGCATTTATCAGGGTTATTGTCT | |
| | | CATGAGCGGATACATATTTGAATGTATTTAGAAAAATAAACAAATAGGGGTTCC | |
| | | GCGCACATTTCCCCGAAAAGTGCCACCTGACGGATCGGGAGATCTCCCGATCCC | |
| | | CTATGGTGCACTCTCAGTACAATCTGCTCTGATGCCGCATAGTTAAGCCAGTAT | |
| | | CTGCTCCCTGCTTGTGTGTTGGAGGTCGCTGAGTAGTGCGCGAGCAAAATTTAA | |
| | | GCTACAACAAGGCAAGGCTTGACCGACAATTGCATGAAGAATCTGCTTAGGGTT | |
| | | AGGCGTTTTGCGCTGCTTCGCGATGTACGGGCCAGATATACCCGTTGACATTGA | |
| | | TTATTGACTAGTTATTAATAGTAATCAATTACGGGGTCATTAGTTCATAGCCCA | |
| | | TATATGGAGTTCCGCGTTACATAACTTACGGTAAATGGCCCGCCTGGCTGACCG | |
| | | CCCAACGACCCCCGCCCATTGACGTCAATAATGACGTATGTTCCCATAGTAACG | |
| | | CCAATAGGGACTTTCCATTGACGTCAATGGGTGGAGTATTTACGGTAAACTGCC | |
| | | CACTTGGCAGTACATCAAGTGTATCATATGCCAAGTACGCCCCCTATTGACGTC | |
| | | AATGACGGTAAATGGCCCGCCTGGCATTATGCCCAGTACATGACCTTATGGGAC | |
| | | TTTCCTACTTGGCAGTACATCTACGTATTAGTCATCGCTATTACCATGGTGATG | |
| | | CGGTTTTGGCAGTACATCAATGGGCGTGGATAGCGGTTTGACTCACGGGGATTT | |
| | | CCAAGTCTCCACCCCATTGACGTCAATGGGAGTTTGTTTTGGCACCAAAATCAA | |
| | | CGGGACTTTCCAAAATGTCGTAACAACTCCGCCCCATTGACGCAAATGGGCGGT | |
| | | AGGCGTGTACGGTGGGAGGTCTATATAAGCAGCGCGTTTTGCCTGTACTGGGTC | |
| | | TCTCTGGTTAGACCAGATCTGAGCCTGGGAGCTCTCTGGCTAACTAGGGAACCC | |
| | | ACTGCTTAAGCCTCAATAAAGCTTGCCTTGAGTGCTTCAAGTAGTGTGTGCCCG | |
| | | TCTGTTGTGTGACTCTGGTAACTAGAGATCCCTCAGACCCTTTTAGTCAGTGTG | |
| | | GAAAATCTCTAGCAGTGGCGCCCGAACAGGGACTTGAAAGCGAAAGGGAAACCA | |
| | | GAGGAGCTCTCTCGACGCAGGACTCGGCTTGCTGAAGCGCGCACGGCAAGAGGC | |
| | | GAGGGGCGGCGACTGGTGAGTACGCCAAAAATTTTGACTAGCGGAGGCTAGAAG | |
| | | GAGAGAGATGGGTGCGAGAGCGTCAGTATTAAGCGGGGGAGAATTAGATCGCGA | |
| | | TGGGAAAAAATTCGGTTAAGGCCAGGGGGAAAGAAAAAATATAAATTAAAACAT | |
| | | ATAGTATGGGCAAGCAGGGAGCTAGAACGATTCGCAGTTAATCCTGGCCTGTTA | |
| | | GAAACATCAGAAGGCTGTAGACAAATACTGGGACAGCTACAACCATCCCTTCAG | |
| | | ACAGGATCAGAAGAACTTAGATCATTATATAATACAGTAGCAACCCTCTATTGT | |
| | | GTGCATCAAAGGATAGAGATAAAAGACACCAAGGAAGCTTTAGACAAGATAGAG | |
| | | GAAGAGCAAAACAAAAGTAAGACCACCGCACAGCAAGCGGCCGCTGATCTTCAG | |
| | | ACCTGGAGGAGGAGATATGAGGGACAATTGGAGAAGTGAATTATATAAATATAA | |
| | | AGTAGTAAAAATTGAACCATTAGGAGTAGCACCCACCAAGGCAAAGAGAAGAGT | |
| | | GGTGCAGAGAGAAAAAAGAGCAGTGGGAATAGGAGCTTTGTTCCTTGGGTTCTT | |
| | | GGGAGCAGCAGGAAGCACTATGGGCGCAGCGTCAATGACGCTGACGGTACAGGC | |
| | | CAGACAATTATTGTCTGGTATAGTGCAGCAGCAGAACAATTTGCTGAGGGCTAT | |
| | | TGAGGCGCAACAGCATCTGTTGCAACTCACAGTCTGGGGCATCAAGCAGCTCCA | |
| | | GGCAAGAATCCTGGCTGTGGAAAGATACCTAAAGGATCAACAGCTCCTGGGGAT | |
| | | TTGGGGTTGCTCTGGAAAACTCATTTGCACCACTGCTGTGCCTTGGAATGCTAG | |
| | | TTGGAGTAATAAATCTCTGGAACAGATTTGGAATCACACGACCTGGATGGAGTG | |
| | | GGACAGAGAAATTAACAATTACACAAGCTTAATACACTCCTTAATTGAAGAATC | |
| | | GCAAAACCAGCAAGAAAAGAATGAACAAGAATTATTGGAATTAGATAAATGGGC | |
| | | AAGTTTGTGGAATTGGTTTAACATAACAAATTGGCTGTGGTATATAAAATTATT | |
| | | CATAATGATAGTAGGAGGCTTGGTAGGTTTAAGAATAGTTTTTGCTGTACTTTC | |
| | | TATAGTGAATAGAGTTAGGCAGGGATATTCACCATTATCGTTTCAGACCCACCT | |
| | | CCCAACCCCGAGGGGACCCGACAGGCCCGAAGGAATAGAAGAAGAAGGTGGAGA | |
| | | GAGAGACAGAGACAGATCCATTCGATTAGTGAACGGATCTCGACGGTATCGGTT | |
| | | AACTTTTAAAAGAAAAGGGGGGATTGGGGGGTACAGTGCAGGGGAAAGAATAGT | |
| | | AGACATAATAGCAACAGACATACAAACTAAAGAATTACAAAAACAAATTACAAA | |
| | | ATTCAAAATTTTATCGATACTAGTATTATGCCCAGTACATGACCTTATGGGACT | |
| | | TTCCTACTTGGCAGTACATCTACAAACCCCGGGTGCAAAGATGGATAAAGTTTT | |
| | | AAACAGAGAGGAATCTTTGCAGCTAATGGACCTTCTAGGTCTTGAAAGGAGTGG | |
| | | GAATTGGCTCCGGTGCCCGTCAGTGGGCAGAGCGCACATCGCCCACAGTCCCCG | |
| | | AGAAGTTGGGGGGAGGGGTCGGCAATTGATCCGGTGCCTAGAGAAGGTGGCGCG | |
| | | GGGTAAACTGGGAAAGTGATGTCGTGTACTGGCTCCGCCTTTTTCCCGAGGGTG | |
| | | GGGGAGAACCGTATATAAGTGCAGTAGTCGCCGTGAACGTTCTTTTTCGCAACG | |
| | | GGTTTGCCGCCAGAACACAGGTAAGTGCCGTGTGTGGTTCCCGCGGGCCTGGCC | |
| | | TCTTTACGGGTTATGGCCCTTGCGTGCCTTGAATTACTTCCACTGGCTGCAGTA | |
| | | CGTGATTCTTGATCCCGAGCTTCGGGTTGGAAGTGGGTGGGAGAGTTCGAGGCC | |
| | | TTGCGCTTAAGGAGCCCCTTCGCCTCGTGCTTGAGTTGAGGCCTGGCCTGGGCG | |
| | | CTGGGGCCGCCGCGTGCGAATCTGGTGGCACCTTCGCGCCTGTCTCGCTGCTTT | |
| | | CGATAAGTCTCTAGCCATTTAAAATTTTTGATGACCTGCTGCGACGCTTTTTTT | |
| | | CTGGCAAGATAGTCTTGTAAATGCGGGCCAAGATCTGCACACTGGTATTTCGGT | |
| | | TTTTGGGGCCGCGGGCGGCGACGGGGCCCGTGCGTCCCAGCGCACATGTTCGGC | |
| | | GAGGCGGGGCCTGCGAGCGCGGCCACCGAGAATCGGACGGGGGTAGTCTCAAGC | |
| | | TGGCCGGCCTGCTCTGGTGCCTGGCCTCGCGCCGCCGTGTATCGCCCCGCCCTG | |
| | | GGCGGCAAGGCTGGCCCGGTCGGCACCAGTTGCGTGAGCGGAAAGATGGCCGCT | |
| | | TCCCGGCCCTGCTGCAGGGAGCTCAAAATGGAGGACGCGGCGCTCGGGAGAGCG | |
| | | GGCGGGTGAGTCACCCACACAAAGGAAAAGGGCCTTTCCGTCCTCAGCCGTCGC | |
| | | TTCATGTGACTCCACGGAGTACCGGGCGCCGTCCAGGCACCTCGATTAGTTCTC | |
| | | GAGCTTTTGGAGTACGTCGTCTTTAGGTTGGGGGGAGGGGTTTTATGCGATGGA | |
| | | GTTTCCCCACACTGAGTGGGTGGAGACTGAAGTTAGGCCAGCTTGGCACTTGAT | |
| | | GTAATTCTCCTTGGAATTTGCCCTTTTTGAGTTTGGATCTTGGTTCATTCTCAA | |
| | | GCCTCAGACAGTGGTTCAAAGTTTTTTTCTTCCATTTCAGGTGTCGTGACGTAG | |
| | | CGGCCCTAATACGACTCACTATAGGGCCCGCGTACGTCTAGACCTCCAGGCATC | |
| | | GATTCCACCATGGCACTCCCCGTCACCGCCCTTCTCTTGCCCCTCGCCCTGCTG | |
| | | CTGCATGCTGCCAGGCCCCAGGTCCAACTGGTGCAATCTGGCGCTGAAGTGAAA | |
| | | AAGCCCGGCGCATCAGTAAAGGTGAGTTGCAAAGCATCTGGGTACACTTTCACT | |
| | | GGCTATTATATGCATTGGGTAAGGCAAGCCCCCGGCCAAGGCCTGGAATGGATG | |
| | | GGATGGATGAATCCGAACTCCGGTAATACAGGGTACGCTCAAAAATTTCAAGGA | |
| | | CGGGTCACAATGACGCGGAACACCAGTATATCTACTGCATATATGGAGCTCAGT | |
| | | TCACTGCGCTCCGAGGATACAGCTGTATACTATTGCGCCAGGGCTCAATGGGAA | |
| | | CCACTGGACTATTGGGGTCAAGGGACACTTGTAACGGTAAGCTCAGGTGGAGGC | |
| | | GGTTCAGGCGGAGGTGGCTCTGGCGGTGGCGCTAGCTCCTATGAGCTTACTCAG | |
| | | CCACCCTCTGTATCTGTATCTCCTGGGCAGACAGCACGCATCACATGTAGTGGC | |
| | | GATGCCCTCCCGAAAAAATACACGTATTGGTATCAACAGAAATCCGGACAGGCA | |
| | | CCGGTTTTGGTGATATACTATGATACGAAAAGACCTAGTGGAATACCTGAGCGG | |
| | | TTTTCTGGAAGTTCTTCTGGTACCATGGCAACTTTGACAATCTCAGGTGCCCAG | |
| | | GTCGAGGACGAGGCCGACTATTATTGTTTGTCTACAAATCCGACAGGTCATGAA | |
| | | TCCGTGTTCGGCGGAGGGACAAAGCTTACCGTCCTTGGGCAGCCTGCGGCCGCA | |
| | | ACCACAACCCCTGCTCCAAGGCCCCCCACACCCGCTCCAACTATAGCCAGCCAA | |
| | | CCATTGAGCCTCAGACCTGAAGCTTGCAGGCCCGCAGCAGGAGGCGCCGTCCAT | |
| | | ACGCGAGGCCTGGACTTCGCGTGTGATATTTATATTTGGGCCCCTTTGGCCGGA | |
| | | ACATGTGGGGTGTTGCTTCTCTCCCTTGTGATCACTCTGTATTGTAAGCGCGGG | |
| | | AGAAAGAAGCTCCTGTACATCTTCAAGCAGCCTTTTATGCGACCTGTGCAAACC | |
| | | ACTCAGGAAGAAGATGGGTGTTCATGCCGCTTCCCCGAGGAGGAAGAAGGAGGG | |
| | | TGTGAACTGAGGGTGAAATTTTCTAGAAGCGCCGATGCTCCCGCATATCAGCAG | |
| | | GGTCAGAATCAGCTCTACAATGAATTGAATCTCGGCAGGCGAGAAGAGTACGAT | |
| | | GTTCTGGACAAGAGACGGGGCAGGGATCCCGAGATGGGGGGAAAGCCCCGGAGA | |
| | | AAAAATCCTCAGGAGGGGTTGTACAATGAGCTGCAGAAGGACAAGATGGCTGAA | |
| | | GCCTATAGCGAGATCGGAATGAAAGGCGAAAGACGCAGAGGCAAGGGGCATGAC | |
| | | GGTCTGTACCAGGGTCTCTCTACAGCCACCAAGGACACTTATGATGCGTTGCAT | |
| | | ATGCAAGCCTTGCCACCCCGCGGATCAGGAGCGACTAACTTCTCTTTGTTGAAG | |
| | | CAAGCGGGTGACGTTGAAGAGAATCCCGGTCCATCCGGGGGATCCATGCGGATC | |
| | | TCCAAGCCCCACCTCCGAAGCATCAGTATCCAGTGTTATCTGTGCCTTCTTCTG | |
| | | AATTCCCATTTTTTGACAGAGGCAGGAATTCATGTATTCATATTGGGTTGTTTC | |
| | | TCCGCGGGGCTTCCTAAAACCGAAGCTAACTGGGTTAATGTCATTTCAGATCTG | |
| | | AAGAAAATTGAAGATCTTATTCAATCAATGCATATCGACGCGACCTTGTACACG | |
| | | GAGAGTGATGTCCACCCGAGCTGCAAGGTAACGGCCATGAAATGCTTCTTGCTG | |
| | | GAATTGCAGGTAATTAGTCTTGAGAGTGGAGATGCGTCTATACACGACACCGTA | |
| | | GAGAATCTCATTATTTTGGCGAACAATTCTCTCAGCAGTAACGGCAATGTTACT | |
| | | GAATCAGGATGTAAGGAGTGTGAAGAGCTGGAAGAAAAGAACATTAAGGAATTT | |
| | | TTGCAGAGCTTCGTACATATCGTCCAAATGTTCATAAATACAAGCTAAGTCGAC | |
| | | GGCCGGCCGCGAGCATCTTACCGCCATTTATACCCATATTTGTTCTGTTTTTCT | |
| | | TGATTTGGGTATACATTTAAATGTTAATAAAACAAAATGGTGGGGCAATCATTT | |
| | | ACATTTTATAGGATATGTAATTACTAGTTCAGGTGTATTGCCACAAGACAAACA | |
| | | TCTTAAGAAACTTTCCCGTTATTTACGCTCTGTTCCTGTTAATCAACCTCTGGA | |
| | | TTACAAAATTTGTGAAAGATTGACTGATATTCTTAACTATGTTGCTCCTTTTAC | |
| | | GCTGTGTGGATATGCTGCTTTAATACCTCTGTATCATACTATTGCTTCCTGTAC | |
| | | GGCTTTCGTTTTCTCCTCCTTGTATAAATCCTGGTTGCTGTCTCTTTATCAGGA | |
| | | GTTGTGGCCCGTTGTCCGTCAACGTGGCGTGGTGTGCTCTGTGTTTGCTGACGC | |
| | | AACCCCCACTGGCTGGGGCATTGCCACCACCTGTCAACTCCTTTCTGGGACTTT | |
| | | CGCTTTCCCCCTCCCGATCGCCACGGCAGAACTCATCGCCGCCTGCCTTGCCCG | |
| | | CTGCTGGACAGGGGCTAGGTTGCTGGGCACTGATAATTCCGTGGTGTTGTCCTG | |
| | | GTACCTTTAAGACCAATGACTTACAAGGCAGCTGTAGATCTTAGCCACTTTTTA | |
| | | AAAGAAAAGGGGGGACTGGAAGGGCTAATTCACTCCCAACGAAGATAAGATCTG | |
| | | CTTTTTGCTTGTACTGGGTCTCTCTGGTTAGACCAGATCTGAGCCTGGGAGCTC | |
| | | TCTGGCTAACTAGGGAACCCACTGCTTAAGCCTCAATAAAGCTTGCCTTGAGTG | |
| | | CTTCAAGTAGTGTGTGCCCGTCTGTTGTGTGACTCTGGTAACTAGAGATCCCTC | |
| | | AGACCCTTTTAGTCAGTGTGGAAAATCTCTAGCAGTAGTAGTTCATGTCATCTT | |
| | | ATTATTCAGTATTTATAACTTGCAAAGAAATGAATATCAGAGAGTGAGAGGAAC | |
| | | TTGTTTATTGCAGCTTATAATGGTTACAAATAAAGCAATAGCATCACAAATTTC | |
| | | ACAAATAAAGCATTTTTTTCACTGCATTCTAGTTGTGGTTTGTCCAAACTCATC | |
| | | AATGTATCTTATCATGTCTGGCTCTAGCTATCCCGCCCCTAACTCCGCCCATCC | |
| | | CGCCCCTAACTCCGCCCAGTTCCGCCCATTCTCCGCCCCATGGCTGACTAATTT | |
| | | TTTTTATTTATGCAGAGGCCGAGGCCGCCTCGGCCTCTGAGCTATTCCAGAAGT | |
| | | AGTGAGGAGGCTTTTTTGGAGGCCTAGACTTTTGCAGAGACCAAATTCGTAATC | |
| | | ATGTCATAGCTGTTTCCTGTGTGAAATTGTTATCCGCTCACAATTCCACACAAC | |
| | | ATACGAGCCGGAAGCATAAAGTGTAAAGCCTGGGGTGCCTAATGAGTGAGCTAA | |
| | | CTCACATTAATTGCGTTGCGCTCACTGCCCGCTTTCCAGTCGGGAAACCTGTCG | |
| | | TGCCAGCTGCATTAATGAATCGGCCAACGCGCGGGGAGAGGCGGTTTGCGTATT | |
| | | GGGCGCTCTTCCGCTTCCTCGCTCACTGACTCGCTGCGCTCGGTCGTTCGGCTG | |
| | | CGGCGAGCGGTATCAGCTCACTCAAAGGCGGTAATACGGTTATCCACAGAATCA | |
| | | GGGGATAACGCAGGAAAGAACATGTGAGCAAAAGGCCAGCAAAAGGCCAGGAAC | |
| | | CGTAAAAAGGCCGCGTTGCTGGCGTTTTTCCATAGGCTCCGCCCCCCTGACGAG | |
| | | CATCACAAAAATCGACGCTCAAGTCAGAGGTGGCGAAACCCGACAGGACTATAA | |
| | | AGATACCAGGCGTTTCCCCCTGGAAGCTCCCTCGTGCGCTCTCCTGTTCCGACC | |
| | | CTGCCGCTTACCGGATACCTGTCCGCCTTTCTCCCTTCGGGAAGCGTGGCGCTT | |
| | | TCTCATAGCTCACGCTGTAGGTATCTCAGTTCGGTGTAGGTCGTTCGCTCCAAG | |
| | | CTGGGCTGTGTGCACGAACCCCC | |
| F6G- | VH | CAGGTTCAACTTGTTCAAAGCGGTGCAGAAGTTAAGAAACCAGGTGCCAGCGTT | 55 |
| Var6 | | AAGGTCAGCTGTAAAGCGAGTGGGTACACTTTCACCGAATATTATATGAAATGG | |
| | | GTACGACAAGCACCAGGGCAGGGACTCGAATGGATGGGGTGGATGAACCCCAAC | |
| | | ACTGGCAAGACGGGCTACGCACAGAAATTCCAAGGGCGAGTGACCATGACCAGA | |
| | | AATACGTCAATTTCAACCGCATACATGGAGCTGTCATCCTTGCGATCCGAGGAC | |
| | | ACCGCGGTATATTATTGCGCGCGCGCTCAATGGGAGCCACTTGATTATTGGGGA | |
| | | CAGGGCACGCTTGTCACTGTATCCTCT | |
| | VL | TCATATGAACTTACTCAGCCGCCTTCTGTTAGTGTAAGCCCTGGGCAGACTGCA | 63 |
| | | CGCATTACATGCAGTGGTGACGCCTTGCCAAAAAAATATACTTATTGGTACCAA | |
| | | CAAAAGAGTGGACAGGCGCCGGTCTTGGTGATATACTATGACACTAAGAGACCC | |
| | | TCTGGGATACCGGAACGCTTTTCTGGTTCAAGTTCCGGAACGATGGCGACCTTG | |
| | | ACGATCAGCGGGGCCCAAGTCGAGGATGAGGCCGACTACTACTGTTTGTCTACA | |
| | | GATCCCACAGGCCAAGAAAGCGTGTTCGGAGGTGGGACTAAACTTACGGTCTTG | |
| | | GGTCAACCA | |
| | scFv | CAGGTTCAACTTGTTCAAAGCGGTGCAGAAGTTAAGAAACCAGGTGCCAGCGTT | 71 |
| | | AAGGTCAGCTGTAAAGCGAGTGGGTACACTTTCACCGAATATTATATGAAATGG | |
| | | GTACGACAAGCACCAGGGCAGGGACTCGAATGGATGGGGTGGATGAACCCCAAC | |
| | | ACTGGCAAGACGGGCTACGCACAGAAATTCCAAGGGCGAGTGACCATGACCAGA | |
| | | AATACGTCAATTTCAACCGCATACATGGAGCTGTCATCCTTGCGATCCGAGGAC | |
| | | ACCGCGGTATATTATTGCGCGCGCGCTCAATGGGAGCCACTTGATTATTGGGGA | |
| | | CAGGGCACGCTTGTCACTGTATCCTCTGGTGGAGGCGGTTCAGGCGGAGGTGGC | |
| | | TCTGGCGGTGGCGCTAGCTCATATGAACTTACTCAGCCGCCTTCTGTTAGTGTA | |
| | | AGCCCTGGGCAGACTGCACGCATTACATGCAGTGGTGACGCCTTGCCAAAAAAA | |
| | | TATACTTATTGGTACCAACAAAAGAGTGGACAGGCGCCGGTCTTGGTGATATAC | |
| | | TATGACACTAAGAGACCCTCTGGGATACCGGAACGCTTTTCTGGTTCAAGTTCC | |
| | | GGAACGATGGCGACCTTGACGATCAGCGGGGCCCAAGTCGAGGATGAGGCCGAC | |
| | | TACTACTGTTTGTCTACAGATCCCACAGGCCAAGAAAGCGTGTTCGGAGGTGGG | |
| | | ACTAAACTTACGGTCTTGGGTCAACCA | |
| | CAR | CAGGTTCAACTTGTTCAAAGCGGTGCAGAAGTTAAGAAACCAGGTGCCAGCGTT | 101 |
| | | AAGGTCAGCTGTAAAGCGAGTGGGTACACTTTCACCGAATATTATATGAAATGG | |
| | | GTACGACAAGCACCAGGGCAGGGACTCGAATGGATGGGGTGGATGAACCCCAAC | |
| | | ACTGGCAAGACGGGCTACGCACAGAAATTCCAAGGGCGAGTGACCATGACCAGA | |
| | | AATACGTCAATTTCAACCGCATACATGGAGCTGTCATCCTTGCGATCCGAGGAC | |
| | | ACCGCGGTATATTATTGCGCGCGCGCTCAATGGGAGCCACTTGATTATTGGGGA | |
| | | CAGGGCACGCTTGTCACTGTATCCTCTGGTGGAGGCGGTTCAGGCGGAGGTGGC | |
| | | TCTGGCGGTGGCGCTAGCTCATATGAACTTACTCAGCCGCCTTCTGTTAGTGTA | |
| | | AGCCCTGGGCAGACTGCACGCATTACATGCAGTGGTGACGCCTTGCCAAAAAAA | |
| | | TATACTTATTGGTACCAACAAAAGAGTGGACAGGCGCCGGTCTTGGTGATATAC | |
| | | TATGACACTAAGAGACCCTCTGGGATACCGGAACGCTTTTCTGGTTCAAGTTCC | |
| | | GGAACGATGGCGACCTTGACGATCAGCGGGGCCCAAGTCGAGGATGAGGCCGAC | |
| | | TACTACTGTTTGTCTACAGATCCCACAGGCCAAGAAAGCGTGTTCGGAGGTGGG | |
| | | ACTAAACTTACGGTCTTGGGTCAACCAGCGGCCGCAACCACAACCCCTGCTCCA | |
| | | AGGCCCCCCACACCCGCTCCAACTATAGCCAGCCAACCATTGAGCCTCAGACCT | |
| | | GAAGCTTGCAGGCCCGCAGCAGGAGGCGCCGTCCATACGCGAGGCCTGGACTTC | |
| | | GCGTGTGATATTTATATTTGGGCCCCTTTGGCCGGAACATGTGGGGTGTTGCTT | |
| | | CTCTCCCTTGTGATCACTCTGTATTGTAAGCGCGGGAGAAAGAAGCTCCTGTAC | |
| | | ATCTTCAAGCAGCCTTTTATGCGACCTGTGCAAACCACTCAGGAAGAAGATGGG | |
| | | TGTTCATGCCGCTTCCCCGAGGAGGAAGAAGGAGGGTGTGAACTGAGGGTGAAA | |
| | | TTTTCTAGAAGCGCCGATGCTCCCGCATATCAGCAGGGTCAGAATCAGCTCTAC | |
| | | AATGAATTGAATCTCGGCAGGCGAGAAGAGTACGATGTTCTGGACAAGAGACGG | |
| | | GGCAGGGATCCCGAGATGGGGGGAAAGCCCCGGAGAAAAAATCCTCAGGAGGGG | |
| | | TTGTACAATGAGCTGCAGAAGGACAAGATGGCTGAAGCCTATAGCGAGATCGGA | |
| | | ATGAAAGGCGAAAGACGCAGAGGCAAGGGGCATGACGGTCTGTACCAGGGTCTC | |
| | | TCTACAGCCACCAAGGACACTTATGATGCGTTGCATATGCAAGCCTTGCCACCC | |
| | | CGC | |
| | Lentivira | cgttcagcccgaccgctgcgccttatccggtaactatcgtcttgagtccaacCC | 79 |
| | 1 vector | GGTAAGACACGACTTATCGCCACTGGCAGCAGCCACTGGTAACAGGATTAGCAG | |
| | | AGCGAGGTATGTAGGCGGTGCTACAGAGTTCTTGAAGTGGTGGCCTAACTACGG | |
| | | CTACACTAGAAGAACAGTATTTGGTATCTGCGCTCTGCTGAAGCCAGTTACCTT | |
| | | CGGAAAAAGAGTTGGTAGCTCTTGATCCGGCAAACAAACCACCGCTGGTAGCGG | |
| | | TGGTTTTTTTGTTTGCAAGCAGCAGATTACGCGCAGAAAAAAAGGATCTCAAGA | |
| | | AGATCCTTTGATCTTTTCTACGGGGTCTGACGCTCAGTGGAACGAAAACTCACG | |
| | | TTAAGGGATTTTGGTCATGAGATTATCAAAAAGGATCTTCACCTAGATCCTTTT | |
| | | AAATTAAAAATGAAGTTTTAAATCAATCTAAAGTATATATGAGTAAACTTGGTC | |
| | | TGACAGTTAGAAAAACTCATCGAGCATCAAATGAAACTGCAATTTATTCATATC | |
| | | AGGATTATCAATACCATATTTTTGAAAAAGCCGTTTCTGTAATGAAGGAGAAAA | |
| | | CTCACCGAGGCAGTTCCATAGGATGGCAAGATCCTGGTATCGGTCTGCGATTCC | |
| | | GACTCGTCCAACATCAATACAACCTATTAATTTCCCCTCGTCAAAAATAAGGTT | |
| | | ATCAAGTGAGAAATCACCATGAGTGACGACTGAATCCGGTGAGAATGGCAAAAG | |
| | | TTTATGCATTTCTTTCCAGACTTGTTCAACAGGCCAGCCATTACGCTCGTCATC | |
| | | AAAATCACTCGCATCAACCAAACCGTTATTCATTCGTGATTGCGCCTGAGCGAG | |
| | | ACGAAATACGCGATCGCTGTTAAAAGGACAATTACAAACAGGAATCGAATGCAA | |
| | | CCGGCGCAGGAACACTGCCAGCGCATCAACAATATTTTCACCTGAATCAGGATA | |
| | | TTCTTCTAATACCTGGAATGCTGTTTTTCCGGGGATCGCAGTGGTGAGTAACCA | |
| | | TGCATCATCAGGAGTACGGATAAAATGCTTGATGGTCGGAAGAGGCATAAATTC | |
| | | CGTCAGCCAGTTTAGTCTGACCATCTCATCTGTAACATCATTGGCAACGCTACC | |
| | | TTTGCCATGTTTCAGAAACAACTCTGGCGCATCGGGCTTCCCATACAAGCGATA | |
| | | GATTGTCGCACCTGATTGCCCGACATTATCGCGAGCCCATTTATACCCATATAA | |
| | | ATCAGCATCCATGTTGGAATTTAATCGCGGCCTCGACGTTTCCCGTTGAATATG | |
| | | GCTCATACTCTTCCTTTTTCAATATTATTGAAGCATTTATCAGGGTTATTGTCT | |
| | | CATGAGCGGATACATATTTGAATGTATTTAGAAAAATAAACAAATAGGGGTTCC | |
| | | GCGCACATTTCCCCGAAAAGTGCCACCTGACGGATCGGGAGATCTCCCGATCCC | |
| | | CTATGGTGCACTCTCAGTACAATCTGCTCTGATGCCGCATAGTTAAGCCAGTAT | |
| | | CTGCTCCCTGCTTGTGTGTTGGAGGTCGCTGAGTAGTGCGCGAGCAAAATTTAA | |
| | | GCTACAACAAGGCAAGGCTTGACCGACAATTGCATGAAGAATCTGCTTAGGGTT | |
| | | AGGCGTTTTGCGCTGCTTCGCGATGTACGGGCCAGATATACCCGTTGACATTGA | |
| | | TTATTGACTAGTTATTAATAGTAATCAATTACGGGGTCATTAGTTCATAGCCCA | |
| | | TATATGGAGTTCCGCGTTACATAACTTACGGTAAATGGCCCGCCTGGCTGACCG | |
| | | CCCAACGACCCCCGCCCATTGACGTCAATAATGACGTATGTTCCCATAGTAACG | |
| | | CCAATAGGGACTTTCCATTGACGTCAATGGGTGGAGTATTTACGGTAAACTGCC | |
| | | CACTTGGCAGTACATCAAGTGTATCATATGCCAAGTACGCCCCCTATTGACGTC | |
| | | AATGACGGTAAATGGCCCGCCTGGCATTATGCCCAGTACATGACCTTATGGGAC | |
| | | TTTCCTACTTGGCAGTACATCTACGTATTAGTCATCGCTATTACCATGGTGATG | |
| | | CGGTTTTGGCAGTACATCAATGGGCGTGGATAGCGGTTTGACTCACGGGGATTT | |
| | | CCAAGTCTCCACCCCATTGACGTCAATGGGAGTTTGTTTTGGCACCAAAATCAA | |
| | | CGGGACTTTCCAAAATGTCGTAACAACTCCGCCCCATTGACGCAAATGGGCGGT | |
| | | AGGCGTGTACGGTGGGAGGTCTATATAAGCAGCGCGTTTTGCCTGTACTGGGTC | |
| | | TCTCTGGTTAGACCAGATCTGAGCCTGGGAGCTCTCTGGCTAACTAGGGAACCC | |
| | | ACTGCTTAAGCCTCAATAAAGCTTGCCTTGAGTGCTTCAAGTAGTGTGTGCCCG | |
| | | TCTGTTGTGTGACTCTGGTAACTAGAGATCCCTCAGACCCTTTTAGTCAGTGTG | |
| | | GAAAATCTCTAGCAGTGGCGCCCGAACAGGGACTTGAAAGCGAAAGGGAAACCA | |
| | | GAGGAGCTCTCTCGACGCAGGACTCGGCTTGCTGAAGCGCGCACGGCAAGAGGC | |
| | | GAGGGGCGGCGACTGGTGAGTACGCCAAAAATTTTGACTAGCGGAGGCTAGAAG | |
| | | GAGAGAGATGGGTGCGAGAGCGTCAGTATTAAGCGGGGGAGAATTAGATCGCGA | |
| | | TGGGAAAAAATTCGGTTAAGGCCAGGGGGAAAGAAAAAATATAAATTAAAACAT | |
| | | ATAGTATGGGCAAGCAGGGAGCTAGAACGATTCGCAGTTAATCCTGGCCTGTTA | |
| | | GAAACATCAGAAGGCTGTAGACAAATACTGGGACAGCTACAACCATCCCTTCAG | |
| | | ACAGGATCAGAAGAACTTAGATCATTATATAATACAGTAGCAACCCTCTATTGT | |
| | | GTGCATCAAAGGATAGAGATAAAAGACACCAAGGAAGCTTTAGACAAGATAGAG | |
| | | GAAGAGCAAAACAAAAGTAAGACCACCGCACAGCAAGCGGCCGCTGATCTTCAG | |
| | | ACCTGGAGGAGGAGATATGAGGGACAATTGGAGAAGTGAATTATATAAATATAA | |
| | | AGTAGTAAAAATTGAACCATTAGGAGTAGCACCCACCAAGGCAAAGAGAAGAGT | |
| | | GGTGCAGAGAGAAAAAAGAGCAGTGGGAATAGGAGCTTTGTTCCTTGGGTTCTT | |
| | | GGGAGCAGCAGGAAGCACTATGGGCGCAGCGTCAATGACGCTGACGGTACAGGC | |
| | | CAGACAATTATTGTCTGGTATAGTGCAGCAGCAGAACAATTTGCTGAGGGCTAT | |
| | | TGAGGCGCAACAGCATCTGTTGCAACTCACAGTCTGGGGCATCAAGCAGCTCCA | |
| | | GGCAAGAATCCTGGCTGTGGAAAGATACCTAAAGGATCAACAGCTCCTGGGGAT | |
| | | TTGGGGTTGCTCTGGAAAACTCATTTGCACCACTGCTGTGCCTTGGAATGCTAG | |
| | | TTGGAGTAATAAATCTCTGGAACAGATTTGGAATCACACGACCTGGATGGAGTG | |
| | | GGACAGAGAAATTAACAATTACACAAGCTTAATACACTCCTTAATTGAAGAATC | |
| | | GCAAAACCAGCAAGAAAAGAATGAACAAGAATTATTGGAATTAGATAAATGGGC | |
| | | AAGTTTGTGGAATTGGTTTAACATAACAAATTGGCTGTGGTATATAAAATTATT | |
| | | CATAATGATAGTAGGAGGCTTGGTAGGTTTAAGAATAGTTTTTGCTGTACTTTC | |
| | | TATAGTGAATAGAGTTAGGCAGGGATATTCACCATTATCGTTTCAGACCCACCT | |
| | | CCCAACCCCGAGGGGACCCGACAGGCCCGAAGGAATAGAAGAAGAAGGTGGAGA | |
| | | GAGAGACAGAGACAGATCCATTCGATTAGTGAACGGATCTCGACGGTATCGGTT | |
| | | AACTTTTAAAAGAAAAGGGGGGATTGGGGGGTACAGTGCAGGGGAAAGAATAGT | |
| | | AGACATAATAGCAACAGACATACAAACTAAAGAATTACAAAAACAAATTACAAA | |
| | | ATTCAAAATTTTATCGATACTAGTATTATGCCCAGTACATGACCTTATGGGACT | |
| | | TTCCTACTTGGCAGTACATCTACAAACCCCGGGTGCAAAGATGGATAAAGTTTT | |
| | | AAACAGAGAGGAATCTTTGCAGCTAATGGACCTTCTAGGTCTTGAAAGGAGTGG | |
| | | GAATTGGCTCCGGTGCCCGTCAGTGGGCAGAGCGCACATCGCCCACAGTCCCCG | |
| | | AGAAGTTGGGGGGAGGGGTCGGCAATTGATCCGGTGCCTAGAGAAGGTGGCGCG | |
| | | GGGTAAACTGGGAAAGTGATGTCGTGTACTGGCTCCGCCTTTTTCCCGAGGGTG | |
| | | GGGGAGAACCGTATATAAGTGCAGTAGTCGCCGTGAACGTTCTTTTTCGCAACG | |
| | | GGTTTGCCGCCAGAACACAGGTAAGTGCCGTGTGTGGTTCCCGCGGGCCTGGCC | |
| | | TCTTTACGGGTTATGGCCCTTGCGTGCCTTGAATTACTTCCACTGGCTGCAGTA | |
| | | CGTGATTCTTGATCCCGAGCTTCGGGTTGGAAGTGGGTGGGAGAGTTCGAGGCC | |
| | | TTGCGCTTAAGGAGCCCCTTCGCCTCGTGCTTGAGTTGAGGCCTGGCCTGGGCG | |
| | | CTGGGGCCGCCGCGTGCGAATCTGGTGGCACCTTCGCGCCTGTCTCGCTGCTTT | |
| | | CGATAAGTCTCTAGCCATTTAAAATTTTTGATGACCTGCTGCGACGCTTTTTTT | |
| | | CTGGCAAGATAGTCTTGTAAATGCGGGCCAAGATCTGCACACTGGTATTTCGGT | |
| | | TTTTGGGGCCGCGGGCGGCGACGGGGCCCGTGCGTCCCAGCGCACATGTTCGGC | |
| | | GAGGCGGGGCCTGCGAGCGCGGCCACCGAGAATCGGACGGGGGTAGTCTCAAGC | |
| | | TGGCCGGCCTGCTCTGGTGCCTGGCCTCGCGCCGCCGTGTATCGCCCCGCCCTG | |
| | | GGCGGCAAGGCTGGCCCGGTCGGCACCAGTTGCGTGAGCGGAAAGATGGCCGCT | |
| | | TCCCGGCCCTGCTGCAGGGAGCTCAAAATGGAGGACGCGGCGCTCGGGAGAGCG | |
| | | GGCGGGTGAGTCACCCACACAAAGGAAAAGGGCCTTTCCGTCCTCAGCCGTCGC | |
| | | TTCATGTGACTCCACGGAGTACCGGGCGCCGTCCAGGCACCTCGATTAGTTCTC | |
| | | GAGCTTTTGGAGTACGTCGTCTTTAGGTTGGGGGGAGGGGTTTTATGCGATGGA | |
| | | GTTTCCCCACACTGAGTGGGTGGAGACTGAAGTTAGGCCAGCTTGGCACTTGAT | |
| | | GTAATTCTCCTTGGAATTTGCCCTTTTTGAGTTTGGATCTTGGTTCATTCTCAA | |
| | | GCCTCAGACAGTGGTTCAAAGTTTTTTTCTTCCATTTCAGGTGTCGTGACGTAG | |
| | | CGGCCCTAATACGACTCACTATAGGGCCCGCGTACGTCTAGACCTCCAGGCATC | |
| | | GATTCCACCATGGCACTCCCCGTCACCGCCCTTCTCTTGCCCCTCGCCCTGCTG | |
| | | CTGCATGCTGCCAGGCCCCAGGTTCAACTTGTTCAAAGCGGTGCAGAAGTTAAG | |
| | | AAACCAGGTGCCAGCGTTAAGGTCAGCTGTAAAGCGAGTGGGTACACTTTCACC | |
| | | GAATATTATATGAAATGGGTACGACAAGCACCAGGGCAGGGACTCGAATGGATG | |
| | | GGGTGGATGAACCCCAACACTGGCAAGACGGGCTACGCACAGAAATTCCAAGGG | |
| | | CGAGTGACCATGACCAGAAATACGTCAATTTCAACCGCATACATGGAGCTGTCA | |
| | | TCCTTGCGATCCGAGGACACCGCGGTATATTATTGCGCGCGCGCTCAATGGGAG | |
| | | CCACTTGATTATTGGGGACAGGGCACGCTTGTCACTGTATCCTCTGGTGGAGGC | |
| | | GGTTCAGGCGGAGGTGGCTCTGGCGGTGGCGCTAGCTCATATGAACTTACTCAG | |
| | | CCGCCTTCTGTTAGTGTAAGCCCTGGGCAGACTGCACGCATTACATGCAGTGGT | |
| | | GACGCCTTGCCAAAAAAATATACTTATTGGTACCAACAAAAGAGTGGACAGGCG | |
| | | CCGGTCTTGGTGATATACTATGACACTAAGAGACCCTCTGGGATACCGGAACGC | |
| | | TTTTCTGGTTCAAGTTCCGGAACGATGGCGACCTTGACGATCAGCGGGGCCCAA | |
| | | GTCGAGGATGAGGCCGACTACTACTGTTTGTCTACAGATCCCACAGGCCAAGAA | |
| | | AGCGTGTTCGGAGGTGGGACTAAACTTACGGTCTTGGGTCAACCAGCGGCCGCA | |
| | | ACCACAACCCCTGCTCCAAGGCCCCCCACACCCGCTCCAACTATAGCCAGCCAA | |
| | | CCATTGAGCCTCAGACCTGAAGCTTGCAGGCCCGCAGCAGGAGGCGCCGTCCAT | |
| | | ACGCGAGGCCTGGACTTCGCGTGTGATATTTATATTTGGGCCCCTTTGGCCGGA | |
| | | ACATGTGGGGTGTTGCTTCTCTCCCTTGTGATCACTCTGTATTGTAAGCGCGGG | |
| | | AGAAAGAAGCTCCTGTACATCTTCAAGCAGCCTTTTATGCGACCTGTGCAAACC | |
| | | ACTCAGGAAGAAGATGGGTGTTCATGCCGCTTCCCCGAGGAGGAAGAAGGAGGG | |
| | | TGTGAACTGAGGGTGAAATTTTCTAGAAGCGCCGATGCTCCCGCATATCAGCAG | |
| | | GGTCAGAATCAGCTCTACAATGAATTGAATCTCGGCAGGCGAGAAGAGTACGAT | |
| | | GTTCTGGACAAGAGACGGGGCAGGGATCCCGAGATGGGGGGAAAGCCCCGGAGA | |
| | | AAAAATCCTCAGGAGGGGTTGTACAATGAGCTGCAGAAGGACAAGATGGCTGAA | |
| | | GCCTATAGCGAGATCGGAATGAAAGGCGAAAGACGCAGAGGCAAGGGGCATGAC | |
| | | GGTCTGTACCAGGGTCTCTCTACAGCCACCAAGGACACTTATGATGCGTTGCAT | |
| | | ATGCAAGCCTTGCCACCCCGCGGATCAGGAGCGACTAACTTCTCTTTGTTGAAG | |
| | | CAAGCGGGTGACGTTGAAGAGAATCCCGGTCCATCCGGGGGATCCATGCGGATC | |
| | | TCCAAGCCCCACCTCCGAAGCATCAGTATCCAGTGTTATCTGTGCCTTCTTCTG | |
| | | AATTCCCATTTTTTGACAGAGGCAGGAATTCATGTATTCATATTGGGTTGTTTC | |
| | | TCCGCGGGGCTTCCTAAAACCGAAGCTAACTGGGTTAATGTCATTTCAGATCTG | |
| | | AAGAAAATTGAAGATCTTATTCAATCAATGCATATCGACGCGACCTTGTACACG | |
| | | GAGAGTGATGTCCACCCGAGCTGCAAGGTAACGGCCATGAAATGCTTCTTGCTG | |
| | | GAATTGCAGGTAATTAGTCTTGAGAGTGGAGATGCGTCTATACACGACACCGTA | |
| | | GAGAATCTCATTATTTTGGCGAACAATTCTCTCAGCAGTAACGGCAATGTTACT | |
| | | GAATCAGGATGTAAGGAGTGTGAAGAGCTGGAAGAAAAGAACATTAAGGAATTT | |
| | | TTGCAGAGCTTCGTACATATCGTCCAAATGTTCATAAATACAAGCTAAGTCGAC | |
| | | GGCCGGCCGCGAGCATCTTACCGCCATTTATACCCATATTTGTTCTGTTTTTCT | |
| | | TGATTTGGGTATACATTTAAATGTTAATAAAACAAAATGGTGGGGCAATCATTT | |
| | | ACATTTTATAGGATATGTAATTACTAGTTCAGGTGTATTGCCACAAGACAAACA | |
| | | TCTTAAGAAACTTTCCCGTTATTTACGCTCTGTTCCTGTTAATCAACCTCTGGA | |
| | | TTACAAAATTTGTGAAAGATTGACTGATATTCTTAACTATGTTGCTCCTTTTAC | |
| | | GCTGTGTGGATATGCTGCTTTAATACCTCTGTATCATACTATTGCTTCCTGTAC | |
| | | GGCTTTCGTTTTCTCCTCCTTGTATAAATCCTGGTTGCTGTCTCTTTATCAGGA | |
| | | GTTGTGGCCCGTTGTCCGTCAACGTGGCGTGGTGTGCTCTGTGTTTGCTGACGC | |
| | | AACCCCCACTGGCTGGGGCATTGCCACCACCTGTCAACTCCTTTCTGGGACTTT | |
| | | CGCTTTCCCCCTCCCGATCGCCACGGCAGAACTCATCGCCGCCTGCCTTGCCCG | |
| | | CTGCTGGACAGGGGCTAGGTTGCTGGGCACTGATAATTCCGTGGTGTTGTCCTG | |
| | | GTACCTTTAAGACCAATGACTTACAAGGCAGCTGTAGATCTTAGCCACTTTTTA | |
| | | AAAGAAAAGGGGGGACTGGAAGGGCTAATTCACTCCCAACGAAGATAAGATCTG | |
| | | CTTTTTGCTTGTACTGGGTCTCTCTGGTTAGACCAGATCTGAGCCTGGGAGCTC | |
| | | TCTGGCTAACTAGGGAACCCACTGCTTAAGCCTCAATAAAGCTTGCCTTGAGTG | |
| | | CTTCAAGTAGTGTGTGCCCGTCTGTTGTGTGACTCTGGTAACTAGAGATCCCTC | |
| | | AGACCCTTTTAGTCAGTGTGGAAAATCTCTAGCAGTAGTAGTTCATGTCATCTT | |
| | | ATTATTCAGTATTTATAACTTGCAAAGAAATGAATATCAGAGAGTGAGAGGAAC | |
| | | TTGTTTATTGCAGCTTATAATGGTTACAAATAAAGCAATAGCATCACAAATTTC | |
| | | ACAAATAAAGCATTTTTTTCACTGCATTCTAGTTGTGGTTTGTCCAAACTCATC | |
| | | AATGTATCTTATCATGTCTGGCTCTAGCTATCCCGCCCCTAACTCCGCCCATCC | |
| | | CGCCCCTAACTCCGCCCAGTTCCGCCCATTCTCCGCCCCATGGCTGACTAATTT | |
| | | TTTTTATTTATGCAGAGGCCGAGGCCGCCTCGGCCTCTGAGCTATTCCAGAAGT | |
| | | AGTGAGGAGGCTTTTTTGGAGGCCTAGACTTTTGCAGAGACCAAATTCGTAATC | |
| | | ATGTCATAGCTGTTTCCTGTGTGAAATTGTTATCCGCTCACAATTCCACACAAC | |
| | | ATACGAGCCGGAAGCATAAAGTGTAAAGCCTGGGGTGCCTAATGAGTGAGCTAA | |
| | | CTCACATTAATTGCGTTGCGCTCACTGCCCGCTTTCCAGTCGGGAAACCTGTCG | |
| | | TGCCAGCTGCATTAATGAATCGGCCAACGCGCGGGGAGAGGCGGTTTGCGTATT | |
| | | GGGCGCTCTTCCGCTTCCTCGCTCACTGACTCGCTGCGCTCGGTCGTTCGGCTG | |
| | | CGGCGAGCGGTATCAGCTCACTCAAAGGCGGTAATACGGTTATCCACAGAATCA | |
| | | GGGGATAACGCAGGAAAGAACATGTGAGCAAAAGGCCAGCAAAAGGCCAGGAAC | |
| | | CGTAAAAAGGCCGCGTTGCTGGCGTTTTTCCATAGGCTCCGCCCCCCTGACGAG | |
| | | CATCACAAAAATCGACGCTCAAGTCAGAGGTGGCGAAACCCGACAGGACTATAA | |
| | | AGATACCAGGCGTTTCCCCCTGGAAGCTCCCTCGTGCGCTCTCCTGTTCCGACC | |
| | | CTGCCGCTTACCGGATACCTGTCCGCCTTTCTCCCTTCGGGAAGCGTGGCGCTT | |
| | | TCTCATAGCTCACGCTGTAGGTATCTCAGTTCGGTGTAGGTCGTTCGCTCCAAG | |
| | | CTGGGCTGTGTGCACGAACCCCC | |
| F1G | VH | CAAGTTCAACTGGTTCAGTCTGGCGCCGAAGTGAAGAAACCTGGCGCCTCTGTG | 56 |
| | | AAGGTGTCCTGCAAGGCCAGCGGCTACACCTTTACCAGCTACGGCATCAGCTGG | |
| | | GTCCGACAGGCTCCTGGACAAGGCTTGGAATGGATGGGCTGGATCAGCGCCTAC | |
| | | AACGGCAACACCTACTACGCCGAGGATCTGCAGGGCAGACTGACCATGACCACC | |
| | | GATACCAGCACCAGCACCGCCTACATGGAACTGAGAAGCCTGAGATCCGACGAC | |
| | | ACCGCCGTGTACTACTGTGCCAGAGAGGGCAGCTACTACGACAGCTCCGGCTAC | |
| | | TACCTGCCTTATTGGGGCCAGGGCACACTGGTCACAGTCTCGAG | |
| | VL | CAATCTGCTCTGACACAGCCCAGAAGCGTGTCCGGATCTCCTGGCCAGAGCGTG | 64 |
| | | ACCATCAGCTGTACAGGCACCAGCTCTGATGTGGGCGCCTACAACTACGTGTCC | |
| | | TGGTATCAGCAGCACCCCGGCAAGGCCCCTAAGCTGATGATCTACGATGTGACC | |
| | | GAGAGGCCTAGCGGCGTGCCCGATAGATTTTCTGGCAGCAAGAGCGGCAACACC | |
| | | GCCAGCCTGACAATTAGCGGACTGCAGGCCGAGGACGAGGCCGATTACTACTGT | |
| | | AGCAGCTACACCAGCAGCTCTATCGCCTTTGGCGGAGGCACCAAGCTGACAGTT | |
| | | CTGGGACAACCT | |
| | scFv | CAAGTTCAACTGGTTCAGTCTGGCGCCGAAGTGAAGAAACCTGGCGCCTCTGTG | 72 |
| | | AAGGTGTCCTGCAAGGCCAGCGGCTACACCTTTACCAGCTACGGCATCAGCTGG | |
| | | GTCCGACAGGCTCCTGGACAAGGCTTGGAATGGATGGGCTGGATCAGCGCCTAC | |
| | | AACGGCAACACCTACTACGCCGAGGATCTGCAGGGCAGACTGACCATGACCACC | |
| | | GATACCAGCACCAGCACCGCCTACATGGAACTGAGAAGCCTGAGATCCGACGAC | |
| | | ACCGCCGTGTACTACTGTGCCAGAGAGGGCAGCTACTACGACAGCTCCGGCTAC | |
| | | TACCTGCCTTATTGGGGCCAGGGCACACTGGTCACAGTCTCGAGTGGTGGAGGC | |
| | | GGTTCAGGCGGAGGTGGCTCTGGCGGTGGCGCTAGCCAATCTGCTCTGACACAG | |
| | | CCCAGAAGCGTGTCCGGATCTCCTGGCCAGAGCGTGACCATCAGCTGTACAGGC | |
| | | ACCAGCTCTGATGTGGGCGCCTACAACTACGTGTCCTGGTATCAGCAGCACCCC | |
| | | GGCAAGGCCCCTAAGCTGATGATCTACGATGTGACCGAGAGGCCTAGCGGCGTG | |
| | | CCCGATAGATTTTCTGGCAGCAAGAGCGGCAACACCGCCAGCCTGACAATTAGC | |
| | | GGACTGCAGGCCGAGGACGAGGCCGATTACTACTGTAGCAGCTACACCAGCAGC | |
| | | TCTATCGCCTTTGGCGGAGGCACCAAGCTGACAGTTCTGGGACAACCT | |
| | CAR | CAAGTTCAACTGGTTCAGTCTGGCGCCGAAGTGAAGAAACCTGGCGCCTCTGTG | 102 |
| | | AAGGTGTCCTGCAAGGCCAGCGGCTACACCTTTACCAGCTACGGCATCAGCTGG | |
| | | GTCCGACAGGCTCCTGGACAAGGCTTGGAATGGATGGGCTGGATCAGCGCCTAC | |
| | | AACGGCAACACCTACTACGCCGAGGATCTGCAGGGCAGACTGACCATGACCACC | |
| | | GATACCAGCACCAGCACCGCCTACATGGAACTGAGAAGCCTGAGATCCGACGAC | |
| | | ACCGCCGTGTACTACTGTGCCAGAGAGGGCAGCTACTACGACAGCTCCGGCTAC | |
| | | TACCTGCCTTATTGGGGCCAGGGCACACTGGTCACAGTCTCGAGTGGTGGAGGC | |
| | | GGTTCAGGCGGAGGTGGCTCTGGCGGTGGCGCTAGCCAATCTGCTCTGACACAG | |
| | | CCCAGAAGCGTGTCCGGATCTCCTGGCCAGAGCGTGACCATCAGCTGTACAGGC | |
| | | ACCAGCTCTGATGTGGGCGCCTACAACTACGTGTCCTGGTATCAGCAGCACCCC | |
| | | GGCAAGGCCCCTAAGCTGATGATCTACGATGTGACCGAGAGGCCTAGCGGCGTG | |
| | | CCCGATAGATTTTCTGGCAGCAAGAGCGGCAACACCGCCAGCCTGACAATTAGC | |
| | | GGACTGCAGGCCGAGGACGAGGCCGATTACTACTGTAGCAGCTACACCAGCAGC | |
| | | TCTATCGCCTTTGGCGGAGGCACCAAGCTGACAGTTCTGGGACAACCTGCGGCC | |
| | | GCAACCACAACCCCTGCTCCAAGGCCCCCCACACCCGCTCCAACTATAGCCAGC | |
| | | CAACCATTGAGCCTCAGACCTGAAGCTTGCAGGCCCGCAGCAGGAGGCGCCGTC | |
| | | CATACGCGAGGCCTGGACTTCGCGTGTGATATTTATATTTGGGCCCCTTTGGCC | |
| | | GGAACATGTGGGGTGTTGCTTCTCTCCCTTGTGATCACTCTGTATTGTAAGCGC | |
| | | GGGAGAAAGAAGCTCCTGTACATCTTCAAGCAGCCTTTTATGCGACCTGTGCAA | |
| | | ACCACTCAGGAAGAAGATGGGTGTTCATGCCGCTTCCCCGAGGAGGAAGAAGGA | |
| | | GGGTGTGAACTGAGGGTGAAATTTTCTAGAAGCGCCGATGCTCCCGCATATCAG | |
| | | CAGGGTCAGAATCAGCTCTACAATGAATTGAATCTCGGCAGGCGAGAAGAGTAC | |
| | | GATGTTCTGGACAAGAGACGGGGCAGGGATCCCGAGATGGGGGGAAAGCCCCGG | |
| | | AGAAAAAATCCTCAGGAGGGGTTGTACAATGAGCTGCAGAAGGACAAGATGGCT | |
| | | GAAGCCTATAGCGAGATCGGAATGAAAGGCGAAAGACGCAGAGGCAAGGGGCAT | |
| | | GACGGTCTGTACCAGGGTCTCTCTACAGCCACCAAGGACACTTATGATGCGTTG | |
| | | CATATGCAAGCCTTGCCACCCCGC | |
| | Lentivira | cgttcagcccgaccgctgcgccttatccggtaactatcgtcttgagtccaacCC | 80 |
| | 1 vector | GGTAAGACACGACTTATCGCCACTGGCAGCAGCCACTGGTAACAGGATTAGCAG | |
| | | AGCGAGGTATGTAGGCGGTGCTACAGAGTTCTTGAAGTGGTGGCCTAACTACGG | |
| | | CTACACTAGAAGAACAGTATTTGGTATCTGCGCTCTGCTGAAGCCAGTTACCTT | |
| | | CGGAAAAAGAGTTGGTAGCTCTTGATCCGGCAAACAAACCACCGCTGGTAGCGG | |
| | | TGGTTTTTTTGTTTGCAAGCAGCAGATTACGCGCAGAAAAAAAGGATCTCAAGA | |
| | | AGATCCTTTGATCTTTTCTACGGGGTCTGACGCTCAGTGGAACGAAAACTCACG | |
| | | TTAAGGGATTTTGGTCATGAGATTATCAAAAAGGATCTTCACCTAGATCCTTTT | |
| | | AAATTAAAAATGAAGTTTTAAATCAATCTAAAGTATATATGAGTAAACTTGGTC | |
| | | TGACAGTTAGAAAAACTCATCGAGCATCAAATGAAACTGCAATTTATTCATATC | |
| | | AGGATTATCAATACCATATTTTTGAAAAAGCCGTTTCTGTAATGAAGGAGAAAA | |
| | | CTCACCGAGGCAGTTCCATAGGATGGCAAGATCCTGGTATCGGTCTGCGATTCC | |
| | | GACTCGTCCAACATCAATACAACCTATTAATTTCCCCTCGTCAAAAATAAGGTT | |
| | | ATCAAGTGAGAAATCACCATGAGTGACGACTGAATCCGGTGAGAATGGCAAAAG | |
| | | TTTATGCATTTCTTTCCAGACTTGTTCAACAGGCCAGCCATTACGCTCGTCATC | |
| | | AAAATCACTCGCATCAACCAAACCGTTATTCATTCGTGATTGCGCCTGAGCGAG | |
| | | ACGAAATACGCGATCGCTGTTAAAAGGACAATTACAAACAGGAATCGAATGCAA | |
| | | CCGGCGCAGGAACACTGCCAGCGCATCAACAATATTTTCACCTGAATCAGGATA | |
| | | TTCTTCTAATACCTGGAATGCTGTTTTTCCGGGGATCGCAGTGGTGAGTAACCA | |
| | | TGCATCATCAGGAGTACGGATAAAATGCTTGATGGTCGGAAGAGGCATAAATTC | |
| | | CGTCAGCCAGTTTAGTCTGACCATCTCATCTGTAACATCATTGGCAACGCTACC | |
| | | TTTGCCATGTTTCAGAAACAACTCTGGCGCATCGGGCTTCCCATACAAGCGATA | |
| | | GATTGTCGCACCTGATTGCCCGACATTATCGCGAGCCCATTTATACCCATATAA | |
| | | ATCAGCATCCATGTTGGAATTTAATCGCGGCCTCGACGTTTCCCGTTGAATATG | |
| | | GCTCATACTCTTCCTTTTTCAATATTATTGAAGCATTTATCAGGGTTATTGTCT | |
| | | CATGAGCGGATACATATTTGAATGTATTTAGAAAAATAAACAAATAGGGGTTCC | |
| | | GCGCACATTTCCCCGAAAAGTGCCACCTGACGGATCGGGAGATCTCCCGATCCC | |
| | | CTATGGTGCACTCTCAGTACAATCTGCTCTGATGCCGCATAGTTAAGCCAGTAT | |
| | | CTGCTCCCTGCTTGTGTGTTGGAGGTCGCTGAGTAGTGCGCGAGCAAAATTTAA | |
| | | GCTACAACAAGGCAAGGCTTGACCGACAATTGCATGAAGAATCTGCTTAGGGTT | |
| | | AGGCGTTTTGCGCTGCTTCGCGATGTACGGGCCAGATATACCCGTTGACATTGA | |
| | | TTATTGACTAGTTATTAATAGTAATCAATTACGGGGTCATTAGTTCATAGCCCA | |
| | | TATATGGAGTTCCGCGTTACATAACTTACGGTAAATGGCCCGCCTGGCTGACCG | |
| | | CCCAACGACCCCCGCCCATTGACGTCAATAATGACGTATGTTCCCATAGTAACG | |
| | | CCAATAGGGACTTTCCATTGACGTCAATGGGTGGAGTATTTACGGTAAACTGCC | |
| | | CACTTGGCAGTACATCAAGTGTATCATATGCCAAGTACGCCCCCTATTGACGTC | |
| | | AATGACGGTAAATGGCCCGCCTGGCATTATGCCCAGTACATGACCTTATGGGAC | |
| | | TTTCCTACTTGGCAGTACATCTACGTATTAGTCATCGCTATTACCATGGTGATG | |
| | | CGGTTTTGGCAGTACATCAATGGGCGTGGATAGCGGTTTGACTCACGGGGATTT | |
| | | CCAAGTCTCCACCCCATTGACGTCAATGGGAGTTTGTTTTGGCACCAAAATCAA | |
| | | CGGGACTTTCCAAAATGTCGTAACAACTCCGCCCCATTGACGCAAATGGGCGGT | |
| | | AGGCGTGTACGGTGGGAGGTCTATATAAGCAGCGCGTTTTGCCTGTACTGGGTC | |
| | | TCTCTGGTTAGACCAGATCTGAGCCTGGGAGCTCTCTGGCTAACTAGGGAACCC | |
| | | ACTGCTTAAGCCTCAATAAAGCTTGCCTTGAGTGCTTCAAGTAGTGTGTGCCCG | |
| | | TCTGTTGTGTGACTCTGGTAACTAGAGATCCCTCAGACCCTTTTAGTCAGTGTG | |
| | | GAAAATCTCTAGCAGTGGCGCCCGAACAGGGACTTGAAAGCGAAAGGGAAACCA | |
| | | GAGGAGCTCTCTCGACGCAGGACTCGGCTTGCTGAAGCGCGCACGGCAAGAGGC | |
| | | GAGGGGCGGCGACTGGTGAGTACGCCAAAAATTTTGACTAGCGGAGGCTAGAAG | |
| | | GAGAGAGATGGGTGCGAGAGCGTCAGTATTAAGCGGGGGAGAATTAGATCGCGA | |
| | | TGGGAAAAAATTCGGTTAAGGCCAGGGGGAAAGAAAAAATATAAATTAAAACAT | |
| | | ATAGTATGGGCAAGCAGGGAGCTAGAACGATTCGCAGTTAATCCTGGCCTGTTA | |
| | | GAAACATCAGAAGGCTGTAGACAAATACTGGGACAGCTACAACCATCCCTTCAG | |
| | | ACAGGATCAGAAGAACTTAGATCATTATATAATACAGTAGCAACCCTCTATTGT | |
| | | GTGCATCAAAGGATAGAGATAAAAGACACCAAGGAAGCTTTAGACAAGATAGAG | |
| | | GAAGAGCAAAACAAAAGTAAGACCACCGCACAGCAAGCGGCCGCTGATCTTCAG | |
| | | ACCTGGAGGAGGAGATATGAGGGACAATTGGAGAAGTGAATTATATAAATATAA | |
| | | AGTAGTAAAAATTGAACCATTAGGAGTAGCACCCACCAAGGCAAAGAGAAGAGT | |
| | | GGTGCAGAGAGAAAAAAGAGCAGTGGGAATAGGAGCTTTGTTCCTTGGGTTCTT | |
| | | GGGAGCAGCAGGAAGCACTATGGGCGCAGCGTCAATGACGCTGACGGTACAGGC | |
| | | CAGACAATTATTGTCTGGTATAGTGCAGCAGCAGAACAATTTGCTGAGGGCTAT | |
| | | TGAGGCGCAACAGCATCTGTTGCAACTCACAGTCTGGGGCATCAAGCAGCTCCA | |
| | | GGCAAGAATCCTGGCTGTGGAAAGATACCTAAAGGATCAACAGCTCCTGGGGAT | |
| | | TTGGGGTTGCTCTGGAAAACTCATTTGCACCACTGCTGTGCCTTGGAATGCTAG | |
| | | TTGGAGTAATAAATCTCTGGAACAGATTTGGAATCACACGACCTGGATGGAGTG | |
| | | GGACAGAGAAATTAACAATTACACAAGCTTAATACACTCCTTAATTGAAGAATC | |
| | | GCAAAACCAGCAAGAAAAGAATGAACAAGAATTATTGGAATTAGATAAATGGGC | |
| | | AAGTTTGTGGAATTGGTTTAACATAACAAATTGGCTGTGGTATATAAAATTATT | |
| | | CATAATGATAGTAGGAGGCTTGGTAGGTTTAAGAATAGTTTTTGCTGTACTTTC | |
| | | TATAGTGAATAGAGTTAGGCAGGGATATTCACCATTATCGTTTCAGACCCACCT | |
| | | CCCAACCCCGAGGGGACCCGACAGGCCCGAAGGAATAGAAGAAGAAGGTGGAGA | |
| | | GAGAGACAGAGACAGATCCATTCGATTAGTGAACGGATCTCGACGGTATCGGTT | |
| | | AACTTTTAAAAGAAAAGGGGGGATTGGGGGGTACAGTGCAGGGGAAAGAATAGT | |
| | | AGACATAATAGCAACAGACATACAAACTAAAGAATTACAAAAACAAATTACAAA | |
| | | ATTCAAAATTTTATCGATACTAGTATTATGCCCAGTACATGACCTTATGGGACT | |
| | | TTCCTACTTGGCAGTACATCTACAAACCCCGGGTGCAAAGATGGATAAAGTTTT | |
| | | AAACAGAGAGGAATCTTTGCAGCTAATGGACCTTCTAGGTCTTGAAAGGAGTGG | |
| | | GAATTGGCTCCGGTGCCCGTCAGTGGGCAGAGCGCACATCGCCCACAGTCCCCG | |
| | | AGAAGTTGGGGGGAGGGGTCGGCAATTGATCCGGTGCCTAGAGAAGGTGGCGCG | |
| | | GGGTAAACTGGGAAAGTGATGTCGTGTACTGGCTCCGCCTTTTTCCCGAGGGTG | |
| | | GGGGAGAACCGTATATAAGTGCAGTAGTCGCCGTGAACGTTCTTTTTCGCAACG | |
| | | GGTTTGCCGCCAGAACACAGGTAAGTGCCGTGTGTGGTTCCCGCGGGCCTGGCC | |
| | | TCTTTACGGGTTATGGCCCTTGCGTGCCTTGAATTACTTCCACTGGCTGCAGTA | |
| | | CGTGATTCTTGATCCCGAGCTTCGGGTTGGAAGTGGGTGGGAGAGTTCGAGGCC | |
| | | TTGCGCTTAAGGAGCCCCTTCGCCTCGTGCTTGAGTTGAGGCCTGGCCTGGGCG | |
| | | CTGGGGCCGCCGCGTGCGAATCTGGTGGCACCTTCGCGCCTGTCTCGCTGCTTT | |
| | | CGATAAGTCTCTAGCCATTTAAAATTTTTGATGACCTGCTGCGACGCTTTTTTT | |
| | | CTGGCAAGATAGTCTTGTAAATGCGGGCCAAGATCTGCACACTGGTATTTCGGT | |
| | | TTTTGGGGCCGCGGGCGGCGACGGGGCCCGTGCGTCCCAGCGCACATGTTCGGC | |
| | | GAGGCGGGGCCTGCGAGCGCGGCCACCGAGAATCGGACGGGGGTAGTCTCAAGC | |
| | | TGGCCGGCCTGCTCTGGTGCCTGGCCTCGCGCCGCCGTGTATCGCCCCGCCCTG | |
| | | GGCGGCAAGGCTGGCCCGGTCGGCACCAGTTGCGTGAGCGGAAAGATGGCCGCT | |
| | | TCCCGGCCCTGCTGCAGGGAGCTCAAAATGGAGGACGCGGCGCTCGGGAGAGCG | |
| | | GGCGGGTGAGTCACCCACACAAAGGAAAAGGGCCTTTCCGTCCTCAGCCGTCGC | |
| | | GAGCTTTTGGAGTACGTCGTCTTTAGGTTGGGGGGAGGGGTTTTATGCGATGGA | |
| | | GTTTCCCCACACTGAGTGGGTGGAGACTGAAGTTAGGCCAGCTTGGCACTTGAT | |
| | | GTAATTCTCCTTGGAATTTGCCCTTTTTGAGTTTGGATCTTGGTTCATTCTCAA | |
| | | GCCTCAGACAGTGGTTCAAAGTTTTTTTCTTCCATTTCAGGTGTCGTGACGTAG | |
| | | CGGCCCTAATACGACTCACTATAGGGCCCGCGTACGTCTAGACCTCCAGGCATC | |
| | | GATTCCACCATGGCACTCCCCGTCACCGCCCTTCTCTTGCCCCTCGCCCTGCTG | |
| | | CTGCATGCTGCCAGGCCCCAAGTTCAACTGGTTCAGTCTGGCGCCGAAGTGAAG | |
| | | AAACCTGGCGCCTCTGTGAAGGTGTCCTGCAAGGCCAGCGGCTACACCTTTACC | |
| | | AGCTACGGCATCAGCTGGGTCCGACAGGCTCCTGGACAAGGCTTGGAATGGATG | |
| | | GGCTGGATCAGCGCCTACAACGGCAACACCTACTACGCCGAGGATCTGCAGGGC | |
| | | AGACTGACCATGACCACCGATACCAGCACCAGCACCGCCTACATGGAACTGAGA | |
| | | AGCCTGAGATCCGACGACACCGCCGTGTACTACTGTGCCAGAGAGGGCAGCTAC | |
| | | TACGACAGCTCCGGCTACTACCTGCCTTATTGGGGCCAGGGCACACTGGTCACA | |
| | | GTCTCGAGTGGTGGAGGCGGTTCAGGCGGAGGTGGCTCTGGCGGTGGCGCTAGC | |
| | | CAATCTGCTCTGACACAGCCCAGAAGCGTGTCCGGATCTCCTGGCCAGAGCGTG | |
| | | ACCATCAGCTGTACAGGCACCAGCTCTGATGTGGGCGCCTACAACTACGTGTCC | |
| | | TGGTATCAGCAGCACCCCGGCAAGGCCCCTAAGCTGATGATCTACGATGTGACC | |
| | | GAGAGGCCTAGCGGCGTGCCCGATAGATTTTCTGGCAGCAAGAGCGGCAACACC | |
| | | GCCAGCCTGACAATTAGCGGACTGCAGGCCGAGGACGAGGCCGATTACTACTGT | |
| | | AGCAGCTACACCAGCAGCTCTATCGCCTTTGGCGGAGGCACCAAGCTGACAGTT | |
| | | CTGGGACAACCTGCGGCCGCAACCACAACCCCTGCTCCAAGGCCCCCCACACCC | |
| | | GCTCCAACTATAGCCAGCCAACCATTGAGCCTCAGACCTGAAGCTTGCAGGCCC | |
| | | GCAGCAGGAGGCGCCGTCCATACGCGAGGCCTGGACTTCGCGTGTGATATTTAT | |
| | | ATTTGGGCCCCTTTGGCCGGAACATGTGGGGTGTTGCTTCTCTCCCTTGTGATC | |
| | | ACTCTGTATTGTAAGCGCGGGAGAAAGAAGCTCCTGTACATCTTCAAGCAGCCT | |
| | | TTTATGCGACCTGTGCAAACCACTCAGGAAGAAGATGGGTGTTCATGCCGCTTC | |
| | | CCCGAGGAGGAAGAAGGAGGGTGTGAACTGAGGGTGAAATTTTCTAGAAGCGCC | |
| | | GATGCTCCCGCATATCAGCAGGGTCAGAATCAGCTCTACAATGAATTGAATCTC | |
| | | GGCAGGCGAGAAGAGTACGATGTTCTGGACAAGAGACGGGGCAGGGATCCCGAG | |
| | | ATGGGGGGAAAGCCCCGGAGAAAAAATCCTCAGGAGGGGTTGTACAATGAGCTG | |
| | | CAGAAGGACAAGATGGCTGAAGCCTATAGCGAGATCGGAATGAAAGGCGAAAGA | |
| | | CGCAGAGGCAAGGGGCATGACGGTCTGTACCAGGGTCTCTCTACAGCCACCAAG | |
| | | GACACTTATGATGCGTTGCATATGCAAGCCTTGCCACCCCGCGGATCAGGAGCG | |
| | | ACTAACTTCTCTTTGTTGAAGCAAGCGGGTGACGTTGAAGAGAATCCCGGTCCA | |
| | | TCCGGGGGATCCATGCGGATCTCCAAGCCCCACCTCCGAAGCATCAGTATCCAG | |
| | | TGTTATCTGTGCCTTCTTCTGAATTCCCATTTTTTGACAGAGGCAGGAATTCAT | |
| | | GTATTCATATTGGGTTGTTTCTCCGCGGGGCTTCCTAAAACCGAAGCTAACTGG | |
| | | GTTAATGTCATTTCAGATCTGAAGAAAATTGAAGATCTTATTCAATCAATGCAT | |
| | | ATCGACGCGACCTTGTACACGGAGAGTGATGTCCACCCGAGCTGCAAGGTAACG | |
| | | GCCATGAAATGCTTCTTGCTGGAATTGCAGGTAATTAGTCTTGAGAGTGGAGAT | |
| | | GCGTCTATACACGACACCGTAGAGAATCTCATTATTTTGGCGAACAATTCTCTC | |
| | | AGCAGTAACGGCAATGTTACTGAATCAGGATGTAAGGAGTGTGAAGAGCTGGAA | |
| | | GAAAAGAACATTAAGGAATTTTTGCAGAGCTTCGTACATATCGTCCAAATGTTC | |
| | | ATAAATACAAGCTAAGTCGACGGCCGGCCGCGAGCATCTTACCGCCATTTATAC | |
| | | CCATATTTGTTCTGTTTTTCTTGATTTGGGTATACATTTAAATGTTAATAAAAC | |
| | | AAAATGGTGGGGCAATCATTTACATTTTATAGGATATGTAATTACTAGTTCAGG | |
| | | TGTATTGCCACAAGACAAACATCTTAAGAAACTTTCCCGTTATTTACGCTCTGT | |
| | | TCCTGTTAATCAACCTCTGGATTACAAAATTTGTGAAAGATTGACTGATATTCT | |
| | | TAACTATGTTGCTCCTTTTACGCTGTGTGGATATGCTGCTTTAATACCTCTGTA | |
| | | TCATACTATTGCTTCCTGTACGGCTTTCGTTTTCTCCTCCTTGTATAAATCCTG | |
| | | GTTGCTGTCTCTTTATCAGGAGTTGTGGCCCGTTGTCCGTCAACGTGGCGTGGT | |
| | | GTGCTCTGTGTTTGCTGACGCAACCCCCACTGGCTGGGGCATTGCCACCACCTG | |
| | | TCAACTCCTTTCTGGGACTTTCGCTTTCCCCCTCCCGATCGCCACGGCAGAACT | |
| | | CATCGCCGCCTGCCTTGCCCGCTGCTGGACAGGGGCTAGGTTGCTGGGCACTGA | |
| | | TAATTCCGTGGTGTTGTCCTGGTACCTTTAAGACCAATGACTTACAAGGCAGCT | |
| | | GTAGATCTTAGCCACTTTTTAAAAGAAAAGGGGGGACTGGAAGGGCTAATTCAC | |
| | | TCCCAACGAAGATAAGATCTGCTTTTTGCTTGTACTGGGTCTCTCTGGTTAGAC | |
| | | CAGATCTGAGCCTGGGAGCTCTCTGGCTAACTAGGGAACCCACTGCTTAAGCCT | |
| | | CAATAAAGCTTGCCTTGAGTGCTTCAAGTAGTGTGTGCCCGTCTGTTGTGTGAC | |
| | | TCTGGTAACTAGAGATCCCTCAGACCCTTTTAGTCAGTGTGGAAAATCTCTAGC | |
| | | AGTAGTAGTTCATGTCATCTTATTATTCAGTATTTATAACTTGCAAAGAAATGA | |
| | | ATATCAGAGAGTGAGAGGAACTTGTTTATTGCAGCTTATAATGGTTACAAATAA | |
| | | AGCAATAGCATCACAAATTTCACAAATAAAGCATTTTTTTCACTGCATTCTAGT | |
| | | TGTGGTTTGTCCAAACTCATCAATGTATCTTATCATGTCTGGCTCTAGCTATCC | |
| | | CGCCCCTAACTCCGCCCATCCCGCCCCTAACTCCGCCCAGTTCCGCCCATTCTC | |
| | | CGCCCCATGGCTGACTAATTTTTTTTATTTATGCAGAGGCCGAGGCCGCCTCGG | |
| | | CCTCTGAGCTATTCCAGAAGTAGTGAGGAGGCTTTTTTGGAGGCCTAGACTTTT | |
| | | GCAGAGACCAAATTCGTAATCATGTCATAGCTGTTTCCTGTGTGAAATTGTTAT | |
| | | CCGCTCACAATTCCACACAACATACGAGCCGGAAGCATAAAGTGTAAAGCCTGG | |
| | | GGTGCCTAATGAGTGAGCTAACTCACATTAATTGCGTTGCGCTCACTGCCCGCT | |
| | | TTCCAGTCGGGAAACCTGTCGTGCCAGCTGCATTAATGAATCGGCCAACGCGCG | |
| | | GGGAGAGGCGGTTTGCGTATTGGGCGCTCTTCCGCTTCCTCGCTCACTGACTCG | |
| | | CTGCGCTCGGTCGTTCGGCTGCGGCGAGCGGTATCAGCTCACTCAAAGGCGGTA | |
| | | ATACGGTTATCCACAGAATCAGGGGATAACGCAGGAAAGAACATGTGAGCAAAA | |
| | | GGCCAGCAAAAGGCCAGGAACCGTAAAAAGGCCGCGTTGCTGGCGTTTTTCCAT | |
| | | AGGCTCCGCCCCCCTGACGAGCATCACAAAAATCGACGCTCAAGTCAGAGGTGG | |
| | | CGAAACCCGACAGGACTATAAAGATACCAGGCGTTTCCCCCTGGAAGCTCCCTC | |
| | | GTGCGCTCTCCTGTTCCGACCCTGCCGCTTACCGGATACCTGTCCGCCTTTCTC | |
| | | CCTTCGGGAAGCGTGGCGCTTTCTCATAGCTCACGCTGTAGGTATCTCAGTTCG | |
| | | GTGTAGGTCGTTCGCTCCAAGCTGGGCTGTGTGCACGAACCCCC | |

In some embodiments, a single-chain antibody (e.g., an anti BCMA scFv) can be prepared via recombinant technology by linking a nucleotide sequence coding for a heavy chain variable region and a nucleotide sequence coding for a light chain variable region. Preferably, a flexible linker is incorporated between the two variable regions.

In some embodiments, an anti-BCMA antibody or antigen binding fragment thereof is produced by expressing: (i) a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 49, and/or a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 57; (ii) a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 50, and/or a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 58; (iii) a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 51, and/or a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 59; (iv) a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 52, and/or a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 60; (v) a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 53, and/or a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 61; (vi) a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 54, and/or a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 62; (vii) a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 55, and/or a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 63; or (viii) a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 56, and/or a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 64.

In some embodiments, an anti-BCMA scFv is produced by expressing a nucleic acid at least 60% (e.g., 60%, 65%, 70%, 75%, 80%, 85%, 90%, 91%, 92%, 93%, 94%, 95%, 96%, 97%, 98%, 99%, or 100%) identical to any one of SEQ ID NOs: 66-72.

In some embodiments, an anti-BCMA CAR is expressed by a cell (e.g., an iNKT cell) by delivering to the cell a nucleic acid at least 60% (e.g., 60%, 65%, 70%, 75%, 80%, 85%, 90%, 91%, 92%, 93%, 94%, 95%, 96%, 97%, 98%, 99%, or 100%) identical to any one of SEQ ID NOs: 95-102.

In some embodiments, the present disclosure also provides vectors (e.g., lentiviral vectors) for expressing the anti-BCMA CAR in a cell (e.g., an iNKT cell). In some embodiments, the lentiviral vector comprises a nucleic acid sequence at least 60% (e.g., 60%, 65%, 70%, 75%, 80%, 85%, 90%, 91%, 92%, 93%, 94%, 95%, 96%, 97%, 98%, 99%, or 100%) identical to any one of SEQ ID NOs: 73-80.

In some aspects, the present disclosure also provides methods for preparing a population of immune cells (e.g., iNKT cells) expressing the CAR (e.g., anti-BCMA CAR). An initial population of immune cells can be obtained from any source, such as peripheral blood mononuclear cells (PBMCs), bone marrow, tissues such as spleen, lymph node, thymus, or tumor tissue. A source suitable for obtaining the type of cell desired would be evident to one of skill in the art. In some embodiments, the population of immune cells (e.g., iNKT cells) is derived from PBMCs. In some embodiments a blood sample or an apheresis is taken from a generally healthy subject. In some embodiments, a blood sample or an apheresis is taken from a generally healthy subject who is at risk of developing a disease, but who has not yet developed a disease, and the cells of interest are isolated and frozen for later use. In some embodiments, the iNKT cells may be expanded, frozen, and used at a later time. In certain embodiments, samples are collected from a patient shortly after diagnosis of a particular disease as described herein but prior to any treatments. In a further embodiment, the cells are isolated from a blood sample or an apheresis from a subject prior to any number of relevant treatment modalities, including but not limited to treatment with agents such as natalizumab, efalizumab, antiviral agents, chemotherapy, radiation, immunosuppressive agents, such as cyclosporin, azathioprine, methotrexate, mycophenolate, and FK506, antibodies, or other immunoablative agents such as CAMPATH®, anti-CD3 antibodies, Cytoxan, fludarabine, cyclosporin, FK506, rapamycin, mycophenolic acid, steroids, FR901228, and irradiation. These drugs inhibit either the calcium dependent phosphatase calcineurin (cyclosporine and FK506) or inhibit the p70S6 kinase that is important for growth factor induced signaling (rapamycin) (Liu et al., Cell 66:807-815, 1991; Henderson et al., Immun. 73:316-321, 1991; Bierer et al., Curr. Opin. Immun. 5:763-773, 1993). In a further embodiment, the cells are isolated for a patient and frozen for later use in conjunction with (e.g., before, simultaneously or following) bone marrow or stem cell transplantation, T cell ablative therapy using either chemotherapy agents such as, fludarabine, external-beam radiation therapy (XRT), cyclophosphamide, or antibodies such as OKT3 or CAMPATH®. In some embodiments, a blood sample or an apheresis are taken from a healthy donor, and the iNKT cells isolated from a third-party healthy donor can be used “off-the-shelf”, e.g., as described by Li et al., Off-the-shelf third-party HSC-engineered iNKT cells for ameliorating GvHD while preserving GvL effect in the treatment of blood cancers, iScience. 2022 Sep. 16; 25 (9): 104859.

In some embodiments, an initial population of the iNKT cells are purified from PBMCs using any suitable methods known in the art (e.g., FACS or MACS). In some embodiments, the initial population of the iNKT cells are stimulated by α-galactosylceramide (α-GalCer) or any modified glycolipid thereof, e.g., as described by Zhang et al., α-GalCer and iNKT Cell-Based Cancer Immunotherapy: Realizing the Therapeutic Potentials, Front Immunol. 2019 June 6; 10:1126; Schafer et al., iNKT cell stimulation by glycolipid ligands modified from α-galactosylceramide results in differential interleukin-2 secretion profiles, J Immunol May 1, 2019, 202 (1 Supplement) 177.1) for activation and expansion. In some embodiments, the iNKT cells are transduced with any one of the lentiviral vector described herein to express an anti-BCMA CAR (e.g., any one of the anti-BCMA CAR as described herein). In some embodiments, after transduction, there are more than 1%, more than 2%, more than 5%, more than 8%, more than 10%, more than 12%, or more than 15%, more than 18%, more than 20%, more than 22%, more than 25%, more than 28%, or more than 30% iNKT cells expressing the anti-BCMA CAR. In some embodiments, after transduction, the iNKT cells are further stimulated and expanded. In some embodiments, further stimulation and expansion comprises stimulating the iNKT cells expressing anti-BCMA CAR with a feeder carrying BCMA. Feeders can be any suitable feeder cell known in the art, e.g., feeder cells, or cell-derived membrane vesicles (Ukrainskaya et al., Antigen-Specific Stimulation and Expansion of CAR-T Cells Using Membrane Vesicles as Target Cell Surrogates, Small. 2021 November; 17 (45): e2102643). In some embodiments, the feeder is a feeder cell (e.g., K562 feeder cell, major histocompatibility complex class I chain-related protein A (MICA) feeder cells, or membrane-bound IL-21 human B-lymphoblastoid cell-line 721.221 (hereinafter, 221)-based artificial feeder (221-mIL-21) cells. In some embodiments, the feeder cell is a K562 feeder cell. Expansion of NK cells using genetically engineered K562 feeder cells have been previously described, see, e.g., Phan et al., Expansion of NK Cells Using Genetically Engineered K562 Feeder Cells, Natural Killer Cells pp 167-174. In some embodiments, the K562 cells are irradiated prior co-culture with the iNKT cell expressing the anti-BCMA CAR. In some embodiments, the K562 cells engineered express BCMA on the cell surface. In some embodiments, after co-culturing with feeder cells, at least 60%, at least 65%, at least 70%, at least 75%, at least 80%, at least 85%, at least 90%, at least 92%, at least 95%, at least 98%, at least 99%, or more iNKT cells express the anti-BCMA CAR.

### IV. Therapeutic Applications

In some embodiments, the present disclosure encompasses a cell (e.g., iNKT cell) modified to express a CAR that combines an antigen recognition domains (e.g., an anti-BCMA scFv), a transmembrane domain (e.g., a CD8 transmembrane domain) and a cytoplasmic domain (e.g., an intracellular domain of CD3-zeta, CD28, OX40, 4-1BB, or any combinations thereof). Therefore, in some embodiments, the transduced immune cell (e.g., iNKT cell) elicits a CAR-mediated immune response (e.g., T-cell response), and/or innate immune response (e.g., NK cell response) in a subject. In some embodiments, the present disclosure provides the use of a CAR to redirect the specificity of a primary iNKT cell to a tumor antigen. Thus, in some embodiments, the present invention also provides a method for stimulating a iNKT cell-mediated immune response to a target cell population or tissue in a mammal comprising the step of administering to the mammal a plurality of iNKT cell that expresses a CAR (e.g., an anti-BCMA CAR), wherein the CAR comprises a binding moiety that specifically interacts with a predetermined target (e.g., BCMA), a zeta chain portion comprising for example the intracellular domain of human CD3zeta, and a costimulatory signaling region. In some embodiments, the present disclosure includes a type of cellular therapy where iNKT cells are genetically modified to express a CAR and the iNKT cell expressing the CAR (e.g., an anti-BCMA CAR) is infused to a recipient in need thereof. The infused cells are able to kill tumor cells in the recipient. Unlike antibody therapies, CAR iNKT cells are able to replicate in vivo enabling long-term persistence that can lead to sustained tumor control. In some embodiments, unlike CAR T therapy, a subject receiving iNKT cell composition does not undergo lymphodepletion prior to receiving the iNKT cells.

In some embodiments, the present disclosure provides a cell (e.g., iNKT cell) modified to express a CAR (e.g., an anti-BCMA CAR), and a cytokine (e.g., sIL-15). In some embodiments, the secretion of IL-15 by the iNKT cell expressing an anti-BCMA CAR enhances iNKT cell persistence in a subject (e.g., relative to a subject administered a CAR not expressing an armoring molecule, such as IL-15).

Without wishing to be bound by any particular theory, the anti-tumor immunity response elicited by the CAR-modified iNKT cells may be an innate or an adaptive immune response. In some embodiments, the CAR mediated immune response may be part of an adoptive immunotherapy approach in which CAR-modified iNKT cells induce an immune response specific to the antigen binding moiety in the CAR. For example, BCMA-specific CAR iNKT cells elicit an immune response specific against cells expressing BCMA. In some embodiments, BCMA-specific CAR iNKT cells described herein kill cancer cells. In some embodiments, BCMA-specific CAR iNKT cells described herein reduce tumor burden. In some embodiments, BCMA-specific CAR iNKT cells described herein recruit other immune cells to the tumor microenvironment for cancer killing. In some embodiments, the antigen binding domain in the CAR of the present disclosure targets a tumor antigen (e.g., BCMA) for the purposes of treating cancer. In some embodiments, the antigen binding moiety portion of the CAR of the invention is designed to treat a particular cancer (e.g., cancer expressing BCMA), such as multiple myeloma.

The CAR-modified iNKT cells of the present disclosure may also serve as a type of vaccine for ex vivo immunization and/or in vivo therapy in a mammal. Preferably, the mammal is a human.

In some embodiments, the iNKT cells as described herein may be utilized in the treatment and prevention of diseases (e.g., cancer) that arise in individuals who are immunocompromised, such as individuals having cancer. In particular, the iNKT cells expressing the anti-BCMA CAR of the present disclosure are used in the treatment of multiple myeloma. In certain embodiments, the iNKT cells expressing the anti-BCMA CAR of the present disclosure are used in the treatment of patients at risk for developing multiple myeloma.

In some embodiments, the iNKT cells expressing the anti-BCMA CAR of the present disclosure, or a composition comprising such cells, may be used, or may be administered to a subject in need thereof, to provide anti-tumor immunity; to treat or prevent cancer; to treat or prevent autoimmune condition; or to treat or prevent an allergic condition. In some embodiments, the cancer is a BCMA-expressing cancer. In some embodiments, the cancer is multiple myeloma, Hodgkin lymphoma, non-Hodgkin lymphoma, a leukemia, or glioblastoma. In some embodiments, the autoimmune condition is myasthenia gravis, systemic lupus erythematosus, rheumatoid arthritis, pemphigus, psoriasis, inflammatory bowel disease, celiac sprue, pernicious anemia, idiopathic thrombocytopenia purpura, sceleroderma, Graves disease, Sjögren syndrome, Goodpasture syndrome, or type 1 diabetes. In some embodiments, the allergic condition is anaphylaxis, asthma, food allergy, stinging insect allergy, drug allergy, allergic rhinitis, urticaria, angioedema, eczema, atopic dermatitis, contact dermatitis, and eosinophilic esophagitis.

In some embodiments, the iNKT cells expressing the anti-BCMA CAR of the present disclosure may be administered either alone, or as a composition (e.g., a pharmaceutical composition) in combination with diluents and/or with other components such as IL-2 or other cytokines or cell populations. Briefly, pharmaceutical compositions of the present invention may comprise a target cell population as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

Pharmaceutical compositions of the present invention may be administered in a manner appropriate to the disease to be treated (or prevented). The quantity and frequency of administration will be determined by such factors as the condition of the patient, and the type and severity of the patient's disease, although appropriate dosages may be determined by clinical trials. In some embodiments, compositions of the present disclosure are formulated for intravenous administration.

When “an immunologically effective amount”, “an anti-tumor effective amount”, “an tumor-inhibiting effective amount”, or “therapeutic amount” is indicated, the precise amount of the compositions of the present invention to be administered can be determined by a physician with consideration of individual differences in age, weight, tumor size, extent of infection or metastasis, and condition of the patient (subject). The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly.

The administration of the subject compositions may be carried out in any convenient manner, including by aerosol inhalation, injection, ingestion, transfusion, implantation or transplantation. The compositions described herein may be administered to a patient subcutaneously, intradermally, intratumorally, intranodally, intramedullary, intramuscularly, by intravenous (i.v.) injection, or intraperitoneally. In some embodiments, the immune cell (e.g., iNKT cell) compositions of the present invention are administered to a patient by intradermal or subcutaneous injection. In another embodiment, the immune cell (e.g., iNK T cell) compositions of the present invention are preferably administered by i.v. injection. The compositions of immune cells (e.g., iNKT cells) may be injected directly into a tumor, lymph node, or site of disease.

In certain embodiments of the present disclosure, cells activated and expanded using the methods described herein, or other methods known in the art where iNKT cells are expanded to therapeutic levels, are administered to a patient in conjunction with (e.g., before, simultaneously or following) any number of relevant treatment modalities, including but not limited to treatment with agents such as antiviral therapy, cidofovir and interleukin-2, Cytarabine (also known as ARA-C) or natalizumab treatment for MS patients or efalizumab treatment for psoriasis patients or other treatments for PML patients. In further embodiments, the iNKT cells of the present disclosure may be used in combination with chemotherapy, radiation, immunosuppressive agents, such as cyclosporin, azathioprine, methotrexate, mycophenolate, and FK506, antibodies, or other immunoablative agents such as CAMPATH, anti-CD3 antibodies or other antibody therapies, cytoxin, fludaribine, cyclosporin, FK506, rapamycin, mycophenolic acid, steroids, FR901228, cytokines, and irradiation. In a further embodiment, the cell compositions of the present disclosure are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. In another embodiment, the cell compositions of the present disclosure are administered following B-cell ablative therapy such as agents that react with CD20, e.g., Rituxan. For example, in some embodiments, subjects may undergo standard treatment with high dose chemotherapy followed by peripheral blood stem cell transplantation. In certain embodiments, following the transplant, subjects receive an infusion of the expanded immune cells (e.g., iNKT cell) of the present disclosure. In an additional embodiment, iNKT cells are administered before or following surgery.

The scaling of dosages for human administration can be performed according to art-accepted practices. The dose for CAMPATH, for example, will generally be in the range 1 to about 100 mg for an adult patient, usually administered daily for a period between 1 and 30 days. The preferred daily dose is 1 to 10 mg per day although in some instances larger doses of up to 40 mg per day may be used (described in U.S. Pat. No. 6,120,766). Strategies for CAR T cell dosing and scheduling have been discussed (Ertl et al., 2011, Cancer Res, 71:3175-81; Junghans, 2010, Journal of Translational Medicine, 8:55).

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

The following examples are provided for illustrative purposes and are not intended to limit the scope of the invention.

### Example 1: Development and Evaluation of Anti-BCMA CAR iNKT Cells

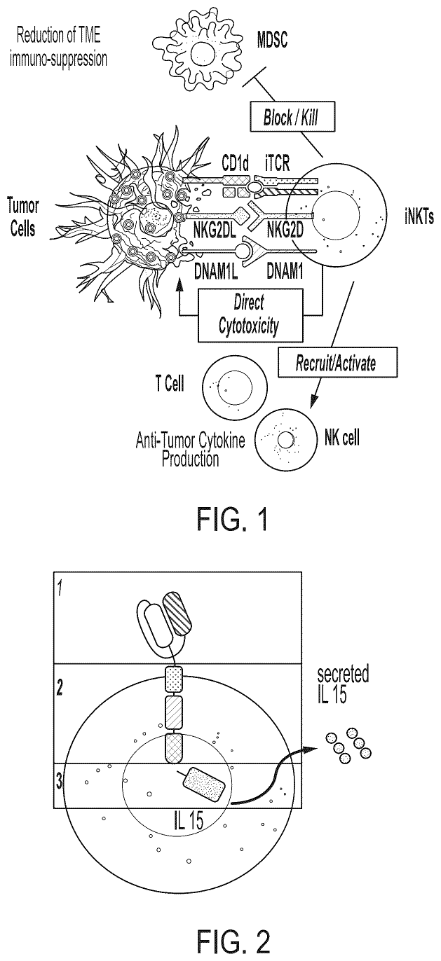

Invariant natural killer T (iNKT) cells act as an anti-cancer mechanism by lysing tumors via T-cell receptor (TCR) dependent and independent mechanisms (FIG. 1). In the direct pathway, the iNKT cells get activated after recognizing lipid-CD1d complexes on the surface of tumor cells via their invariant T cell receptor (iTCR) leading to the subsequent lysis of tumor cells. Similarly, iNKT cell-mediated killing can be triggered when stress molecules on tumor cells are recognized by the Natural Killer (NK) cell activating receptors (e.g., NKG2DL and DNAM) expressed by iNKT cells. In the indirect activation pathway, iNKT cells recognize lipid-CD1d complexes on the surface of the tumor microenvironment (TME) antigen presenting cells (APCs) or tissue associated macrophages (TAMs). This interaction leads to the iNKT-cell mediated killing of immunosuppressive TAMs enabling better function of NK cells. In the case the CD1d is expressed by APCs, iNKT cells can activate the APCs and stimulate production of IL-12, helping resident effector cells like NK cells to overcome the immunosuppressive TME.

The present disclosure, at least in part, describes an anti-BCMA CAR iNKT cell and the uses thereof (e.g., cancer immunotherapy). Generally, a CAR iNKT cell of a present disclosure includes a chimeric antigen receptor and is capable of secreting IL-15 (FIG. 2). Generally, iNKT cells were lentivirally transduced to express a second-generation CAR specifically recognizing BCMA via its fully human scFv (1). The scFv is followed by a CD8 transmembrane domain, a 4-1bb signaling domain and a CD3z activating domain (2). The CAR-iNKT cells also secrete soluble IL-15, as the lentivirus cassette contains the IL-15 gene as well (3).

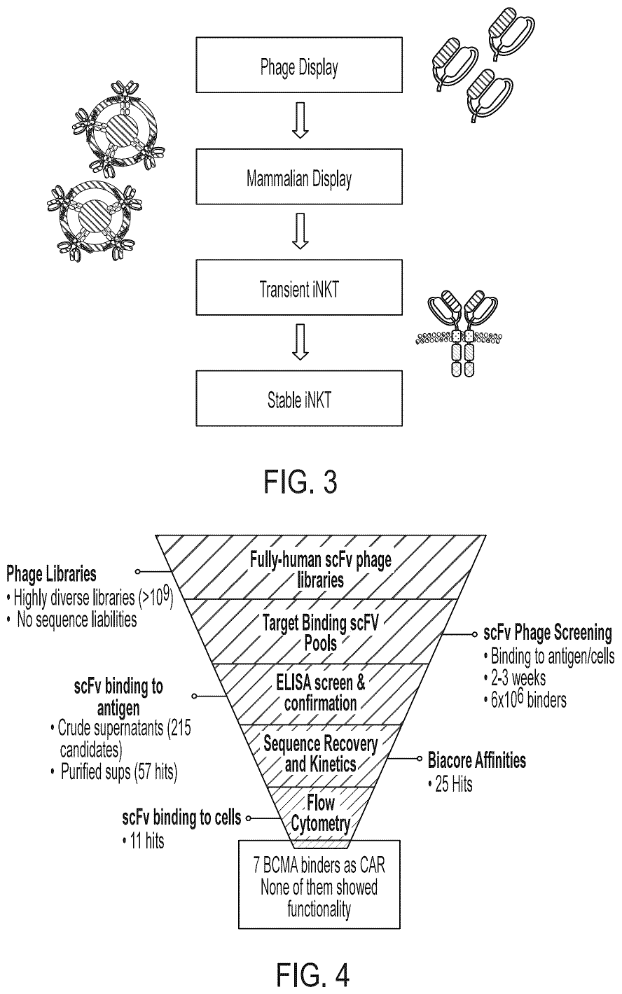

FIG. 3 shows an overview of the screening process of an anti-BCMA CAR. The CARDIS™ platform is a 2-stage high-throughput discovery approach, where the screening of highly diverse scFv libraries via phage display is followed by functional activity screening of mammalian display libraries in a CAR format. Promising candidates are transiently transfected to iNKT cells and are tested in functional assays such as killing, and activation and most potent CARs are then being stably transfected to iNKT cells for more comprehensive, long-term assays such as repetitive stimulations.

FIG. 4 shows a traditional phage campaign to select BCMA binders, which is a schematic representation of early BCMA phage campaign funnel. A highly diverse, naïve, human scFv phage display library containing above 1010 different scFv clones was used for screening against BCMA antigen. After two rounds of selections, 219 single colonies from the selection output were tested by ELISA and 57 binders were identified. Elisa binders were then further tested in Biacore. Out of those binders, 25 clones with favourable kinetics were selected and further cell binding assay narrowed down the candidates to 11. Out of those 11, 7 were still binders as CARs but none of them showed functionality as measured by BCMA positive, target cells killing.

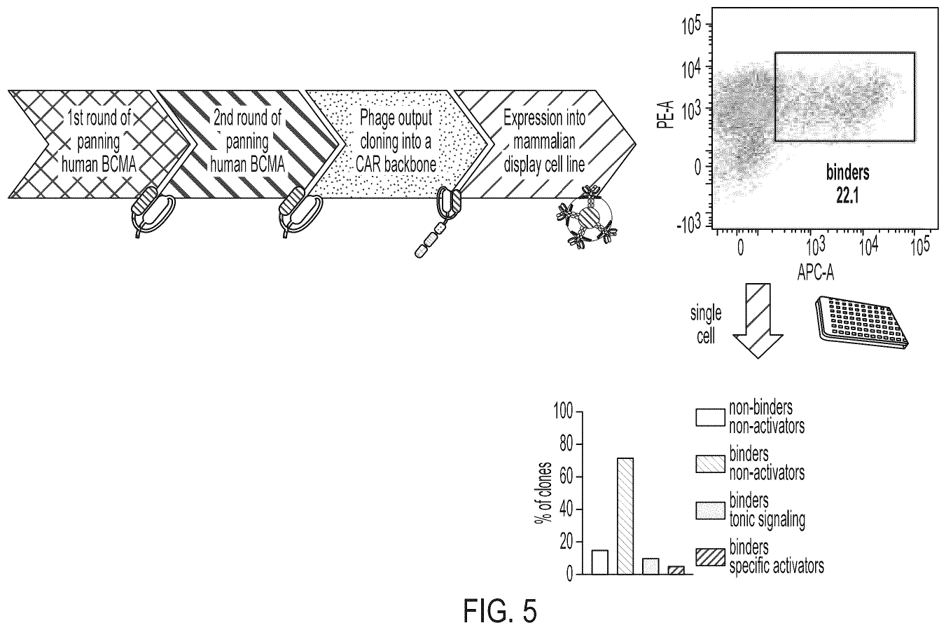

FIG. 5 shows a mammalian screening strategy to identify highly functional CARs. Phage selection outputs were converted into CAR expression libraries and transduced into AKD mammalian cells for functional screening. Binding assay in mammalian cells libraries showed that only 20-25% of CAR-expressing cells bind BCMA antigen in a CAR format. BCMA-binders were single-cell sorted, and binding was confirmed on 85% of clones. A series of activation assays performed on single-sorted cells resulted in only 4% of specific activators, while over 9% were non-specific tonic activators. The majority of the clones were binders but not activators (71%). This highlights the importance of screening CAR for functionality in a format as close as possible to the final application. After the mammalian screening, a few candidates (e.g., F1, F6) were selected for further development.

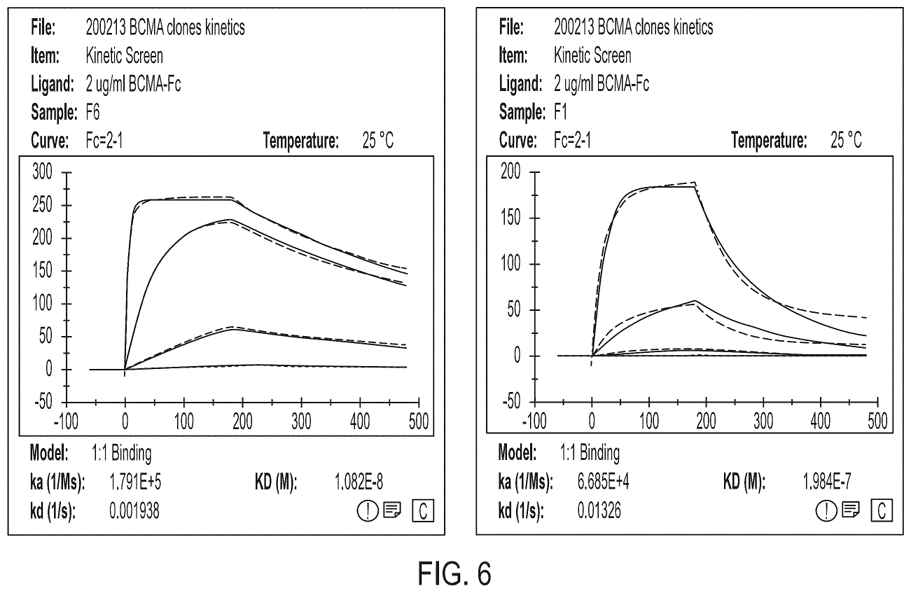

Affinity of F6 and F1 scFvs to BCMA was measured by Biacore T200 (FIG. 6). BCMA-Fc antigen was immobilized on a protein A chip, and BCMA clones expressed as scFv were flowed over the chip at 0, 1, 10, 100 and 1000 nM concentrations. Experiment was run on Biacore T200. Both F1 and F6 were capable of binding BCMA with F6 having higher affinity.

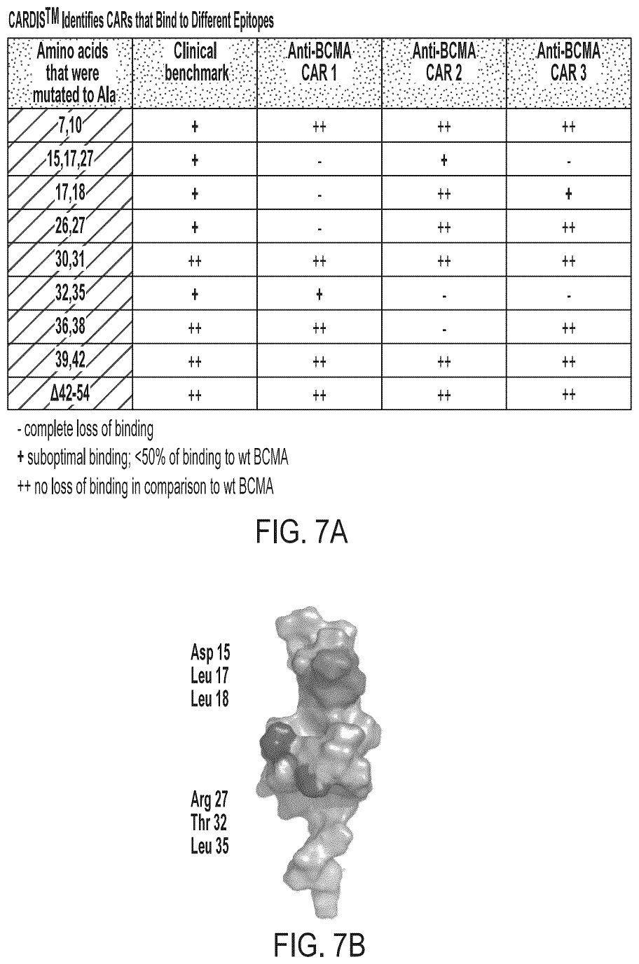

To map the epitope of anti-BCMA CAR candidates, accessible BCMA amino acids were mutated to Alanine and loss of binding in comparison to wild type was quantified via Biacore. FIG. 7A shows the loss of binding to various BCMA antigen mutants by various anti-BCMA CARs. FIG. 7B shows 3D structure demonstrating the binding site of F6G (germlined F6) to BCMA based on A (Asp15, Leu17, Leu18 on the top, Arg27, Thr32 and Leu35 at the bottom).

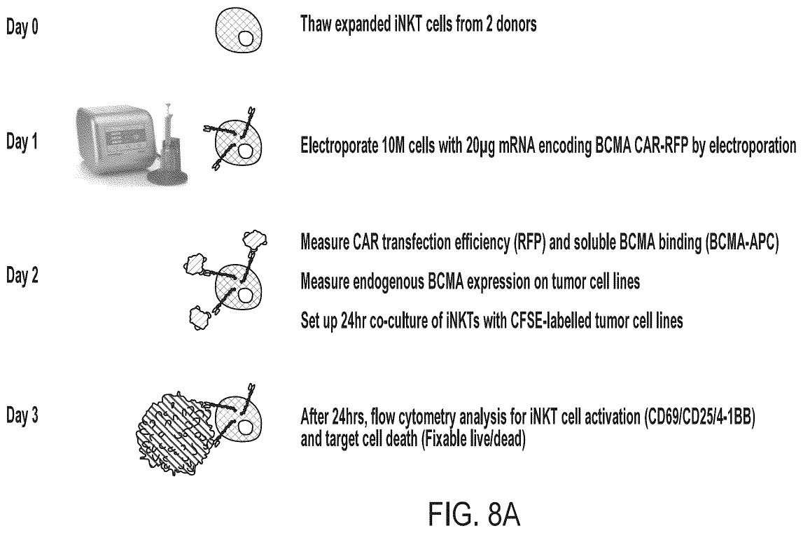

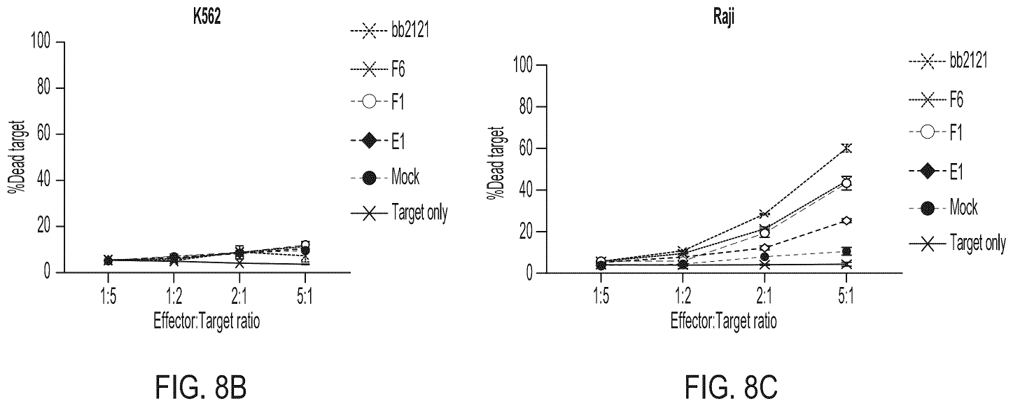

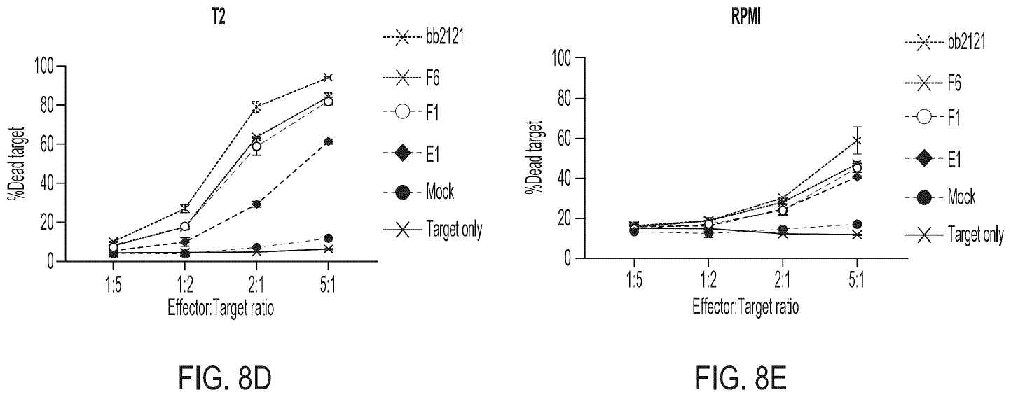

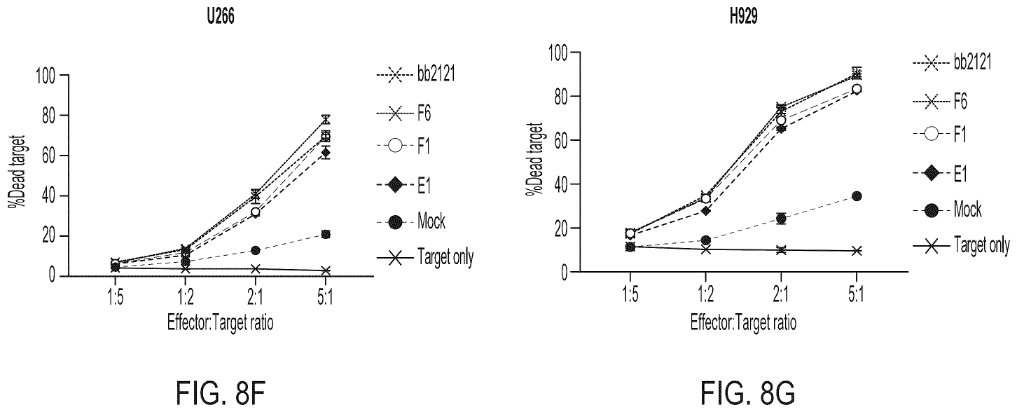

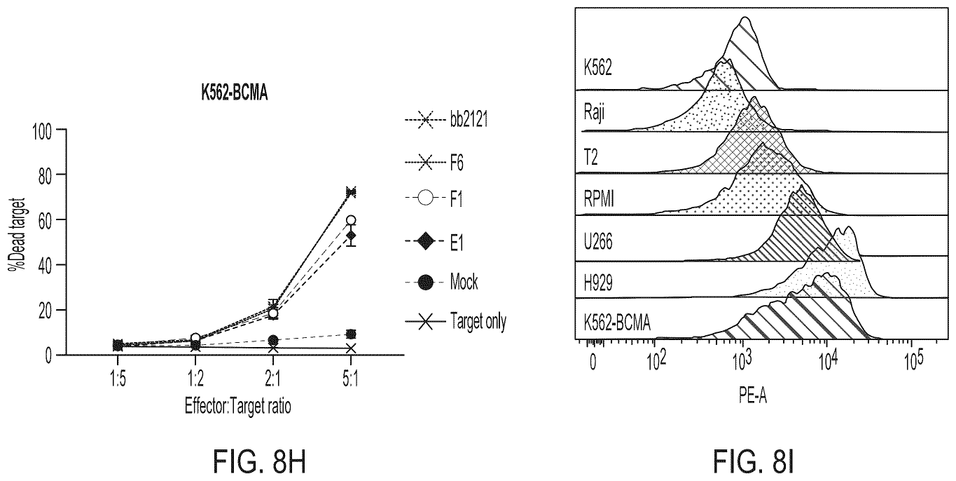

Candidate anti-BCMA scFvs were transiently expressed as anti-BCMA CARs in iNKT cells and were tested in cytotoxicity assays. FIG. 8A shows a schematic of cytotoxicity assays mediated by iNKT cells transiently transfected with BCMA-CARs. iNKT cells expressing different BCMA-CAR candidates or a clinical benchmark (bb2121) were co-cultured with tumor cell lines expressing BCMA for 24 hours at 3 different effector-to-target ratios. The percentage of dead tumor cells was assessed by flow cytometry. K562 cells wild type or stably transduced with BCMA were also included in the assay (FIGS. 8B-8H). The level of endogenous BCMA expression for each tumor line was assessed by flow cytometry (FIG. 81). The results show that in Raji, and T2 cells, bb2121 performs better than the three candidates and in RPMI, U266, H929, and K562 cells the three candidates perform similar to bb2121. The results suggests that improvements need to be done to the candidates to enhance function of the BCMA binders. F6 was selected for further development.

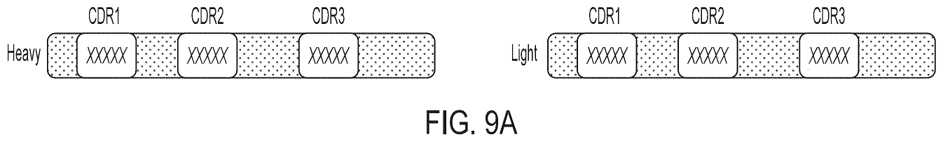

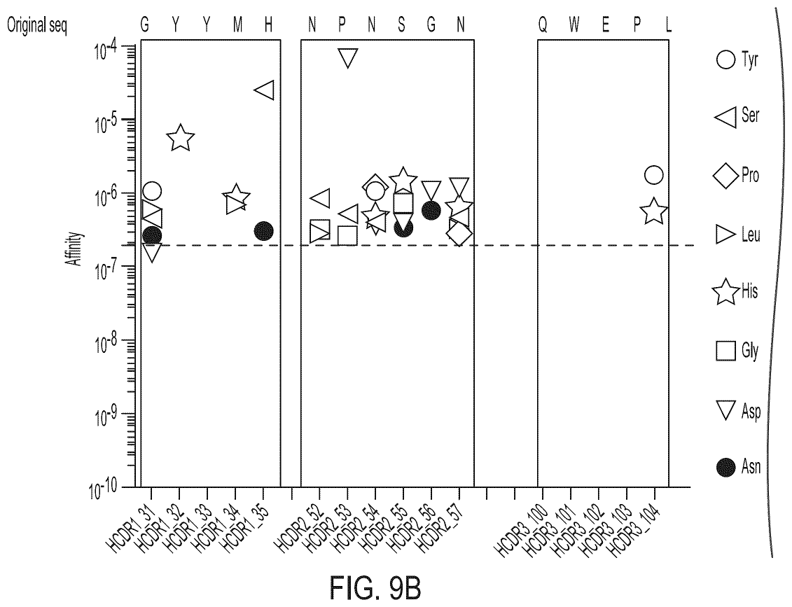

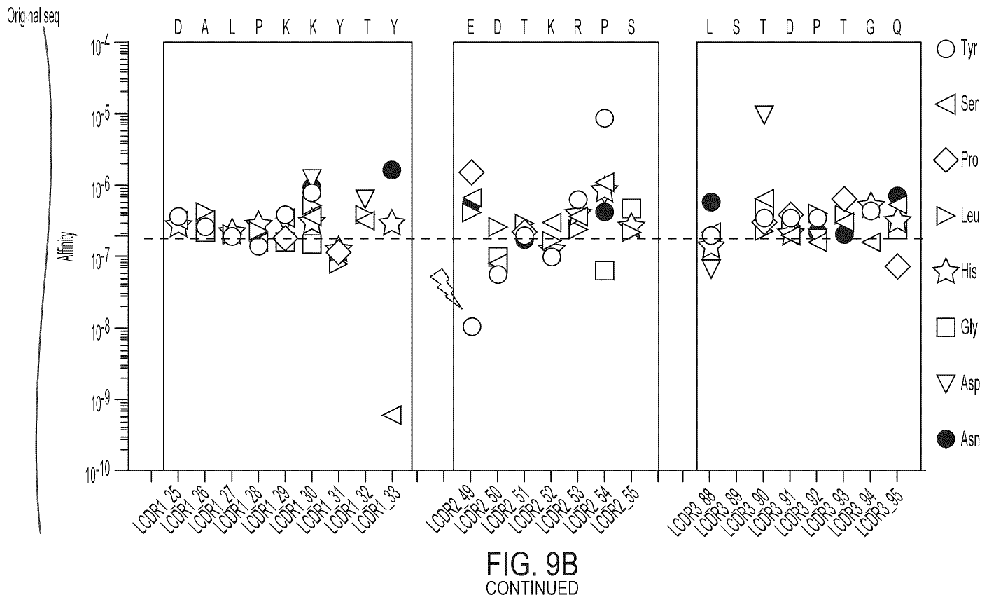

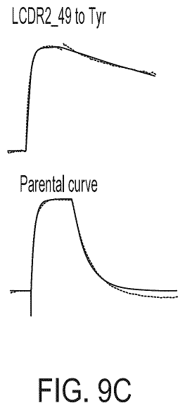

Further, a germlined version of F6 (F6G) was affinity matured by a combination of two methods: Accelerated Drug Discovery (ADD) approach and affinity maturation of by phage libraries. FIGS. 9A-9C describe the ADD approach. FIG. 9A is a schematic representation of the CDR positions undergone mutagenesis. Each amino acid in the heavy and light CDRs was substituted with 8 different amino acids; Tyr, Ser, Pro, Leu, his, Gly, Asp, Asn. Fragments containing single amino acids substitutions were generated by mutagenesis PCR. The PCR fragments were then used as a template to express the scFv in a cell free system. Affinity K_{D} (M) values of mutant scFvs binding to BCMA antigen. Experiment was run in Biacore 4000. The dotted line indicates the binding of the parental F6G clone. K_{D} below the dotted line means improvement in affinity. E to Y substitution in position 49 of light CDRL2 performs significantly better than others (FIG. 9B). Individual Biacore sensorgrams for E to Y substitution in position 49 of CDRL2 are shown (FIG. 9C).

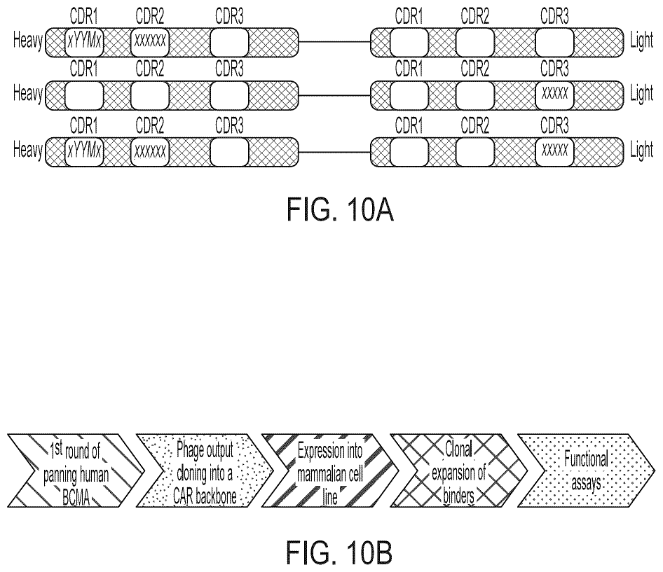

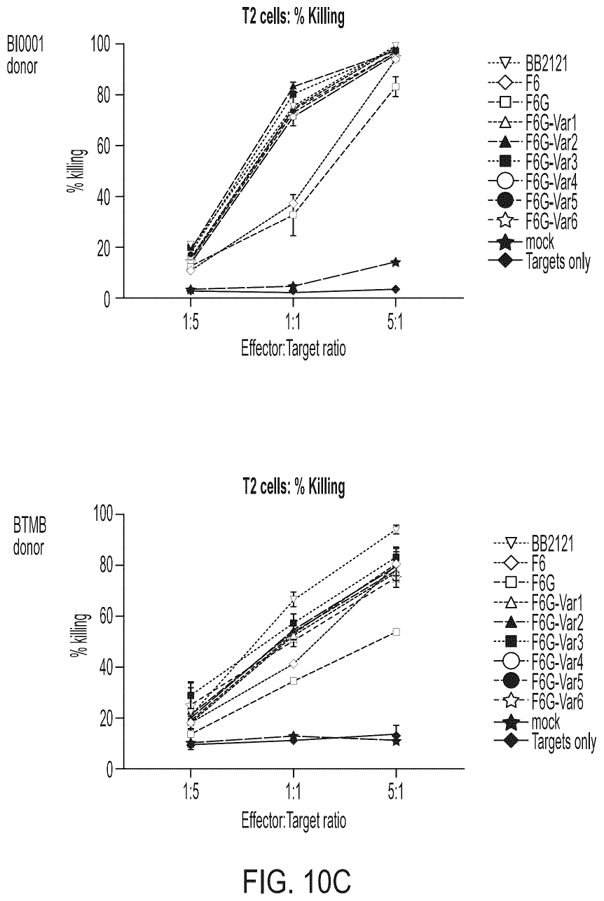

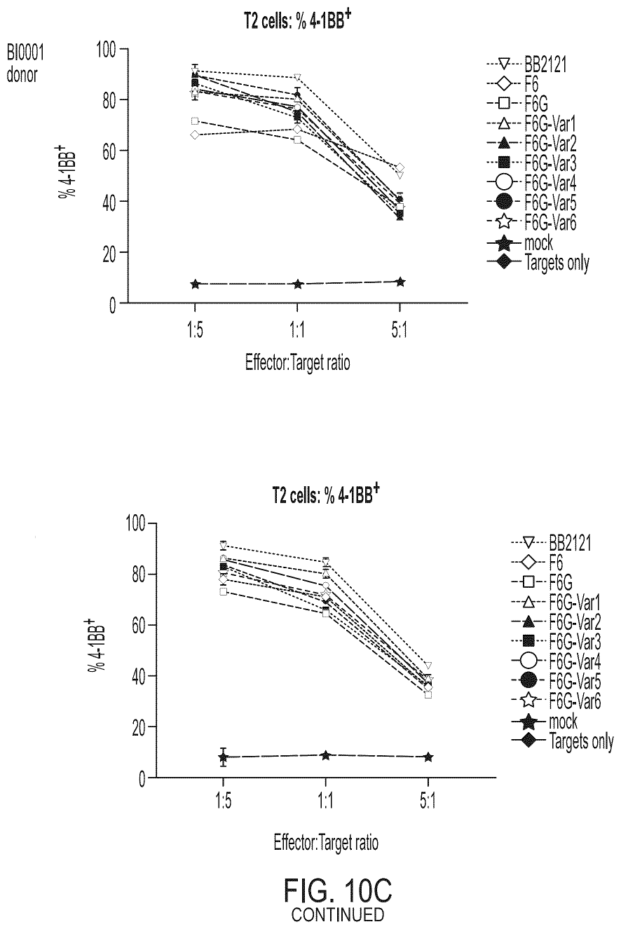

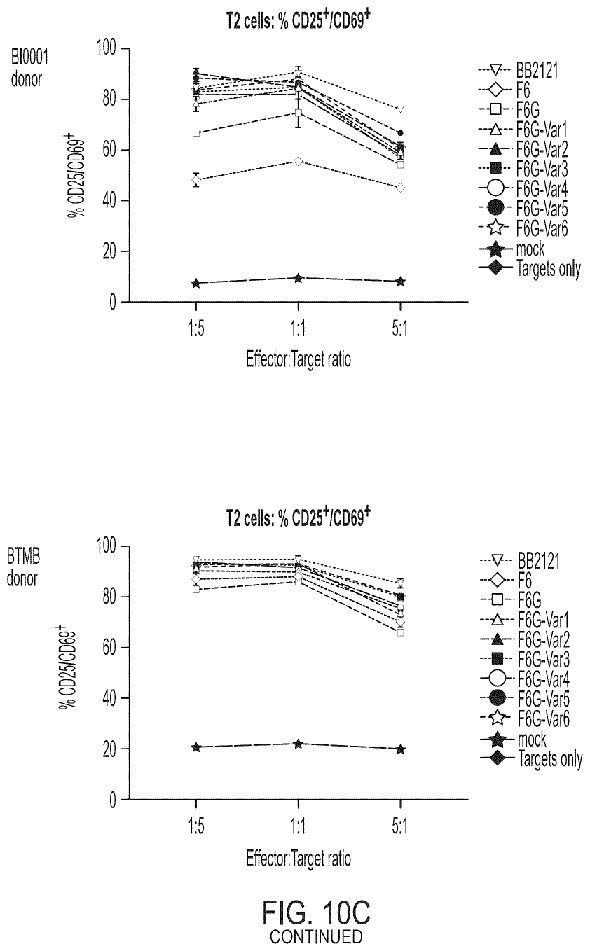

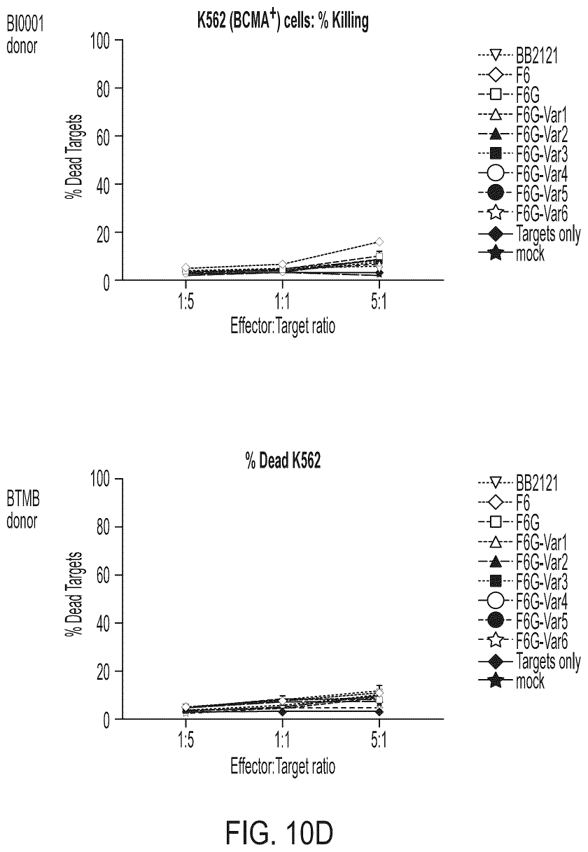

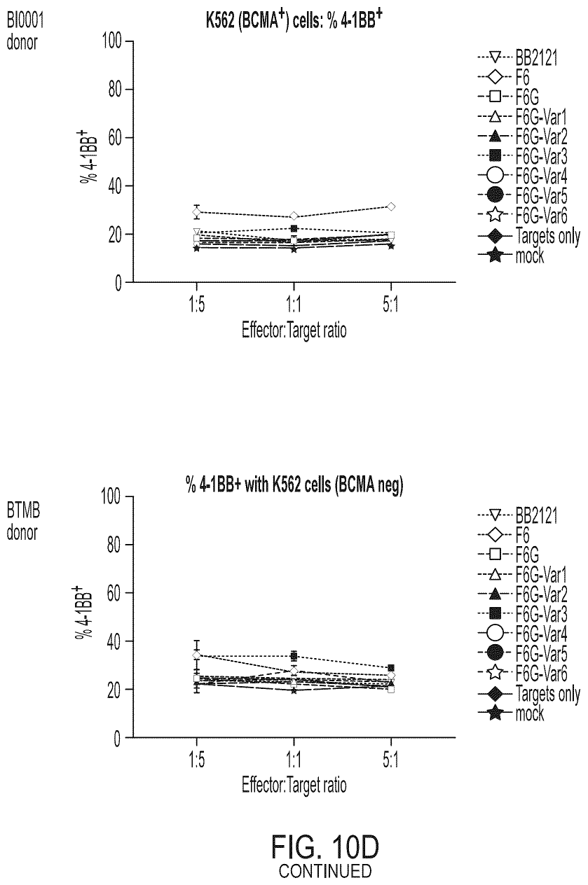

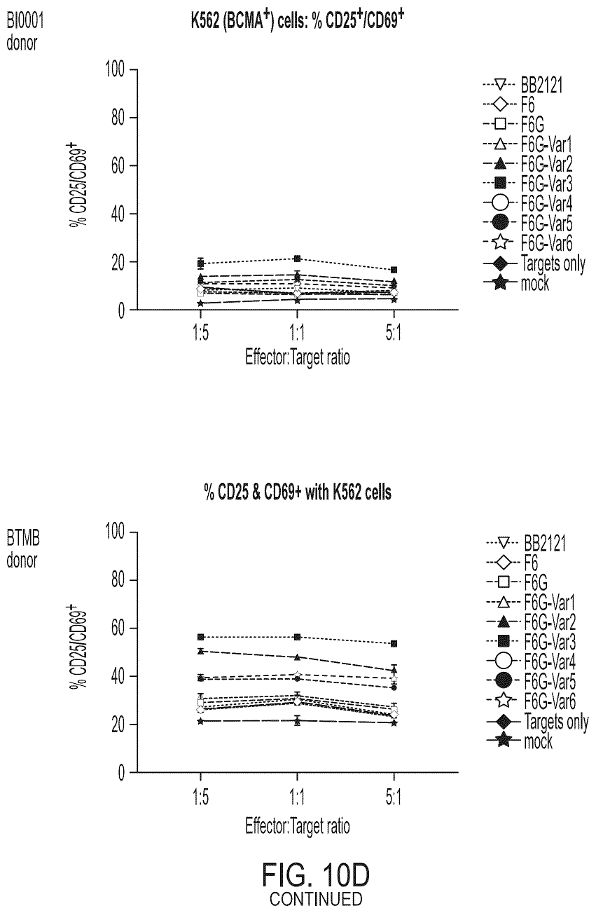

F6G was also affinity matured by phage libraries. FIG. 10A shows a schematic representation of the phage display affinity maturation libraries strategy. The CDR amino acids marked as x were mutated to any other amino acid and the libraries were used for phage panning followed by mammalian display (FIG. 10B).

Based on the combined results of the ADD substitutions and the affinity maturation libraries, a few combinations were designed and tested in killing and activation in functional assays, and the results are shown in FIGS. 10C-10D. iNKT cells expressing different BCMA-CAR candidates or a clinical benchmark (bb2121) were co-cultured for 24 hours with T2 cells which endogenously express BCMA at 3 different effector-to-target ratios. The percentage of dead target cells alongside activation markers 4-1BB, CD25 and CD69 was assessed by flow cytometry. F6G variants showed similar cytotoxic and activation activity to the reference benchmark bb2121. Cytotoxicity towards BCMA negative K562 cells was negligible and similar to the reference clinical candidate. Activation post co-culture with BCMA negative K562 cells was negligible and similar to bb2121 for most F6G variants (FIG. 10D).

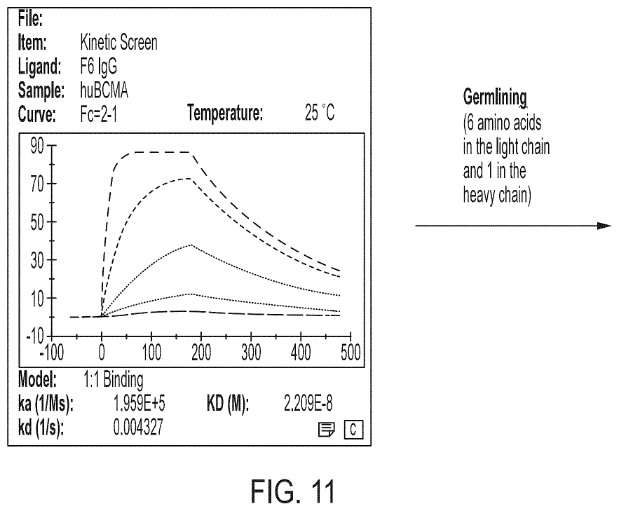

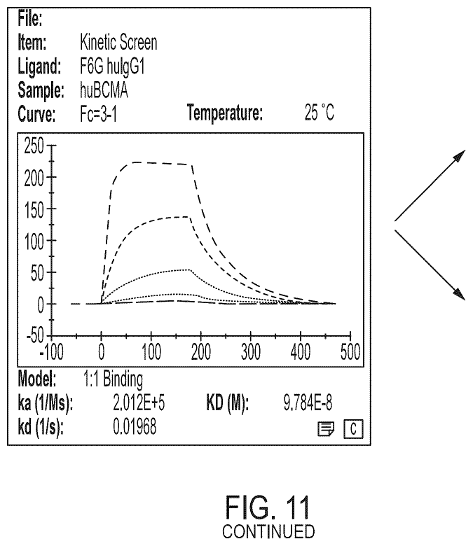

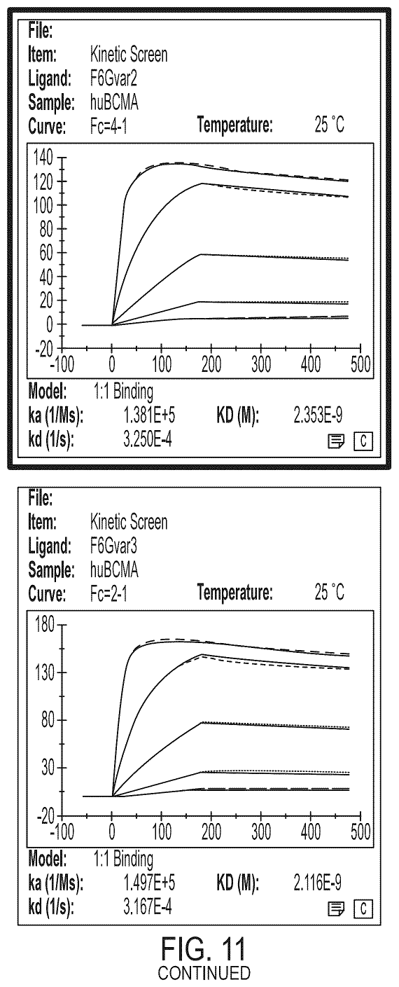

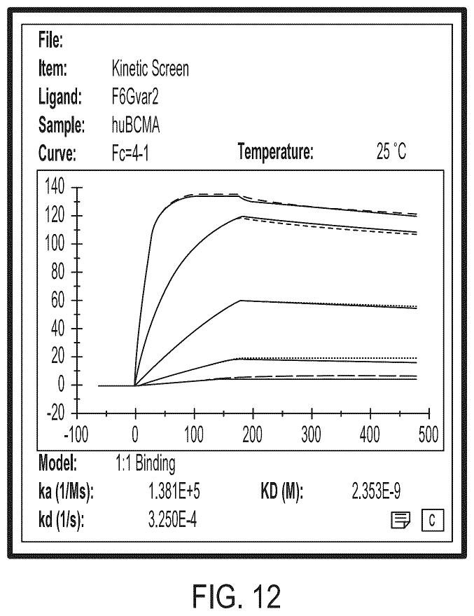

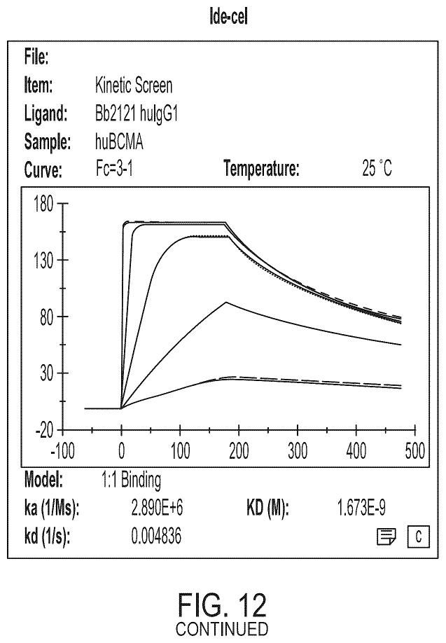

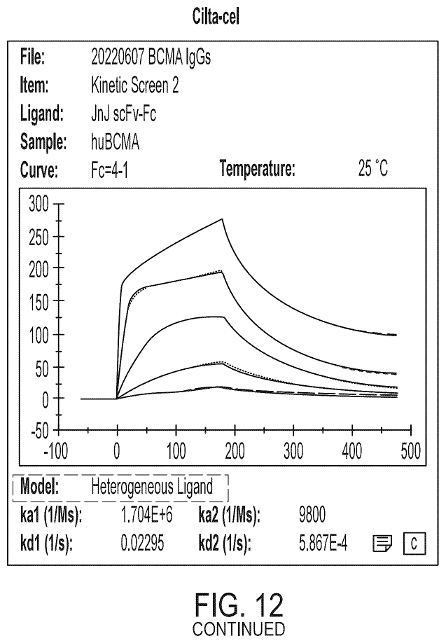

To test the affinity of BCMA F6G variants their binding domains were expressed as IgGs and captured to a protein A chip. BCMA antigen was flowed over the chip at 400 mM, 100 mM, 25 mM, 6.25 mM, 1.25 mM and 0 mM concentrations. Experiment was run on Biacore T200. Affinity mature clones had 10-fold improved affinity in comparison to the original F6 clone and almost 50-fold improved affinity in comparison to the germline F6G (FIG. 11). FIG. 12 shows the affinity of F6G-Var2 and two known anti-BCMA CARs ide-cel and cilta-cel. The CARs were expressed as IgGs and captured to a protein A chip. BCMA antigen was flowed over the chip at 400 mM, 100 mM, 25 mM, 6.25 mM, 1.25 mM and 0. concentrations. Experiment was run on Biacore T200.

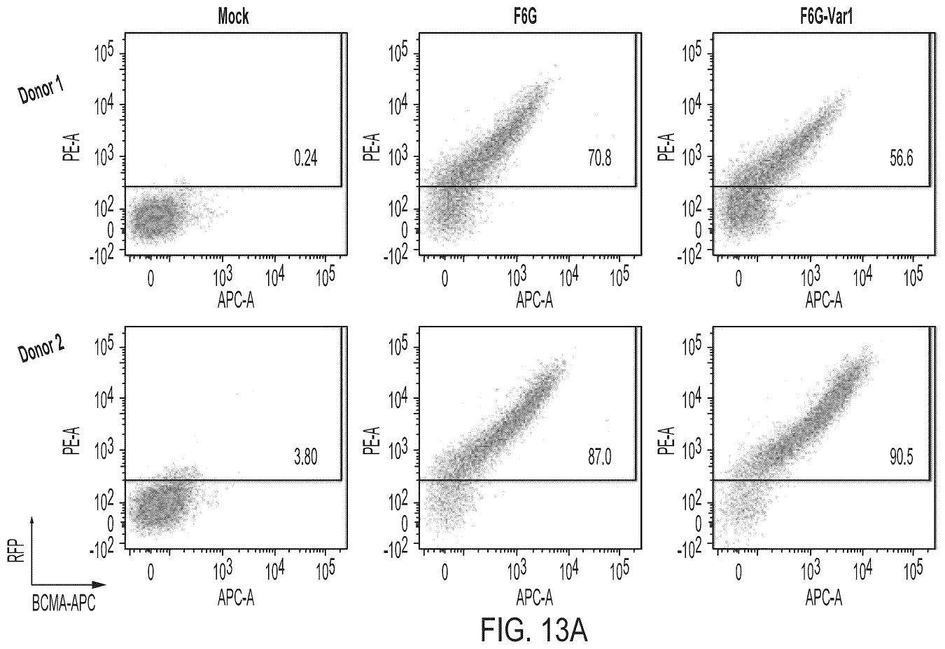

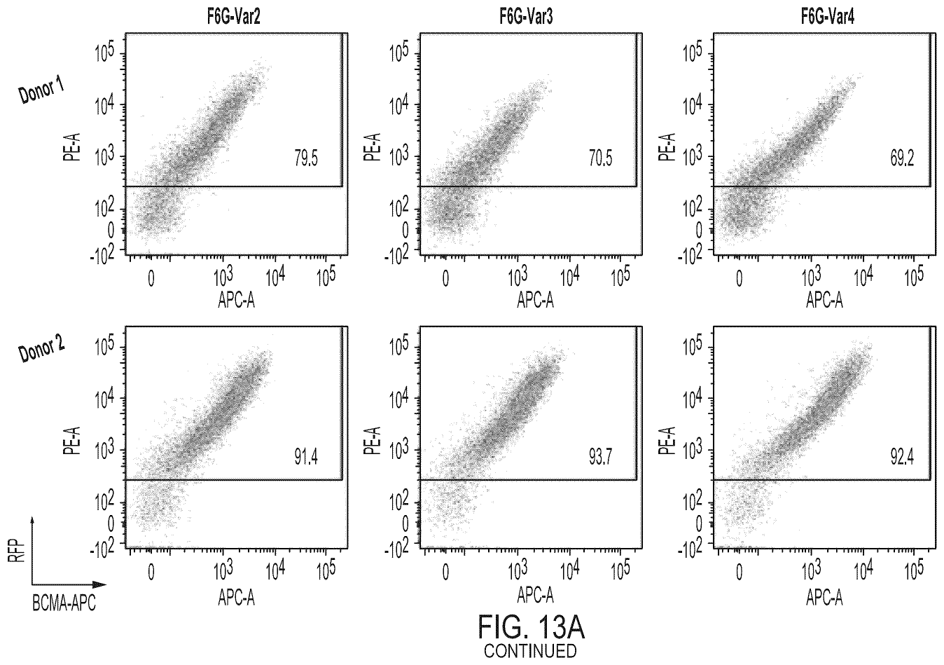

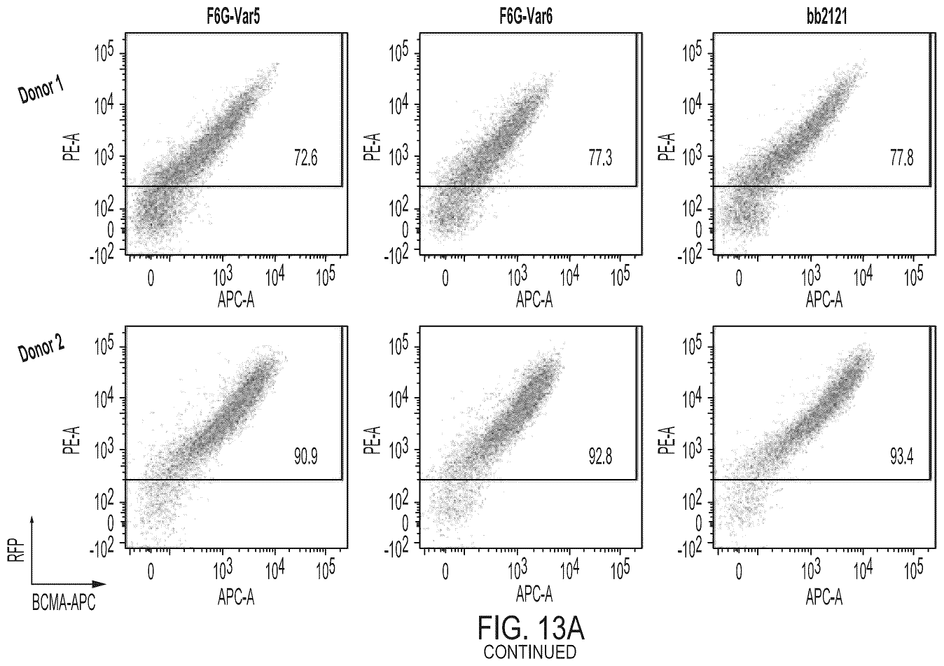

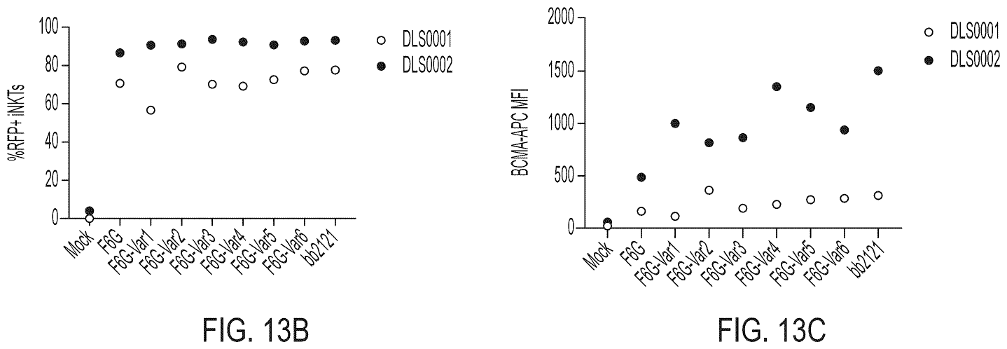

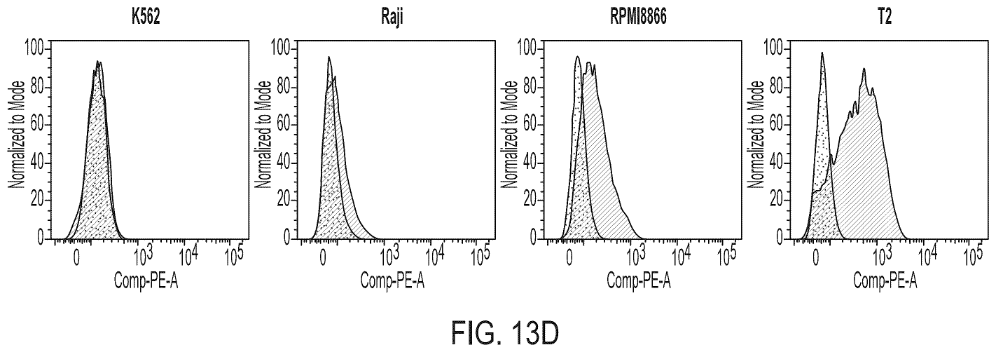

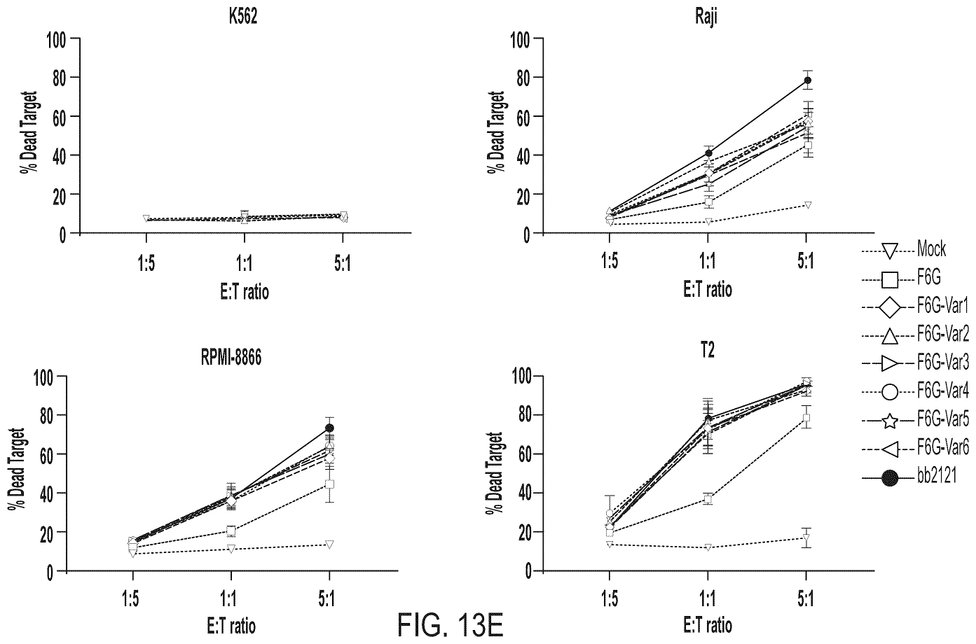

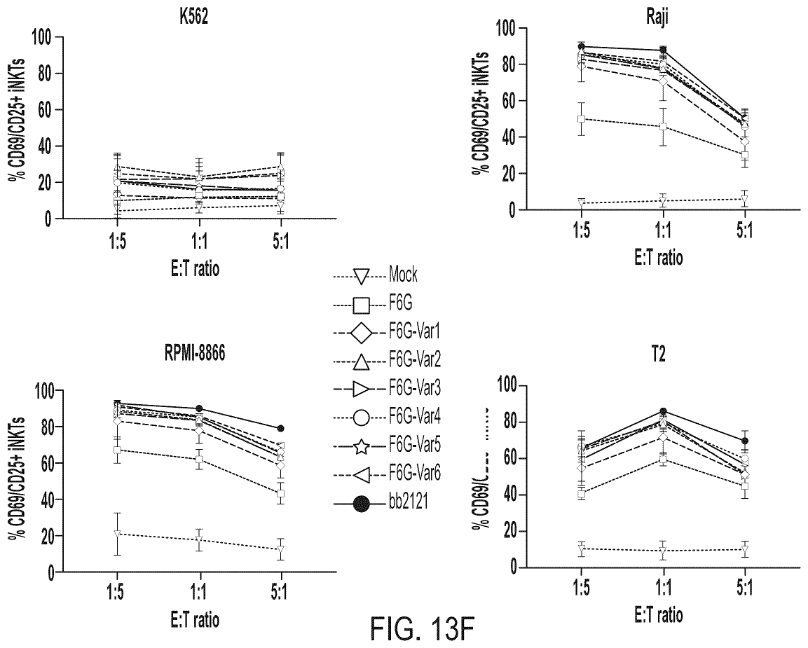

INKT cells from two donors were transiently transfected with F6G variants and anti-BCMA-CAR iNKT cell-mediated killing of tumor cells were tested. Flow cytometry analysis of RFP expression to show CAR expression and antigen binding with BCMA-APC in BCMA-CAR iNKTs (FIGS. 13A-13C). The level of BCMA expression on each tumor cell line was assessed by flow cytometry where left peak show isotype staining and right peak show BCMA expression (FIG. 13D). iNKT cells expressing anti-BCMA CAR (as shown in FIG. 13A) were co-cultured with tumor cell lines endogenously expressing BCMA or K562 for 24 hrs at 3 different effector-to-target ratios. After 24 hr co-coculture, cytotoxicity assay was analyzed by flow cytometry for percentage of dead target cells (FIG. 13E), percentage of CD69/CD25 co-expressing iNKT cells (FIG. 13F) and percentage of 4-1BB expressing iNKT cells (FIG. 13G).

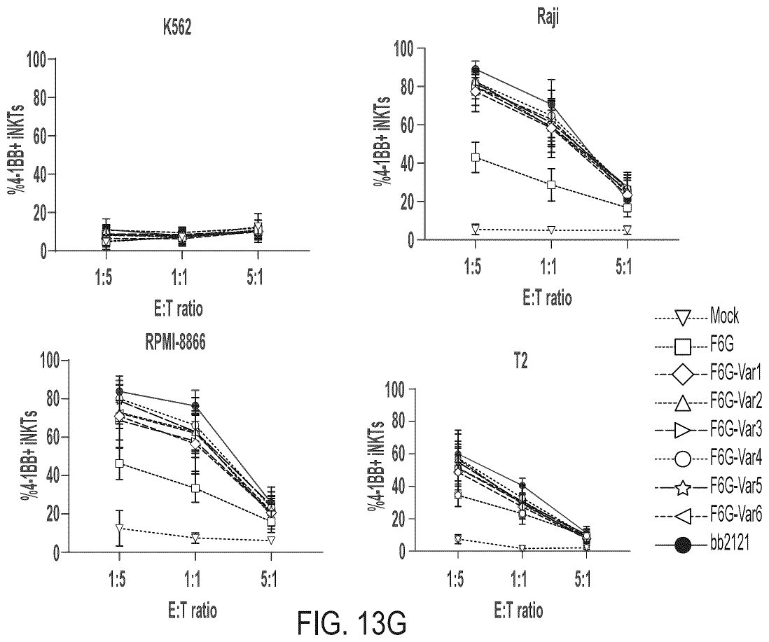

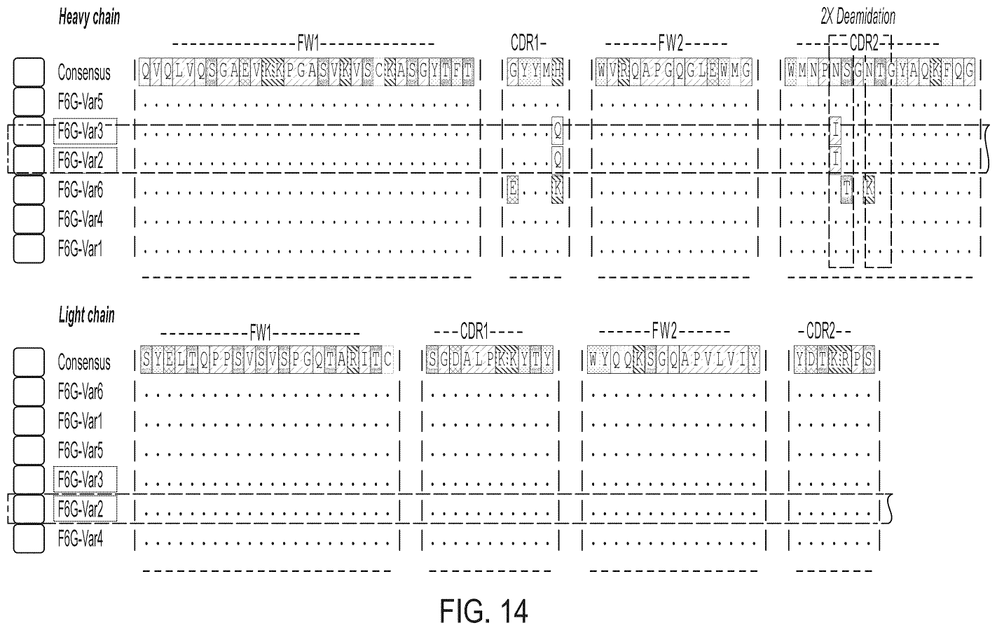

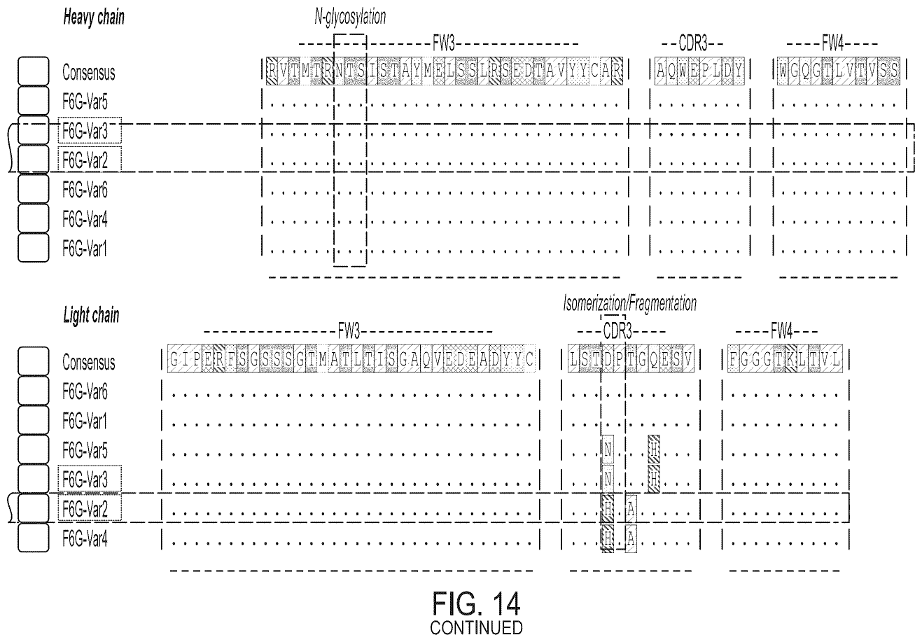

F6G variants heavy and light chain variable region sequence alignment is shown in FIG. 14. Immunogenicity of F6G variants were analyzed. An in house in-silico immunogenicity scoring algorithm based on NetMHCpanII v.4 was used to rank the risk of the candidate BCMA CARs to other biotherapeutics. The protein sequence was evaluated in terms of overlapping 9-mer peptides and a cumulative score calculated. The value of the calculated score is only relevant when it is compared to the values in a representative reference set. In this case a set of 420 sets of Fv's (variable domains) from therapeutic antibodies from Thera-SabDab were used. The 420 Fv sequences were divided into 4 sets [Human (hu), Humanized (zu) and Murine/Chimeric (mu/xi), All (Total)]. The in-silico immunogenicity scores where then plotted on the X-axis, with the cumulative percentage %) of the set on the Y-axis. For example 79% of all 197 Human (hu) Fv's had a score of 100 or less. Lower scores are better.

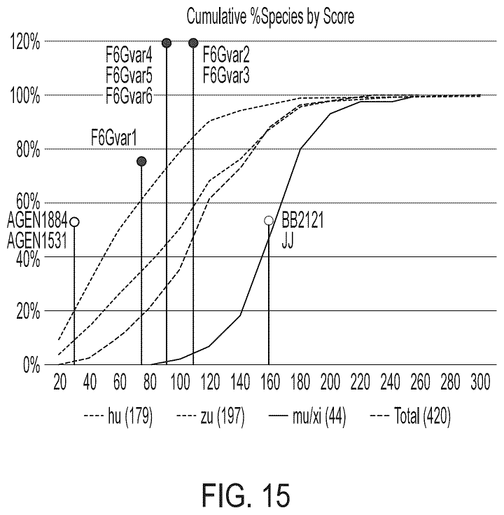

The in-silico immunogenicity scores of the full length BCMA CARs, including the transmembrane and intra-cellular domain regions were compared to the reference set of Thera-SabDab antibody Fv scores. Internal MiNK Therapeutics thresholds labels proteins with a score of 100 or less as having a standard risk, compared to human and humanized antibodies, as ˜80% (79%) of the Human and ˜40% (36%) of Humanized Fv's have a score better than 100. An further internal threshold score of 140, against this reference set, indicates proteins with an acceptable risk. 4 of the BCMA CARs fell into the standard risk category and 2 in the acceptable category (FIG. 15). All BCMA CARs had lower risk scores than competitor CAR products ide-cel and cilta-cel which scored 202.9 and 195.4 respectively.

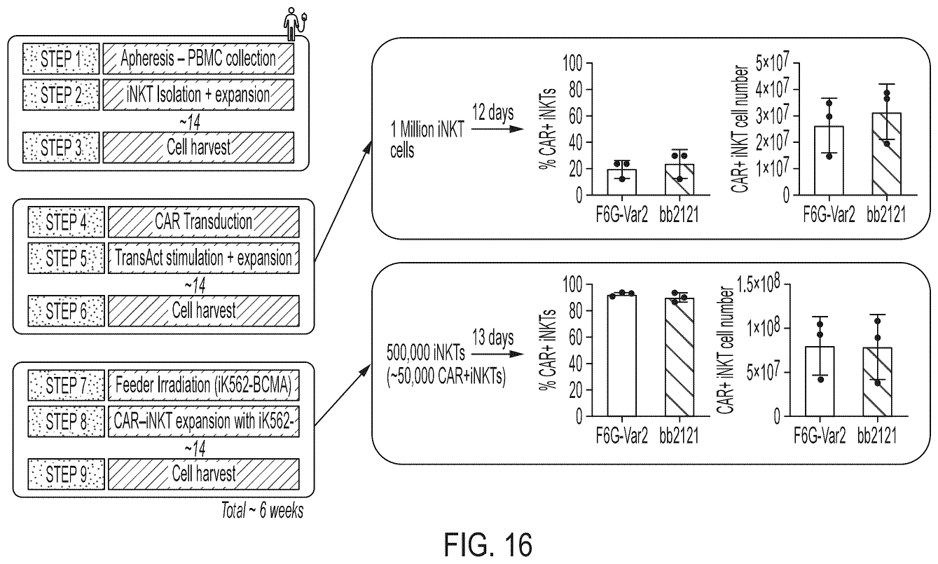

iNKT cells stably expressing anti-BCMA CAR F6G-Var2 were produced by a method described in FIG. 16 (left panel). One million iNKT cells (3 donors) were transduced with F6G-Var2 or bb2121 lentivirus and expanded for 12 days when purity and number of CAR-iNKT cells were assessed by flow cytometry and cell counting (Top right panel). 500,000 iNKT cells normalized to 10% CAR+ cells were then co-cultured with 500,000 irradiated K562-BCMA feeders and expanded for 13 days. Purity and number of CAR-iNKTs was assessed by flow cytometry and cell counting (Top right panel).

Purified and a-GalCer expanded iNKT cells from 2 donors were stimulated with TransAct (Miltenyi) and transduced with lentivirus (MOI 3) encoding for various 2nd Generation BCMA-CAR molecule and secreted IL-15. Constructs including F6G-Var2 IL-15, F6G variant 2 (no IL-15), F1G-IL-15, bb2121-IL-15, and CD19-CAR-IL-15 were tested. Cells were expanded for 10 to 12 days then transduced cells were co-cultured with K562 cells expressing BCMA at a ratio iNKT:K562 of 1:1. Cells were cultured and enriched with media supplemented with supporting cytokines IL-2 (100 IU/mL), IL-15 (100 UI/mL) and IL-21 (20 IU/mL). Media was changed on Day 7 by removing 70% of old media and replenishing with fresh enrichment media, Cell culture was supplemented with cytokines every 48-hours or split into new wells depending on cell numbers until cryopreservation.

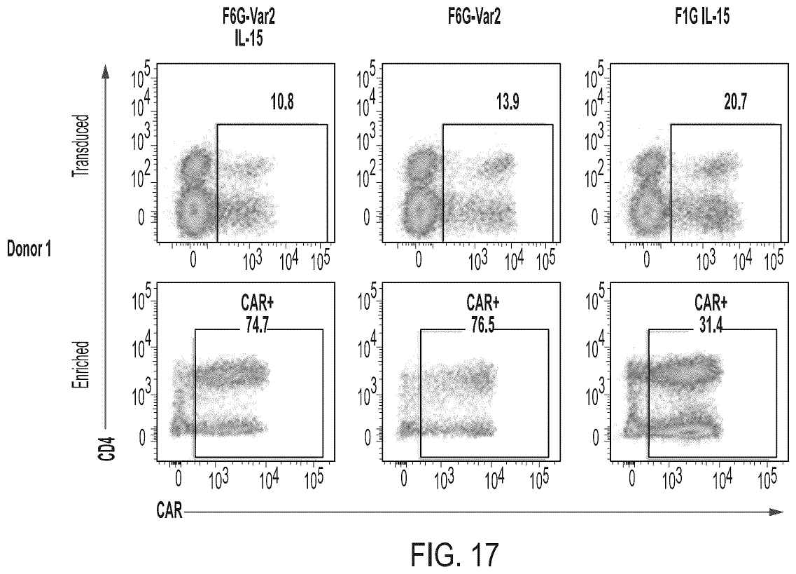

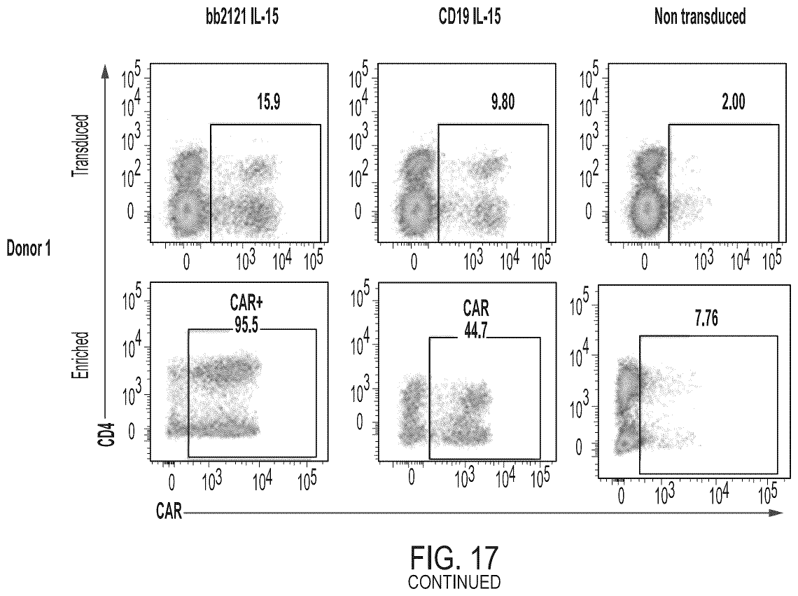

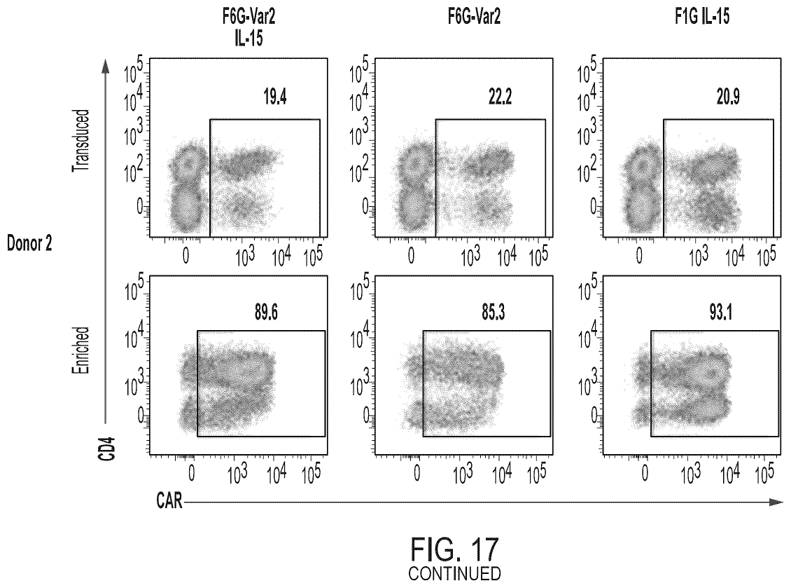

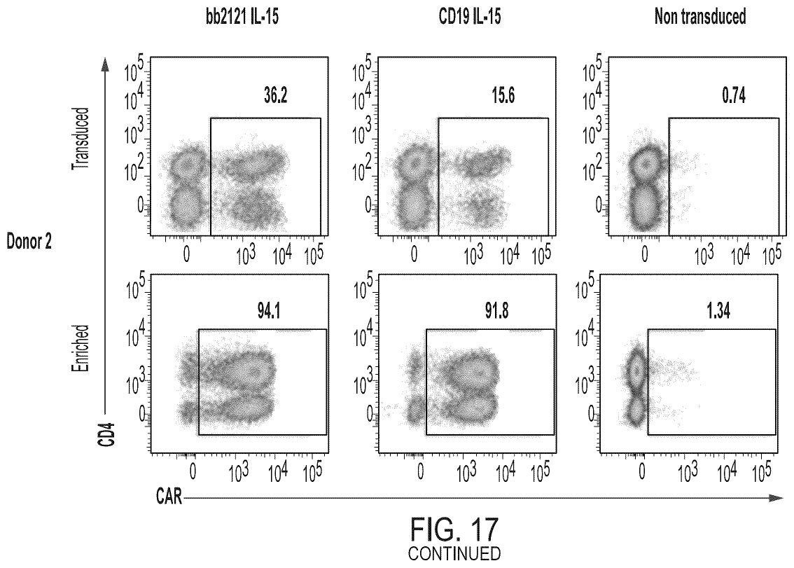

Transduced cells pre-co-culture and enriched cells post co-culture with K562-BCMA were analyzed for CAR expression by flow cytometry (FIG. 17). iNKT cells were incubated with a LIVE/DEAD (Biolegend) reagents to separate live from dead cells and then stained for cell surface markers BCMA-CAR or CD19-CAR, BUV395 CD8 (BD, clone RPA-T8) and BUV563 CD4 (BD, clone OKT4). BCMA-CAR expression was detected with an unconjugated, primary BCMA-Fc reagent (AcroBiosystems) while CD19 CAR expression was detected with an unconjugated, primary CD19-Fc reagent (AcroBiosystems). Both were then stained with an A647 conjugated, secondary donkey anti-human IgG, Fc (JacksonImmuno) antibody.

Invariant NKT cells expressing BCMA-CAR-IL-15 (F6G-Var2 IL-15, F1G-IL-15, BB2121-I1-15) or BCMA-CAR (F6G-Var2), or CD19-CAR-IL-15 were co-cultured with BCMA expressing multiple myeloma tumor lines MM.1s, OPM2 and RPMI8226 and Burkitt's lymphoma Raji. In addition, wild type K562 and K562 overexpressing BCMA were used as negative and positive control respectively. Tumor lines and CAR-iNKT cells from 2 iNKT donors were co-cultured for 16 to 20 H at various effector (iNKT) to Target (Tumor) ratio (E:T ratio). Briefly, a constant number of tumor cells (25,000 in 100 μL volume) were seeded in a 96 U-bottom plate then a variable number of CAR-iNKT (100 μL volume) was added depending on the E:T ratio. At E:T ratio of 1:10, 1:5, 1:1 and 5:1, a total of 1000, 5000, 25000 and 125000 CAR-iNKT cells were added. Tumor lines were engineered to express nano-luciferase to enable bioluminescent read-out of cytotoxicity assay. Post co-culture, cells were washed with PBS, and the remaining cells were resuspended in assay media and lysed furimazine-lysing buffer solution mixture to release the luciferase enzyme. Bioluminescent signal from remaining live and lysed cells was read with a bioluminescence reader (PerkinElmer® En Vision® Multimode Plate Reader). This method allows quantitation of cytotoxic activity of BCMA-CAR iNKT cells; A decrease of bioluminescence compared to a condition target alone indicates that target cells have been lysed by effector cells.

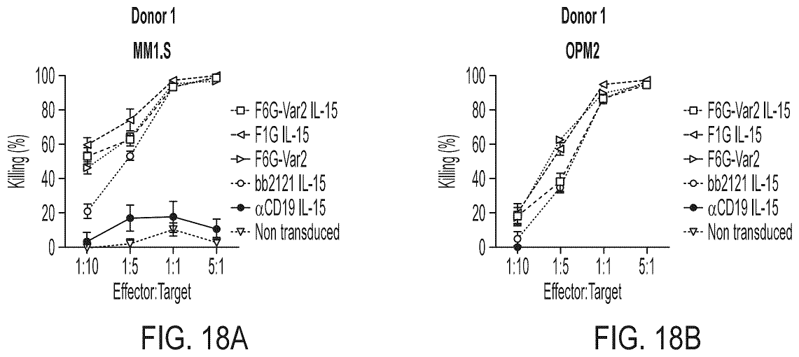

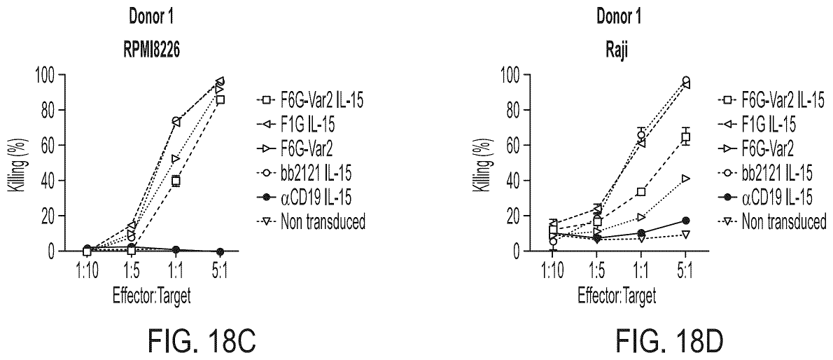

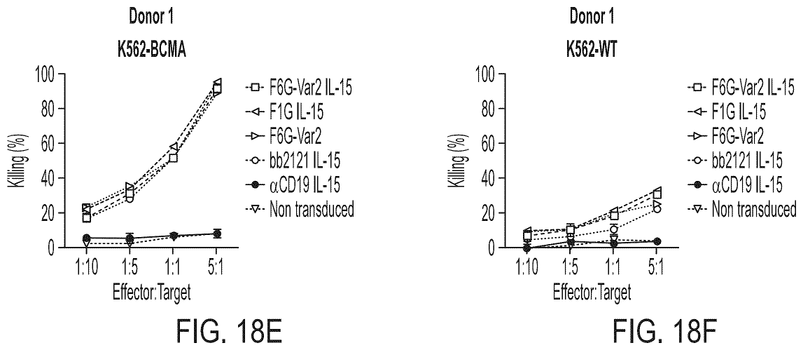

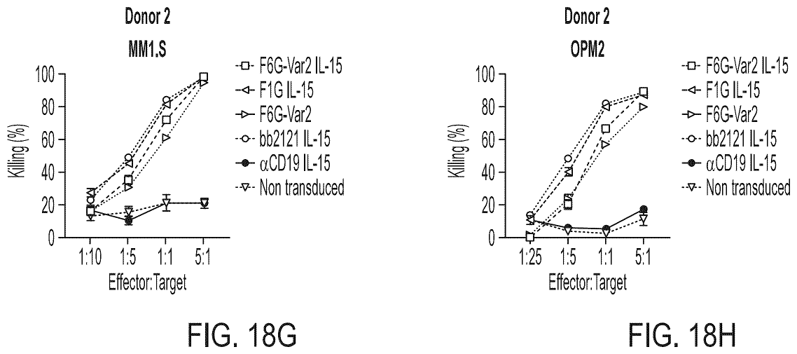

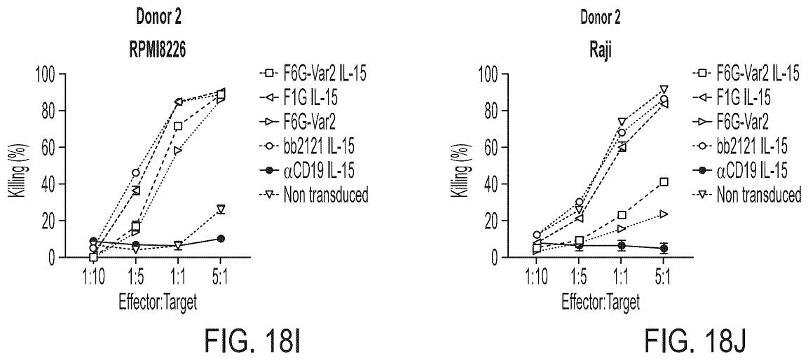

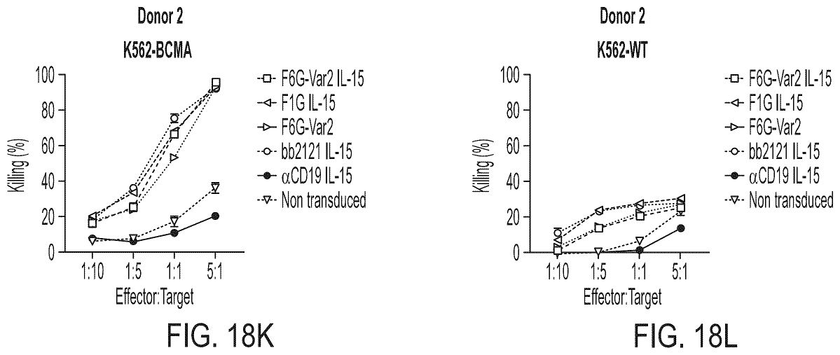

F6G-Var2 IL-15, F1G IL-15, BB2121 Il-15 and F6G-Var2 showed cytotoxic activity toward BCMA expressing tumor lines and K562 overexpressing BCMA while non transduced iNKT and CD19-CAR iNKT cells show limited to no cytotoxicity to BCMA expressing cells. When challenge with wild type K562, cytotoxic activity of BCMA-CAR iNKT was strongly reduced compared to K562-BCMA (FIGS. 18A-18F for donor 1; and FIGS. 18G-18L for donor 2).

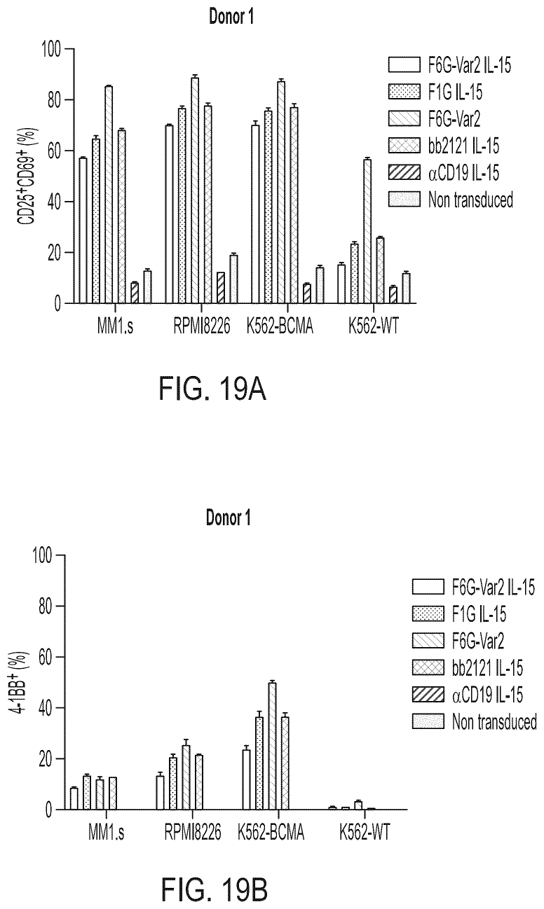

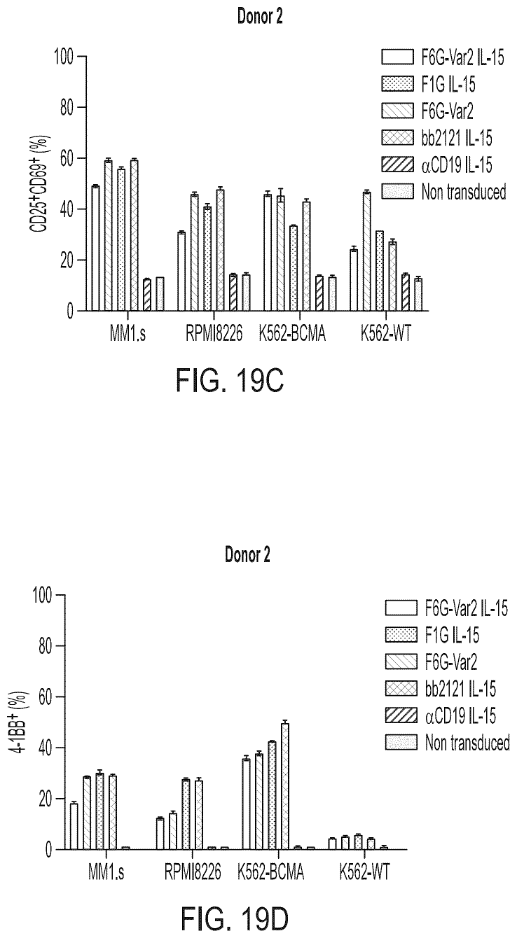

Antigen specific activation of BCMA-CAR-IL-15 iNKT cells were also evaluated. Invariant NKT cells expressing BCMA-CAR-IL-15 (F6G-Var2 IL-15, F1G-IL-15, BB2121-IL-15), BCMA-CAR, (F6G-Var2), CD19-CAR-IL-15 and non-transduced iNKT cells were co-cultured with BCMA expressing tumor lines MM.1S and RPMI-8226, K562 cells overexpressing BCMA and wild type K562 cells at a E:T ratio of 1:1 for 16 to 20 H. Post-coculture, cells were washed and incubated first with LIVE/DEAD reagent to discriminate live cells. Cells were then stained for the cell surface markers CD25 (Biolegend, BV510 clone BC96), CD69 (Biolegend, PerCP-Cy5.5 clone FN50) and 4-1BB (Biolegend, PE-Cy-7 clone 4B4-1) and analyzed by flow cytometry (BD LSR Fortessa). When challenged with BCMA expressing tumor lines, BCMA-CAR iNKT cells from both donors showed increased cell surface expression of the markers CD25, CD69 and 4-1BB compared to non-transduced cells and CD19-CAR-IL-15 iNKT cells (FIGS. 19A-19D).

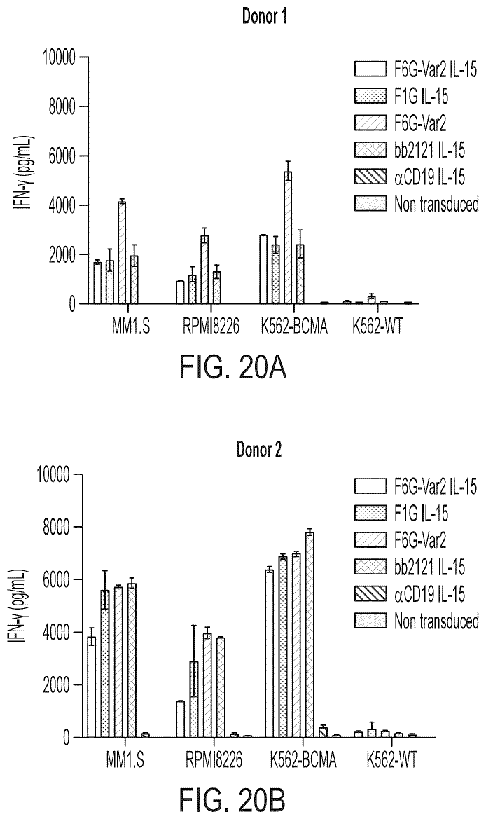

Further, BCMA-CAR-IL-15 iNKT cells show increase IFN-γ production when challenged with BCMA expressing tumor lines (FIGS. 20A-20B). Supernatants from BCMA-CAR iNKT, CD19-CAR iNKT, untreated iNKT co-cultured with MM.1S, RPMI-8226, K562-BCMA and K562 wildtype at an E:T ratio of 1:1, were collected and IFN-γ was quantified by Luminex-based immunoassay. When challenged with BCMA expressing tumors, BCMA-CAR iNKT cells showed increased production of the pro-inflammatory cytokine IFN-γ compared to CD19-CAR iNKT cells and untreated cells indicating specific antigen iNKT activation.

Anti-BCMA CAR iNKT cells armored with soluble IL-15 showed increased in vivo persistence of the anti-BCMA CAR iNKT cells.

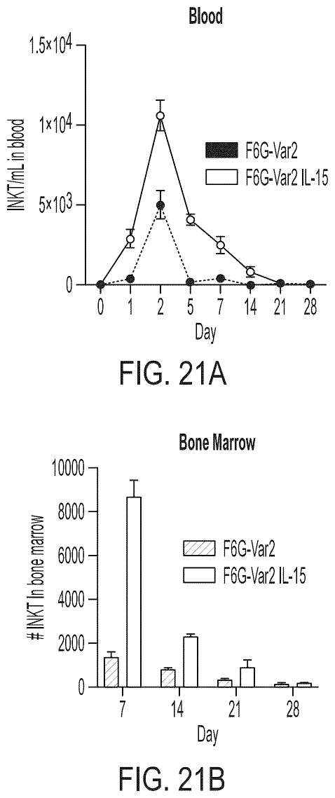

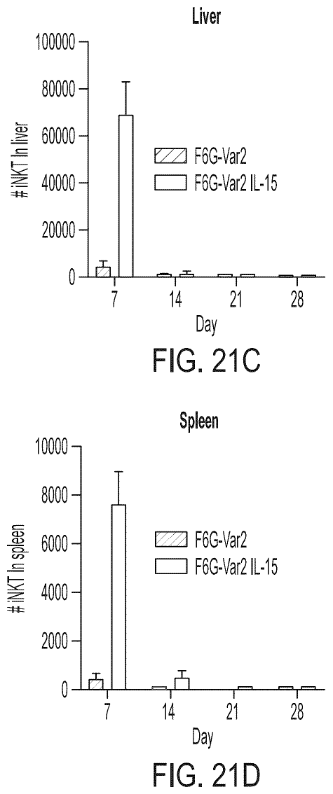

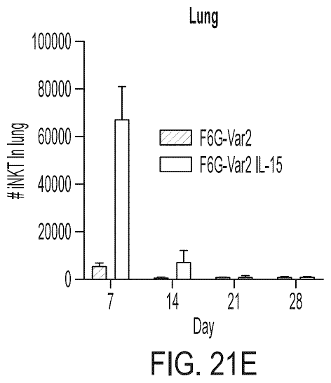

Anti-BCMA-CAR F6G-Var 2 IL-15 iNKT cell and its non-IL-15 counterpart, F6G-Var2 were adoptively transferred to NCG (NOD-Prkdc^{em26Cd52}Il2rg^{em26Cd22}/NjuCrl) immunocompromised mice (Charles River Laboratories). 10 million BCMA-CAR+ iNKT were administered intravenously per mouse. Bleeds were collected at day 1, 2 and 5 post infusion and at days 7, 14, 21 and 28, bone marrow (FIG. 21B), lungs (FIG. 21E), liver (FIG. 21C), blood (FIG. 21A) and spleen (FIG. 21D) were collected to be analyzed by flow cytometry for iNKT detection and quantitation.

Briefly, organs were harvested and processed to obtain a single cell suspension. Cells were then incubated with LIVE/DEAD and then stained for human and mouse CD45 to identify human iNKT cells. INKT numbers were quantitated using counting beads (Invitrogen, 123count eBeads™ Counting Beads).

Anti-BCMA-CAR F6G-Var 2 IL-15 iNKT cells were found in increased number in blood, bone marrow, lung, liver and spleen compared to F6G-Var2 CAR iNKT cells that did not express IL-15.

Adoptive cell transfer of BCMA-CAR-IL-15 iNKT delayed engraftment of multiple myeloma MM1.s tumors in xenograft mice

10 million CAR+ BCMA-CAR-IL-15 (MiNK-413, F1G-IL-15, BB2121-IL-15), BCMA-CAR (F6G variant 2) and CD19-IL-15 expressing iNKT were adoptively transferred into NCG (NOD-Prkdc^{em26Cd52}Il2rg^{em26Cd22}/NjuCrl) immunocompromised mice (Charles River Laboratories) injected intravenously with 5 million MM1.s-GFP-nanoluciferase 5 days prior. On Day 15 and Day 33 after tumor injection, 3 mice were euthanized from each group and blood, bone marrow, spine and spleen were processed to make single-cell suspension for flow-cytometric analysis.

Briefly, organs were harvested and processed to obtain a single cell suspension. Cells are then incubated with LIVE/DEAD to discriminate against dead cells. To detect CAR expression, samples were first blocked with mouse and human FcX blocking antibody (Biolegend), and without washing the block, the cells were stained with primary BCMA-Fc or CD19-Fc. After washing the primary antibody, an A647-conjugated donkey anti-human IgG, Fc secondary antibody was added along with the remaining panel that included CD4 (BD, BUV563 OKT4) CD8 (BD, BUV395 RPA-T8), CD25 (Biolegend, BV510 BC96), CD69 (Biolegend, PE-Cy-7 FN50), TIGIT (eBioscience, PerCP-eFlour™ 710), PD-1 (Biolegend, BV605 EH12.2H7) to analyze cell surface markers. This was then followed with cellular fixation and permeabilization (eBioscience, Foxp3/Transcription Factor Staining Buffer Set) to stain for Ki-67 (BD, PE B56). After the cells have been stained for the desired markers, the samples were run in the flow-cytometer (BD Fortessa LSR). To quantify iNKT cell numbers, counting beads were added in flow-cytometry buffer (Invitrogen, 123count eBeads™ Counting Beads).

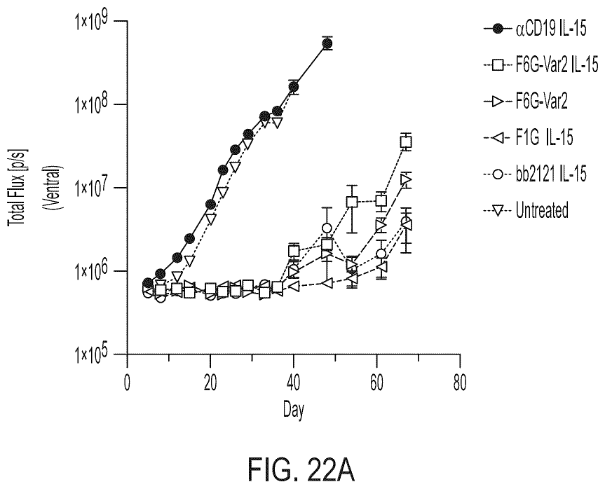

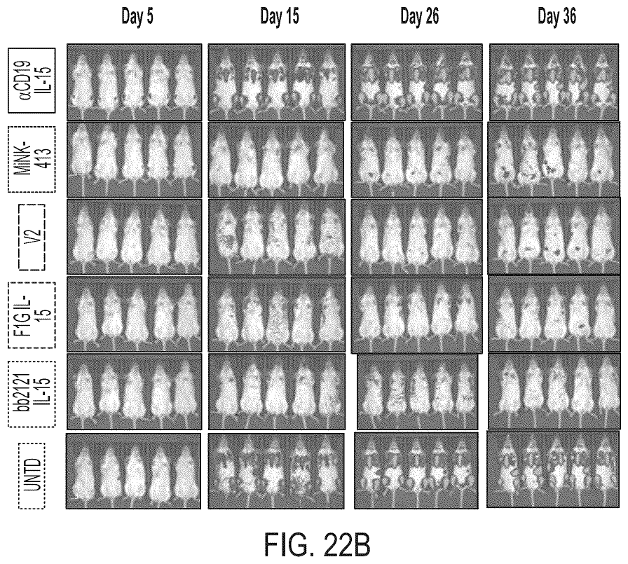

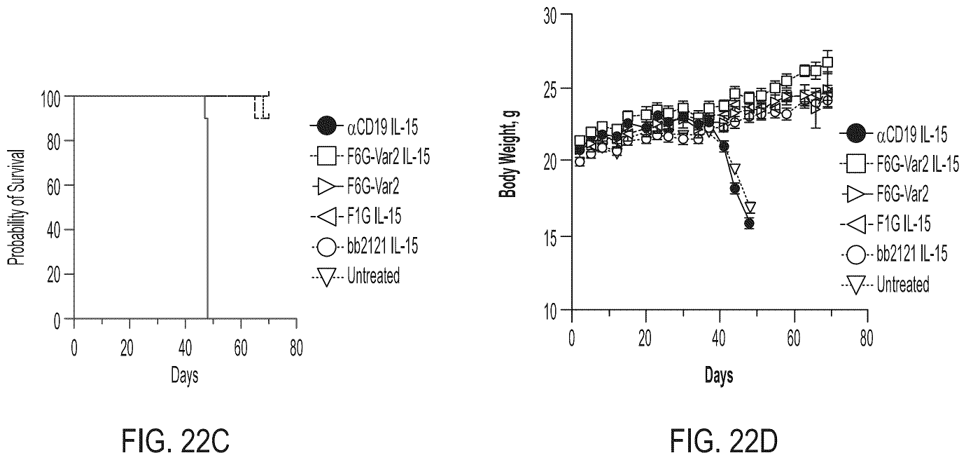

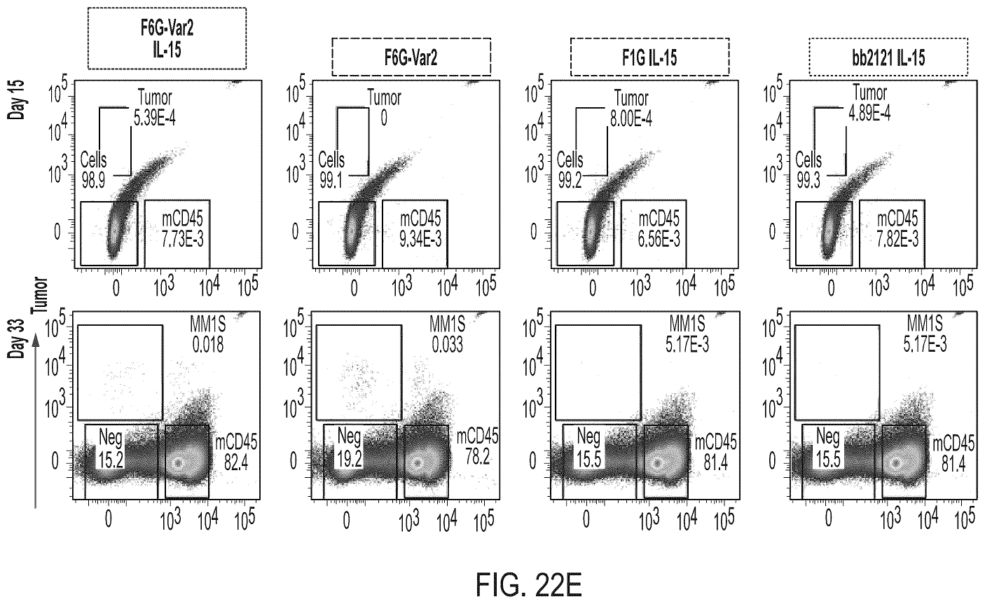

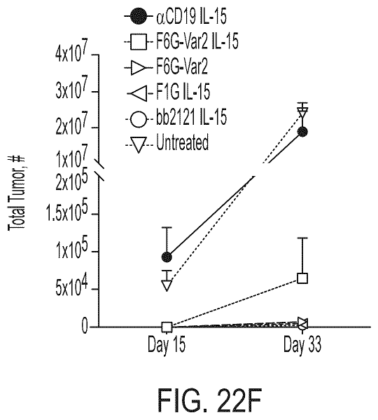

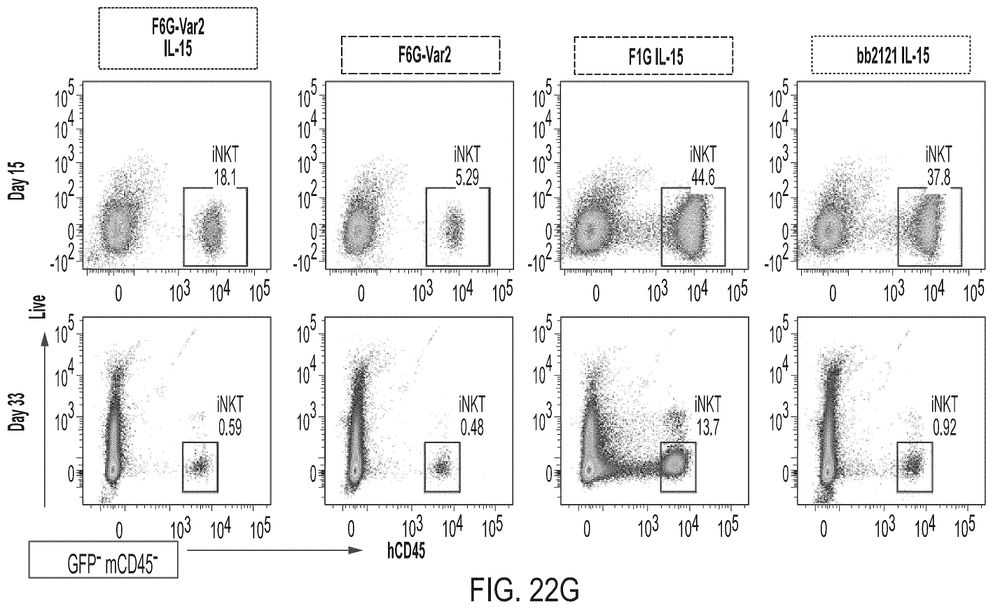

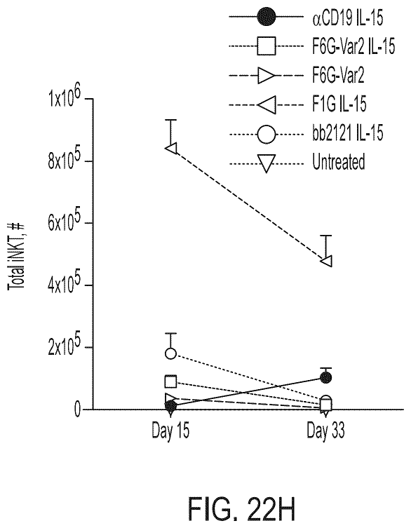

Anti-BCMA-CAR-IL-15 iNKT cells were tested in vivo for their anti-tumor properties. Anti-BCMA-CAR-IL-15 iNKT cells showed delayed tumor engraftment for 3 weeks after adoptive transfer (FIG. 22A). Survival of mice was enhanced for all BCMA-CAR-IL-15 cohorts (around 10-weeks) while control cohorts (CD19-IL-15 and Untreated cohorts) succumbed to tumor engraftment around 6-weeks post tumor-injection. Images of mice assessing for tumor engraftment/progression by IVIS imaging showed BCMA-CAR-IL-15 delaying tumor progression, but not for control cohorts (FIG. 22B).

Kaplan-Meier Survival graph showed significant survival benefit in mice treated with BCMA-CAR-IL-15 iNKT compared to CD19-IL-15 CAR and untreated mice cohorts (FIG. 22C). In addition, BCMA-CAR-IL-15 treated mice showed no CAR associated toxicity body weight loss (FIG. 22D). Flow-cytometric analysis of bone marrow tissues from all mice showed that at both harvest time-points, samples showed little/no traces of GFP-positive MM1.s indicating significant tumor control (FIGS. 22E-22F). Flow-cytometric analysis of bone marrow show persistence of BCMA-CAR-IL-15 at both harvest time-points. IL-15 as expressed in anti-BCMA CAR F6G-Var2 IL-15 iNKT cells compared to its non-IL-15 counterpart F6G Var. 6 indicate benefit in iNKT persistence at early time-point (FIGS. 22G-22H).

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B”, the disclosure also contemplates the alternative embodiments “a composition consisting of A and B” and “a composition consisting essentially of A and B”.

What is claimed is:

1. An invariant natural killer T (iNKT) cell engineered to express a chimeric antigen receptor comprising a B Cell Maturation Antigen (BCMA) binding moiety and IL-15.

2. The iNKT cell of claim 1, wherein the IL-15 is a soluble IL-15.

3. The iNKT cell of claim 1, wherein the IL-15 is a human IL-15.

4. The iNKT cell of claim 1, wherein the iNKT cell kills BCMA-expressing cells.

5. The iNKT cell of claim 4, wherein the iNKT cell kills BCMA expressing cells directly.

6. The iNKT cell of claim 4, wherein the iNKT cell kills BCMA expressing cells indirectly.

7. The iNKT cell of claim 1, wherein the iNKT cell retains a response to CDId and/or NK receptor ligand.

8. The iNKT cell of claim 1, wherein the iNKT cell comprises a CAR comprising any one of the anti-BCMA antibodies set forth in Table 3.

9. The iNKT cell of claim 1, wherein the iNKT cell comprises a CAR comprising any one of the CAR set forth in Table 4.

10. A method for killing a cell, the method comprising contacting the cell with the iNKT cell of claim 1.

11. A method for treating a tumor in a subject, the method comprising administering to a subject having or suspected of having cancer, the iNKT cell of claim 1.

12. A method for reducing tumor growth, the method comprising contacting the tumor in a subject with the iNKT cell of claim 1.

13. The method of claim 11, wherein the tumor cell express BCMA.

14. The method of claim 11, wherein the administration of the iNKT cells leads to reduced tumor burden relative to tumor burden in the subject prior to the administration.

15. The method of claim 11, wherein the administration of the iNKT cells leads to resistance to T cell exhaustion, enhancement of tissue homing of anti-BCMA iNKT cells, selective cytotoxicity towards M2 macrophages, and/or stimulation of dendritic cell maturation.

16. The method of claim 11, wherein the subject does not undergo lymphodepletion prior to administration of the iNKT cells.

17. An antibody or antigen binding fragment that specifically binds to an amino acid sequence having at least 85% identity to SEQ ID NOs: 38-48.

18. The antibody or antigen binding fragment of claim 17, wherein the antibody specifically binds an amino acid sequence set forth as: SEQ ID NOs: 38-48.

19. An antibody or antigen binding fragment that comprises a heavy chain variable region having the sequence set forth as in any one of SEQ ID NOs: 7, 12, 19 or 21.

20. The antibody or antigen binding fragment of claim 19, wherein the antibody comprises a heavy chain variable region having the sequence set forth as: SEQ ID NOs: 7, 12, 19 or 21 and a light chain variable region having a sequence set forth as: SEQ ID NO: 8, 10, 13, 15, or 22.

21. The antibody or antigen binding fragment of claim 17, wherein the antibody comprises:
(i) heavy chain variable region having the sequence set forth in SEQ ID NO: 7 and the light chain variable region having a sequence set forth in SEQ ID NO: 8;
(ii) heavy chain variable region having the sequence set forth in SEQ ID NO: 7 and the light chain variable region having a sequence set forth in SEQ ID NO: 10;
(iii) heavy chain variable region having the sequence set forth in SEQ ID NO: 12 and a light chain variable region having the sequence set forth in SEQ ID NO: 13;
(iv) heavy chain variable region having the sequence set forth in SEQ ID NO: 12 and a light chain variable region having the sequence set forth in SEQ ID NO: 15;
(v) heavy chain variable region having the sequence set forth in SEQ ID NO: 7 and a light chain variable region having the sequence set forth in SEQ ID NO: 13;
(vi) heavy chain variable region having the sequence set forth in SEQ ID NO: 7 and a light chain variable region having the sequence set forth in SEQ ID NO: 15;
(vii) heavy chain variable region having the sequence set forth in SEQ ID NO: 19 and a light chain variable region having the sequence set forth in SEQ ID NO: 10; or
(viii) heavy chain variable region having the sequence set forth in SEQ ID NO: 21 and the light chain variable region having a sequence set forth in SEQ ID NO: 22.

22. An antibody or antigen binding fragment that comprises a variable heavy chain region comprising a complementarity determining region 3 (CDRH3) having the sequence set forth in SEQ ID NOs: 3 or 33.

23. The antibody or antigen binding fragment of claim 22 further comprising a variable light chain region comprising a complementarity determining region 3 (CDRL3) having the sequence set forth in any one of SEQ ID NOs: 6, 29, 30, or 36.

24. An antibody or antigen binding fragment that comprises six complementarity determining regions (CDRs): CDRH1, CDRH2, CDRH3, CDRL1, CDRL2, and CDRL3, wherein
(i) CDRH1 comprises the sequence as set forth in SEQ ID NO: 1, CDRH2 comprises the sequence as set forth in SEQ ID NO: 2, CDRH3 comprises the sequence as set forth in SEQ ID NO: 3, CDRL1 comprises the sequence as set forth in SEQ ID NO: 4, CDRL2 comprises the sequence as set forth in SEQ ID NO: 5, and CDRL3 comprises the sequence as set forth in SEQ ID NO: 6;
(ii) CDRH1 comprises the sequence as set forth in SEQ ID NO: 1, CDRH2 comprises the sequence as set forth in SEQ ID NO: 2, CDRH3 comprises the sequence as set forth in SEQ ID NO: 3, CDRL1 comprises the sequence as set forth in SEQ ID NO: 4, CDRL2 comprises the sequence as set forth in SEQ ID NO: 5, and CDRL3 comprises the sequence as set forth in SEQ ID NO: 6;
(iii) CDRH1 comprises the sequence as set forth in SEQ ID NO: 25, CDRH2 comprises the sequence as set forth in SEQ ID NO: 27, CDRH3 comprises the sequence as set forth in SEQ ID NO: 3, CDRL1 comprises the sequence as set forth in SEQ ID NO: 4, CDRL2 comprises the sequence as set forth in SEQ ID NO: 24, and CDRL3 comprises the sequence as set forth in SEQ ID NO: 30;
(iv) CDRH1 comprises the sequence as set forth in SEQ ID NO: 25, CDRH2 comprises the sequence as set forth in SEQ ID NO: 27, CDRH3 comprises the sequence as set forth in SEQ ID NO: 3, CDRL1 comprises the sequence as set forth in SEQ ID NO: 4, CDRL2 comprises the sequence as set forth in SEQ ID NO: 24, and CDRL3 comprises the sequence as set forth in SEQ ID NO: 29;
(v) CDRH1 comprises the sequence as set forth in SEQ ID NO: 1, CDRH2 comprises the sequence as set forth in SEQ ID NO: 2, CDRH3 comprises the sequence as set forth in SEQ ID NO: 3, CDRL1 comprises the sequence as set forth in SEQ ID NO: 4, CDRL2 comprises the sequence as set forth in SEQ ID NO: 24, and CDRL3 comprises the sequence as set forth in SEQ ID NO: 30;
(vi) CDRH1 comprises the sequence as set forth in SEQ ID NO: 1, CDRH2 comprises the sequence as set forth in SEQ ID NO: 2, CDRH3 comprises the sequence as set forth in SEQ ID NO: 3, CDRL1 comprises the sequence as set forth in SEQ ID NO: 4, CDRL2 comprises the sequence as set forth in SEQ ID NO: 24, and CDRL3 comprises the sequence as set forth in SEQ ID NO: 29;
(vii) CDRH1 comprises the sequence as set forth in SEQ ID NO: 26, CDRH2 comprises the sequence as set forth in SEQ ID NO: 28, CDRH3 comprises the sequence as set forth in SEQ ID NO: 3, CDRL1 comprises the sequence as set forth in SEQ ID NO: 4, CDRL2 comprises the sequence as set forth in SEQ ID NO: 24, and CDRL3 comprises the sequence as set forth in SEQ ID NO: 6; or
(viii) CDRH1 comprises the sequence as set forth in SEQ ID NO: 31, CDRH2 comprises the sequence as set forth in SEQ ID NO: 32, CDRH3 comprises the sequence as set forth in SEQ ID NO: 33, CDRL1 comprises the sequence as set forth in SEQ ID NO: 34, CDRL2 comprises the sequence as set forth in SEQ ID NO: 35, and CDRL3 comprises the sequence as set forth in SEQ ID NO: 36.

25. The antibody or antigen binding fragment of claim 17, wherein the antibody or antigen binding fragment is chimeric.

26. The antibody or antigen binding fragment of claim 17, wherein the antibody or antigen binding fragment is humanized.

27. The antibody or antigen binding fragment of claim 17, wherein the antibody or antigen binding fragment is a single-chain variable fragment (scFv).

28. The antibody or antigen binding fragment of claim 27, wherein the scFv comprises the amino acid sequence set forth of any one of SEQ ID NOs: 9, 11, 14, 16, 17, 18, 20, or 23.

29. An isolated nucleic acid encoding the antibody or antigen binding fragment of claim 17.

30. The isolated nucleic acid of claim 29 comprising the sequence set forth in any one of SEQ ID NOs: 49-64.

31. The isolated nucleic acid of claim 29 comprising the sequence set forth in any one of SEQ ID NOs: 65-72.

32. A vector comprising the isolated nucleic acid of claim 29.

33. The vector of claim 32, wherein the vector is a viral vector.

34. The vector of claim 33, wherein the viral vector is a lentiviral vector.

35. The vector of claim 34, wherein the lentiviral vector comprises the sequence set forth in any one of SEQ ID NOs: 73-80.

36. A host cell comprising the antibody or antigen binding fragment of claim 17.

37. The host cell of claim 36, wherein the cell is a mammalian cell, bacterial cell, yeast cell, or insect cell.

38. The host cell of claim 36, wherein the cell is a hybridoma cell.

39. A chimeric antigen receptor (CAR) comprising the antigen binding fragment of claim 17.

40. The CAR of claim 39, wherein the CAR further comprises a hinge region.

41. The CAR of claim 40, wherein the hinge region comprises an amino acid sequence at least 80% identical to SEQ ID NO: 82.

42. The CAR of claim 39, wherein the CAR further comprises a transmembrane domain.

43. The CAR of claim 39, wherein the transmembrane domain comprises an amino acid sequence at least 80% identical to SEQ ID NO: 83.

44. The CAR of claim 39, wherein the CAR further comprises a cytoplasmic domain.

45. The CAR of claim 44, wherein the cytoplasmic domain comprises an amino acid sequence at least 80% identical to SEQ ID NO: 84.

46. The CAR of claim 39, wherein the CAR further comprises a costimulatory domain.

47. The CAR of claim 46, wherein the co-stimulatory domain comprises an amino acid sequence at least 80% identical to SEQ ID NO: 85.

48. The CAR of claim 47, wherein the CAR comprises the amino acid sequence of any one of SEQ ID NOs: 86-93.

49. An immune cell comprising the CAR of claim 39.

50. The immune cell of claim 49, wherein the immune cell is a natural killer (NK) cell or a T cell.

51. The immune cell of claim 50, wherein the immune cell is an invariant natural killer T (iNKT) cell.

52. The immune cell of claim 49, wherein the immune cell is engineered to express one or more immunoregulatory gene products.

53. The immune cell of claim 52, wherein the one or more immunoregulatory gene products comprises IL-15, IL-12, CD40L, or 4-IBB.

54. A pharmaceutical composition comprising the antibody or antigen binding fragment of claim 17 and a pharmaceutically acceptable excipient.

55. A method for killing a cell, the method comprising contacting the cell with the antibody or antigen binding fragment of claim 17.

56. The method of claim 55, wherein the cell is a mammalian cell.

57. The method of claim 55, wherein the cell is a human cell.

58. The method of claim 55, wherein the cell is a cancer cell.

59. The method of claim 58, wherein the cancer is a blood cancer.

60. The method of claim 59, wherein the blood cancer is a leukemia or lymphoma.

61. The method of claim 55, wherein the cancer is multiple myeloma.

62. A method for treating cancer in a subject, the method comprising administering to a subject having or suspected of having cancer, the immune cell of claim 49.

63. A method for reducing tumor growth, the method comprising contacting the tumor in a subject with the antibody or antigen binding fragment of claim 17.

64. The method of claim 62, wherein the subject is a human.

65. The method of claim 62, wherein the cancer is a blood cancer.

66. The method of claim 65, wherein the cancer is a leukemia or lymphoma.

67. The method of claim 62, wherein the cancer is multiple myeloma.

68. The method of claim 62, wherein the administration is via injection.

69. The method of claim 62, wherein the subject does not undergo lymphodepletion prior to administration of the iNKT cells.


SEQUENCE LISTING (111 sequences)

SEQ ID NO: 1 - protein, 5 aa, synthetic construct
GYYMH

SEQ ID NO: 2 - protein, 17 aa, synthetic construct
WMNPNSGNTGYAQKFQG

SEQ ID NO: 3 - protein, 8 aa, synthetic construct
AQWEPLDY

SEQ ID NO: 4 - protein, 10 aa, synthetic construct
SGDALPKKYT

SEQ ID NO: 5 - protein, 7 aa, synthetic construct
EDTKRPS

SEQ ID NO: 6 - protein, 11 aa, synthetic construct
LSTDPTGQESV

SEQ ID NO: 7 - protein, 117 aa, synthetic construct
QVQLVQSGAEVKKPGASVKVSCKASGYTFTGYYMHWVRQAPGQGLEWMGWMNPNSGNTGYAQKFQGRVTMTRNTSISTAYMELSSLRSEDTAVYYCARAQWEPLDYWGQGTLVTVSS

SEQ ID NO: 8 - protein, 111 aa, synthetic construct
SYELTQPPSVSVSPGQTARITCSGDALPKKYTYWYQQKSGQAPVLVIYEDTKRPSGIPERFSGSSSGTMATLTISGAQVEDEADYYCLSTDPTGQESVFGGGTKLTVLGQP

SEQ ID NO: 9 - protein, 243 aa, synthetic construct
QVQLVQSGAEVKKPGASVKVSCKASGYTFTGYYMHWVRQAPGQGLEWMGWMNPNSGNTGYAQKFQGRVTMTRNTSISTAYMELSSLRSEDTAVYYCARAQWEPLDYWGQGTLVTVSSGGGGSGGGGSGGGASSYELTQPPSVSVSPGQTARITCSGDALPKKYTYWYQQKSGQAPVLVIYEDTKRPSGIPERFSGSSSGTMATLTISGAQVEDEADYYCLSTDPTGQESVFGGGTKLTVLGQP

SEQ ID NO: 10 - protein, 111 aa, synthetic construct
SYELTQPPSVSVSPGQTARITCSGDALPKKYTYWYQQKSGQAPVLVIYYDTKRPSGIPERFSGSSSGTMATLTISGAQVEDEADYYCLSTDPTGQESVFGGGTKLTVLGQP

SEQ ID NO: 11 - protein, 243 aa, synthetic construct
QVQLVQSGAEVKKPGASVKVSCKASGYTFTGYYMHWVRQAPGQGLEWMGWMNPNSGNTGYAQKFQGRVTMTRNTSISTAYMELSSLRSEDTAVYYCARAQWEPLDYWGQGTLVTVSSGGGGSGGGGSGGGASSYELTQPPSVSVSPGQTARITCSGDALPKKYTYWYQQKSGQAPVLVIYYDTKRPSGIPERFSGSSSGTMATLTISGAQVEDEADYYCLSTDPTGQESVFGGGTKLTVLGQP

SEQ ID NO: 12 - protein, 117 aa, synthetic construct
QVQLVQSGAEVKKPGASVKVSCKASGYTFTGYYMQWVRQAPGQGLEWMGWMNPISGNTGYAQKFQGRVTMTRNTSISTAYMELSSLRSEDTAVYYCARAQWEPLDYWGQGTLVTVSS

SEQ ID NO: 13 - protein, 111 aa, synthetic construct
SYELTQPPSVSVSPGQTARITCSGDALPKKYTYWYQQKSGQAPVLVIYYDTKRPSGIPERFSGSSSGTMATLTISGAQVEDEADYYCLSTHPAGQESVFGGGTKLTVLGQP

SEQ ID NO: 14 - protein, 243 aa, synthetic construct
QVQLVQSGAEVKKPGASVKVSCKASGYTFTGYYMQWVRQAPGQGLEWMGWMNPISGNTGYAQKFQGRVTMTRNTSISTAYMELSSLRSEDTAVYYCARAQWEPLDYWGQGTLVTVSSGGGGSGGGGSGGGASSYELTQPPSVSVSPGQTARITCSGDALPKKYTYWYQQKSGQAPVLVIYYDTKRPSGIPERFSGSSSGTMATLTISGAQVEDEADYYCLSTHPAGQESVFGGGTKLTVLGQP

SEQ ID NO: 15 - protein, 111 aa, synthetic construct
SYELTQPPSVSVSPGQTARITCSGDALPKKYTYWYQQKSGQAPVLVIYYDTKRPSGIPERFSGSSSGTMATLTISGAQVEDEADYYCLSTNPTGHESVFGGGTKLTVLGQP

SEQ ID NO: 16 - protein, 243 aa, synthetic construct
QVQLVQSGAEVKKPGASVKVSCKASGYTFTGYYMQWVRQAPGQGLEWMGWMNPISGNTGYAQKFQGRVTMTRNTSISTAYMELSSLRSEDTAVYYCARAQWEPLDYWGQGTLVTVSSGGGGSGGGGSGGGASSYELTQPPSVSVSPGQTARITCSGDALPKKYTYWYQQKSGQAPVLVIYYDTKRPSGIPERFSGSSSGTMATLTISGAQVEDEADYYCLSTNPTGHESVFGGGTKLTVLGQP

SEQ ID NO: 17 - protein, 243 aa, synthetic construct
QVQLVQSGAEVKKPGASVKVSCKASGYTFTGYYMHWVRQAPGQGLEWMGWMNPNSGNTGYAQKFQGRVTMTRNTSISTAYMELSSLRSEDTAVYYCARAQWEPLDYWGQGTLVTVSSGGGGSGGGGSGGGASSYELTQPPSVSVSPGQTARITCSGDALPKKYTYWYQQKSGQAPVLVIYYDTKRPSGIPERFSGSSSGTMATLTISGAQVEDEADYYCLSTHPAGQESVFGGGTKLTVLGQP

SEQ ID NO: 18 - protein, 243 aa, synthetic construct
QVQLVQSGAEVKKPGASVKVSCKASGYTFTGYYMHWVRQAPGQGLEWMGWMNPNSGNTGYAQKFQGRVTMTRNTSISTAYMELSSLRSEDTAVYYCARAQWEPLDYWGQGTLVTVSSGGGGSGGGGSGGGASSYELTQPPSVSVSPGQTARITCSGDALPKKYTYWYQQKSGQAPVLVIYYDTKRPSGIPERFSGSSSGTMATLTISGAQVEDEADYYCLSTNPTGHESVFGGGTKLTVLGQP

SEQ ID NO: 19 - protein, 117 aa, synthetic construct
QVQLVQSGAEVKKPGASVKVSCKASGYTFTEYYMKWVRQAPGQGLEWMGWMNPNTGKTGYAQKFQGRVTMTRNTSISTAYMELSSLRSEDTAVYYCARAQWEPLDYWGQGTLVTVSS

SEQ ID NO: 20 - protein, 243 aa, synthetic construct
QVQLVQSGAEVKKPGASVKVSCKASGYTFTEYYMKWVRQAPGQGLEWMGWMNPNTGKTGYAQKFQGRVTMTRNTSISTAYMELSSLRSEDTAVYYCARAQWEPLDYWGQGTLVTVSSGGGGSGGGGSGGGASSYELTQPPSVSVSPGQTARITCSGDALPKKYTYWYQQKSGQAPVLVIYYDTKRPSGIPERFSGSSSGTMATLTISGAQVEDEADYYCLSTDPTGQESVFGGGTKLTVLGQP

SEQ ID NO: 21 - protein, 123 aa, synthetic construct
QVQLVQSGAEVKKPGASVKVSCKASGYTFTSYGISWVRQAPGQGLEWMGWISAYNGNTYYAEDLQGRLTMTTDTSTSTAYMELRSLRSDDTAVYYCAREGSYYDSSGYYLPYWGQGTLVTVSS

SEQ ID NO: 22 - protein, 112 aa, synthetic construct
QSALTQPRSVSGSPGQSVTISCTGTSSDVGAYNYVSWYQQHPGKAPKLMIYDVTERPSGVPDRFSGSKSGNTASLTISGLQAEDEADYYCSSYTSSSIAFGGGTKLTVLGQP

SEQ ID NO: 23 - protein, 250 aa, synthetic construct
QVQLVQSGAEVKKPGASVKVSCKASGYTFTSYGISWVRQAPGQGLEWMGWISAYNGNTYYAEDLQGRLTMTTDTSTSTAYMELRSLRSDDTAVYYCAREGSYYDSSGYYLPYWGQGTLVTVSSGGGGSGGGGSGGGASQSALTQPRSVSGSPGQSVTISCTGTSSDVGAYNYVSWYQQHPGKAPKLMIYDVTERPSGVPDRFSGSKSGNTASLTISGLQAEDEADYYCSSYTSSSIAFGGGTKLTVLGQP

SEQ ID NO: 24 - protein, 7 aa, synthetic construct
YDTKRPS

SEQ ID NO: 25 - protein, 5 aa, synthetic construct
GYYMQ

SEQ ID NO: 26 - protein, 5 aa, synthetic construct
EYYMK

SEQ ID NO: 27 - protein, 17 aa, synthetic construct
WMNPISGNTGYAQKFQG

SEQ ID NO: 28 - protein, 17 aa, synthetic construct
WMNPNTGKTGYAQKFQG

SEQ ID NO: 29 - protein, 11 aa, synthetic construct
LSTNPTGHESV

SEQ ID NO: 30 - protein, 11 aa, synthetic construct
LSTHPAGQESV

SEQ ID NO: 31 - protein, 5 aa, synthetic construct
SYGIS

SEQ ID NO: 32 - protein, 17 aa, synthetic construct
WISAYNGNTYYAEDLQG

SEQ ID NO: 33 - protein, 14 aa, synthetic construct
EGSYYDSSGYYLPY

SEQ ID NO: 34 - protein, 14 aa, synthetic construct
TGTSSDVGAYNYVS

SEQ ID NO: 35 - protein, 7 aa, synthetic construct
DVTERPS

SEQ ID NO: 36 - protein, 9 aa, synthetic construct
SSYTSSSIA

SEQ ID NO: 37 - skipped

SEQ ID NO: 38 - protein, 54 aa, synthetic construct
MLQMAGQCSQNEYFASALHACIPCQLRCSSNTPPLTCQRYCNASVTNSVKGTNA

SEQ ID NO: 39 - protein, 54 aa, synthetic construct
MLQMAGACSANEYFDSLLHACIPCQLRCSSNTPPLTCQRYCNASVTNSVKGTNA

SEQ ID NO: 40 - protein, 54 aa, synthetic construct
MLQMAGQCSQNEYFAALLHACIPCQLRCSSNTPPLTCQRYCNASVTNSVKGTNA

SEQ ID NO: 41 - protein, 54 aa, synthetic construct
MLQMAGQCSQNEYFDSAAHACIPCQLRCSSNTPPLTCQRYCNASVTNSVKGTNA

SEQ ID NO: 42 - protein, 54 aa, synthetic construct
MLQMAGQCSQNEYFDSLLHACIPCQAACSSNTPPLTCQRYCNASVTNSVKGTNA

SEQ ID NO: 43 - protein, 54 aa, synthetic construct
MLQMAGQCSQNEYFDSLLHACIPCQLRCSAATPPLTCQRYCNASVTNSVKGTNA

SEQ ID NO: 44 - protein, 54 aa, synthetic construct
MLQMAGQCSQNEYFDSLLHACIPCQLRCSSNAPPATCQRYCNASVTNSVKGTNA

SEQ ID NO: 45 - protein, 54 aa, synthetic construct
MLQMAGQCSQNEYFDSLLHACIPCQLRCSSNTPPLACARYCNASVTNSVKGTNA

SEQ ID NO: 46 - protein, 54 aa, synthetic construct
MLQMAGQCSQNEYFDSLLHACIPCQLRCSSNTPPLTCQAYCAASVTNSVKGTNA

SEQ ID NO: 47 - protein, 42 aa, synthetic construct
MLQMAGQCSQNEYFDSLLHACIPCQLRCSSNTPPLTCQRYCN

SEQ ID NO: 48 - protein, 54 aa, Homo sapiens
MLQMAGQCSQNEYFDSLLHACIPCQLRCSSNTPPLTCQRYCNASVTNSVKGTNA

SEQ ID NO: 49 - DNA, 350 bp, synthetic construct
  source 1..350: mol_type = other DNA
caagttcaactggttcagtctggcgccgaagtgaagaaacctggcgcctctgtgaaggtgtcctgcaaggccagcggctacacctttaccggctactacatgcactgggtccgacaggctccaggccaaggacttgaatggatgggctggatgaaccccaacagcggcaataccggctatgcccagaaattccagggcagagtgaccatgaccagaaacaccagcatcagcaccgcctacatggaactgagcagcctgagaagcgaggacaccgccgtgtactactgtgctagagcccagtgggagcccctggattattggggacagggcacactggtcacagtctcgag

SEQ ID NO: 50 - DNA, 350 bp, synthetic construct; identical to SEQ ID NO: 49
  source 1..350: mol_type = other DNA

SEQ ID NO: 51 - DNA, 350 bp, synthetic construct
  source 1..350: mol_type = other DNA
caggtgcaattggtgcaatcaggggccgaagtcaagaaaccgggtgcttcagtcaaggtttcttgtaaagcatctggctatacgtttactgggtactacatgcaatgggtgagacaagctcctggccaagggcttgaatggatggggtggatgaatccaatctctggaaatacaggctacgcccaaaagttccagggaagagttaccatgacacggaatacgtccatttccacggcatacatggaactgtcctcccttcggagcgaggacactgcagtatattactgcgcgagagcacaatgggaaccgcttgattattggggtcaaggtaccttggttactgtgtcctc

SEQ ID NO: 52 - DNA, 350 bp, synthetic construct
  source 1..350: mol_type = other DNA
caggtccaattggtccaatctggcgccgaggtcaagaagcctggcgcatccgtaaaagtgtcttgtaaagcctctggttatacgtttacggggtattatatgcaatgggttcggcaagcccctggtcaaggacttgaatggatgggctggatgaaccctatctctggtaatacgggctacgcccaaaagttccagggtagagtcacaatgacgcgcaacacgtcaatatcaaccgcttacatggagcttagttctttgaggtcagaggacaccgcggtctattattgtgctcgcgcccaatgggaaccgcttgattactgggggcaaggtaccttggttaccgtctcatc

SEQ ID NO: 53 - DNA, 350 bp, synthetic construct
  source 1..350: mol_type = other DNA
caggtccagctggtacagtctggcgctgaggtaaagaagcctggtgcttcagtgaaggtgtcttgcaaagcctctggctatactttcacaggttattatatgcactgggtgcgccaggcccccggtcagggccttgaatggatgggatggatgaatccaaattcaggcaataccgggtatgctcaaaagtttcagggccgcgtaacaatgacccgaaatacaagtatttcaactgcttatatggaactcagttctctcagaagtgaagatacggccgtatattattgcgcgcgggcgcaatgggaaccacttgactattggggacaaggcacactggttacggtgtcatc

SEQ ID NO: 54 - DNA, 350 bp, synthetic construct
  source 1..350: mol_type = other DNA
caggtccaactggtgcaatctggcgctgaagtgaaaaagcccggcgcatcagtaaaggtgagttgcaaagcatctgggtacactttcactggctattatatgcattgggtaaggcaagcccccggccaaggcctggaatggatgggatggatgaatccgaactccggtaatacagggtacgctcaaaaatttcaaggacgggtcacaatgacgcggaacaccagtatatctactgcatatatggagctcagttcactgcgctccgaggatacagctgtatactattgcgccagggctcaatgggaaccactggactattggggtcaagggacacttgtaacggtaagctc

SEQ ID NO: 55 - DNA, 351 bp, synthetic construct
  source 1..351: mol_type = other DNA
caggttcaacttgttcaaagcggtgcagaagttaagaaaccaggtgccagcgttaaggtcagctgtaaagcgagtgggtacactttcaccgaatattatatgaaatgggtacgacaagcaccagggcagggactcgaatggatggggtggatgaaccccaacactggcaagacgggctacgcacagaaattccaagggcgagtgaccatgaccagaaatacgtcaatttcaaccgcatacatggagctgtcatccttgcgatccgaggacaccgcggtatattattgcgcgcgcgctcaatgggagccacttgattattggggacagggcacgcttgtcactgtatcctct

SEQ ID NO: 56 - DNA, 368 bp, synthetic construct
  source 1..368: mol_type = other DNA
caagttcaactggttcagtctggcgccgaagtgaagaaacctggcgcctctgtgaaggtgtcctgcaaggccagcggctacacctttaccagctacggcatcagctgggtccgacaggctcctggacaaggcttggaatggatgggctggatcagcgcctacaacggcaacacctactacgccgaggatctgcagggcagactgaccatgaccaccgataccagcaccagcaccgcctacatggaactgagaagcctgagatccgacgacaccgccgtgtactactgtgccagagagggcagctactacgacagctccggctactacctgccttattggggccagggcacactggtcacagtctcgag

SEQ ID NO: 57 - DNA, 333 bp, synthetic construct
  source 1..333: mol_type = other DNA
tcttatgaactgacccagcctccaagcgtgtccgtgtctcctggacagaccgccagaatcacatgtagcggagatgccctgcctaagaagtacacctactggtatcagcagaagtccggacaggcccctgtgctggtcatctacgaggataccaaacggcccagcggcatccccgagagattttctggaagcagcagcggcacaatggccacactgacaattagcggagcccaggtggaagatgaggccgactactactgcctgagcaccgatcctacaggccaagagtccgtttttggcggaggcaccaagctgacagtgctgggacaacct

SEQ ID NO: 58 - DNA, 333 bp, synthetic construct
  source 1..333: mol_type = other DNA
tcttatgaactgacccagcctccaagcgtgtccgtgtctcctggacagaccgccagaatcacatgtagcggagatgccctgcctaagaagtacacctactggtatcagcagaagtccggacaggcccctgtgctggtcatctactatgataccaaacggcccagcggcatccccgagagattttctggaagcagcagcggcacaatggccacactgacaattagcggagcccaggtggaagatgaggccgactactactgcctgagcaccgatcctacaggccaagagtccgtttttggcggaggcaccaagctgacagtgctgggacaacct

SEQ ID NO: 59 - DNA, 333 bp, synthetic construct
  source 1..333: mol_type = other DNA
agctatgaactgactcagccaccgagtgtatctgtaagtccgggtcagacagcgcggatcacttgctcaggggacgcccttcccaaaaaatacacttactggtatcaacagaagagtgggcaggctcctgtgttggtaatatactatgacacgaaacggcctagcgggatcccggagcggttctcaggttcctccagtgggacgatggctacactgacgatcagcggggcccaagtggaggacgaagctgattattattgccttagtacccaccccgccggacaggagtctgtgtttggtggcggcaccaagcttacggtactcggacagcct

SEQ ID NO: 60 - DNA, 333 bp, synthetic construct
  source 1..333: mol_type = other DNA
agctacgaactgacacagccgccctctgttagcgttagtcctggacagaccgccaggataacgtgctccggtgacgcattgccgaagaaatatacgtactggtaccagcagaagtccgggcaagcacctgtactcgtgatctattatgatacaaaacggccgagcggtataccagaacgctttagcggctcaagcagcggaacgatggccacgttgacgattagcggcgcgcaggtcgaggacgaagctgactactattgcctcagtactaacccaacagggcacgaatcagtttttggagggggaaccaagcttactgtacttggtcagccg

SEQ ID NO: 61 - DNA, 333 bp, synthetic construct
  source 1..333: mol_type = other DNA
tcatatgaactcacacagccgccatctgttagcgtttctccagggcagactgcccgaattacatgctcaggcgatgccctcccgaaaaaatatacctactggtaccaacaaaaatccggccaggcacctgtgttggtgatatattatgatacaaaacgcccttcagggatcccggagagattcagtgggtcaagtagcggaacaatggcaacacttactataagcggggcacaggttgaggacgaggctgactactactgcctgtctactcatccggccggacaggagagtgttttcgggggtggaactaaactcacagtactgggacagccc

SEQ ID NO: 62 - DNA, 333 bp, synthetic construct
  source 1..333: mol_type = other DNA
tcctatgagcttactcagccaccctctgtatctgtatctcctgggcagacagcacgcatcacatgtagtggcgatgccctcccgaaaaaatacacgtattggtatcaacagaaatccggacaggcaccggttttggtgatatactatgatacgaaaagacctagtggaatacctgagcggttttctggaagttcttctggtaccatggcaactttgacaatctcaggtgcccaggtcgaggacgaggccgactattattgtttgtctacaaatccgacaggtcatgaatccgtgttcggcggagggacaaagcttaccgtccttgggcagcct

SEQ ID NO: 63 - DNA, 333 bp, synthetic construct
  source 1..333: mol_type = other DNA
tcatatgaacttactcagccgccttctgttagtgtaagccctgggcagactgcacgcattacatgcagtggtgacgccttgccaaaaaaatatacttattggtaccaacaaaagagtggacaggcgccggtcttggtgatatactatgacactaagagaccctctgggataccggaacgcttttctggttcaagttccggaacgatggcgaccttgacgatcagcggggcccaagtcgaggatgaggccgactactactgtttgtctacagatcccacaggccaagaaagcgtgttcggaggtgggactaaacttacggtcttgggtcaacca

SEQ ID NO: 64 - DNA, 336 bp, synthetic construct
  source 1..336: mol_type = other DNA
caatctgctctgacacagcccagaagcgtgtccggatctcctggccagagcgtgaccatcagctgtacaggcaccagctctgatgtgggcgcctacaactacgtgtcctggtatcagcagcaccccggcaaggcccctaagctgatgatctacgatgtgaccgagaggcctagcggcgtgcccgatagattttctggcagcaagagcggcaacaccgccagcctgacaattagcggactgcaggccgaggacgaggccgattactactgtagcagctacaccagcagctctatcgcctttggcggaggcaccaagctgacagttctgggacaacct

SEQ ID NO: 65 - DNA, 729 bp, synthetic construct
  source 1..729: mol_type = other DNA
caagttcaactggttcagtctggcgccgaagtgaagaaacctggcgcctctgtgaaggtgtcctgcaaggccagcggctacacctttaccggctactacatgcactgggtccgacaggctccaggccaaggacttgaatggatgggctggatgaaccccaacagcggcaataccggctatgcccagaaattccagggcagagtgaccatgaccagaaacaccagcatcagcaccgcctacatggaactgagcagcctgagaagcgaggacaccgccgtgtactactgtgctagagcccagtgggagcccctggattattggggacagggcacactggtcacagtctcgagtggtggaggcggttcaggcggaggtggctctggcggtggcgctagctcttatgaactgacccagcctccaagcgtgtccgtgtctcctggacagaccgccagaatcacatgtagcggagatgccctgcctaagaagtacacctactggtatcagcagaagtccggacaggcccctgtgctggtcatctacgaggataccaaacggcccagcggcatccccgagagattttctggaagcagcagcggcacaatggccacactgacaattagcggagcccaggtggaagatgaggccgactactactgcctgagcaccgatcctacaggccaagagtccgtttttggcggaggcaccaagctgacagtgctgggacaacct

SEQ ID NO: 66 - DNA, 729 bp, synthetic construct
  source 1..729: mol_type = other DNA
caagttcaactggttcagtctggcgccgaagtgaagaaacctggcgcctctgtgaaggtgtcctgcaaggccagcggctacacctttaccggctactacatgcactgggtccgacaggctccaggccaaggacttgaatggatgggctggatgaaccccaacagcggcaataccggctatgcccagaaattccagggcagagtgaccatgaccagaaacaccagcatcagcaccgcctacatggaactgagcagcctgagaagcgaggacaccgccgtgtactactgtgctagagcccagtgggagcccctggattattggggacagggcacactggtcacagtctcgagtggtggaggcggttcaggcggaggtggctctggcggtggcgctagctcttatgaactgacccagcctccaagcgtgtccgtgtctcctggacagaccgccagaatcacatgtagcggagatgccctgcctaagaagtacacctactggtatcagcagaagtccggacaggcccctgtgctggtcatctactatgataccaaacggcccagcggcatccccgagagattttctggaagcagcagcggcacaatggccacactgacaattagcggagcccaggtggaagatgaggccgactactactgcctgagcaccgatcctacaggccaagagtccgtttttggcggaggcaccaagctgacagtgctgggacaacct

SEQ ID NO: 67 - DNA, 729 bp, synthetic construct
  source 1..729: mol_type = other DNA
caggtgcaattggtgcaatcaggggccgaagtcaagaaaccgggtgcttcagtcaaggtttcttgtaaagcatctggctatacgtttactgggtactacatgcaatgggtgagacaagctcctggccaagggcttgaatggatggggtggatgaatccaatctctggaaatacaggctacgcccaaaagttccagggaagagttaccatgacacggaatacgtccatttccacggcatacatggaactgtcctcccttcggagcgaggacactgcagtatattactgcgcgagagcacaatgggaaccgcttgattattggggtcaaggtaccttggttactgtgtcctcaggtggaggcggttcaggcggaggtggctctggcggtggcgctagcagctatgaactgactcagccaccgagtgtatctgtaagtccgggtcagacagcgcggatcacttgctcaggggacgcccttcccaaaaaatacacttactggtatcaacagaagagtgggcaggctcctgtgttggtaatatactatgacacgaaacggcctagcgggatcccggagcggttctcaggttcctccagtgggacgatggctacactgacgatcagcggggcccaagtggaggacgaagctgattattattgccttagtacccaccccgccggacaggagtctgtgtttggtggcggcaccaagcttacggtactcggacagcct

SEQ ID NO: 68 - DNA, 729 bp, synthetic construct
  source 1..729: mol_type = other DNA
caggtccaattggtccaatctggcgccgaggtcaagaagcctggcgcatccgtaaaagtgtcttgtaaagcctctggttatacgtttacggggtattatatgcaatgggttcggcaagcccctggtcaaggacttgaatggatgggctggatgaaccctatctctggtaatacgggctacgcccaaaagttccagggtagagtcacaatgacgcgcaacacgtcaatatcaaccgcttacatggagcttagttctttgaggtcagaggacaccgcggtctattattgtgctcgcgcccaatgggaaccgcttgattactgggggcaaggtaccttggttaccgtctcatcaggtggaggcggttcaggcggaggtggctctggcggtggcgctagcagctacgaactgacacagccgccctctgttagcgttagtcctggacagaccgccaggataacgtgctccggtgacgcattgccgaagaaatatacgtactggtaccagcagaagtccgggcaagcacctgtactcgtgatctattatgatacaaaacggccgagcggtataccagaacgctttagcggctcaagcagcggaacgatggccacgttgacgattagcggcgcgcaggtcgaggacgaagctgactactattgcctcagtactaacccaacagggcacgaatcagtttttggagggggaaccaagcttactgtacttggtcagccg

SEQ ID NO: 69 - DNA, 729 bp, synthetic construct
  source 1..729: mol_type = other DNA
caggtccagctggtacagtctggcgctgaggtaaagaagcctggtgcttcagtgaaggtgtcttgcaaagcctctggctatactttcacaggttattatatgcactgggtgcgccaggcccccggtcagggccttgaatggatgggatggatgaatccaaattcaggcaataccgggtatgctcaaaagtttcagggccgcgtaacaatgacccgaaatacaagtatttcaactgcttatatggaactcagttctctcagaagtgaagatacggccgtatattattgcgcgcgggcgcaatgggaaccacttgactattggggacaaggcacactggttacggtgtcatctggtggaggcggttcaggcggaggtggctctggcggtggcgctagctcatatgaactcacacagccgccatctgttagcgtttctccagggcagactgcccgaattacatgctcaggcgatgccctcccgaaaaaatatacctactggtaccaacaaaaatccggccaggcacctgtgttggtgatatattatgatacaaaacgcccttcagggatcccggagagattcagtgggtcaagtagcggaacaatggcaacacttactataagcggggcacaggttgaggacgaggctgactactactgcctgtctactcatccggccggacaggagagtgttttcgggggtggaactaaactcacagtactgggacagccc

SEQ ID NO: 70 - DNA, 729 bp, synthetic construct
  source 1..729: mol_type = other DNA
caggtccaactggtgcaatctggcgctgaagtgaaaaagcccggcgcatcagtaaaggtgagttgcaaagcatctgggtacactttcactggctattatatgcattgggtaaggcaagcccccggccaaggcctggaatggatgggatggatgaatccgaactccggtaatacagggtacgctcaaaaatttcaaggacgggtcacaatgacgcggaacaccagtatatctactgcatatatggagctcagttcactgcgctccgaggatacagctgtatactattgcgccagggctcaatgggaaccactggactattggggtcaagggacacttgtaacggtaagctcaggtggaggcggttcaggcggaggtggctctggcggtggcgctagctcctatgagcttactcagccaccctctgtatctgtatctcctgggcagacagcacgcatcacatgtagtggcgatgccctcccgaaaaaatacacgtattggtatcaacagaaatccggacaggcaccggttttggtgatatactatgatacgaaaagacctagtggaatacctgagcggttttctggaagttcttctggtaccatggcaactttgacaatctcaggtgcccaggtcgaggacgaggccgactattattgtttgtctacaaatccgacaggtcatgaatccgtgttcggcggagggacaaagcttaccgtccttgggcagcct

SEQ ID NO: 71 - DNA, 729 bp, synthetic construct
  source 1..729: mol_type = other DNA
caggttcaacttgttcaaagcggtgcagaagttaagaaaccaggtgccagcgttaaggtcagctgtaaagcgagtgggtacactttcaccgaatattatatgaaatgggtacgacaagcaccagggcagggactcgaatggatggggtggatgaaccccaacactggcaagacgggctacgcacagaaattccaagggcgagtgaccatgaccagaaatacgtcaatttcaaccgcatacatggagctgtcatccttgcgatccgaggacaccgcggtatattattgcgcgcgcgctcaatgggagccacttgattattggggacagggcacgcttgtcactgtatcctctggtggaggcggttcaggcggaggtggctctggcggtggcgctagctcatatgaacttactcagccgccttctgttagtgtaagccctgggcagactgcacgcattacatgcagtggtgacgccttgccaaaaaaatatacttattggtaccaacaaaagagtggacaggcgccggtcttggtgatatactatgacactaagagaccctctgggataccggaacgcttttctggttcaagttccggaacgatggcgaccttgacgatcagcggggcccaagtcgaggatgaggccgactactactgtttgtctacagatcccacaggccaagaaagcgtgttcggaggtgggactaaacttacggtcttgggtcaacca

SEQ ID NO: 72 - DNA, 750 bp, synthetic construct
  source 1..750: mol_type = other DNA
caagttcaactggttcagtctggcgccgaagtgaagaaacctggcgcctctgtgaaggtgtcctgcaaggccagcggctacacctttaccagctacggcatcagctgggtccgacaggctcctggacaaggcttggaatggatgggctggatcagcgcctacaacggcaacacctactacgccgaggatctgcagggcagactgaccatgaccaccgataccagcaccagcaccgcctacatggaactgagaagcctgagatccgacgacaccgccgtgtactactgtgccagagagggcagctactacgacagctccggctactacctgccttattggggccagggcacactggtcacagtctcgagtggtggaggcggttcaggcggaggtggctctggcggtggcgctagccaatctgctctgacacagcccagaagcgtgtccggatctcctggccagagcgtgaccatcagctgtacaggcaccagctctgatgtgggcgcctacaactacgtgtcctggtatcagcagcaccccggcaaggcccctaagctgatgatctacgatgtgaccgagaggcctagcggcgtgcccgatagattttctggcagcaagagcggcaacaccgccagcctgacaattagcggactgcaggccgaggacgaggccgattactactgtagcagctacaccagcagctctatcgcctttggcggaggcaccaagctgacagttctgggacaacct

SEQ ID NO: 73 - DNA, 9365 bp, synthetic construct
  source 1..9365: mol_type = other DNA
cgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacccgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgatactagtattatgcccagtacatgaccttatgggactttcctacttggcagtacatctacaaaccccgggtgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacgtagcggccctaatacgactcactatagggcccgcgtacgtctagacctccaggcatcgattccaccatggcactccccgtcaccgcccttctcttgcccctcgccctgctgctgcatgctgccaggccccaagttcaactggttcagtctggcgccgaagtgaagaaacctggcgcctctgtgaaggtgtcctgcaaggccagcggctacacctttaccggctactacatgcactgggtccgacaggctccaggccaaggacttgaatggatgggctggatgaaccccaacagcggcaataccggctatgcccagaaattccagggcagagtgaccatgaccagaaacaccagcatcagcaccgcctacatggaactgagcagcctgagaagcgaggacaccgccgtgtactactgtgctagagcccagtgggagcccctggattattggggacagggcacactggtcacagtctcgagtggtggaggcggttcaggcggaggtggctctggcggtggcgctagctcttatgaactgacccagcctccaagcgtgtccgtgtctcctggacagaccgccagaatcacatgtagcggagatgccctgcctaagaagtacacctactggtatcagcagaagtccggacaggcccctgtgctggtcatctacgaggataccaaacggcccagcggcatccccgagagattttctggaagcagcagcggcacaatggccacactgacaattagcggagcccaggtggaagatgaggccgactactactgcctgagcaccgatcctacaggccaagagtccgtttttggcggaggcaccaagctgacagtgctgggacaacctgcggccgcaaccacaacccctgctccaaggccccccacacccgctccaactatagccagccaaccattgagcctcagacctgaagcttgcaggcccgcagcaggaggcgccgtccatacgcgaggcctggacttcgcgtgtgatatttatatttgggcccctttggccggaacatgtggggtgttgcttctctcccttgtgatcactctgtattgtaagcgcgggagaaagaagctcctgtacatcttcaagcagccttttatgcgacctgtgcaaaccactcaggaagaagatgggtgttcatgccgcttccccgaggaggaagaaggagggtgtgaactgagggtgaaattttctagaagcgccgatgctcccgcatatcagcagggtcagaatcagctctacaatgaattgaatctcggcaggcgagaagagtacgatgttctggacaagagacggggcagggatcccgagatggggggaaagccccggagaaaaaatcctcaggaggggttgtacaatgagctgcagaaggacaagatggctgaagcctatagcgagatcggaatgaaaggcgaaagacgcagaggcaaggggcatgacggtctgtaccagggtctctctacagccaccaaggacacttatgatgcgttgcatatgcaagccttgccaccccgcggatcaggagcgactaacttctctttgttgaagcaagcgggtgacgttgaagagaatcccggtccatccgggggatccatgcggatctccaagccccacctccgaagcatcagtatccagtgttatctgtgccttcttctgaattcccattttttgacagaggcaggaattcatgtattcatattgggttgtttctccgcggggcttcctaaaaccgaagctaactgggttaatgtcatttcagatctgaagaaaattgaagatcttattcaatcaatgcatatcgacgcgaccttgtacacggagagtgatgtccacccgagctgcaaggtaacggccatgaaatgcttcttgctggaattgcaggtaattagtcttgagagtggagatgcgtctatacacgacaccgtagagaatctcattattttggcgaacaattctctcagcagtaacggcaatgttactgaatcaggatgtaaggagtgtgaagagctggaagaaaagaacattaaggaatttttgcagagcttcgtacatatcgtccaaatgttcataaatacaagctaagtcgacggccggccgcgagcatcttaccgccatttatacccatatttgttctgtttttcttgatttgggtatacatttaaatgttaataaaacaaaatggtggggcaatcatttacattttataggatatgtaattactagttcaggtgtattgccacaagacaaacatcttaagaaactttcccgttatttacgctctgttcctgttaatcaacctctggattacaaaatttgtgaaagattgactgatattcttaactatgttgctccttttacgctgtgtggatatgctgctttaatacctctgtatcatactattgcttcctgtacggctttcgttttctcctccttgtataaatcctggttgctgtctctttatcaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcctggtacctttaagaccaatgacttacaaggcagctgtagatcttagccactttttaaaagaaaaggggggactggaagggctaattcactcccaacgaagataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagacttttgcagagaccaaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccc

SEQ ID NO: 74 - DNA, 9365 bp, synthetic construct
  source 1..9365: mol_type = other DNA
cgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacccgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgatactagtattatgcccagtacatgaccttatgggactttcctacttggcagtacatctacaaaccccgggtgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacgtagcggccctaatacgactcactatagggcccgcgtacgtctagacctccaggcatcgattccaccatggcactccccgtcaccgcccttctcttgcccctcgccctgctgctgcatgctgccaggccccaagttcaactggttcagtctggcgccgaagtgaagaaacctggcgcctctgtgaaggtgtcctgcaaggccagcggctacacctttaccggctactacatgcactgggtccgacaggctccaggccaaggacttgaatggatgggctggatgaaccccaacagcggcaataccggctatgcccagaaattccagggcagagtgaccatgaccagaaacaccagcatcagcaccgcctacatggaactgagcagcctgagaagcgaggacaccgccgtgtactactgtgctagagcccagtgggagcccctggattattggggacagggcacactggtcacagtctcgagtggtggaggcggttcaggcggaggtggctctggcggtggcgctagctcttatgaactgacccagcctccaagcgtgtccgtgtctcctggacagaccgccagaatcacatgtagcggagatgccctgcctaagaagtacacctactggtatcagcagaagtccggacaggcccctgtgctggtcatctactatgataccaaacggcccagcggcatccccgagagattttctggaagcagcagcggcacaatggccacactgacaattagcggagcccaggtggaagatgaggccgactactactgcctgagcaccgatcctacaggccaagagtccgtttttggcggaggcaccaagctgacagtgctgggacaacctgcggccgcaaccacaacccctgctccaaggccccccacacccgctccaactatagccagccaaccattgagcctcagacctgaagcttgcaggcccgcagcaggaggcgccgtccatacgcgaggcctggacttcgcgtgtgatatttatatttgggcccctttggccggaacatgtggggtgttgcttctctcccttgtgatcactctgtattgtaagcgcgggagaaagaagctcctgtacatcttcaagcagccttttatgcgacctgtgcaaaccactcaggaagaagatgggtgttcatgccgcttccccgaggaggaagaaggagggtgtgaactgagggtgaaattttctagaagcgccgatgctcccgcatatcagcagggtcagaatcagctctacaatgaattgaatctcggcaggcgagaagagtacgatgttctggacaagagacggggcagggatcccgagatggggggaaagccccggagaaaaaatcctcaggaggggttgtacaatgagctgcagaaggacaagatggctgaagcctatagcgagatcggaatgaaaggcgaaagacgcagaggcaaggggcatgacggtctgtaccagggtctctctacagccaccaaggacacttatgatgcgttgcatatgcaagccttgccaccccgcggatcaggagcgactaacttctctttgttgaagcaagcgggtgacgttgaagagaatcccggtccatccgggggatccatgcggatctccaagccccacctccgaagcatcagtatccagtgttatctgtgccttcttctgaattcccattttttgacagaggcaggaattcatgtattcatattgggttgtttctccgcggggcttcctaaaaccgaagctaactgggttaatgtcatttcagatctgaagaaaattgaagatcttattcaatcaatgcatatcgacgcgaccttgtacacggagagtgatgtccacccgagctgcaaggtaacggccatgaaatgcttcttgctggaattgcaggtaattagtcttgagagtggagatgcgtctatacacgacaccgtagagaatctcattattttggcgaacaattctctcagcagtaacggcaatgttactgaatcaggatgtaaggagtgtgaagagctggaagaaaagaacattaaggaatttttgcagagcttcgtacatatcgtccaaatgttcataaatacaagctaagtcgacggccggccgcgagcatcttaccgccatttatacccatatttgttctgtttttcttgatttgggtatacatttaaatgttaataaaacaaaatggtggggcaatcatttacattttataggatatgtaattactagttcaggtgtattgccacaagacaaacatcttaagaaactttcccgttatttacgctctgttcctgttaatcaacctctggattacaaaatttgtgaaagattgactgatattcttaactatgttgctccttttacgctgtgtggatatgctgctttaatacctctgtatcatactattgcttcctgtacggctttcgttttctcctccttgtataaatcctggttgctgtctctttatcaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcctggtacctttaagaccaatgacttacaaggcagctgtagatcttagccactttttaaaagaaaaggggggactggaagggctaattcactcccaacgaagataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagacttttgcagagaccaaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccc

SEQ ID NO: 75 - DNA, 9365 bp, synthetic construct
  source 1..9365: mol_type = other DNA
cgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacccgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgatactagtattatgcccagtacatgaccttatgggactttcctacttggcagtacatctacaaaccccgggtgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacgtagcggccctaatacgactcactatagggcccgcgtacgtctagacctccaggcatcgattccaccatggcactccccgtcaccgcccttctcttgcccctcgccctgctgctgcatgctgccaggccccaggtgcaattggtgcaatcaggggccgaagtcaagaaaccgggtgcttcagtcaaggtttcttgtaaagcatctggctatacgtttactgggtactacatgcaatgggtgagacaagctcctggccaagggcttgaatggatggggtggatgaatccaatctctggaaatacaggctacgcccaaaagttccagggaagagttaccatgacacggaatacgtccatttccacggcatacatggaactgtcctcccttcggagcgaggacactgcagtatattactgcgcgagagcacaatgggaaccgcttgattattggggtcaaggtaccttggttactgtgtcctcaggtggaggcggttcaggcggaggtggctctggcggtggcgctagcagctatgaactgactcagccaccgagtgtatctgtaagtccgggtcagacagcgcggatcacttgctcaggggacgcccttcccaaaaaatacacttactggtatcaacagaagagtgggcaggctcctgtgttggtaatatactatgacacgaaacggcctagcgggatcccggagcggttctcaggttcctccagtgggacgatggctacactgacgatcagcggggcccaagtggaggacgaagctgattattattgccttagtacccaccccgccggacaggagtctgtgtttggtggcggcaccaagcttacggtactcggacagcctgcggccgcaaccacaacccctgctccaaggccccccacacccgctccaactatagccagccaaccattgagcctcagacctgaagcttgcaggcccgcagcaggaggcgccgtccatacgcgaggcctggacttcgcgtgtgatatttatatttgggcccctttggccggaacatgtggggtgttgcttctctcccttgtgatcactctgtattgtaagcgcgggagaaagaagctcctgtacatcttcaagcagccttttatgcgacctgtgcaaaccactcaggaagaagatgggtgttcatgccgcttccccgaggaggaagaaggagggtgtgaactgagggtgaaattttctagaagcgccgatgctcccgcatatcagcagggtcagaatcagctctacaatgaattgaatctcggcaggcgagaagagtacgatgttctggacaagagacggggcagggatcccgagatggggggaaagccccggagaaaaaatcctcaggaggggttgtacaatgagctgcagaaggacaagatggctgaagcctatagcgagatcggaatgaaaggcgaaagacgcagaggcaaggggcatgacggtctgtaccagggtctctctacagccaccaaggacacttatgatgcgttgcatatgcaagccttgccaccccgcggatcaggagcgactaacttctctttgttgaagcaagcgggtgacgttgaagagaatcccggtccatccgggggatccatgcggatctccaagccccacctccgaagcatcagtatccagtgttatctgtgccttcttctgaattcccattttttgacagaggcaggaattcatgtattcatattgggttgtttctccgcggggcttcctaaaaccgaagctaactgggttaatgtcatttcagatctgaagaaaattgaagatcttattcaatcaatgcatatcgacgcgaccttgtacacggagagtgatgtccacccgagctgcaaggtaacggccatgaaatgcttcttgctggaattgcaggtaattagtcttgagagtggagatgcgtctatacacgacaccgtagagaatctcattattttggcgaacaattctctcagcagtaacggcaatgttactgaatcaggatgtaaggagtgtgaagagctggaagaaaagaacattaaggaatttttgcagagcttcgtacatatcgtccaaatgttcataaatacaagctaagtcgacggccggccgcgagcatcttaccgccatttatacccatatttgttctgtttttcttgatttgggtatacatttaaatgttaataaaacaaaatggtggggcaatcatttacattttataggatatgtaattactagttcaggtgtattgccacaagacaaacatcttaagaaactttcccgttatttacgctctgttcctgttaatcaacctctggattacaaaatttgtgaaagattgactgatattcttaactatgttgctccttttacgctgtgtggatatgctgctttaatacctctgtatcatactattgcttcctgtacggctttcgttttctcctccttgtataaatcctggttgctgtctctttatcaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcctggtacctttaagaccaatgacttacaaggcagctgtagatcttagccactttttaaaagaaaaggggggactggaagggctaattcactcccaacgaagataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagacttttgcagagaccaaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccc

SEQ ID NO: 76 - DNA, 9365 bp, synthetic construct
  source 1..9365: mol_type = other DNA
cgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacccgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgatactagtattatgcccagtacatgaccttatgggactttcctacttggcagtacatctacaaaccccgggtgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacgtagcggccctaatacgactcactatagggcccgcgtacgtctagacctccaggcatcgattccaccatggcactccccgtcaccgcccttctcttgcccctcgccctgctgctgcatgctgccaggccccaggtccaattggtccaatctggcgccgaggtcaagaagcctggcgcatccgtaaaagtgtcttgtaaagcctctggttatacgtttacggggtattatatgcaatgggttcggcaagcccctggtcaaggacttgaatggatgggctggatgaaccctatctctggtaatacgggctacgcccaaaagttccagggtagagtcacaatgacgcgcaacacgtcaatatcaaccgcttacatggagcttagttctttgaggtcagaggacaccgcggtctattattgtgctcgcgcccaatgggaaccgcttgattactgggggcaaggtaccttggttaccgtctcatcaggtggaggcggttcaggcggaggtggctctggcggtggcgctagcagctacgaactgacacagccgccctctgttagcgttagtcctggacagaccgccaggataacgtgctccggtgacgcattgccgaagaaatatacgtactggtaccagcagaagtccgggcaagcacctgtactcgtgatctattatgatacaaaacggccgagcggtataccagaacgctttagcggctcaagcagcggaacgatggccacgttgacgattagcggcgcgcaggtcgaggacgaagctgactactattgcctcagtactaacccaacagggcacgaatcagtttttggagggggaaccaagcttactgtacttggtcagccggcggccgcaaccacaacccctgctccaaggccccccacacccgctccaactatagccagccaaccattgagcctcagacctgaagcttgcaggcccgcagcaggaggcgccgtccatacgcgaggcctggacttcgcgtgtgatatttatatttgggcccctttggccggaacatgtggggtgttgcttctctcccttgtgatcactctgtattgtaagcgcgggagaaagaagctcctgtacatcttcaagcagccttttatgcgacctgtgcaaaccactcaggaagaagatgggtgttcatgccgcttccccgaggaggaagaaggagggtgtgaactgagggtgaaattttctagaagcgccgatgctcccgcatatcagcagggtcagaatcagctctacaatgaattgaatctcggcaggcgagaagagtacgatgttctggacaagagacggggcagggatcccgagatggggggaaagccccggagaaaaaatcctcaggaggggttgtacaatgagctgcagaaggacaagatggctgaagcctatagcgagatcggaatgaaaggcgaaagacgcagaggcaaggggcatgacggtctgtaccagggtctctctacagccaccaaggacacttatgatgcgttgcatatgcaagccttgccaccccgcggatcaggagcgactaacttctctttgttgaagcaagcgggtgacgttgaagagaatcccggtccatccgggggatccatgcggatctccaagccccacctccgaagcatcagtatccagtgttatctgtgccttcttctgaattcccattttttgacagaggcaggaattcatgtattcatattgggttgtttctccgcggggcttcctaaaaccgaagctaactgggttaatgtcatttcagatctgaagaaaattgaagatcttattcaatcaatgcatatcgacgcgaccttgtacacggagagtgatgtccacccgagctgcaaggtaacggccatgaaatgcttcttgctggaattgcaggtaattagtcttgagagtggagatgcgtctatacacgacaccgtagagaatctcattattttggcgaacaattctctcagcagtaacggcaatgttactgaatcaggatgtaaggagtgtgaagagctggaagaaaagaacattaaggaatttttgcagagcttcgtacatatcgtccaaatgttcataaatacaagctaagtcgacggccggccgcgagcatcttaccgccatttatacccatatttgttctgtttttcttgatttgggtatacatttaaatgttaataaaacaaaatggtggggcaatcatttacattttataggatatgtaattactagttcaggtgtattgccacaagacaaacatcttaagaaactttcccgttatttacgctctgttcctgttaatcaacctctggattacaaaatttgtgaaagattgactgatattcttaactatgttgctccttttacgctgtgtggatatgctgctttaatacctctgtatcatactattgcttcctgtacggctttcgttttctcctccttgtataaatcctggttgctgtctctttatcaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcctggtacctttaagaccaatgacttacaaggcagctgtagatcttagccactttttaaaagaaaaggggggactggaagggctaattcactcccaacgaagataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagacttttgcagagaccaaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccc

SEQ ID NO: 77 - DNA, 9365 bp, synthetic construct
  source 1..9365: mol_type = other DNA
cgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacccgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgatactagtattatgcccagtacatgaccttatgggactttcctacttggcagtacatctacaaaccccgggtgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacgtagcggccctaatacgactcactatagggcccgcgtacgtctagacctccaggcatcgattccaccatggcactccccgtcaccgcccttctcttgcccctcgccctgctgctgcatgctgccaggccccaggtccagctggtacagtctggcgctgaggtaaagaagcctggtgcttcagtgaaggtgtcttgcaaagcctctggctatactttcacaggttattatatgcactgggtgcgccaggcccccggtcagggccttgaatggatgggatggatgaatccaaattcaggcaataccgggtatgctcaaaagtttcagggccgcgtaacaatgacccgaaatacaagtatttcaactgcttatatggaactcagttctctcagaagtgaagatacggccgtatattattgcgcgcgggcgcaatgggaaccacttgactattggggacaaggcacactggttacggtgtcatctggtggaggcggttcaggcggaggtggctctggcggtggcgctagctcatatgaactcacacagccgccatctgttagcgtttctccagggcagactgcccgaattacatgctcaggcgatgccctcccgaaaaaatatacctactggtaccaacaaaaatccggccaggcacctgtgttggtgatatattatgatacaaaacgcccttcagggatcccggagagattcagtgggtcaagtagcggaacaatggcaacacttactataagcggggcacaggttgaggacgaggctgactactactgcctgtctactcatccggccggacaggagagtgttttcgggggtggaactaaactcacagtactgggacagcccgcggccgcaaccacaacccctgctccaaggccccccacacccgctccaactatagccagccaaccattgagcctcagacctgaagcttgcaggcccgcagcaggaggcgccgtccatacgcgaggcctggacttcgcgtgtgatatttatatttgggcccctttggccggaacatgtggggtgttgcttctctcccttgtgatcactctgtattgtaagcgcgggagaaagaagctcctgtacatcttcaagcagccttttatgcgacctgtgcaaaccactcaggaagaagatgggtgttcatgccgcttccccgaggaggaagaaggagggtgtgaactgagggtgaaattttctagaagcgccgatgctcccgcatatcagcagggtcagaatcagctctacaatgaattgaatctcggcaggcgagaagagtacgatgttctggacaagagacggggcagggatcccgagatggggggaaagccccggagaaaaaatcctcaggaggggttgtacaatgagctgcagaaggacaagatggctgaagcctatagcgagatcggaatgaaaggcgaaagacgcagaggcaaggggcatgacggtctgtaccagggtctctctacagccaccaaggacacttatgatgcgttgcatatgcaagccttgccaccccgcggatcaggagcgactaacttctctttgttgaagcaagcgggtgacgttgaagagaatcccggtccatccgggggatccatgcggatctccaagccccacctccgaagcatcagtatccagtgttatctgtgccttcttctgaattcccattttttgacagaggcaggaattcatgtattcatattgggttgtttctccgcggggcttcctaaaaccgaagctaactgggttaatgtcatttcagatctgaagaaaattgaagatcttattcaatcaatgcatatcgacgcgaccttgtacacggagagtgatgtccacccgagctgcaaggtaacggccatgaaatgcttcttgctggaattgcaggtaattagtcttgagagtggagatgcgtctatacacgacaccgtagagaatctcattattttggcgaacaattctctcagcagtaacggcaatgttactgaatcaggatgtaaggagtgtgaagagctggaagaaaagaacattaaggaatttttgcagagcttcgtacatatcgtccaaatgttcataaatacaagctaagtcgacggccggccgcgagcatcttaccgccatttatacccatatttgttctgtttttcttgatttgggtatacatttaaatgttaataaaacaaaatggtggggcaatcatttacattttataggatatgtaattactagttcaggtgtattgccacaagacaaacatcttaagaaactttcccgttatttacgctctgttcctgttaatcaacctctggattacaaaatttgtgaaagattgactgatattcttaactatgttgctccttttacgctgtgtggatatgctgctttaatacctctgtatcatactattgcttcctgtacggctttcgttttctcctccttgtataaatcctggttgctgtctctttatcaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcctggtacctttaagaccaatgacttacaaggcagctgtagatcttagccactttttaaaagaaaaggggggactggaagggctaattcactcccaacgaagataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagacttttgcagagaccaaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccc

SEQ ID NO: 78 - DNA, 9365 bp, synthetic construct
  source 1..9365: mol_type = other DNA
cgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacccgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgatactagtattatgcccagtacatgaccttatgggactttcctacttggcagtacatctacaaaccccgggtgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacgtagcggccctaatacgactcactatagggcccgcgtacgtctagacctccaggcatcgattccaccatggcactccccgtcaccgcccttctcttgcccctcgccctgctgctgcatgctgccaggccccaggtccaactggtgcaatctggcgctgaagtgaaaaagcccggcgcatcagtaaaggtgagttgcaaagcatctgggtacactttcactggctattatatgcattgggtaaggcaagcccccggccaaggcctggaatggatgggatggatgaatccgaactccggtaatacagggtacgctcaaaaatttcaaggacgggtcacaatgacgcggaacaccagtatatctactgcatatatggagctcagttcactgcgctccgaggatacagctgtatactattgcgccagggctcaatgggaaccactggactattggggtcaagggacacttgtaacggtaagctcaggtggaggcggttcaggcggaggtggctctggcggtggcgctagctcctatgagcttactcagccaccctctgtatctgtatctcctgggcagacagcacgcatcacatgtagtggcgatgccctcccgaaaaaatacacgtattggtatcaacagaaatccggacaggcaccggttttggtgatatactatgatacgaaaagacctagtggaatacctgagcggttttctggaagttcttctggtaccatggcaactttgacaatctcaggtgcccaggtcgaggacgaggccgactattattgtttgtctacaaatccgacaggtcatgaatccgtgttcggcggagggacaaagcttaccgtccttgggcagcctgcggccgcaaccacaacccctgctccaaggccccccacacccgctccaactatagccagccaaccattgagcctcagacctgaagcttgcaggcccgcagcaggaggcgccgtccatacgcgaggcctggacttcgcgtgtgatatttatatttgggcccctttggccggaacatgtggggtgttgcttctctcccttgtgatcactctgtattgtaagcgcgggagaaagaagctcctgtacatcttcaagcagccttttatgcgacctgtgcaaaccactcaggaagaagatgggtgttcatgccgcttccccgaggaggaagaaggagggtgtgaactgagggtgaaattttctagaagcgccgatgctcccgcatatcagcagggtcagaatcagctctacaatgaattgaatctcggcaggcgagaagagtacgatgttctggacaagagacggggcagggatcccgagatggggggaaagccccggagaaaaaatcctcaggaggggttgtacaatgagctgcagaaggacaagatggctgaagcctatagcgagatcggaatgaaaggcgaaagacgcagaggcaaggggcatgacggtctgtaccagggtctctctacagccaccaaggacacttatgatgcgttgcatatgcaagccttgccaccccgcggatcaggagcgactaacttctctttgttgaagcaagcgggtgacgttgaagagaatcccggtccatccgggggatccatgcggatctccaagccccacctccgaagcatcagtatccagtgttatctgtgccttcttctgaattcccattttttgacagaggcaggaattcatgtattcatattgggttgtttctccgcggggcttcctaaaaccgaagctaactgggttaatgtcatttcagatctgaagaaaattgaagatcttattcaatcaatgcatatcgacgcgaccttgtacacggagagtgatgtccacccgagctgcaaggtaacggccatgaaatgcttcttgctggaattgcaggtaattagtcttgagagtggagatgcgtctatacacgacaccgtagagaatctcattattttggcgaacaattctctcagcagtaacggcaatgttactgaatcaggatgtaaggagtgtgaagagctggaagaaaagaacattaaggaatttttgcagagcttcgtacatatcgtccaaatgttcataaatacaagctaagtcgacggccggccgcgagcatcttaccgccatttatacccatatttgttctgtttttcttgatttgggtatacatttaaatgttaataaaacaaaatggtggggcaatcatttacattttataggatatgtaattactagttcaggtgtattgccacaagacaaacatcttaagaaactttcccgttatttacgctctgttcctgttaatcaacctctggattacaaaatttgtgaaagattgactgatattcttaactatgttgctccttttacgctgtgtggatatgctgctttaatacctctgtatcatactattgcttcctgtacggctttcgttttctcctccttgtataaatcctggttgctgtctctttatcaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcctggtacctttaagaccaatgacttacaaggcagctgtagatcttagccactttttaaaagaaaaggggggactggaagggctaattcactcccaacgaagataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagacttttgcagagaccaaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccc

SEQ ID NO: 79 - DNA, 9365 bp, synthetic construct
  source 1..9365: mol_type = other DNA
cgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacccgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgatactagtattatgcccagtacatgaccttatgggactttcctacttggcagtacatctacaaaccccgggtgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacgtagcggccctaatacgactcactatagggcccgcgtacgtctagacctccaggcatcgattccaccatggcactccccgtcaccgcccttctcttgcccctcgccctgctgctgcatgctgccaggccccaggttcaacttgttcaaagcggtgcagaagttaagaaaccaggtgccagcgttaaggtcagctgtaaagcgagtgggtacactttcaccgaatattatatgaaatgggtacgacaagcaccagggcagggactcgaatggatggggtggatgaaccccaacactggcaagacgggctacgcacagaaattccaagggcgagtgaccatgaccagaaatacgtcaatttcaaccgcatacatggagctgtcatccttgcgatccgaggacaccgcggtatattattgcgcgcgcgctcaatgggagccacttgattattggggacagggcacgcttgtcactgtatcctctggtggaggcggttcaggcggaggtggctctggcggtggcgctagctcatatgaacttactcagccgccttctgttagtgtaagccctgggcagactgcacgcattacatgcagtggtgacgccttgccaaaaaaatatacttattggtaccaacaaaagagtggacaggcgccggtcttggtgatatactatgacactaagagaccctctgggataccggaacgcttttctggttcaagttccggaacgatggcgaccttgacgatcagcggggcccaagtcgaggatgaggccgactactactgtttgtctacagatcccacaggccaagaaagcgtgttcggaggtgggactaaacttacggtcttgggtcaaccagcggccgcaaccacaacccctgctccaaggccccccacacccgctccaactatagccagccaaccattgagcctcagacctgaagcttgcaggcccgcagcaggaggcgccgtccatacgcgaggcctggacttcgcgtgtgatatttatatttgggcccctttggccggaacatgtggggtgttgcttctctcccttgtgatcactctgtattgtaagcgcgggagaaagaagctcctgtacatcttcaagcagccttttatgcgacctgtgcaaaccactcaggaagaagatgggtgttcatgccgcttccccgaggaggaagaaggagggtgtgaactgagggtgaaattttctagaagcgccgatgctcccgcatatcagcagggtcagaatcagctctacaatgaattgaatctcggcaggcgagaagagtacgatgttctggacaagagacggggcagggatcccgagatggggggaaagccccggagaaaaaatcctcaggaggggttgtacaatgagctgcagaaggacaagatggctgaagcctatagcgagatcggaatgaaaggcgaaagacgcagaggcaaggggcatgacggtctgtaccagggtctctctacagccaccaaggacacttatgatgcgttgcatatgcaagccttgccaccccgcggatcaggagcgactaacttctctttgttgaagcaagcgggtgacgttgaagagaatcccggtccatccgggggatccatgcggatctccaagccccacctccgaagcatcagtatccagtgttatctgtgccttcttctgaattcccattttttgacagaggcaggaattcatgtattcatattgggttgtttctccgcggggcttcctaaaaccgaagctaactgggttaatgtcatttcagatctgaagaaaattgaagatcttattcaatcaatgcatatcgacgcgaccttgtacacggagagtgatgtccacccgagctgcaaggtaacggccatgaaatgcttcttgctggaattgcaggtaattagtcttgagagtggagatgcgtctatacacgacaccgtagagaatctcattattttggcgaacaattctctcagcagtaacggcaatgttactgaatcaggatgtaaggagtgtgaagagctggaagaaaagaacattaaggaatttttgcagagcttcgtacatatcgtccaaatgttcataaatacaagctaagtcgacggccggccgcgagcatcttaccgccatttatacccatatttgttctgtttttcttgatttgggtatacatttaaatgttaataaaacaaaatggtggggcaatcatttacattttataggatatgtaattactagttcaggtgtattgccacaagacaaacatcttaagaaactttcccgttatttacgctctgttcctgttaatcaacctctggattacaaaatttgtgaaagattgactgatattcttaactatgttgctccttttacgctgtgtggatatgctgctttaatacctctgtatcatactattgcttcctgtacggctttcgttttctcctccttgtataaatcctggttgctgtctctttatcaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcctggtacctttaagaccaatgacttacaaggcagctgtagatcttagccactttttaaaagaaaaggggggactggaagggctaattcactcccaacgaagataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagacttttgcagagaccaaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccc

SEQ ID NO: 80 - DNA, 9386 bp, synthetic construct
  source 1..9386: mol_type = other DNA
cgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacccgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgatactagtattatgcccagtacatgaccttatgggactttcctacttggcagtacatctacaaaccccgggtgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacgtagcggccctaatacgactcactatagggcccgcgtacgtctagacctccaggcatcgattccaccatggcactccccgtcaccgcccttctcttgcccctcgccctgctgctgcatgctgccaggccccaagttcaactggttcagtctggcgccgaagtgaagaaacctggcgcctctgtgaaggtgtcctgcaaggccagcggctacacctttaccagctacggcatcagctgggtccgacaggctcctggacaaggcttggaatggatgggctggatcagcgcctacaacggcaacacctactacgccgaggatctgcagggcagactgaccatgaccaccgataccagcaccagcaccgcctacatggaactgagaagcctgagatccgacgacaccgccgtgtactactgtgccagagagggcagctactacgacagctccggctactacctgccttattggggccagggcacactggtcacagtctcgagtggtggaggcggttcaggcggaggtggctctggcggtggcgctagccaatctgctctgacacagcccagaagcgtgtccggatctcctggccagagcgtgaccatcagctgtacaggcaccagctctgatgtgggcgcctacaactacgtgtcctggtatcagcagcaccccggcaaggcccctaagctgatgatctacgatgtgaccgagaggcctagcggcgtgcccgatagattttctggcagcaagagcggcaacaccgccagcctgacaattagcggactgcaggccgaggacgaggccgattactactgtagcagctacaccagcagctctatcgcctttggcggaggcaccaagctgacagttctgggacaacctgcggccgcaaccacaacccctgctccaaggccccccacacccgctccaactatagccagccaaccattgagcctcagacctgaagcttgcaggcccgcagcaggaggcgccgtccatacgcgaggcctggacttcgcgtgtgatatttatatttgggcccctttggccggaacatgtggggtgttgcttctctcccttgtgatcactctgtattgtaagcgcgggagaaagaagctcctgtacatcttcaagcagccttttatgcgacctgtgcaaaccactcaggaagaagatgggtgttcatgccgcttccccgaggaggaagaaggagggtgtgaactgagggtgaaattttctagaagcgccgatgctcccgcatatcagcagggtcagaatcagctctacaatgaattgaatctcggcaggcgagaagagtacgatgttctggacaagagacggggcagggatcccgagatggggggaaagccccggagaaaaaatcctcaggaggggttgtacaatgagctgcagaaggacaagatggctgaagcctatagcgagatcggaatgaaaggcgaaagacgcagaggcaaggggcatgacggtctgtaccagggtctctctacagccaccaaggacacttatgatgcgttgcatatgcaagccttgccaccccgcggatcaggagcgactaacttctctttgttgaagcaagcgggtgacgttgaagagaatcccggtccatccgggggatccatgcggatctccaagccccacctccgaagcatcagtatccagtgttatctgtgccttcttctgaattcccattttttgacagaggcaggaattcatgtattcatattgggttgtttctccgcggggcttcctaaaaccgaagctaactgggttaatgtcatttcagatctgaagaaaattgaagatcttattcaatcaatgcatatcgacgcgaccttgtacacggagagtgatgtccacccgagctgcaaggtaacggccatgaaatgcttcttgctggaattgcaggtaattagtcttgagagtggagatgcgtctatacacgacaccgtagagaatctcattattttggcgaacaattctctcagcagtaacggcaatgttactgaatcaggatgtaaggagtgtgaagagctggaagaaaagaacattaaggaatttttgcagagcttcgtacatatcgtccaaatgttcataaatacaagctaagtcgacggccggccgcgagcatcttaccgccatttatacccatatttgttctgtttttcttgatttgggtatacatttaaatgttaataaaacaaaatggtggggcaatcatttacattttataggatatgtaattactagttcaggtgtattgccacaagacaaacatcttaagaaactttcccgttatttacgctctgttcctgttaatcaacctctggattacaaaatttgtgaaagattgactgatattcttaactatgttgctccttttacgctgtgtggatatgctgctttaatacctctgtatcatactattgcttcctgtacggctttcgttttctcctccttgtataaatcctggttgctgtctctttatcaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcctggtacctttaagaccaatgacttacaaggcagctgtagatcttagccactttttaaaagaaaaggggggactggaagggctaattcactcccaacgaagataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagacttttgcagagaccaaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccc

SEQ ID NO: 81 - protein, 15 aa, synthetic construct
GGGGSGGGGSGGGAS

SEQ ID NO: 82 - protein, 45 aa, synthetic construct
TTTPAPRPPTPAPTIASQPLSLRPEACRPAAGGAVHTRGLDFACD

SEQ ID NO: 83 - protein, 24 aa, synthetic construct
IYIWAPLAGTCGVLLLSLVITLYC

SEQ ID NO: 84 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 85 - protein, 42 aa, synthetic construct
KRGRKKLLYIFKQPFMRPVQTTQEEDGCSCRFPEEEEGGCEL

SEQ ID NO: 86 - protein, 469 aa, synthetic construct
QVQLVQSGAEVKKPGASVKVSCKASGYTFTGYYMHWVRQAPGQGLEWMGWMNPNSGNTGYAQKFQGRVTMTRNTSISTAYMELSSLRSEDTAVYYCARAQWEPLDYWGQGTLVTVSSGGGGSGGGGSGGGASSYELTQPPSVSVSPGQTARITCSGDALPKKYTYWYQQKSGQAPVLVIYEDTKRPSGIPERFSGSSSGTMATLTISGAQVEDEADYYCLSTDPTGQESVFGGGTKLTVLGQPAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 87 - protein, 469 aa, synthetic construct
QVQLVQSGAEVKKPGASVKVSCKASGYTFTGYYMHWVRQAPGQGLEWMGWMNPNSGNTGYAQKFQGRVTMTRNTSISTAYMELSSLRSEDTAVYYCARAQWEPLDYWGQGTLVTVSSGGGGSGGGGSGGGASSYELTQPPSVSVSPGQTARITCSGDALPKKYTYWYQQKSGQAPVLVIYYDTKRPSGIPERFSGSSSGTMATLTISGAQVEDEADYYCLSTDPTGQESVFGGGTKLTVLGQPAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 88 - protein, 469 aa, synthetic construct
QVQLVQSGAEVKKPGASVKVSCKASGYTFTGYYMQWVRQAPGQGLEWMGWMNPISGNTGYAQKFQGRVTMTRNTSISTAYMELSSLRSEDTAVYYCARAQWEPLDYWGQGTLVTVSSGGGGSGGGGSGGGASSYELTQPPSVSVSPGQTARITCSGDALPKKYTYWYQQKSGQAPVLVIYYDTKRPSGIPERFSGSSSGTMATLTISGAQVEDEADYYCLSTHPAGQESVFGGGTKLTVLGQPAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 89 - protein, 469 aa, synthetic construct
QVQLVQSGAEVKKPGASVKVSCKASGYTFTGYYMQWVRQAPGQGLEWMGWMNPISGNTGYAQKFQGRVTMTRNTSISTAYMELSSLRSEDTAVYYCARAQWEPLDYWGQGTLVTVSSGGGGSGGGGSGGGASSYELTQPPSVSVSPGQTARITCSGDALPKKYTYWYQQKSGQAPVLVIYYDTKRPSGIPERFSGSSSGTMATLTISGAQVEDEADYYCLSTNPTGHESVFGGGTKLTVLGQPAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 90 - protein, 469 aa, synthetic construct
QVQLVQSGAEVKKPGASVKVSCKASGYTFTGYYMHWVRQAPGQGLEWMGWMNPNSGNTGYAQKFQGRVTMTRNTSISTAYMELSSLRSEDTAVYYCARAQWEPLDYWGQGTLVTVSSGGGGSGGGGSGGGASSYELTQPPSVSVSPGQTARITCSGDALPKKYTYWYQQKSGQAPVLVIYYDTKRPSGIPERFSGSSSGTMATLTISGAQVEDEADYYCLSTHPAGQESVFGGGTKLTVLGQPAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 91 - protein, 469 aa, synthetic construct
QVQLVQSGAEVKKPGASVKVSCKASGYTFTGYYMHWVRQAPGQGLEWMGWMNPNSGNTGYAQKFQGRVTMTRNTSISTAYMELSSLRSEDTAVYYCARAQWEPLDYWGQGTLVTVSSGGGGSGGGGSGGGASSYELTQPPSVSVSPGQTARITCSGDALPKKYTYWYQQKSGQAPVLVIYYDTKRPSGIPERFSGSSSGTMATLTISGAQVEDEADYYCLSTNPTGHESVFGGGTKLTVLGQPAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 92 - protein, 469 aa, synthetic construct
QVQLVQSGAEVKKPGASVKVSCKASGYTFTEYYMKWVRQAPGQGLEWMGWMNPNTGKTGYAQKFQGRVTMTRNTSISTAYMELSSLRSEDTAVYYCARAQWEPLDYWGQGTLVTVSSGGGGSGGGGSGGGASSYELTQPPSVSVSPGQTARITCSGDALPKKYTYWYQQKSGQAPVLVIYYDTKRPSGIPERFSGSSSGTMATLTISGAQVEDEADYYCLSTDPTGQESVFGGGTKLTVLGQPAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 93 - protein, 476 aa, synthetic construct
QVQLVQSGAEVKKPGASVKVSCKASGYTFTSYGISWVRQAPGQGLEWMGWISAYNGNTYYAEDLQGRLTMTTDTSTSTAYMELRSLRSDDTAVYYCAREGSYYDSSGYYLPYWGQGTLVTVSSGGGGSGGGGSGGGASQSALTQPRSVSGSPGQSVTISCTGTSSDVGAYNYVSWYQQHPGKAPKLMIYDVTERPSGVPDRFSGSKSGNTASLTISGLQAEDEADYYCSSYTSSSIAFGGGTKLTVLGQPAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 94 - DNA, 486 bp, synthetic construct
  source 1..486: mol_type = other DNA
atgcggatctccaagccccacctccgaagcatcagtatccagtgttatctgtgccttcttctgaattcccattttttgacagaggcaggaattcatgtattcatattgggttgtttctccgcggggcttcctaaaaccgaagctaactgggttaatgtcatttcagatctgaagaaaattgaagatcttattcaatcaatgcatatcgacgcgaccttgtacacggagagtgatgtccacccgagctgcaaggtaacggccatgaaatgcttcttgctggaattgcaggtaattagtcttgagagtggagatgcgtctatacacgacaccgtagagaatctcattattttggcgaacaattctctcagcagtaacggcaatgttactgaatcaggatgtaaggagtgtgaagagctggaagaaaagaacattaaggaatttttgcagagcttcgtacatatcgtccaaatgttcataaatacaagc

SEQ ID NO: 95 - DNA, 1407 bp, synthetic construct
  source 1..1407: mol_type = other DNA
caagttcaactggttcagtctggcgccgaagtgaagaaacctggcgcctctgtgaaggtgtcctgcaaggccagcggctacacctttaccggctactacatgcactgggtccgacaggctccaggccaaggacttgaatggatgggctggatgaaccccaacagcggcaataccggctatgcccagaaattccagggcagagtgaccatgaccagaaacaccagcatcagcaccgcctacatggaactgagcagcctgagaagcgaggacaccgccgtgtactactgtgctagagcccagtgggagcccctggattattggggacagggcacactggtcacagtctcgagtggtggaggcggttcaggcggaggtggctctggcggtggcgctagctcttatgaactgacccagcctccaagcgtgtccgtgtctcctggacagaccgccagaatcacatgtagcggagatgccctgcctaagaagtacacctactggtatcagcagaagtccggacaggcccctgtgctggtcatctacgaggataccaaacggcccagcggcatccccgagagattttctggaagcagcagcggcacaatggccacactgacaattagcggagcccaggtggaagatgaggccgactactactgcctgagcaccgatcctacaggccaagagtccgtttttggcggaggcaccaagctgacagtgctgggacaacctgcggccgcaaccacaacccctgctccaaggccccccacacccgctccaactatagccagccaaccattgagcctcagacctgaagcttgcaggcccgcagcaggaggcgccgtccatacgcgaggcctggacttcgcgtgtgatatttatatttgggcccctttggccggaacatgtggggtgttgcttctctcccttgtgatcactctgtattgtaagcgcgggagaaagaagctcctgtacatcttcaagcagccttttatgcgacctgtgcaaaccactcaggaagaagatgggtgttcatgccgcttccccgaggaggaagaaggagggtgtgaactgagggtgaaattttctagaagcgccgatgctcccgcatatcagcagggtcagaatcagctctacaatgaattgaatctcggcaggcgagaagagtacgatgttctggacaagagacggggcagggatcccgagatggggggaaagccccggagaaaaaatcctcaggaggggttgtacaatgagctgcagaaggacaagatggctgaagcctatagcgagatcggaatgaaaggcgaaagacgcagaggcaaggggcatgacggtctgtaccagggtctctctacagccaccaaggacacttatgatgcgttgcatatgcaagccttgccaccccgc

SEQ ID NO: 96 - DNA, 1407 bp, synthetic construct
  source 1..1407: mol_type = other DNA
caagttcaactggttcagtctggcgccgaagtgaagaaacctggcgcctctgtgaaggtgtcctgcaaggccagcggctacacctttaccggctactacatgcactgggtccgacaggctccaggccaaggacttgaatggatgggctggatgaaccccaacagcggcaataccggctatgcccagaaattccagggcagagtgaccatgaccagaaacaccagcatcagcaccgcctacatggaactgagcagcctgagaagcgaggacaccgccgtgtactactgtgctagagcccagtgggagcccctggattattggggacagggcacactggtcacagtctcgagtggtggaggcggttcaggcggaggtggctctggcggtggcgctagctcttatgaactgacccagcctccaagcgtgtccgtgtctcctggacagaccgccagaatcacatgtagcggagatgccctgcctaagaagtacacctactggtatcagcagaagtccggacaggcccctgtgctggtcatctactatgataccaaacggcccagcggcatccccgagagattttctggaagcagcagcggcacaatggccacactgacaattagcggagcccaggtggaagatgaggccgactactactgcctgagcaccgatcctacaggccaagagtccgtttttggcggaggcaccaagctgacagtgctgggacaacctgcggccgcaaccacaacccctgctccaaggccccccacacccgctccaactatagccagccaaccattgagcctcagacctgaagcttgcaggcccgcagcaggaggcgccgtccatacgcgaggcctggacttcgcgtgtgatatttatatttgggcccctttggccggaacatgtggggtgttgcttctctcccttgtgatcactctgtattgtaagcgcgggagaaagaagctcctgtacatcttcaagcagccttttatgcgacctgtgcaaaccactcaggaagaagatgggtgttcatgccgcttccccgaggaggaagaaggagggtgtgaactgagggtgaaattttctagaagcgccgatgctcccgcatatcagcagggtcagaatcagctctacaatgaattgaatctcggcaggcgagaagagtacgatgttctggacaagagacggggcagggatcccgagatggggggaaagccccggagaaaaaatcctcaggaggggttgtacaatgagctgcagaaggacaagatggctgaagcctatagcgagatcggaatgaaaggcgaaagacgcagaggcaaggggcatgacggtctgtaccagggtctctctacagccaccaaggacacttatgatgcgttgcatatgcaagccttgccaccccgc

SEQ ID NO: 97 - DNA, 1974 bp, synthetic construct
  source 1..1974: mol_type = other DNA
caggtgcaattggtgcaatcaggggccgaagtcaagaaaccgggtgcttcagtcaaggtttcttgtaaagcatctggctatacgtttactgggtactacatgcaatgggtgagacaagctcctggccaagggcttgaatggatggggtggatgaatccaatctctggaaatacaggctacgcccaaaagttccagggaagagttaccatgacacggaatacgtccatttccacggcatacatggaactgtcctcccttcggagcgaggacactgcagtatattactgcgcgagagcacaatgggaaccgcttgattattggggtcaaggtaccttggttactgtgtcctcaggtggaggcggttcaggcggaggtggctctggcggtggcgctagcagctatgaactgactcagccaccgagtgtatctgtaagtccgggtcagacagcgcggatcacttgctcaggggacgcccttcccaaaaaatacacttactggtatcaacagaagagtgggcaggctcctgtgttggtaatatactatgacacgaaacggcctagcgggatcccggagcggttctcaggttcctccagtgggacgatggctacactgacgatcagcggggcccaagtggaggacgaagctgattattattgccttagtacccaccccgccggacaggagtctgtgtttggtggcggcaccaagcttacggtactcggacagcctgcggccgcaaccacaacccctgctccaaggccccccacacccgctccaactatagccagccaaccattgagcctcagacctgaagcttgcaggcccgcagcaggaggcgccgtccatacgcgaggcctggacttcgcgtgtgatatttatatttgggcccctttggccggaacatgtggggtgttgcttctctcccttgtgatcactctgtattgtaagcgcgggagaaagaagctcctgtacatcttcaagcagccttttatgcgacctgtgcaaaccactcaggaagaagatgggtgttcatgccgcttccccgaggaggaagaaggagggtgtgaactgagggtgaaattttctagaagcgccgatgctcccgcatatcagcagggtcagaatcagctctacaatgaattgaatctcggcaggcgagaagagtacgatgttctggacaagagacggggcagggatcccgagatggggggaaagccccggagaaaaaatcctcaggaggggttgtacaatgagctgcagaaggacaagatggctgaagcctatagcgagatcggaatgaaaggcgaaagacgcagaggcaaggggcatgacggtctgtaccagggtctctctacagccaccaaggacacttatgatgcgttgcatatgcaagccttgccaccccgcggatcaggagcgactaacttctctttgttgaagcaagcgggtgacgttgaagagaatcccggtccatccgggggatccatgcggatctccaagccccacctccgaagcatcagtatccagtgttatctgtgccttcttctgaattcccattttttgacagaggcaggaattcatgtattcatattgggttgtttctccgcggggcttcctaaaaccgaagctaactgggttaatgtcatttcagatctgaagaaaattgaagatcttattcaatcaatgcatatcgacgcgaccttgtacacggagagtgatgtccacccgagctgcaaggtaacggccatgaaatgcttcttgctggaattgcaggtaattagtcttgagagtggagatgcgtctatacacgacaccgtagagaatctcattattttggcgaacaattctctcagcagtaacggcaatgttactgaatcaggatgtaaggagtgtgaagagctggaagaaaagaacattaaggaatttttgcagagcttcgtacatatcgtccaaatgttcataaatacaagctaa

SEQ ID NO: 98 - DNA, 1407 bp, synthetic construct
  source 1..1407: mol_type = other DNA
caggtccaattggtccaatctggcgccgaggtcaagaagcctggcgcatccgtaaaagtgtcttgtaaagcctctggttatacgtttacggggtattatatgcaatgggttcggcaagcccctggtcaaggacttgaatggatgggctggatgaaccctatctctggtaatacgggctacgcccaaaagttccagggtagagtcacaatgacgcgcaacacgtcaatatcaaccgcttacatggagcttagttctttgaggtcagaggacaccgcggtctattattgtgctcgcgcccaatgggaaccgcttgattactgggggcaaggtaccttggttaccgtctcatcaggtggaggcggttcaggcggaggtggctctggcggtggcgctagcagctacgaactgacacagccgccctctgttagcgttagtcctggacagaccgccaggataacgtgctccggtgacgcattgccgaagaaatatacgtactggtaccagcagaagtccgggcaagcacctgtactcgtgatctattatgatacaaaacggccgagcggtataccagaacgctttagcggctcaagcagcggaacgatggccacgttgacgattagcggcgcgcaggtcgaggacgaagctgactactattgcctcagtactaacccaacagggcacgaatcagtttttggagggggaaccaagcttactgtacttggtcagccggcggccgcaaccacaacccctgctccaaggccccccacacccgctccaactatagccagccaaccattgagcctcagacctgaagcttgcaggcccgcagcaggaggcgccgtccatacgcgaggcctggacttcgcgtgtgatatttatatttgggcccctttggccggaacatgtggggtgttgcttctctcccttgtgatcactctgtattgtaagcgcgggagaaagaagctcctgtacatcttcaagcagccttttatgcgacctgtgcaaaccactcaggaagaagatgggtgttcatgccgcttccccgaggaggaagaaggagggtgtgaactgagggtgaaattttctagaagcgccgatgctcccgcatatcagcagggtcagaatcagctctacaatgaattgaatctcggcaggcgagaagagtacgatgttctggacaagagacggggcagggatcccgagatggggggaaagccccggagaaaaaatcctcaggaggggttgtacaatgagctgcagaaggacaagatggctgaagcctatagcgagatcggaatgaaaggcgaaagacgcagaggcaaggggcatgacggtctgtaccagggtctctctacagccaccaaggacacttatgatgcgttgcatatgcaagccttgccaccccgc

SEQ ID NO: 99 - DNA, 1407 bp, synthetic construct
  source 1..1407: mol_type = other DNA
caggtccagctggtacagtctggcgctgaggtaaagaagcctggtgcttcagtgaaggtgtcttgcaaagcctctggctatactttcacaggttattatatgcactgggtgcgccaggcccccggtcagggccttgaatggatgggatggatgaatccaaattcaggcaataccgggtatgctcaaaagtttcagggccgcgtaacaatgacccgaaatacaagtatttcaactgcttatatggaactcagttctctcagaagtgaagatacggccgtatattattgcgcgcgggcgcaatgggaaccacttgactattggggacaaggcacactggttacggtgtcatctggtggaggcggttcaggcggaggtggctctggcggtggcgctagctcatatgaactcacacagccgccatctgttagcgtttctccagggcagactgcccgaattacatgctcaggcgatgccctcccgaaaaaatatacctactggtaccaacaaaaatccggccaggcacctgtgttggtgatatattatgatacaaaacgcccttcagggatcccggagagattcagtgggtcaagtagcggaacaatggcaacacttactataagcggggcacaggttgaggacgaggctgactactactgcctgtctactcatccggccggacaggagagtgttttcgggggtggaactaaactcacagtactgggacagcccgcggccgcaaccacaacccctgctccaaggccccccacacccgctccaactatagccagccaaccattgagcctcagacctgaagcttgcaggcccgcagcaggaggcgccgtccatacgcgaggcctggacttcgcgtgtgatatttatatttgggcccctttggccggaacatgtggggtgttgcttctctcccttgtgatcactctgtattgtaagcgcgggagaaagaagctcctgtacatcttcaagcagccttttatgcgacctgtgcaaaccactcaggaagaagatgggtgttcatgccgcttccccgaggaggaagaaggagggtgtgaactgagggtgaaattttctagaagcgccgatgctcccgcatatcagcagggtcagaatcagctctacaatgaattgaatctcggcaggcgagaagagtacgatgttctggacaagagacggggcagggatcccgagatggggggaaagccccggagaaaaaatcctcaggaggggttgtacaatgagctgcagaaggacaagatggctgaagcctatagcgagatcggaatgaaaggcgaaagacgcagaggcaaggggcatgacggtctgtaccagggtctctctacagccaccaaggacacttatgatgcgttgcatatgcaagccttgccaccccgc

SEQ ID NO: 100 - DNA, 1407 bp, synthetic construct
  source 1..1407: mol_type = other DNA
caggtccaactggtgcaatctggcgctgaagtgaaaaagcccggcgcatcagtaaaggtgagttgcaaagcatctgggtacactttcactggctattatatgcattgggtaaggcaagcccccggccaaggcctggaatggatgggatggatgaatccgaactccggtaatacagggtacgctcaaaaatttcaaggacgggtcacaatgacgcggaacaccagtatatctactgcatatatggagctcagttcactgcgctccgaggatacagctgtatactattgcgccagggctcaatgggaaccactggactattggggtcaagggacacttgtaacggtaagctcaggtggaggcggttcaggcggaggtggctctggcggtggcgctagctcctatgagcttactcagccaccctctgtatctgtatctcctgggcagacagcacgcatcacatgtagtggcgatgccctcccgaaaaaatacacgtattggtatcaacagaaatccggacaggcaccggttttggtgatatactatgatacgaaaagacctagtggaatacctgagcggttttctggaagttcttctggtaccatggcaactttgacaatctcaggtgcccaggtcgaggacgaggccgactattattgtttgtctacaaatccgacaggtcatgaatccgtgttcggcggagggacaaagcttaccgtccttgggcagcctgcggccgcaaccacaacccctgctccaaggccccccacacccgctccaactatagccagccaaccattgagcctcagacctgaagcttgcaggcccgcagcaggaggcgccgtccatacgcgaggcctggacttcgcgtgtgatatttatatttgggcccctttggccggaacatgtggggtgttgcttctctcccttgtgatcactctgtattgtaagcgcgggagaaagaagctcctgtacatcttcaagcagccttttatgcgacctgtgcaaaccactcaggaagaagatgggtgttcatgccgcttccccgaggaggaagaaggagggtgtgaactgagggtgaaattttctagaagcgccgatgctcccgcatatcagcagggtcagaatcagctctacaatgaattgaatctcggcaggcgagaagagtacgatgttctggacaagagacggggcagggatcccgagatggggggaaagccccggagaaaaaatcctcaggaggggttgtacaatgagctgcagaaggacaagatggctgaagcctatagcgagatcggaatgaaaggcgaaagacgcagaggcaaggggcatgacggtctgtaccagggtctctctacagccaccaaggacacttatgatgcgttgcatatgcaagccttgccaccccgc

SEQ ID NO: 101 - DNA, 1407 bp, synthetic construct
  source 1..1407: mol_type = other DNA
caggttcaacttgttcaaagcggtgcagaagttaagaaaccaggtgccagcgttaaggtcagctgtaaagcgagtgggtacactttcaccgaatattatatgaaatgggtacgacaagcaccagggcagggactcgaatggatggggtggatgaaccccaacactggcaagacgggctacgcacagaaattccaagggcgagtgaccatgaccagaaatacgtcaatttcaaccgcatacatggagctgtcatccttgcgatccgaggacaccgcggtatattattgcgcgcgcgctcaatgggagccacttgattattggggacagggcacgcttgtcactgtatcctctggtggaggcggttcaggcggaggtggctctggcggtggcgctagctcatatgaacttactcagccgccttctgttagtgtaagccctgggcagactgcacgcattacatgcagtggtgacgccttgccaaaaaaatatacttattggtaccaacaaaagagtggacaggcgccggtcttggtgatatactatgacactaagagaccctctgggataccggaacgcttttctggttcaagttccggaacgatggcgaccttgacgatcagcggggcccaagtcgaggatgaggccgactactactgtttgtctacagatcccacaggccaagaaagcgtgttcggaggtgggactaaacttacggtcttgggtcaaccagcggccgcaaccacaacccctgctccaaggccccccacacccgctccaactatagccagccaaccattgagcctcagacctgaagcttgcaggcccgcagcaggaggcgccgtccatacgcgaggcctggacttcgcgtgtgatatttatatttgggcccctttggccggaacatgtggggtgttgcttctctcccttgtgatcactctgtattgtaagcgcgggagaaagaagctcctgtacatcttcaagcagccttttatgcgacctgtgcaaaccactcaggaagaagatgggtgttcatgccgcttccccgaggaggaagaaggagggtgtgaactgagggtgaaattttctagaagcgccgatgctcccgcatatcagcagggtcagaatcagctctacaatgaattgaatctcggcaggcgagaagagtacgatgttctggacaagagacggggcagggatcccgagatggggggaaagccccggagaaaaaatcctcaggaggggttgtacaatgagctgcagaaggacaagatggctgaagcctatagcgagatcggaatgaaaggcgaaagacgcagaggcaaggggcatgacggtctgtaccagggtctctctacagccaccaaggacacttatgatgcgttgcatatgcaagccttgccaccccgc

SEQ ID NO: 102 - DNA, 1428 bp, synthetic construct
  source 1..1428: mol_type = other DNA
caagttcaactggttcagtctggcgccgaagtgaagaaacctggcgcctctgtgaaggtgtcctgcaaggccagcggctacacctttaccagctacggcatcagctgggtccgacaggctcctggacaaggcttggaatggatgggctggatcagcgcctacaacggcaacacctactacgccgaggatctgcagggcagactgaccatgaccaccgataccagcaccagcaccgcctacatggaactgagaagcctgagatccgacgacaccgccgtgtactactgtgccagagagggcagctactacgacagctccggctactacctgccttattggggccagggcacactggtcacagtctcgagtggtggaggcggttcaggcggaggtggctctggcggtggcgctagccaatctgctctgacacagcccagaagcgtgtccggatctcctggccagagcgtgaccatcagctgtacaggcaccagctctgatgtgggcgcctacaactacgtgtcctggtatcagcagcaccccggcaaggcccctaagctgatgatctacgatgtgaccgagaggcctagcggcgtgcccgatagattttctggcagcaagagcggcaacaccgccagcctgacaattagcggactgcaggccgaggacgaggccgattactactgtagcagctacaccagcagctctatcgcctttggcggaggcaccaagctgacagttctgggacaacctgcggccgcaaccacaacccctgctccaaggccccccacacccgctccaactatagccagccaaccattgagcctcagacctgaagcttgcaggcccgcagcaggaggcgccgtccatacgcgaggcctggacttcgcgtgtgatatttatatttgggcccctttggccggaacatgtggggtgttgcttctctcccttgtgatcactctgtattgtaagcgcgggagaaagaagctcctgtacatcttcaagcagccttttatgcgacctgtgcaaaccactcaggaagaagatgggtgttcatgccgcttccccgaggaggaagaaggagggtgtgaactgagggtgaaattttctagaagcgccgatgctcccgcatatcagcagggtcagaatcagctctacaatgaattgaatctcggcaggcgagaagagtacgatgttctggacaagagacggggcagggatcccgagatggggggaaagccccggagaaaaaatcctcaggaggggttgtacaatgagctgcagaaggacaagatggctgaagcctatagcgagatcggaatgaaaggcgaaagacgcagaggcaaggggcatgacggtctgtaccagggtctctctacagccaccaaggacacttatgatgcgttgcatatgcaagccttgccaccccgc

SEQ ID NO: 103 - protein, 184 aa, Homo sapiens
MLQMAGQCSQNEYFDSLLHACIPCQLRCSSNTPPLTCQRYCNASVTNSVKGTNAILWTCLGLSLIISLAVFVLMFLLRKINSEPLKDEFKNTGSGLLGMANIDLEKSRTGDEIILPRGLEYTVEECTCEDCIKSKPKVDSDHCFPLPAMEEGATILVTTKTNDYCKSLPAALSATEIEKSISAR

SEQ ID NO: 104 - protein, 184 aa, Homo sapiens [sequence omitted]

SEQ ID NO: 105 - protein, 6 aa, synthetic construct
NPNSGN

SEQ ID NO: 106 - protein, 5 aa, synthetic construct
QWEPL

SEQ ID NO: 107 - protein, 9 aa, synthetic construct
DALPKKYTY

SEQ ID NO: 108 - protein, 8 aa, synthetic construct
LSTDPTGQ

SEQ ID NO: 109 - protein, 108 aa, synthetic construct
SYELTQPPSVSVSPGQTARITCSGDALPKKYTYWYQQKSGQAPVLVIYYDTKRPSGIPERFSGSSSGTMATLTISGAQVEDEADYYCLSTDPTGQESVFGGGTKLTVL

SEQ ID NO: 110 - protein, 108 aa, synthetic construct
SYELTQPPSVSVSPGQTARITCSGDALPKKYTYWYQQKSGQAPVLVIYYDTKRPSGIPERFSGSSSGTMATLTISGAQVEDEADYYCLSTNPTGHESVFGGGTKLTVL

SEQ ID NO: 111 - protein, 108 aa, synthetic construct
SYELTQPPSVSVSPGQTARITCSGDALPKKYTYWYQQKSGQAPVLVIYYDTKRPSGIPERFSGSSSGTMATLTISGAQVEDEADYYCLSTHPAGQESVFGGGTKLTVL